(19) United States
(12) Patent Application Publication
(10) Pub. No.: US 2024/0124501 A1
(43) Pub. Date: Apr. 18, 2024

(54) VANADIUM COMPOUNDS AND METHODS OF MAKING AND USING THEREOF

(71) Applicants: COLORADO STATE UNIVERSITY RESEARCH FOUNDATION, Fort Collins, CO (US); THE UNIVERSITY OF SYDNEY, Sydney (AU)

(72) Inventors: Debbie C. CRANS, Fort Collins, CO (US); Peter A. LAY, Darlington, NSW (AU); Heide MURAKAMI, Fort Collins, CO (US); Aviva LEVINA, Darlington, NSW (AU); Kateryna KOSTENKOVA, Fort Collins, CO (US); Andrew BATES, Fort Collins, CO (US); John MANGANARO, Fort Collins, CO (US); Josef GRUNDY, Fort Collins, CO (US); Skyler MARKHAM, Fort Collins, CO (US); Kameron KLUGH, Fort Collins, CO (US)

(21) Appl. No.: 18/453,027

(22) Filed: Aug. 21, 2023

Related U.S. Application Data
(60) Provisional application No. 63/432,311, filed on Dec. 13, 2022.
(60) Provisional application No. 63/399,716, filed on Aug. 21, 2022.

(51) Int. Cl. C07F 9/00

(52) U.S. Cl. CPC C07F 9/005

(57) ABSTRACT

Disclosed are vanadium (V) compounds and pharmaceutical compositions comprising these vanadium compounds. Also disclosed are methods of making and using these vanadium compounds, for example, to treat cancer (e.g., including brain cancer).

### CROSS-REFERENCE TO RELATED APPLICATIONS

This application claims benefit of U.S. Provisional Application No. 63/399,716, filed Aug. 21, 2022, and U.S. Provisional Application No. 63/432,311, filed Dec. 13, 2022, each of which is hereby incorporated herein by reference in its entirety.

### BACKGROUND

Cancer is a major disease that continues as one of the leading causes of death at any age. In the United States alone, it is estimated that more than 600,000 Americans died of cancer in 2022. Currently, radiotherapy and chemotherapy are two important methods used in the treatment of cancer.

Considerable efforts are underway to develop new chemotherapeutic agents for more potent and specific anti-cancer therapy, presenting effective and efficient cytotoxicity against tumor cells, with minimal interference with normal cell function. There is an urgent need for the development and analysis of novel, effective anti-cancer agents.

### SUMMARY

Provided herein are vanadium compounds, pharmaceutical compositions comprising these vanadium compounds, as well as methods of making and using these vanadium compounds (e.g., to treat cancer).

For example, provided herein are vanadium complexes defined by Formula A

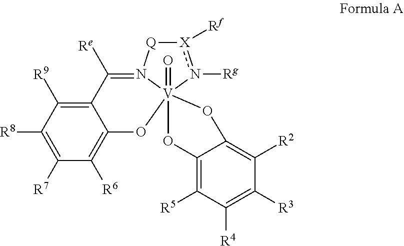

wherein
Q is selected from

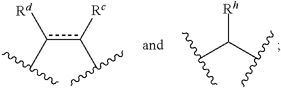

X is absent, or is selected from N, C, or CH when Q is

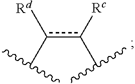

or X is selected from N, C, or CH when Q is

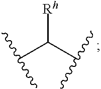

R^{h} is selected from H, ═O, halo, C₁₋₆ alkyl, or C₁₋₄ haloalkyl, or R^{h} and R^{f}, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R^{c} and R^{d} are independently selected from H, ═O, halo, C₁₋₆ alkyl, or C₁₋₄haloalkyl; or R^{c} and R^{d} and/or R^{e} and R^{g}, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R^{e} is selected from H, C₁-6 alkyl, C₁₋₄haloalkyl;
[symbol] represents individually for each occurrence, as valence permits, a single bond or a double bond; [symbol]
R^{f} and R^{g}, together with the atoms to which they are attached, form a 3-10 membered heterocycloalkyl group or a 5-10 membered heteroaryl group, each optionally substituted with 1, 2, or 3 independently selected R^{A} groups;
R², R³, R⁴, and R⁵ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R² and R³, R³ and R⁴, and/or R⁴ and R⁵, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R⁶, R⁷, R⁸, and R⁹ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R⁶ and R⁷, R⁷ and R⁸, and/or R⁸ and R⁹, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups; and
each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆alkyl)aminosulfonyl, aminosulfonylamino, C₁₋₆ alkylaminosulfonylamino, di(C₁₋₆alkyl)aminosulfonylamino, aminocarbonylamino, C₁₋₆ alkylaminocarbonylamino, and di(C₁₋₆alkyl)aminocarbonylamino.

In some aspects of Formula A, Q is

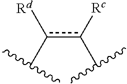

and X is absent, or selected from N, C, or CH.

In some aspects of Formula A, [symbol] represents a single bond bond. In other aspects of Formula A, [symbol] represents a double bond bond.

In some aspects of Formula A, R^{c} and R^{d}, together with the atoms to which they are attached, form a 3-10 membered cycloalkyl group, or a 6-10 membered aryl group, each optionally substituted with 1, 2, or 3 independently selected R^{A} groups.

In some aspects of Formula A, X is absent.

In some aspects of Formula A, R^{c} and R^{g}, together with the atoms to which they are attached, form a 3-10 membered heterocycloalkyl group or a 5-10 membered heteroaryl group, each optionally substituted with 1, 2, or 3 independently selected R^{A} groups.

In some aspects of Formula A, Q is

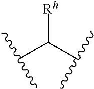

and X is selected from N, C, or CH.

In some aspects of Formula A, R^{h} is hydrogen.

In some aspects of Formula A, R^{e} is selected from H and C₁₋₆ alkyl. In certain aspects of Formula A, R^{e} is H.

In some aspects of Formula A, R^{f} and R^{g}, together with the atoms to which they are attached, form an imidazole ring, a pyridine ring, a pyrimidine ring, a pyrazine ring, a pyrrole ring, a thiazole ring, an indole ring, a purine ring, or a pteridine ring, each optionally substituted with 1, 2, or 3 independently selected R^{A} groups.

In certain aspects of Formula A, R^{f} and R^{g}, together with the atoms to which they are attached, form an imidazole ring optionally substituted with 1, 2, or 3 independently selected R^{A} groups. In certain aspects of Formula A, R^{f} and R^{g}, together with the atoms to which they are attached, form a pyridine ring optionally substituted with 1, 2, or 3 independently selected R^{A} groups.

In some aspects of Formula A, one or more of R⁶, R⁷, R⁸, and R⁹ is not hydrogen.

In some aspects of Formula A, one or more of R⁶, R⁷, R⁸, and R⁹ is halo, such as chloro.

In some aspects of Formula A, one or more of R⁶, R⁷, R⁸, and R⁹ is C₁₋₆ alkyl, such as t-butyl.

In some aspects of Formula A, one or more of R⁶, R⁷, R⁸, and R⁹ is di(C₁₋₆ alkyl)amino, such as diethylamino.

In some aspects of Formula A, one or more of R⁶, R⁷, R⁸, and R⁹ is C₁₋₆ alkoxy, such as methoxy.

In some aspects of Formula A, R⁸ is halo, such as chloro.

In some aspects of Formula A, R⁶ and R⁸ are each C₁₋₆ alkyl, such as t-butyl.

In some aspects of Formula A, R⁷ is di(C₁₋₆ alkyl)amino, such as diethylamino.

In some aspects of Formula A, R⁶ is C₁₋₆ alkoxy, such as methoxy.

In some aspects of Formula A, one or more of R², R³, R⁴, and R⁵ is not hydrogen.

In some aspects of Formula A, one or more of R², R³, R⁴, and R⁵ is C₁₋₆ alkyl, such as t-butyl.

In some aspects of Formula A, one or more of R², R³, R⁴, and R⁵ is a 3-10 membered cycloalkyl group, such as adamantly or norbornyl.

In some aspects of Formula A, R³ and R⁵ are C₁₋₆ alkyl, such as t-butyl.

In some aspects of Formula A, R³ and R⁵ are a 3-10 membered cycloalkyl group, such as adamantly or norbornyl.

In some aspects of Formula A, the complex is not one of the complexes below

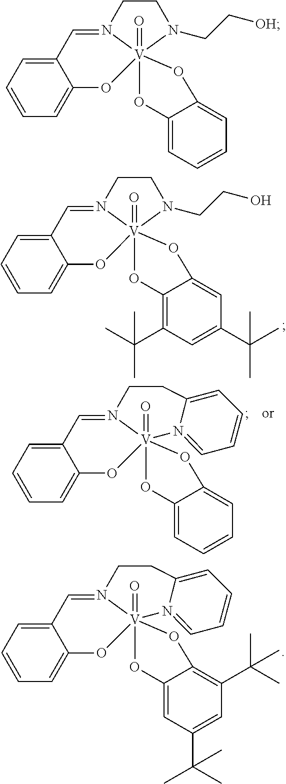

Also provided are vanadium complexes defined by Formula B, Formula C, or Formula D

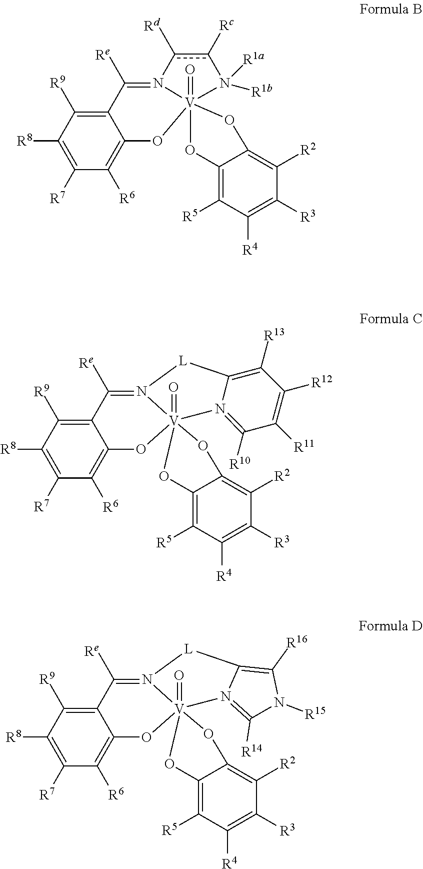

wherein
L is selected from

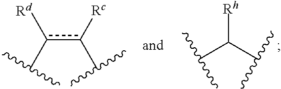

R^{h} is selected from H, ═O, halo, C₁₋₆ alkyl, or C₁₋₄ haloalkyl;
R^{c} and R^{d} are independently selected from H, ═O, halo, C₁₋₆ alkyl, or C₁₋₄haloalkyl; or R^{c} and R^{d} and/or R^{e} and R^{g}, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R^{e} is selected from H, C₁₋₆ alkyl, C₁₋₄haloalkyl;
[symbol] represents individually for each occurence, as valence permits, a single bond or a double bond;
R^{1a} is selected from C₁₋₆ alkyl, C₁₋₄haloalkyl, C₂₋₆ alkenyl, C₂₋₆ alkynyl, C₃₋₁₀ cycloalkyl, C₃₋₁₀ cycloalkyl-C₁₋₄ alkylene, 6-10 membered aryl, and 6-10 membered aryl-C₁₋₄ alkylene, each of which is optionally substituted by 1, 2, 3, or 4 independently selected R^{A} groups;
R^{1b} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄ haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups;
R², R³, R⁴, and R⁵ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R² and R³, R³ and R⁴, and/or R⁴ and R⁵, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R⁶, R⁷, R⁸, and R⁹ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R⁶ and R⁷, R⁷ and R⁸, and/or R⁸ and R⁹, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁰, R¹¹, R¹², and R¹³ are independently selected from H, OH, NO₂, CN, halo, C₁₋₆ alkyl, C₂₋₆ alkenyl, C₂₋₆ alkynyl, C₁₋₄haloalkyl, C₃₋₁₀cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R¹⁰ and R¹¹, R¹¹ and R¹², and/or R¹² and R¹³, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁴ and R¹⁶ are independently selected from H, OH, NO₂, CN, halo, C₁₋₆ alkyl, C₂₋₆ alkenyl, C₂₋₆ alkynyl, C₁₋₄haloalkyl, C₃₋₁₀cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups;
R¹⁵ is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄ haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups; and
   each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁-6 alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino
   In some aspects of Formula B, Formula C, and Formula D, L is

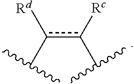

In some aspects of Formula B, Formula C, and Formula D, [symbol] represents a single bond bond. In other aspects of Formula B, Formula C, and Formula D, [symbol] represents a double bond bond.

In some aspects of Formula B, Formula C, and Formula D, R^{c} and R^{d}, together with the atoms to which they are attached, form a 3-10 membered cycloalkyl group, or a 6-10 membered aryl group, each optionally substituted with 1, 2, or 3 independently selected R^{A} groups.

In some aspects of Formula B, Formula C, and Formula D, L is

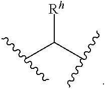

In some aspects of Formula B, Formula C, and Formula D, R^{h} is hydrogen.

In some aspects of Formula B, Formula C, and Formula D, R^{e} is selected from H and C₁₋₆ alkyl. In certain aspects of Formula B, Formula C, and Formula D, R^{e} is H.

In some aspects of Formula B, Formula C, and Formula D, one or more of R⁶, R⁷, R⁸, and R⁹ is not hydrogen.

In some aspects of Formula B, Formula C, and Formula D, one or more of R⁶, R⁷, R⁸, and R⁹ is halo, such as chloro.

In some aspects of Formula B, Formula C, and Formula D, one or more of R⁶, R⁷, R⁸, and R⁹ is C₁₋₆ alkyl, such as t-butyl.

In some aspects of Formula B, Formula C, and Formula D, one or more of R⁶, R⁷, R⁸, and R⁹ is di(C₁₋₆ alkyl)amino, such as diethylamino.

In some aspects of Formula B, Formula C, and Formula D, one or more of R⁶, R⁷, R⁸, and R⁹ is C₁₋₆ alkoxy, such as methoxy.

In some aspects of Formula B, Formula C, and Formula D, R⁸ is halo, such as chloro.

In some aspects of Formula B, Formula C, and Formula D, R⁶ and R⁸ are each C₁₋₆ alkyl, such as t-butyl.

In some aspects of Formula B, Formula C, and Formula D, R⁷ is di(C₁₋₆ alkyl)amino, such as diethylamino.

In some aspects of Formula B, Formula C, and Formula D, R⁶ is C₁₋₆ alkoxy, such as methoxy.

In some aspects of Formula B, Formula C, and Formula D, one or more of R², R³, R⁴, and R⁵ is not hydrogen.

In some aspects of Formula B, Formula C, and Formula D, one or more of R², R³, R⁴, and R⁵ is C₁₋₆ alkyl, such as t-butyl.

In some aspects of Formula B, Formula C, and Formula D, one or more of R², R³, R⁴, and R⁵ is a 3-10 membered cycloalkyl group, such as adamantly or norbornyl.

In some aspects of Formula B, Formula C, and Formula D, R³ and R⁵ are C₁₋₆ alkyl, such as t-butyl.

In some aspects of Formula B, Formula C, and Formula D, R³ and R⁵ are a 3-10 membered cycloalkyl group, such as adamantly or norbornyl.

In some aspects of Formula B, R^{1b} is hydrogen.

In some aspects of Formula B, R^{1a} is a C₁₋₆ alkyl group substituted by a hydroxy group.

In some aspects of Formula C, one or more of R¹⁰, R¹¹, R¹², and R¹³ are all hydrogen.

In some aspects of Formula D, one or more of R¹⁴, R¹⁵, and R¹⁶ are all hydrogen.

In some aspects of Formula B, Formula C, and Formula D, the complex is not one of the complexes below

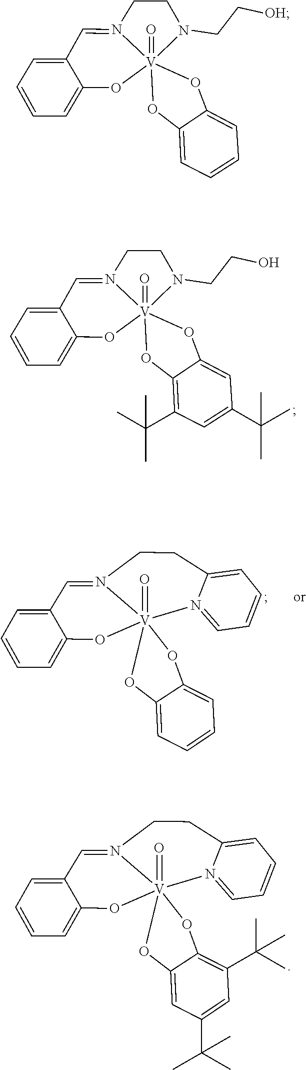

In some aspects, the vanadium complex is defined by Formula I, Formula II, or Formula III below

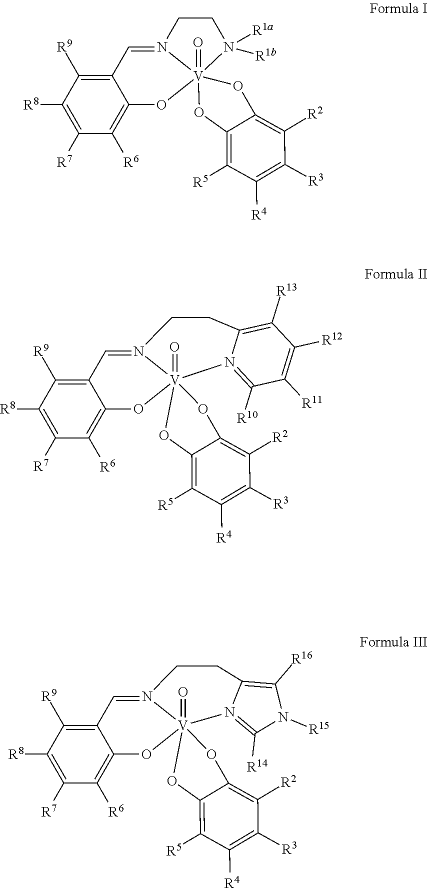

wherein
R^{1a} is selected from C₁₋₆ alkyl, C₁₋₄haloalkyl, C₂₋₆ alkenyl, C₂₋₆ alkynyl, C₃₋₁₀ cycloalkyl, C₃₋₁₀ cycloalkyl-C₁₋₄ alkylene, 6-10 membered aryl, and 6-10 membered aryl-C₁₋₄ alkylene, each of which is optionally substituted by 1, 2, 3, or 4 independently selected R^{A} groups;
R^{1b} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄ haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups;
R², R³, R⁴, and R⁵ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R² and R³, R³ and R⁴, and/or R⁴ and R⁵, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R⁶, R⁷, R⁸, and R⁹ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R⁶ and R⁷, R⁷ and R⁸, and/or R⁸ and R⁹, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁰, R¹¹, R¹², and R¹³ are independently selected from H, OH, NO₂, CN, halo, C₁₋₆ alkyl, C₂₋₆ alkenyl, C₂₋₆ alkynyl, C₁₋₄haloalkyl, C₃₋₁₀cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R¹⁰ and R¹¹, R¹¹ and R¹², and/or R¹² and R¹³, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁴ and R¹⁶ are independently selected from H, OH, NO₂, CN, halo, C₁₋₆ alkyl, C₂₋₆ alkenyl, C₂₋₆ alkynyl, C₁₋₄haloalkyl, C₃₋₁₀cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups;
R¹⁵ is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄ haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups; and
each R^{A} is independently selected from OH, NO₂, CN, halo, C₁₋₆ alkyl, C₂₋₆ alkenyl, C₂-6 alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some aspects of Formula I, Formula II, and Formula III, one or more of R⁶, R⁷, R⁸, and R⁹ is not hydrogen.

In some aspects of Formula I, Formula II, and Formula III, one or more of R⁶, R⁷, R⁸, and R⁹ is halo, such as chloro. In certain aspects of Formula I, Formula II, and Formula III, R⁸ is halo, such as chloro.

In some aspects of Formula I, Formula II, and Formula III, one or more of R⁶, R⁷, R⁸, and R⁹ is C₁₋₆ alkyl, such as t-butyl.

In some aspects of Formula I, Formula II, and Formula III, one or more of R⁶, R⁷, R⁸, and R⁹ is di(C₁₋₆ alkyl)amino, such as diethylamino. In certain aspects of Formula I, Formula II, and Formula III, R⁷ is di(C₁₋₆ alkyl)amino, such as diethylamino.

In some aspects of Formula I, Formula II, and Formula III, one or more of R⁶, R⁷, R⁸, and R⁹ is C₁₋₆ alkoxy, such as methoxy. In certain aspects of Formula I, Formula II, and Formula III, R⁶ and R⁸ are each C₁₋₆ alkyl, such as t-butyl.

In some aspects of Formula I, Formula II, and Formula III, R⁶ is C₁₋₆ alkoxy, such as methoxy.

In some aspects of Formula I, Formula II, and Formula III, one or more of R², R³, R⁴, and R⁵ is not hydrogen.

In some aspects of Formula I, Formula II, and Formula III, one or more of R², R³, R⁴, and R⁵ is C₁₋₆ alkyl, such as t-butyl.

In some aspects of Formula I, Formula II, and Formula III, one or more of R², R³, R⁴, and R⁵ is a 3-10 membered cycloalkyl group, such as adamantly or norbornyl.

In some aspects of Formula I, Formula II, and Formula III, R³ and R⁵ are C₁₋₆ alkyl, such as t-butyl.

In some aspects of Formula I, Formula II, and Formula III, R³ and R⁵ are a 3-10 membered cycloalkyl group, such as adamantly or norbornyl.

In some aspects of Formula I, R^{1b} is hydrogen.

In some aspects of Formula I, R^{1a} is a C₁₋₆ alkyl group substituted by a hydroxy group.

In some aspects of Formula II, one or more of R¹⁰, R¹¹, R¹², and R¹³ are all hydrogen.

In some aspects of Formula III, one or more of R¹⁴, R¹⁵, and R¹⁶ are all hydrogen.

In some aspects of Formula I, Formula II, and Formula III, the complex is not one of the complexes below

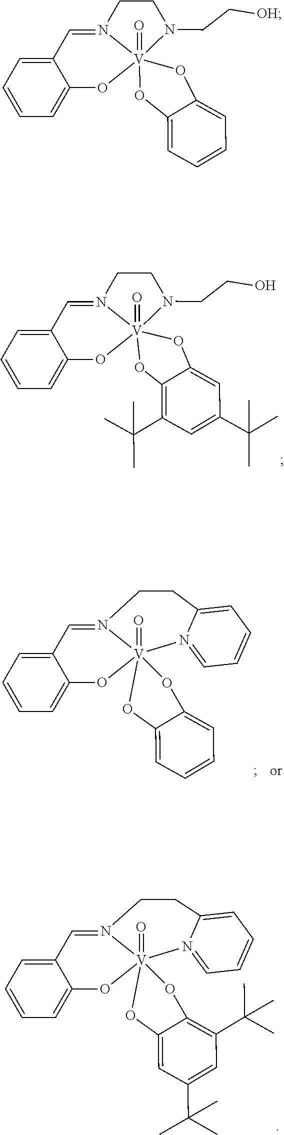

In some aspects of Formula I, Formula II, and Formula III, the complex is one of the complexes shown below

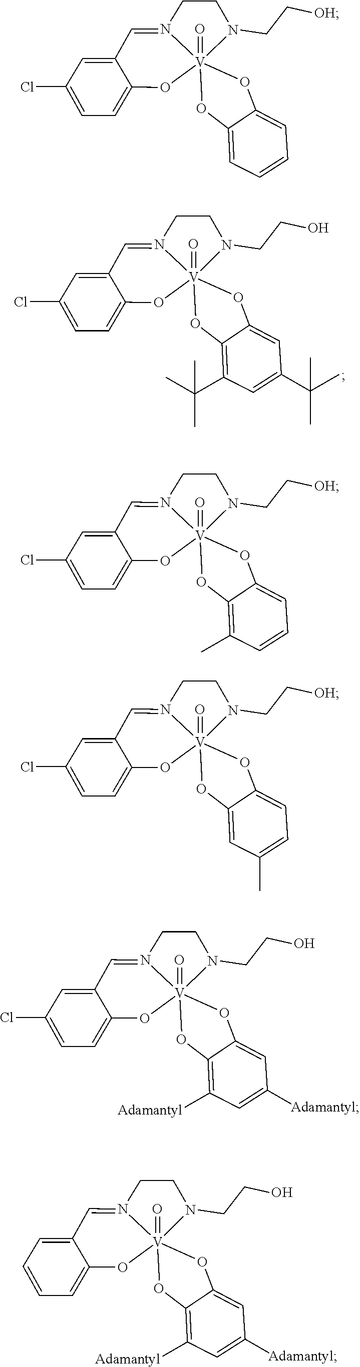

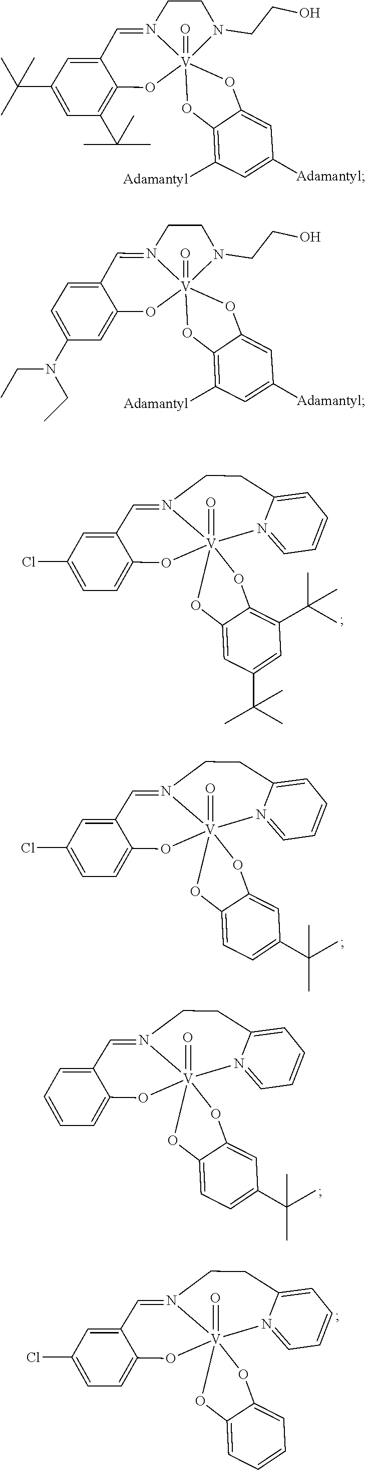

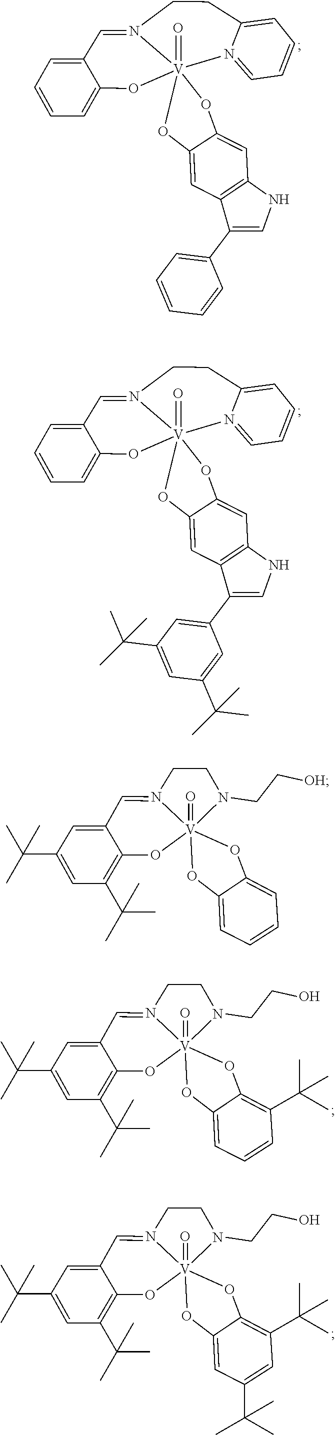

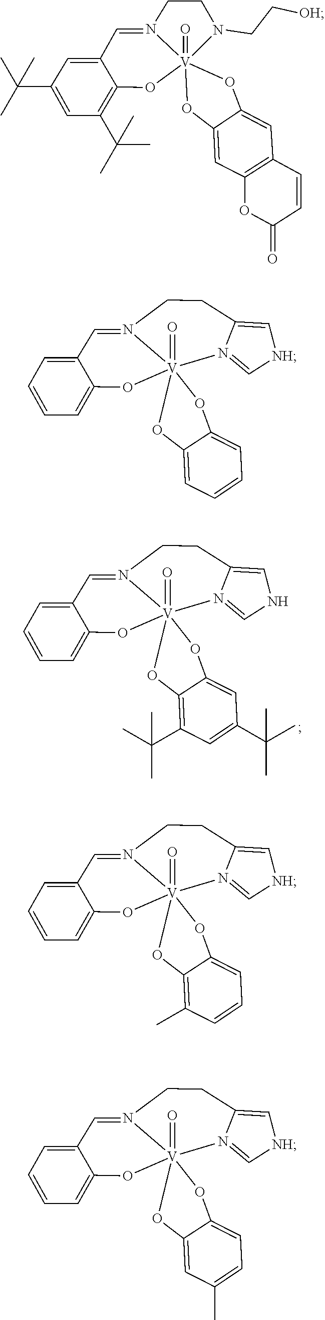

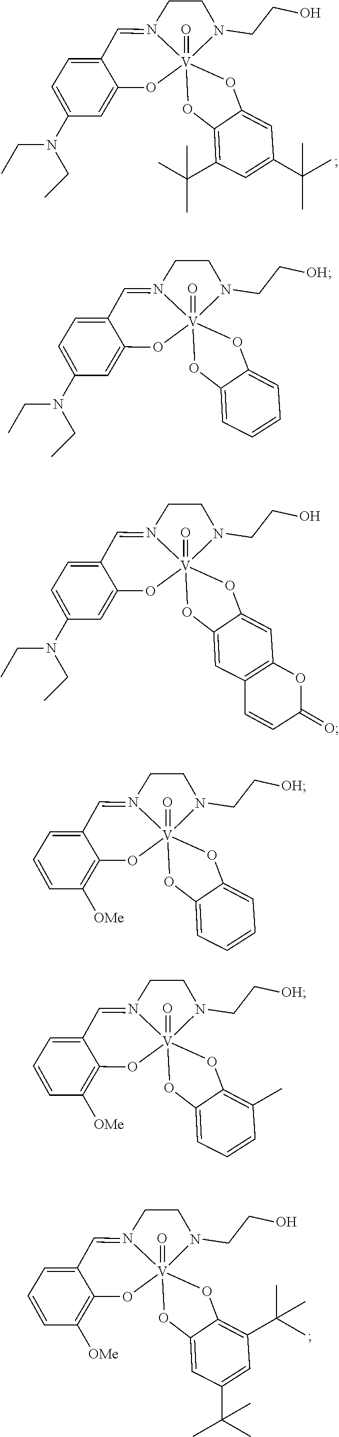

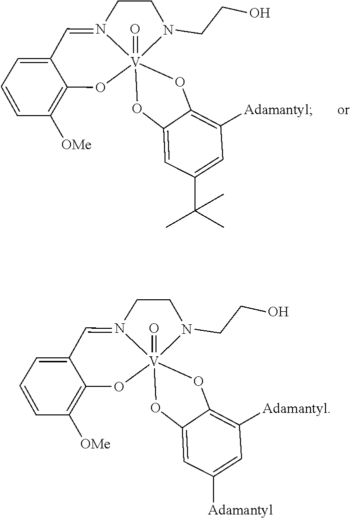

Also provided are the vanadium complexes shown below

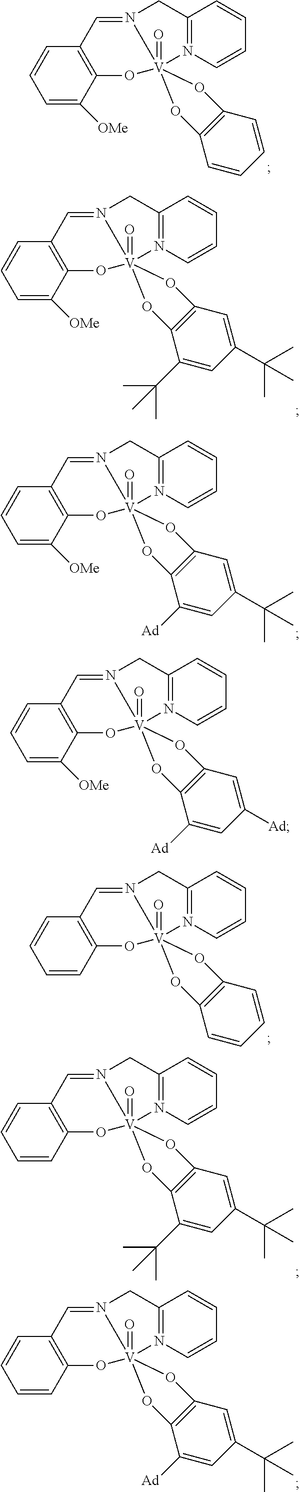

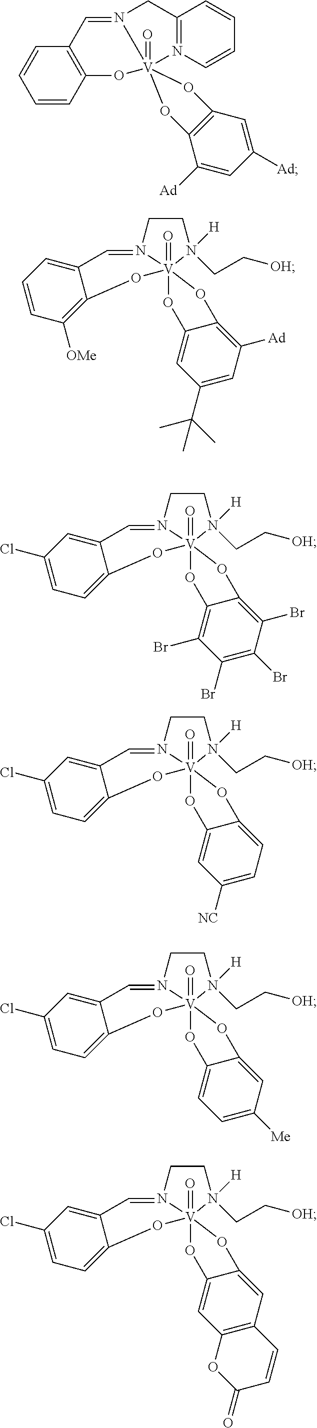

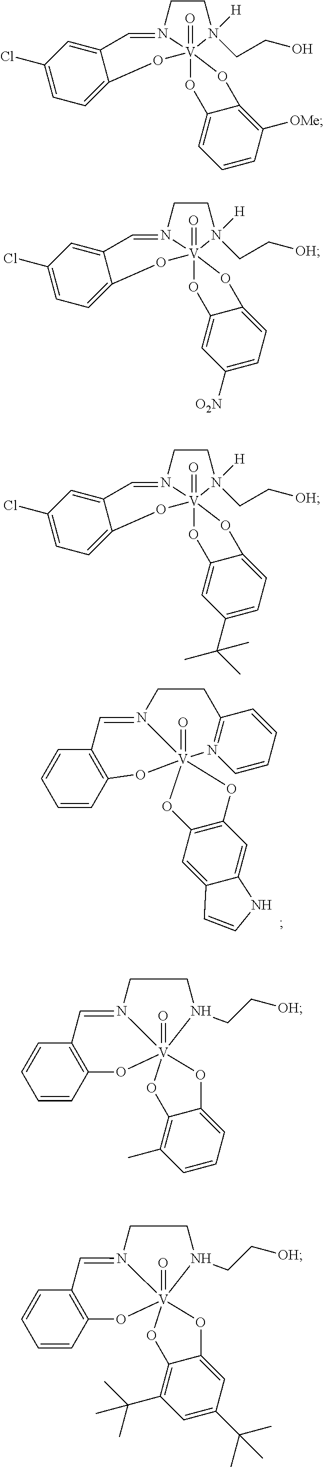

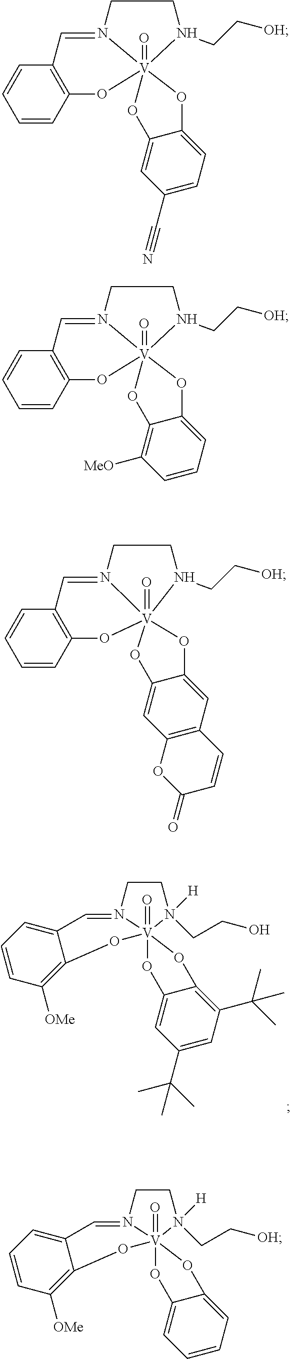

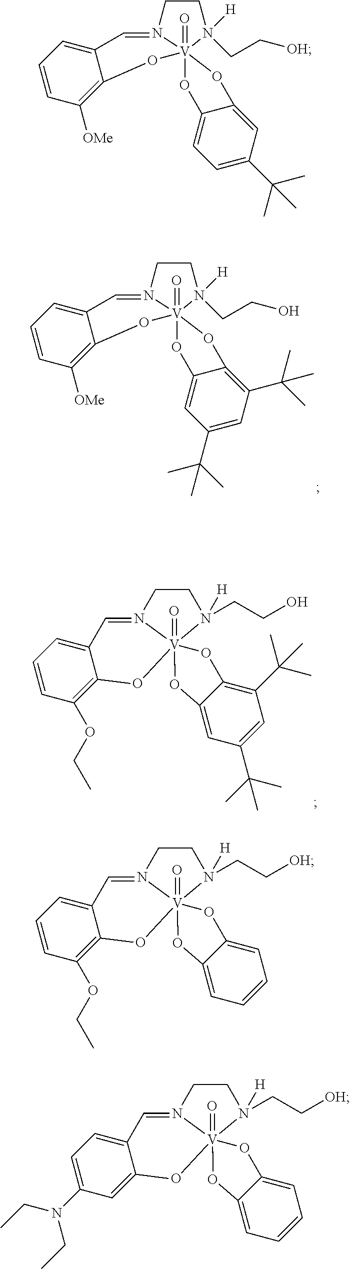

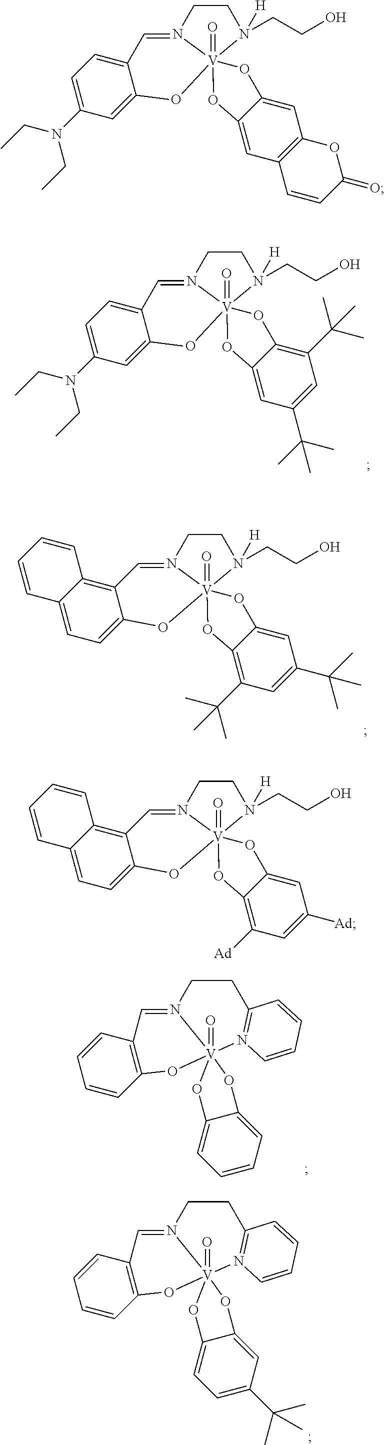

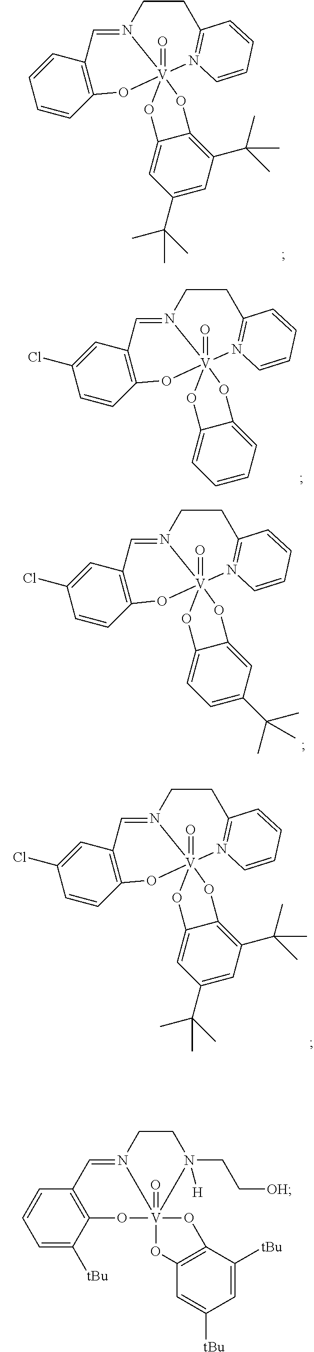

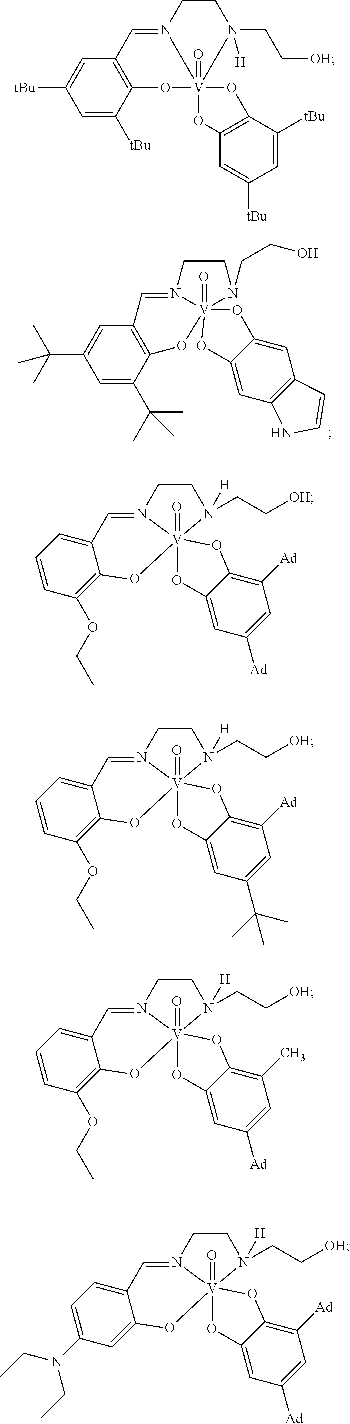

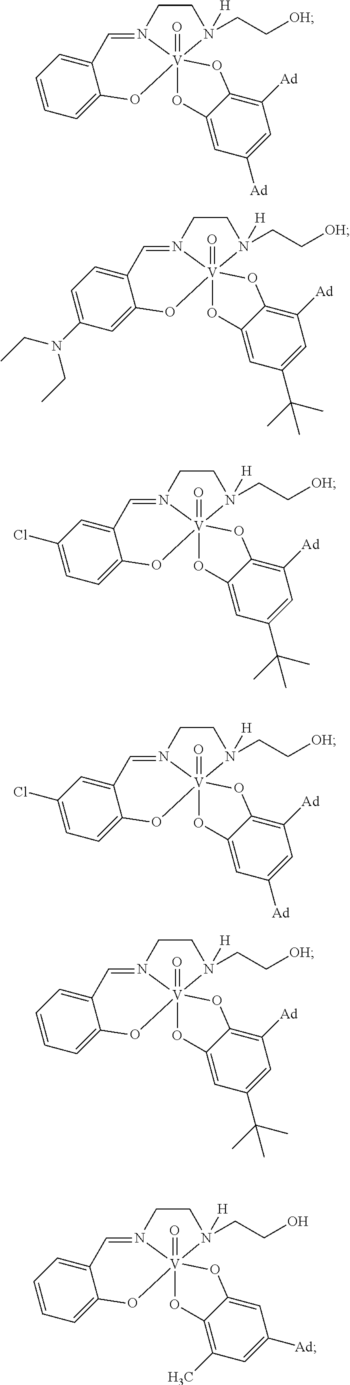

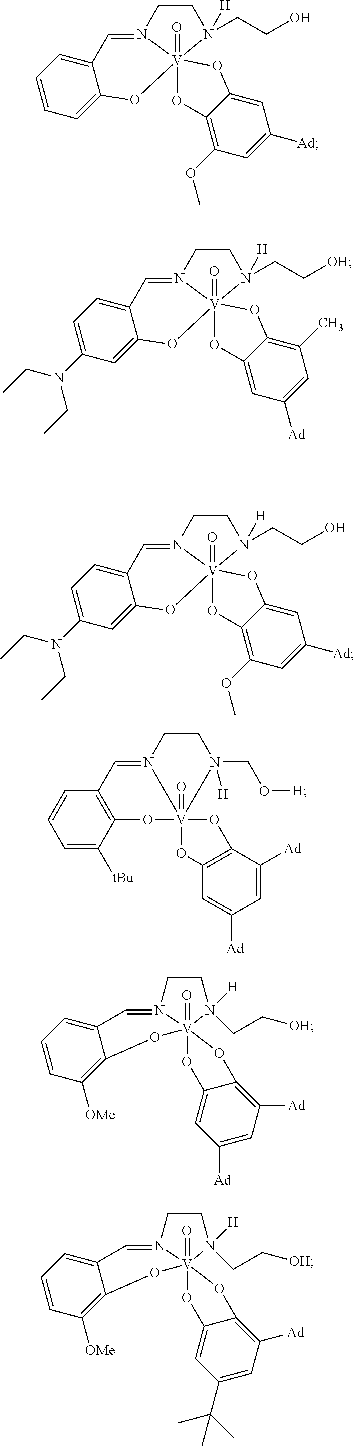

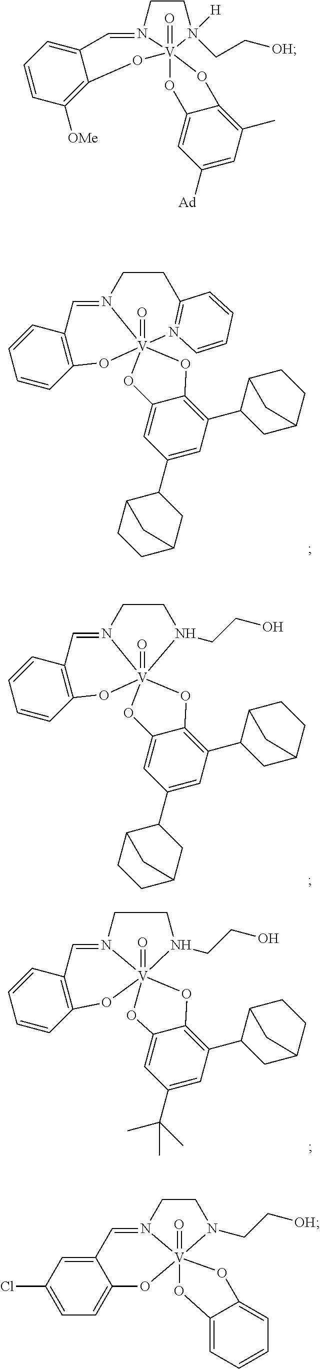

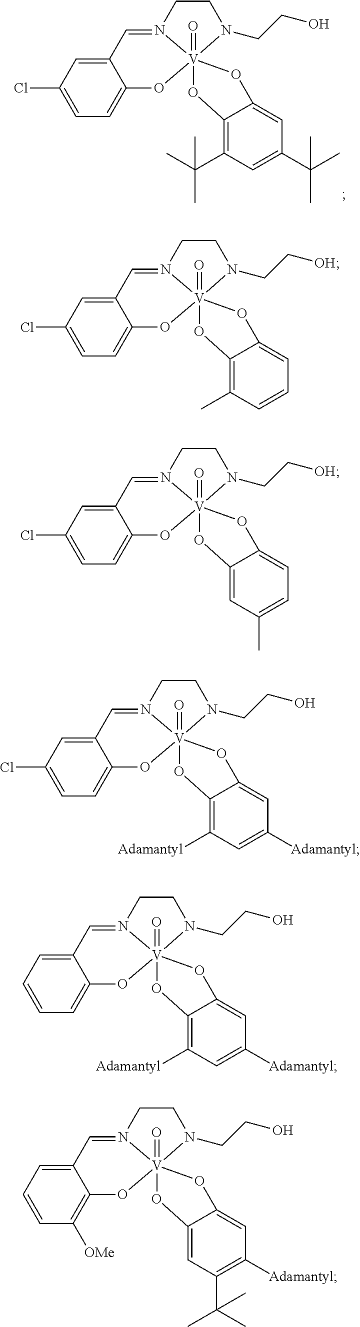

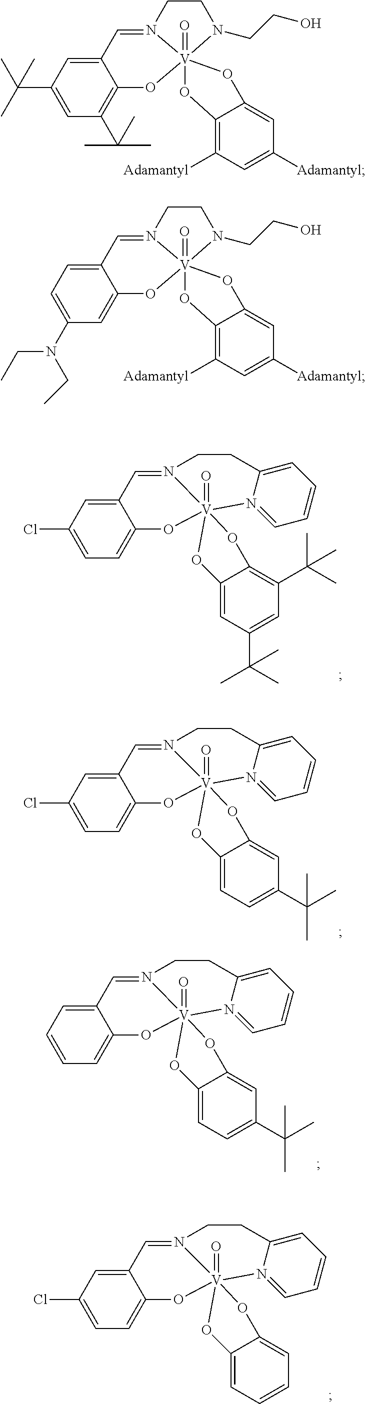

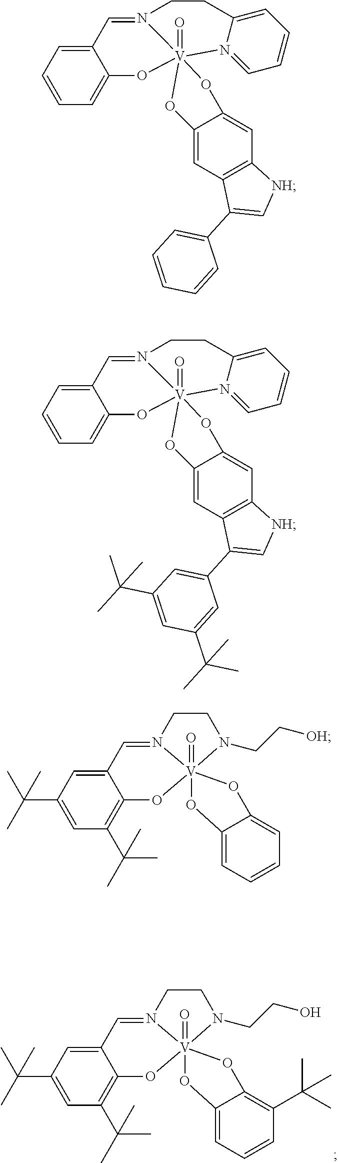

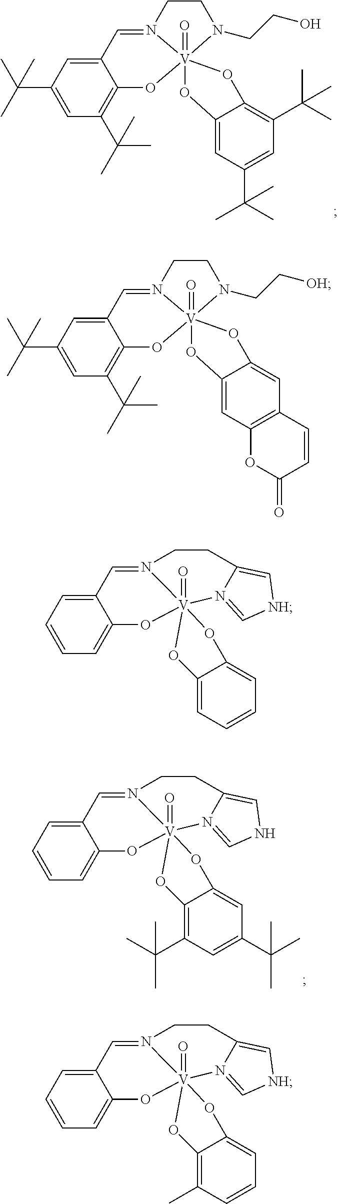

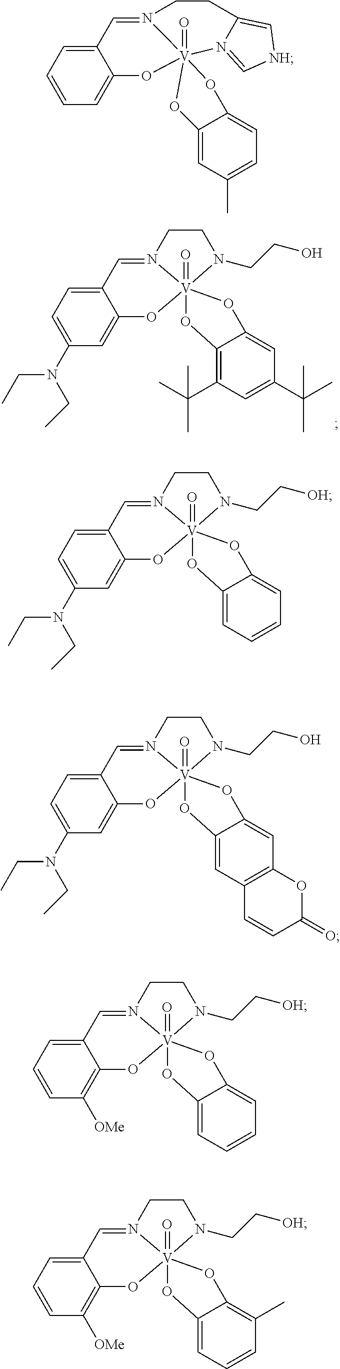

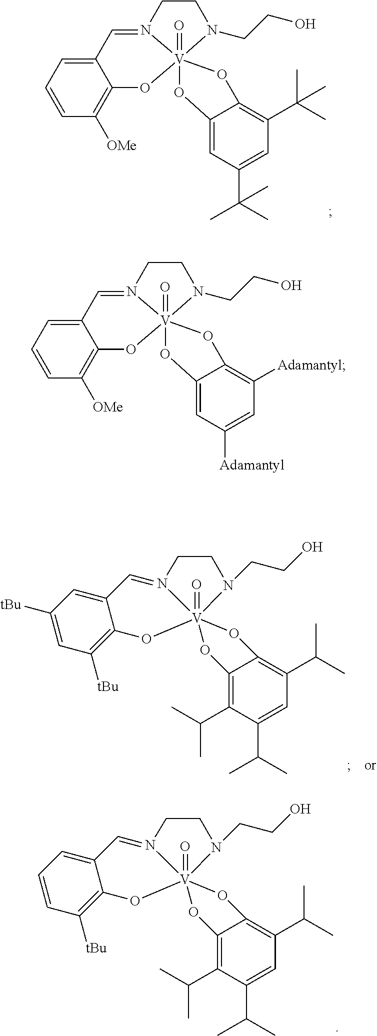

Also provided herein are pharmaceutical compositions comprising a therapeutically effective amount of a vanadium complex described herein. In some embodiments, the vanadium complex can be non-covalently associated with human serum albumin

Further provided herein are methods of treating cancer using the vanadium complexes described herein. These methods can comprise administering to a subjected in need thereof a therapeutically effective amount of a vanadium complex described herein or a pharmaceutical composition comprising a therapeutically effective amount of a vanadium complex described herein.

In certain embodiments, the cancer can comprise brain cancer (e.g., a glioma).

In certain embodiments, the administration can comprise intratumoral injection.

### DESCRIPTION OF DRAWINGS

FIG. 1. Example anticancer metal-complexes used in the clinic, or under investigation.

FIG. 2. The reaction scheme for the synthesis of [VO(HSHED)Cat] and derivatives.

FIG. 3. A qualitative molecular orbital diagram demonstrating the impact of different ligands in free and vanadium(V) coordinated catechol.

FIG. 4. Dose-response curves of fresh [VO(HSHED)dtbCat], aged [VO(HSHED)dtbCat], fresh cisplatin, and aged cisplatin in T98 g cells (72 h treatments). “Fresh” solutions were added to cell culture medium less than 60 s prior to cell treatment. “Aged” solutions were incubated in cell culture medium under a 5% CO₂ atmosphere for 24 hrs at 310K before addition to cells.

FIG. 5. The structure of the classes of example vanadium coordination complexes where activity of both the complex and ligand was investigated in bacteria.

FIGS. 6A-6B. The schematic formulas for example (FIG. 6A) vanadium complexes and (FIG. 6B) free catechol ligands.

FIGS. 7A-7C. Histograms showing the growth of M. smeg treated with (FIG. 7A) [VO(Hshed)(dtb)] and H₂dtb, (FIG. 7B) [VO(Hshed)(tbc)] and H₂tbc, and (FIG. 7C) [VO(Hshed)(cat)] and H₂cat using concentrations ranging from 0.004 to 2.00 mM. Error bars are the standard deviation for triplicate measurements.

FIG. 8. UV-Vis spectra for all of the complexes at 0.250 mM in 7H9:DMSO as a function of time (0, 1, 5, 24 h). The compounds from which the spectra (panels a-g) are listed above each of the spectra.

FIGS. 9A-9B. ⁵¹V NMR spectra of 10 mM [VO(Hshed)(dtb)] at 0, 1, 5, and 24 h time points in (FIG. 9A) 50:50 H₂O:DMSO and in (FIG. 9B) 50:50 7H9 growth medium: DMSO.

FIGS. 10A-10B. ⁵¹V NMR spectra of 10 mM [VO(Hshed)(cat)] at 0, 1, 5, and 24 h time points in (FIG. 10A) 50:50 H₂O:DMSO or in (FIG. 10B) 50:50 7H9 growth medium:DMSO.

FIGS. 11A-11B. ⁵V NMR spectra of 10 mM [V(O)₂(Hshed)] at 0, 1, 5, and 24 h time points in (FIG. 11A) 50:50 H₂O:DMSO or in (FIG. 11B) 50:50 7H9 growth medium:DMSO.

FIG. 12. Illustration of the principle of the use of reactive and unstable metal complexes in intratumoral injections. Designations: M is the metal ion; and L are the ligands.

FIG. 13. Proposed mechanisms of cytotoxic activity and deactivation of example V(V) complexes: (a) a complex with hydrolytically unstable Schiff base ligand (1); and (b) a complex with stable and cytotoxic salan-type ligand (2). Potential beneficial activities of the decomposition products are shown as well. Designations: Tf is apo-transferrin; ROS are reactive oxygen species and ^{t}Bu is tert-butyl.

FIG. 14. Example pharmaceutical formulations for intratumoral injections of V(V) complexes (1, 15-18): (a) hydrophobic micelles; (b) protein tyrosine phosphatase (PTP)-targeting graphene quantum dots; (c) adducts with human serum albumin (HSA); (d) pH-controlled liposomes; and (e) co-administration with oncolytic viruses. ^{t}Bu is tert-butyl.

FIG. 15. Structures of two vanadium Schiff bases complexes (when X═H) (a) [VO(X—HSHED)(DTB)], (b) [VO(X—HSHED)(Cat)], and a third complex (c) [VO(X—HSHED)(3-MeCat)]. The three chloro-substituted Schiff base complexes were designed with X═Cl and ([VO(HSHED)(R₂Cat)] (R₂ ═H,H; H,Me; or tBu,tBu) (HSHED=N-(salicylideneaminato)-N′-(2-hydroxyethyl)-1,2-ethanediamine, Cl-HSHED=4-chloro-N-(salicylideneaminato)-N′-(2-hydroxyethyl)-1,2-ethanediamine, Cat=pyrocatecholato, 3-MeCat=3-methylcatecholato, DTB=3,5-di-t-butylcatecholato). The hydrophobicity of the three chloro-substituted Schiff base complexes is illustrated using space filling models in (d-f), and the different hydrolytic stability are illustrated by the blue and orange arrows showing access of H₂O molecules.

FIG. 16. Relative hydrophobicity was estimated using log P values from Chemicalize calculations, by ChemAxiom. These calculations required the assumption that a covalent bond description can approximate a coordinate bond in these metal complexes. The box shows hydrophobicity regions where most complexes are found to penetrate living cells based on Lipinski's rule of five.

FIG. 17. UV-vis spectra of the halogenated V(V) complexes [VO(Cl-HSHED)(R)] and the parent complexes [VO(HSHED)(R)] recorded at 0.10 mM V in DMSO.

FIG. 18. Typical positive-ion low-resolution ESI-MS data for V(V)-Schiff base-catecholato complexes (a-g); ˜10 μM solutions in acetonitrile, m/z values in the positive-ion mode and (h) experimental (lines) and fitted (points) isotopic distributions for selected signals. The assignment of the marked signals is shown in Table 5.

FIG. 19. ⁵¹V NMR (400 MHz) of [VO(Cl-HSHED)(Cat)], [VO(Cl-HSHED)(3-MeCat)], and [VO(Cl-HSHED)(DTB)] in CD₃CN and DMSO-d₆.

FIG. 20. Four of the possible isomers of [VO(Cl-HSHED)(DTB)] are shown with the major isomer illustrated as structure (A). The structures vary with how the Schiff base is coordinated to the vanadium and the stereocenter of the alkyl amine. Note that each of the isomers, as shown in FIG. 20, are chiral, and racemic mixtures form in solution, even though only one isomer is shown.

FIG. 21. Structure of [VO(Cl-HSHED)(Cat)] with the three spin systems X, Y, and Z is shown. ¹H-¹H COSY and ¹H-¹H NOESY NMR (400 MHz) spectra of 10 mM [VO(Cl-HSHED)(Cat)] in CD₃CN at 26° C. (A) Full ¹H-¹H COSY NMR spectrum of [VO(Cl-HSHED)(Cat)] in CD₃CN. (B) Zoom in of the aromatic region ¹H-¹H COSY NMR spectrum of [VO(Cl-HSHED)(Cat)]. (C) Zoom in of the aliphatic region ¹H-¹H COSY NMR spectrum of [VO(Cl-HSHED)(Cat)]. (D) Full ¹H-¹H NOESY NMR spectrum of [VO(Cl-HSHED)(Cat)] in CD₃CN. Blue intensity contours represent negative NOEs, and red intensity contours represent positive NOEs. A standard NOESY sequence was used consisting of 200-256 transients with 16 scans in the f1 domain using a 500 ms mixing time and a 1.5 s relaxation delay. The structure of [VO(Cl-HSHED)(Cat)] is shown with a proton-labeling scheme.

FIG. 22. ¹H NMR (400 MHz) spectrum of 1 mM [VO(Cl-HSHED)(DTB)] from the 2.0-4.5 ppm in CD₃CN (bottom) and in CD₃CN with one drop of D₂O (top).

FIG. 23. Structure of [VO(Cl-HSHED)(DTB)] with the three spin systems X, Y, and Z is shown. ¹H-¹H COSY and ¹H-¹H NOESY NMR (400 MHz) spectra of 10 mM [VO(Cl-HSHED)(DTB)] in CD₃CN and a D₂O spike at 26° C. (A) Full ¹H-¹H COSY NMR spectrum of [VO(Cl-HSHED)(DTB)] in CD₃CN. (B) Zoom of the aromatic region ¹H-¹H COSY NMR spectrum of [VO(Cl-HSHED)(DTB)]. (C) Zoom of the aliphatic region ¹H-¹H COSY NMR spectrum of [VO(Cl-HSHED)(DTB)]. Zoom of the aromatic region ¹H-¹H COSY NMR spectrum of [VO(Cl-HSHED)(DTB)]. (D) Full ¹H-¹H NOESY NMR spectrum of [VO(Cl-HSHED)(DTB)]. Blue intensity contours represent negative NOEs, and red intensity contours represent positive NOEs. A standard NOESY sequence was used consisting of 200-256 transients with 16 scans in the f1 domain using a 500 ms mixing time and a 1.5 s relaxation delay. The structures of [VO(Cl-HSHED)(DTB)] are shown with a proton labeling key.

FIG. 24. Superimposed CVs of 2.0 mM [VO(Cl-HSHED)(Cat)], [VO(Cl-HSHED)(3-MeCat)], [VO(Cl-HSHED)(DTB)], 100 mM TBAP in CH₃CN, run at 100 mV s⁻¹.

FIG. 25. A) Syntheses of the [VO₂(X-SALIEP)](W═H, Cl) precursors using modified procedures from the literature. B) Syntheses of the [VO(W-SALIEP)L] complexes where W═H, Cl and X, Y and Z represent H or t-Bu substituents on the catecholato ligand.

FIG. 26. Stacked ⁵¹V NMR plots of the [VO(SALIEP)L] and [VO(Cl-SALIEP)L]series in 10.0 mM d6-DMSO solutions.

FIG. 27. Potential geometric isomers of [VVO(SALIEP)(DTB)] that could form in solution.

FIG. 28. A)¹H NMR assignments of the [VO(SALIEP)(DTB)] complex. B) Partial ¹H-¹H 2D NOESY NMR spectrum of the of the [VO(SALIEP)(DTB)] complex. C) 3D model of the major isomer of the [VO(SALIEP)(DTB)] complex, as evidenced by the ¹H-¹H 2D NOESY NMR spectrum. D) Full ¹H-¹H 2D NOESY NMR spectrum of the of the [VO(SALIEP)(DTB)] complex.

FIGS. 29A-29B. Room temperature stacked EPR spectra of (FIG. 29A) [VIVO(SALIEP)(Cat)] and (FIG. 29B) [VIVO(Cl-SALIEP)(Cat)] in 10.0 mM DMSO solutions with added Zn(OAc)₂ to trap the resulting semiquinone radicals.

FIG. 30. Cyclic voltammograms of 2.0 mM dry and degassed MeCN solutions of [VO(SALIEP)(4 TB)] (A), [VO(SALIEP)(DTB)] (B), [VO(Cl-SALIEP)(4 TB)] (C) and [VO(Cl-SALIEP)(DTB)] (D) analogs run at 100 mV s⁻¹ at a 3 mm diameter glassy carbon electrode.

FIG. 31. UV-Vis spectra of the parent complexes [VO(SALIEP)L] and the halogenated complexes [VO(Cl-SALIEP)L] recorded in 0.10 mM solutions of dry MeCN.

FIG. 32. Current design strategies for temperature sensitivity of metal-nucleus NMR chemical shifts in metal complexes.

FIG. 33. Crystal structure of [VO(EtHshed)(tbad)] (1). Pink, red, blue, and gray spheres represent vanadium, oxygen, nitrogen, and carbon atoms, respectively. H atoms are omitted for clarity.

FIG. 34. UV-Vis spectra for 1, 2, and 3. Inset: close up of low-energy charge transfer bands for 1 and 2. Data were collected on solutions of 1-3 in CH₃CN with 0.1-0.3 mM concentrations.

FIG. 35A. Variable-temperature ⁵¹V NMR spectra for 1 collected with a 400 MHz (¹H) system and concentration of 10 mM in CH₃CN. The four observed peaks are different isomers of the complex and are labeled.

FIG. 35B. Relative chemical shift changes with temperature for 1, 2, and 3, all normalized toward a 10° C. ⁵¹V δ of 0 ppm. Solid lines are fits as a result of linear regression to give Δδ/ΔT values described in the text.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

At various places in the present specification, divalent linking substituents are described. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons.

Example alkoxy groups include methoxy, ethoxy, propoxy (e.g., n-propoxy and isopropoxy), tert-butoxy, and the like. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)O— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms.

In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “thio” refers to a group of formula —SH.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to about 20 carbon atoms, from 6 to about 15 carbon atoms, or from 6 to about 10 carbon atoms. In some embodiments, the aryl group is a substituted or unsubstituted phenyl.

As used herein, the term “carbamyl” to a group of formula —C(O)NH₂.

As used herein, the term “carbonyl”, employed alone or in combination with other terms, refers to a —C(═O)— group, which may also be written as C(O).

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “halo” refers to F, Cl, Br, or I. In some embodiments, a halo is F, Cl, or Br. In some embodiments, a halo is F or Cl.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2 s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, 6, 7, 8, 9, or 10 ring-forming carbons (C₃₋₁₀). Ring-forming carbon atoms of a cycloalkyl group can be optionally substituted by oxo or sulfido (e.g., C(O) or C(S)). Cycloalkyl groups also include cycloalkylidenes. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl (NOR), norpinyl, norcarnyl, and the like. In some embodiments, cycloalkyl is cyclopropyl, cyclobutyl, cyclopentyl, cyclohexyl, cyclopentyl, or adamantly (AD). In some embodiments, the cycloalkyl has 6-10 ring-forming carbon atoms. In some embodiments, cycloalkyl is adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membereted heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, the compounds described herein can contain one or more asymmetric centers and thus occur as racemates and racemic mixtures, enantiomerically enriched mixtures, single enantiomers, individual diastereomers and diastereomeric mixtures (e.g., including (R)- and (S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms can be present in a substituent, such as an alkyl group. All such isomeric forms, as well as mixtures thereof, of these compounds are expressly included in the present description. The compounds described herein can also or further contain linkages wherein bond rotation is restricted about that particular linkage, e.g. restriction resulting from the presence of a ring or double bond (e.g., carbon-carbon bonds, carbon-nitrogen bonds such as amide bonds). Accordingly, all cis/trans and E/Z isomers and rotational isomers are expressly included in the present description. Unless otherwise mentioned or indicated, the chemical designation of a compound encompasses the mixture of all possible stereochemically isomeric forms of that compound.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, IN 1972), each of which is incorporated herein by reference in their entireties. It is also understood that the compounds described herein include all possible regioisomers, and mixtures thereof, which can be obtained in pure form by standard separation procedures known to those skilled in the art, and include, but are not limited to, column chromatography, thin-layer chromatography, and high-performance liquid chromatography.

Unless specifically defined, compounds provided herein can also include all isotopes of atoms occurring in the intermediates or final compounds. Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

When a disclosed compound is named or depicted by structure, it is to be understood that the compound, including solvates thereof, may exist in crystalline forms, non-crystalline forms or a mixture thereof. The compounds or solvates may also exhibit polymorphism (i.e. the capacity to occur in different crystalline forms). These different crystalline forms are typically known as “polymorphs.” It is to be understood that when named or depicted by structure, the disclosed compounds and solvates (e.g., hydrates) also include all polymorphs thereof. As used herein, the term “polymorph” means solid crystalline forms of a compound or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it. In addition, one polymorph may spontaneously convert to another polymorph under certain conditions.

When a disclosed compound is named or depicted by structure, it is to be understood that clathrates (“inclusion compounds”) of the compound or its pharmaceutically acceptable salts, solvates or polymorphs are also included. As used herein, the term “clathrate” means a compound of the present invention or a salt thereof in the form of a crystal lattice that contains spaces (e.g., channels) that have a guest molecule (e.g., a solvent or water) trapped within.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

Vanadium Complexes

Described herein are hydrophobic Schiff base catecholate vanadium complexes. These complexes can exhibit anti-cancer activity, and thus be suitable for use in chemotherapy. In some embodiments, these complexes can have activities superior to cis-platin.

These compounds are related to the [VO(HSHED)(DTB)] complex where HSHED is N-(salicylideneaminato)-N′-(2-hydroxyethyl)-1,2-ethanediamine and the non-innocent catecholato ligand is the di-tert-butyl-catecholate, DTB, has higher stability compared to simpler catecholate complexes.

For example, chloro-substituted Schiff base complexes of vanadium(V) with substituted catecholates as co-ligands were synthesized for comparison with their non-chlorinated Schiff base vanadium complexes. Isomers for each complex were identified in organic solvents using ⁵¹V and ¹H NMR spectroscopies. Spectroscopy was used to characterize the structure of the major isomer in solution, and to demonstrate that the observed isomers are exchanging in solution. All three chloro-substituted Schiff base vanadium(V) complexes with substituted catecholates were characterized by UV-vis spectroscopy, mass spectrometry and electrochemistry. The vanadium complexes with the chloro-substituted Schiff base were more hydrophobic, more hydrolytically stable and more easily reduced compared to their corresponding parent counterparts. Upon testing in human glioblastoma multiforme (T98 g) cells as an in vitro model of brain gliomas, the most sterically hindered, hydrophobic and stable compound was superior to the two other complexes and upon aging formed less toxic decomposition products. This will make this complex a candidate for pre-clinical investigations of intratumoral injections against late-stage cancers. These newly designed sterically hindered complexes the second non-innocent vanadium Schiff base complex with potent in vitro anticancer properties that are superior to cisplatin.

For example, provided herein are vanadium complexes defined by Formula A

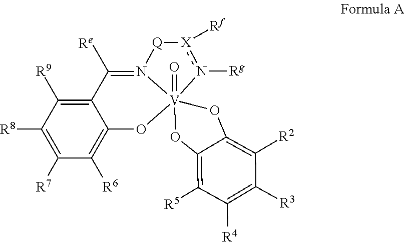

wherein
Q is selected from

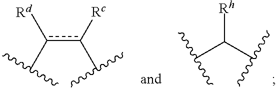

X is absent, or selected from N, C, or CH when Q is

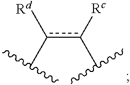

or X is selected from N, C, or CH when Q is

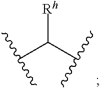

R^{h} is selected from H, ═O, halo, C₁₋₆ alkyl, or C₁₋₄ haloalkyl, or R^{h} and R^{f}, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R^{c} and R^{d} are independently selected from H, ═O, halo, C₁₋₆ alkyl, or C₁₋₄haloalkyl; or R^{c} and R^{d} and/or R^{e} and R^{g}, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R^{e} is selected from H, C₁₋₆ alkyl, C₁₋₄haloalkyl;
[symbol] represents individually for each occurrence, as valence permits, a single bond or a double bond;
R^{f} and R^{g}, together with the atoms to which they are attached, form a 3-10 membered heterocycloalkyl group or a 5-10 membered heteroaryl group, each optionally substituted with 1, 2, or 3 independently selected R^{A} groups;
R², R³, R⁴, and R⁵ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R² and R³, R³ and R⁴, and/or R⁴ and R⁵, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R⁶, R⁷, R⁸, and R⁹ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R⁶ and R⁷, R⁷ and R⁸, and/or R⁸ and R⁹, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups; and
each R^{A} is independently selected from OH, NO₂, CN, halo, C₁₋₆ alkyl, C₂₋₆ alkenyl, C₂-6 alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some aspects of Formula A, Q is

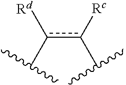

and X is absent, or selected from N, C, or CH.

In some aspects of Formula A, [symbol] represents a single bond bond. In other aspects of Formula A, [symbol] represents a double bond bond.

In some aspects of Formula A, R^{c} and R^{d}, together with the atoms to which they are attached, form a 3-10 membered cycloalkyl group, or a 6-10 membered aryl group, each optionally substituted with 1, 2, or 3 independently selected R^{A} groups.

In some aspects of Formula A, X is absent.

In some aspects of Formula A, R^{c} and R^{g}, together with the atoms to which they are attached, form a 3-10 membered heterocycloalkyl group or a 5-10 membered heteroaryl group, each optionally substituted with 1, 2, or 3 independently selected R^{A} groups.

In some aspects of Formula A, Q is

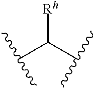

and X is selected from N, C, or CH.

In some aspects of Formula A, R^{h} is hydrogen.

In some aspects of Formula A, R^{e} is selected from H and C₁₋₆ alkyl. In certain aspects of Formula A, R^{e} is H.

In some aspects of Formula A, R^{f} and R^{g}, together with the atoms to which they are attached, form an imidazole ring, a pyridine ring, a pyrimidine ring, a pyrazine ring, a pyrrole ring, a thiazole ring, an indole ring, a purine ring, or a pteridine ring, each optionally substituted with 1, 2, or 3 independently selected R^{A} groups.

In certain aspects of Formula A, R^{f} and R^{g}, together with the atoms to which they are attached, form an imidazole ring optionally substituted with 1, 2, or 3 independently selected R^{A} groups. In certain aspects of Formula A, R^{f} and R^{g}, together with the atoms to which they are attached, form a pyridine ring optionally substituted with 1, 2, or 3 independently selected R^{A} groups.

In some aspects of Formula A, one or more of R⁶, R⁷, R⁸, and R⁹ is not hydrogen.

In some aspects of Formula A, one or more of R⁶, R⁷, R⁸, and R⁹ is halo, such as chloro.

In some aspects of Formula A, one or more of R⁶, R⁷, R⁸, and R⁹ is C₁₋₆ alkyl, such as t-butyl.

In some aspects of Formula A, one or more of R⁶, R⁷, R⁸, and R⁹ is di(C₁₋₆ alkyl)amino, such as diethylamino.

In some aspects of Formula A, one or more of R⁶, R⁷, R⁸, and R⁹ is C₁₋₆ alkoxy, such as methoxy.

In some aspects of Formula A, R⁸ is halo, such as chloro.

In some aspects of Formula A, R⁶ and R⁸ are each C₁₋₆ alkyl, such as t-butyl.

In some aspects of Formula A, R⁷ is di(C₁₋₆ alkyl)amino, such as diethylamino.

In some aspects of Formula A, R⁶ is C₁₋₆ alkoxy, such as methoxy.

In some aspects of Formula A, one or more of R², R³, R⁴, and R⁵ is not hydrogen.

In some aspects of Formula A, one or more of R², R³, R⁴, and R⁵ is C₁₋₆ alkyl, such as t-butyl.

In some aspects of Formula A, one or more of R², R³, R⁴, and R⁵ is a 3-10 membered cycloalkyl group, such as adamantly or norbornyl.

In some aspects of Formula A, R³ and R⁵ are C₁₋₆ alkyl, such as t-butyl.

In some aspects of Formula A, R³ and R⁵ are a 3-10 membered cycloalkyl group, such as adamantly or norbornyl.

In some aspects of Formula A, the complex is not one of the complexes below

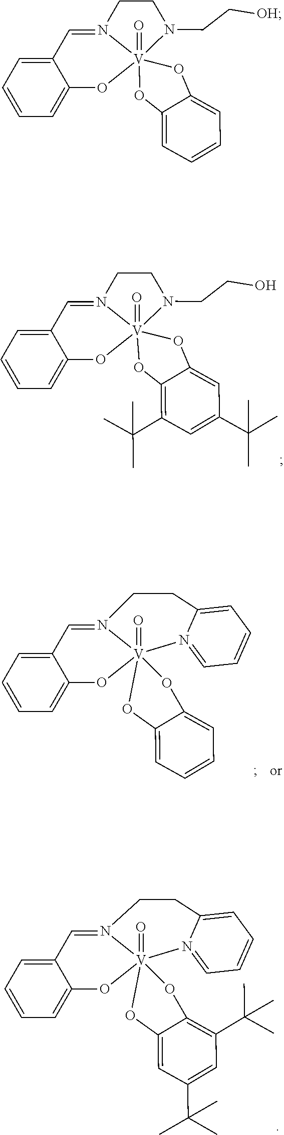

Also provided are vanadium complexes defined by Formula B, Formula C, or Formula D

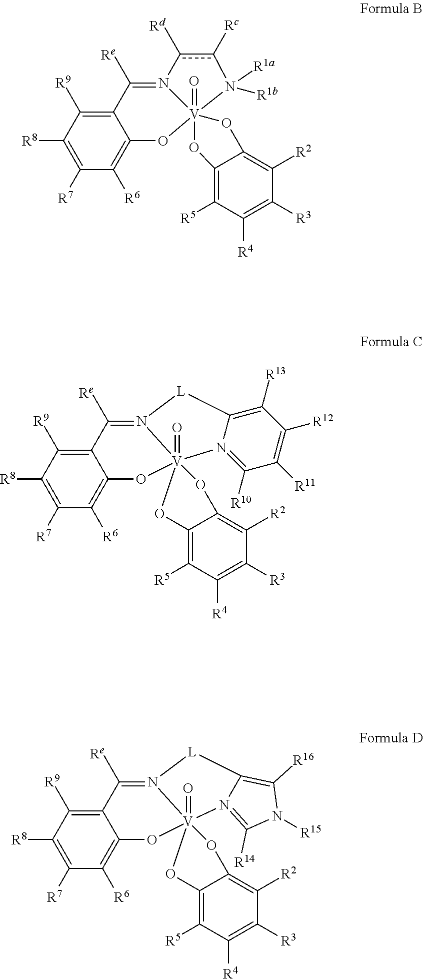

wherein
L is selected from

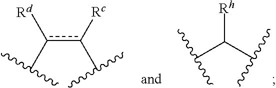

R^{h} is selected from H, ═O, halo, C₁₋₆ alkyl, or C₁₋₄ haloalkyl;
R^{c} and R^{d} are independently selected from H, ═O, halo, C₁₋₆ alkyl, or C₁₋₄haloalkyl; or R^{c} and R^{d} and/or R^{e} and R^{g}, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R^{e} is selected from H, C₁₋₆ alkyl, C₁₋₄haloalkyl;
[symbol] represents individually for each occurence, as valence permits, a single bond or a double bond;
R^{1a} is selected from C₁₋₆ alkyl, C₁₋₄haloalkyl, C₂₋₆ alkenyl, C₂₋₆ alkynyl, C₃₋₁₀ cycloalkyl, C₃₋₁₀ cycloalkyl-C₁₋₄ alkylene, 6-10 membered aryl, and 6-10 membered aryl-C₁₋₄ alkylene, each of which is optionally substituted by 1, 2, 3, or 4 independently selected R^{A} groups;
R^{1b} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄ haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups;
R², R³, R⁴, and R⁵ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R² and R³, R³ and R⁴, and/or R⁴ and R⁵, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R⁶, R⁷, R⁸, and R⁹ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R⁶ and R⁷, R⁷ and R⁸, and/or R⁸ and R⁹, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁰, R¹¹, R¹², and R¹³ are independently selected from H, OH, NO₂, CN, halo, C₁₋₆ alkyl, C₂₋₆ alkenyl, C₂₋₆ alkynyl, C₁₋₄haloalkyl, C₃₋₁₀cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R¹⁰ and R¹¹, R¹¹ and R¹², and/or R¹² and R¹³, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁴ and R¹⁶ are independently selected from H, OH, NO₂, CN, halo, C₁₋₆ alkyl, C₂₋₆ alkenyl, C₂₋₆ alkynyl, C₁₋₄haloalkyl, C₃₋₁₀cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups;
R¹⁵ is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄ haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups; and
   each R^{A} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁-6 alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino

In some aspects of Formula B, Formula C, and Formula D, L is

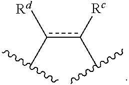

In some aspects of Formula B, Formula C, and Formula D, [symbol] represents a single bond bond. In other aspects of Formula B, Formula C, and Formula D, [symbol] represents a double bond bond.

[symbol]

In some aspects of Formula B, Formula C, and Formula D, R^{c} and R^{d}, together with the atoms to which they are attached, form a 3-10 membered cycloalkyl group, or a 6-10 membered aryl group, each optionally substituted with 1, 2, or 3 independently selected R^{A} groups.

In some aspects of Formula B, Formula C, and Formula D, L is

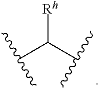

In some aspects of Formula B, Formula C, and Formula D, R^{h} is hydrogen.

In some aspects of Formula B, Formula C, and Formula D, R^{e} is selected from H and C₁₋₆ alkyl. In certain aspects of Formula B, Formula C, and Formula D, R^{e} is H.

In some aspects of Formula B, Formula C, and Formula D, one or more of R⁶, R⁷, R⁸, and R⁹ is not hydrogen.

In some aspects of Formula B, Formula C, and Formula D, one or more of R⁶, R⁷, R⁸, and R⁹ is halo, such as chloro.

In some aspects of Formula B, Formula C, and Formula D, one or more of R⁶, R⁷, R⁸, and R⁹ is C₁₋₆ alkyl, such as t-butyl.

In some aspects of Formula B, Formula C, and Formula D, one or more of R⁶, R⁷, R⁸, and R⁹ is di(C₁₋₆ alkyl)amino, such as diethylamino.

In some aspects of Formula B, Formula C, and Formula D, one or more of R⁶, R⁷, R⁸, and R⁹ is C₁₋₆ alkoxy, such as methoxy.

In some aspects of Formula B, Formula C, and Formula D, R⁸ is halo, such as chloro.

In some aspects of Formula B, Formula C, and Formula D, R⁶ and R⁸ are each C₁₋₆ alkyl, such as t-butyl.

In some aspects of Formula B, Formula C, and Formula D, R⁷ is di(C₁₋₆ alkyl)amino, such as diethylamino.

In some aspects of Formula B, Formula C, and Formula D, R⁶ is C₁₋₆ alkoxy, such as methoxy.

In some aspects of Formula B, Formula C, and Formula D, one or more of R², R³, R⁴, and R⁵ is not hydrogen.

In some aspects of Formula B, Formula C, and Formula D, one or more of R², R³, R⁴, and R⁵ is C₁₋₆ alkyl, such as t-butyl.

In some aspects of Formula B, Formula C, and Formula D, one or more of R², R³, R⁴, and R⁵ is a 3-10 membered cycloalkyl group, such as adamantly or norbornyl.

In some aspects of Formula B, Formula C, and Formula D, R³ and R⁵ are C₁₋₆ alkyl, such as t-butyl.

In some aspects of Formula B, Formula C, and Formula D, R³ and R⁵ are a 3-10 membered cycloalkyl group, such as adamantly or norbornyl.

In some aspects of Formula B, R^{1b} is hydrogen.

In some aspects of Formula B, R^{1a} is a C₁₋₆ alkyl group substituted by a hydroxy group.

In some aspects of Formula C, one or more of R¹⁰, R¹¹, R¹², and R¹³ are all hydrogen.

In some aspects of Formula D, one or more of R¹⁴, R¹⁵, and R¹⁶ are all hydrogen.

In some aspects of Formula B, Formula C, and Formula D, the complex is not one of the complexes below

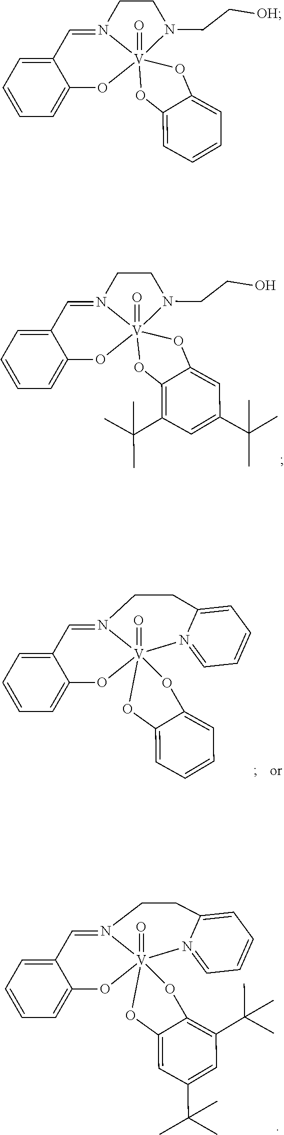

In some aspects, the vanadium complex is defined by Formula I, Formula II, or Formula III below

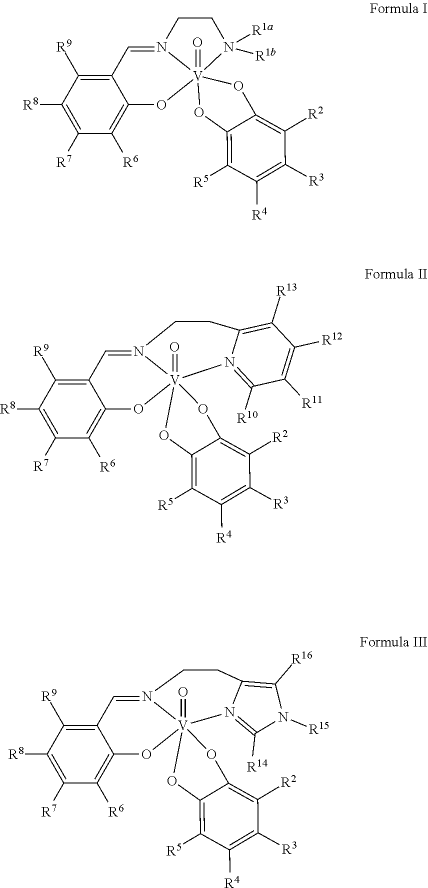

wherein
R^{1a} is selected from C₁₋₆ alkyl, C₁₋₄haloalkyl, C₂₋₆ alkenyl, C₂₋₆ alkynyl, C₃₋₁₀ cycloalkyl, C₃₋₁₀cycloalkyl-C₁₋₄ alkylene, 6-10 membered aryl, and 6-10 membered aryl-C₁₋₄ alkylene, each of which is optionally substituted by 1, 2, 3, or 4 independently selected R^{A} groups;
R^{1b} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄ haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups;
R², R³, R⁴, and R⁵ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R² and R³, R³ and R⁴, and/or R⁴ and R⁵, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R⁶, R⁷, R⁸, and R⁹ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R⁶ and R⁷, R⁷ and R⁸, and/or R⁸ and R⁹, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁰, R¹¹, R¹², and R¹³ are independently selected from H, OH, NO₂, CN, halo, C₁₋₆ alkyl, C₂₋₆ alkenyl, C₂₋₆ alkynyl, C₁₋₄haloalkyl, C₃₋₁₀cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R¹⁰ and R¹¹, R¹¹ and R¹², and/or R¹² and R¹³, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁴ and R¹⁶ are independently selected from H, OH, NO₂, CN, halo, C₁₋₆ alkyl, C₂₋₆ alkenyl, C₂₋₆ alkynyl, C₁₋₄haloalkyl, C₃₋₁₀cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups;
R¹⁵ is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄ haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups; and
each R^{A} is independently selected from OH, NO₂, CN, halo, C₁₋₆ alkyl, C₂₋₆ alkenyl, C₂-6 alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some aspects of Formula I, Formula II, and Formula III, one or more of R⁶, R⁷, R⁸, and R⁹ is not hydrogen.

In some aspects of Formula I, Formula II, and Formula III, one or more of R⁶, R⁷, R⁸, and R⁹ is halo, such as chloro. In certain aspects of Formula I, Formula II, and Formula III, R⁸ is halo, such as chloro.

In some aspects of Formula I, Formula II, and Formula III, one or more of R⁶, R⁷, R⁸, and R⁹ is C₁₋₆ alkyl, such as t-butyl.

In some aspects of Formula I, Formula II, and Formula III, one or more of R⁶, R⁷, R⁸, and R⁹ is di(C₁₋₆ alkyl)amino, such as diethylamino. In certain aspects of Formula I, Formula II, and Formula III, R⁷ is di(C₁₋₆ alkyl)amino, such as diethylamino.

In some aspects of Formula I, Formula II, and Formula III, one or more of R⁶, R⁷, R⁸, and R⁹ is C₁₋₆ alkoxy, such as methoxy. In certain aspects of Formula I, Formula II, and Formula III, R⁶ and R⁸ are each C₁₋₆ alkyl, such as t-butyl.

In some aspects of Formula I, Formula II, and Formula III, R⁶ is C₁₋₆ alkoxy, such as methoxy.

In some aspects of Formula I, Formula II, and Formula III, one or more of R², R³, R⁴, and R⁵ is not hydrogen.

In some aspects of Formula I, Formula II, and Formula III, one or more of R², R³, R⁴, and R⁵ is C₁₋₆ alkyl, such as t-butyl.

In some aspects of Formula I, Formula II, and Formula III, one or more of R², R³, R⁴, and R⁵ is a 3-10 membered cycloalkyl group, such as adamantly or norbornyl.

In some aspects of Formula I, Formula II, and Formula III, R³ and R⁵ are C₁₋₆ alkyl, such as t-butyl.

In some aspects of Formula I, Formula II, and Formula III, R³ and R⁵ are a 3-10 membered cycloalkyl group, such as adamantly or norbornyl.

In some aspects of Formula I, R^{1b} is hydrogen.

In some aspects of Formula I, R^{1a} is a C₁₋₆ alkyl group substituted by a hydroxy group.

In some aspects of Formula II, one or more of R¹⁰, R¹¹, R¹², and R¹³ are all hydrogen.

In some aspects of Formula III, one or more of R¹⁴, R¹⁵, and R¹⁶ are all hydrogen.

In some aspects of Formula I, Formula II, and Formula III, the complex is not one of the complexes below

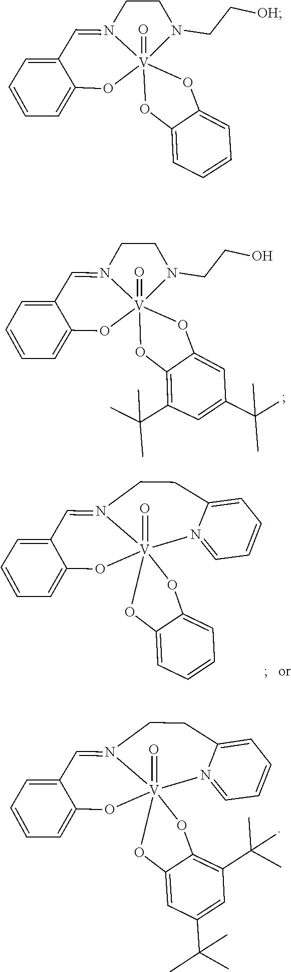

In some aspects of Formula I, Formula II, and Formula III, the complex is one of the complexes shown below

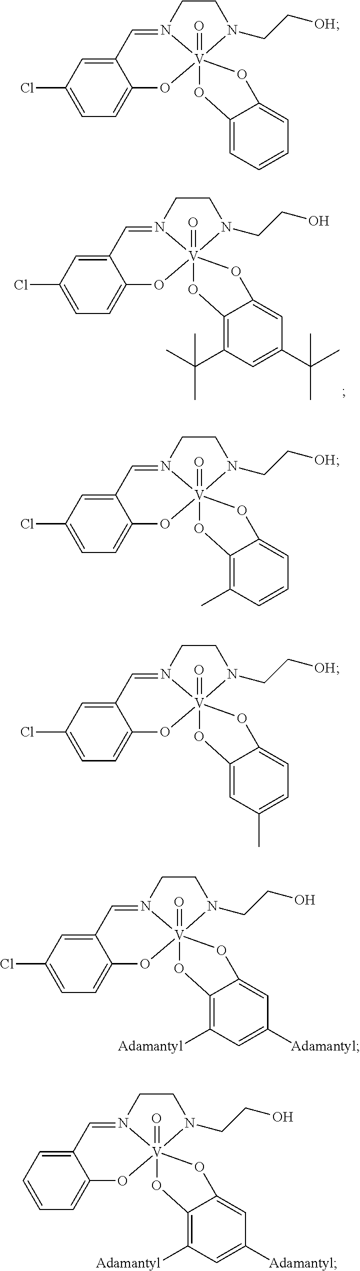

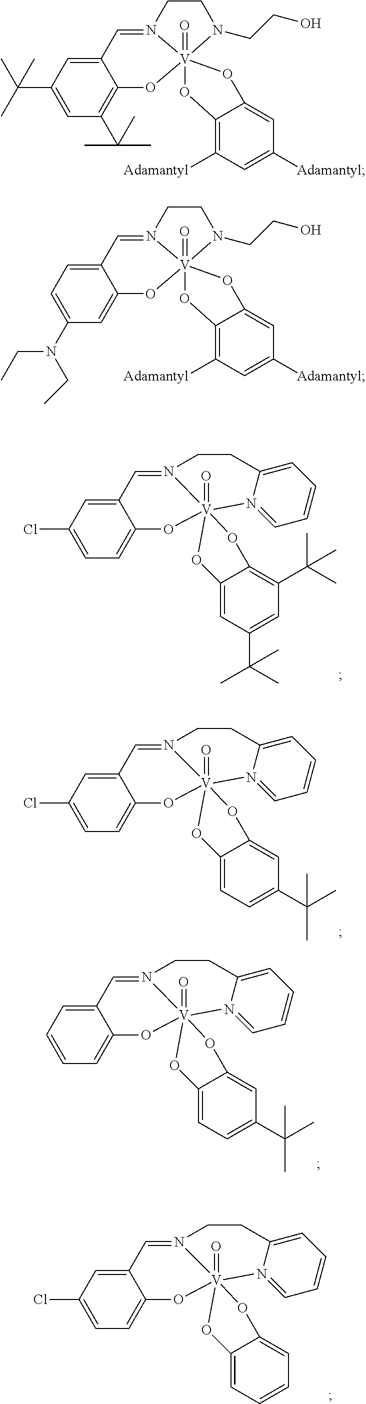

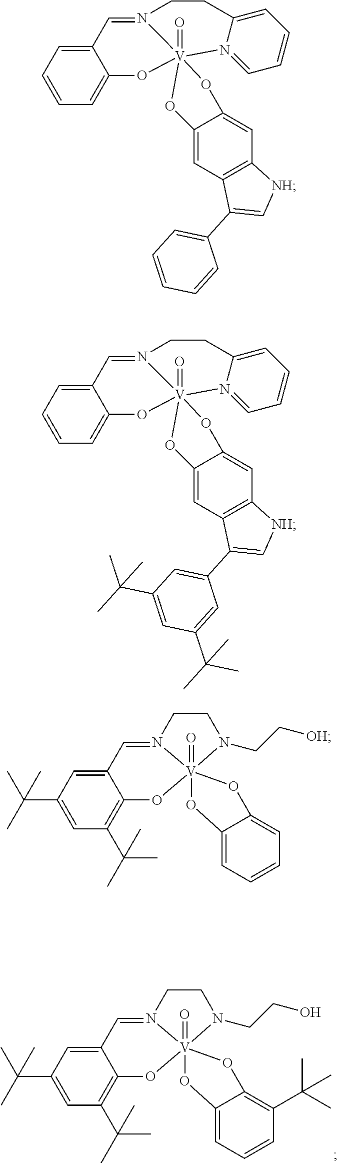

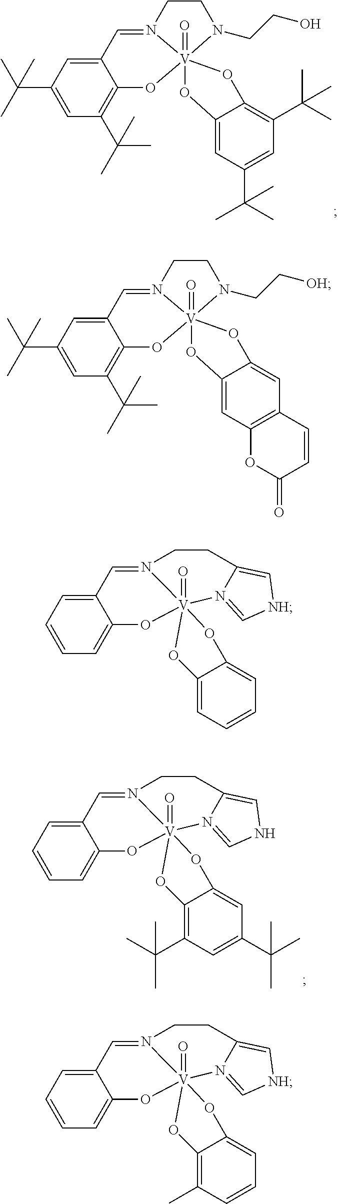

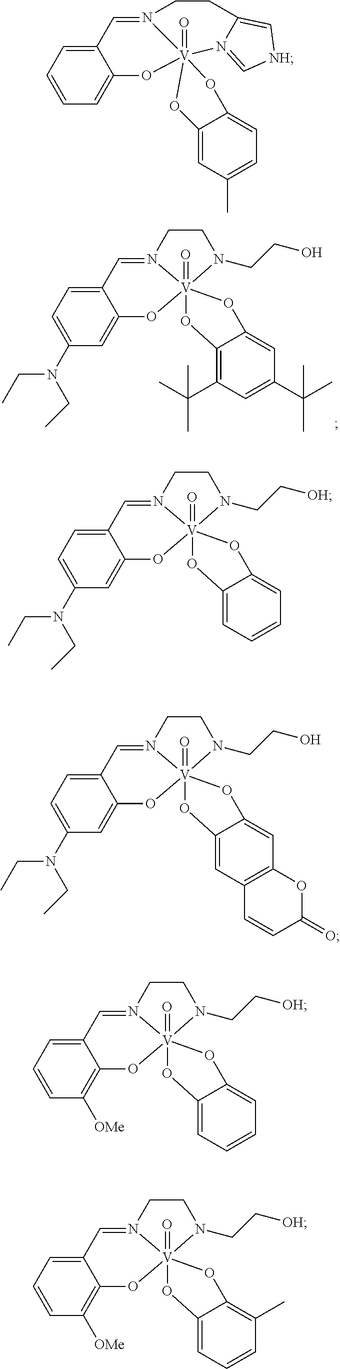

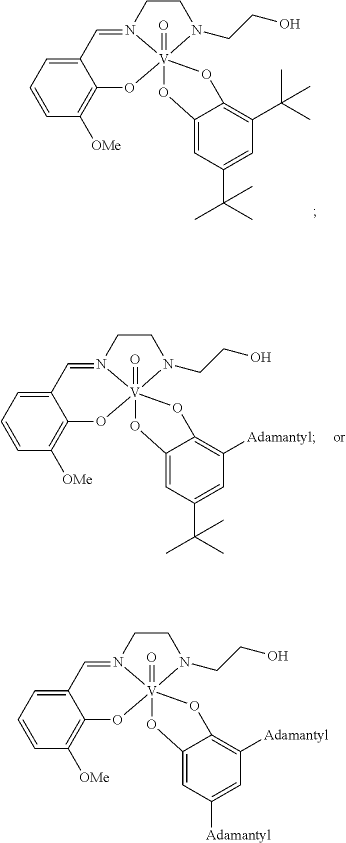

vanadium complex shown below

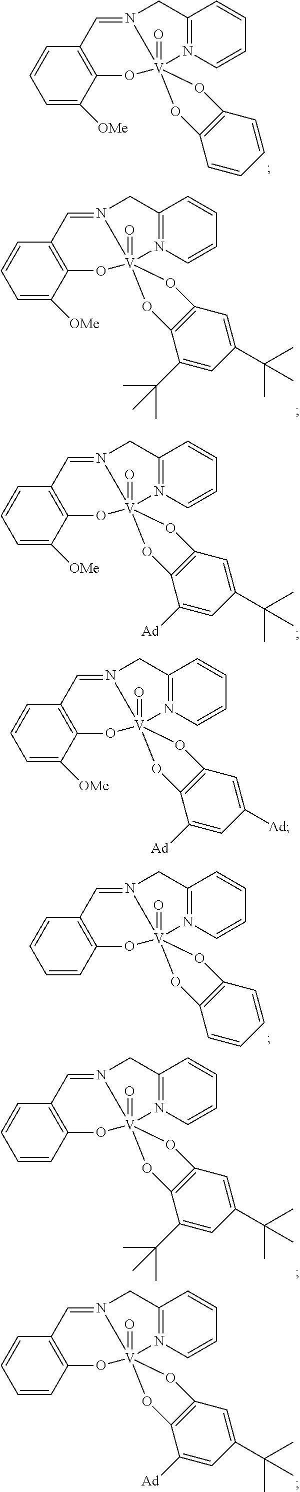

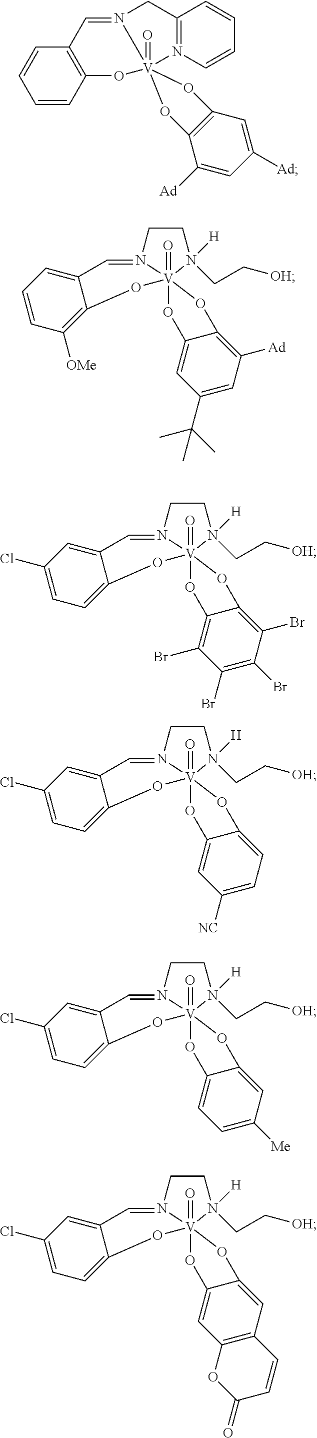

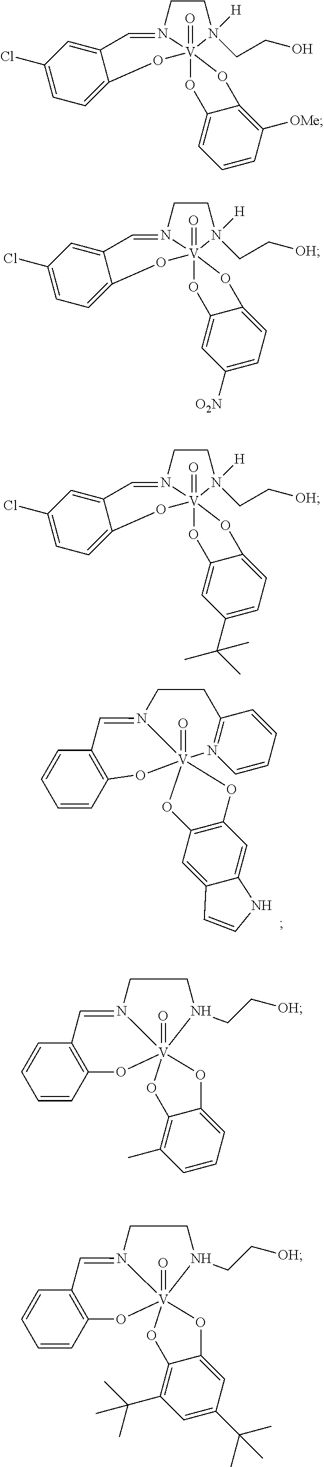

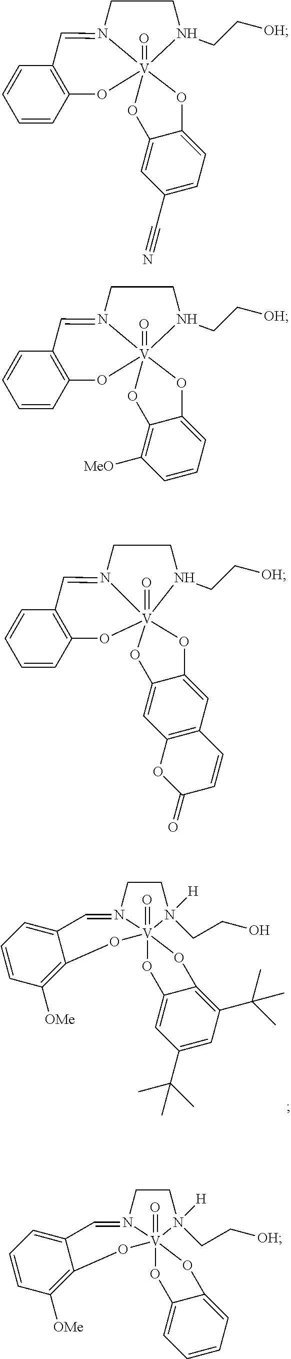

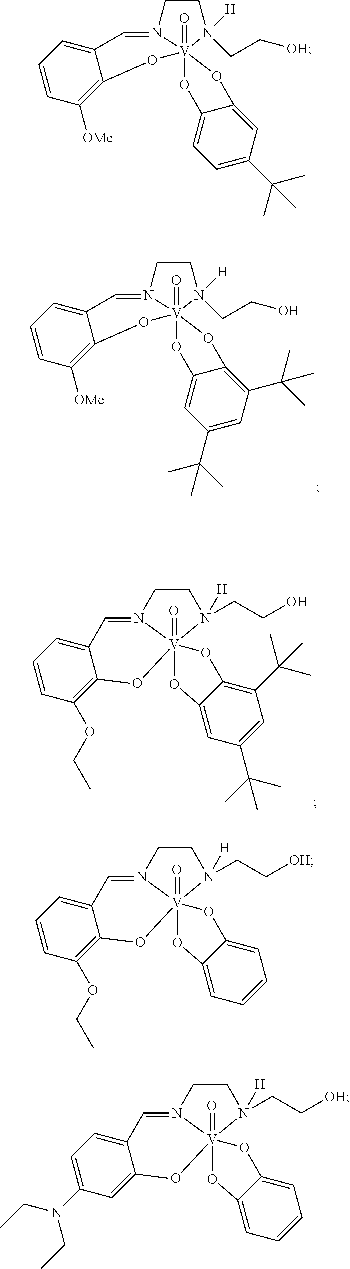

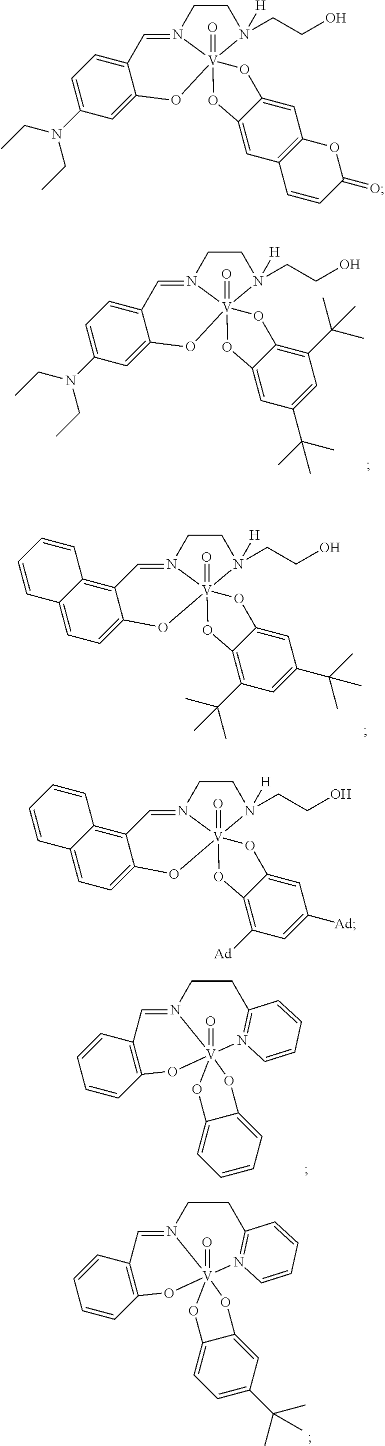

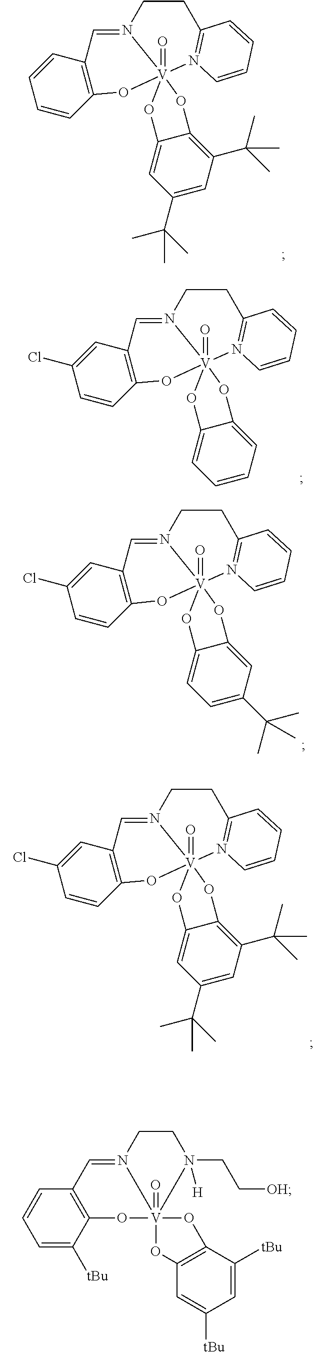

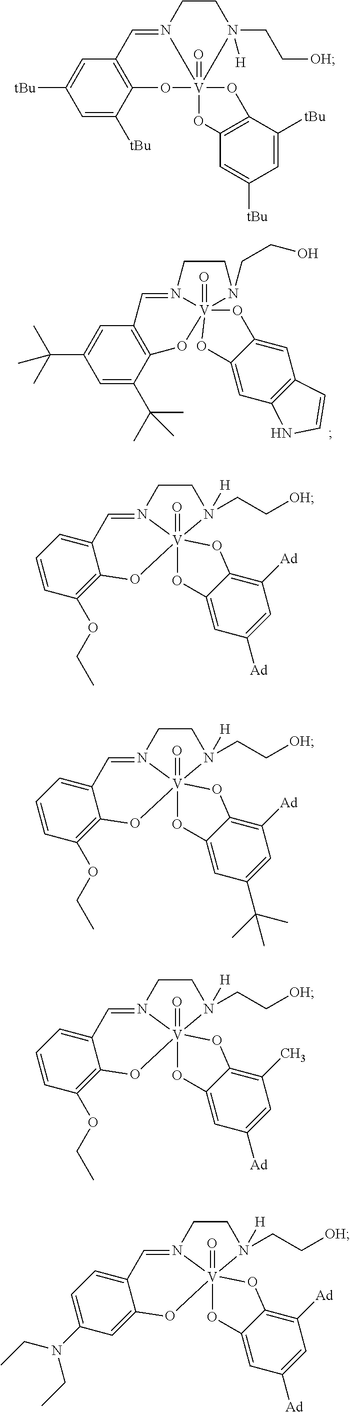

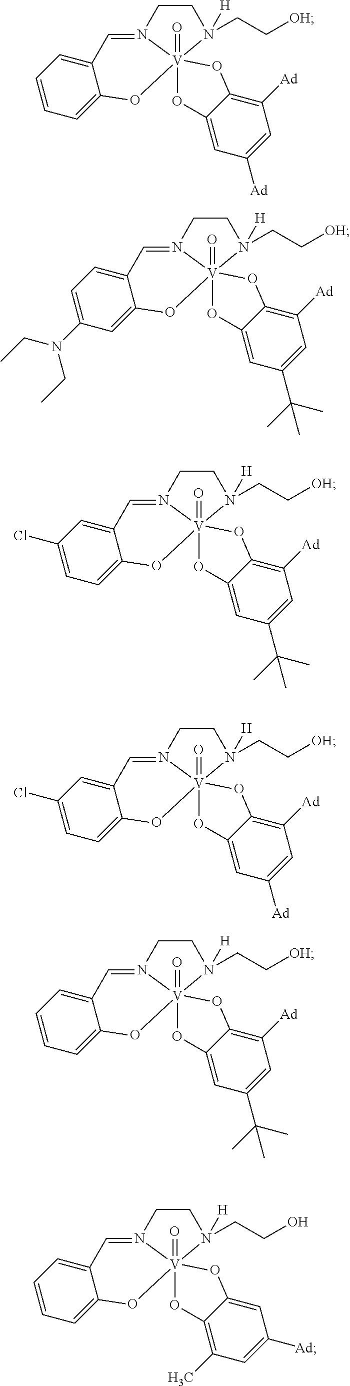

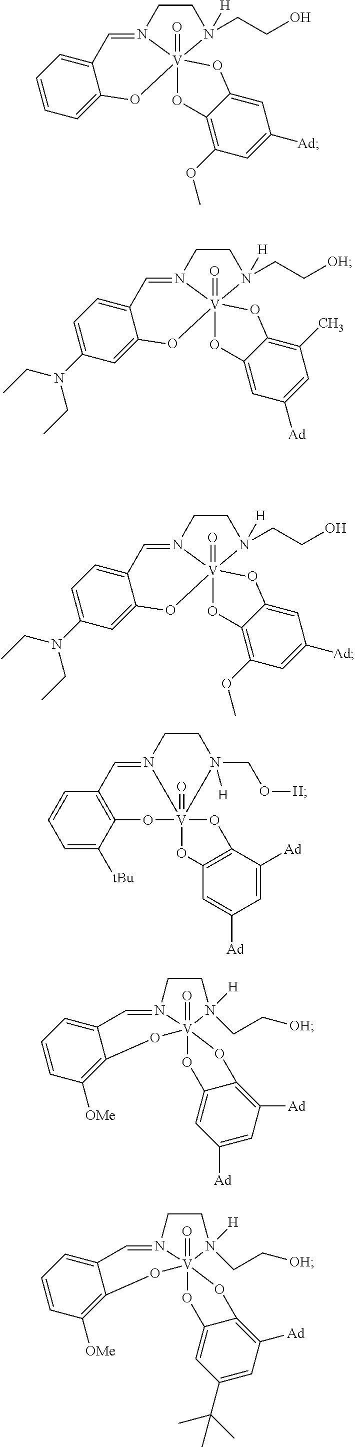

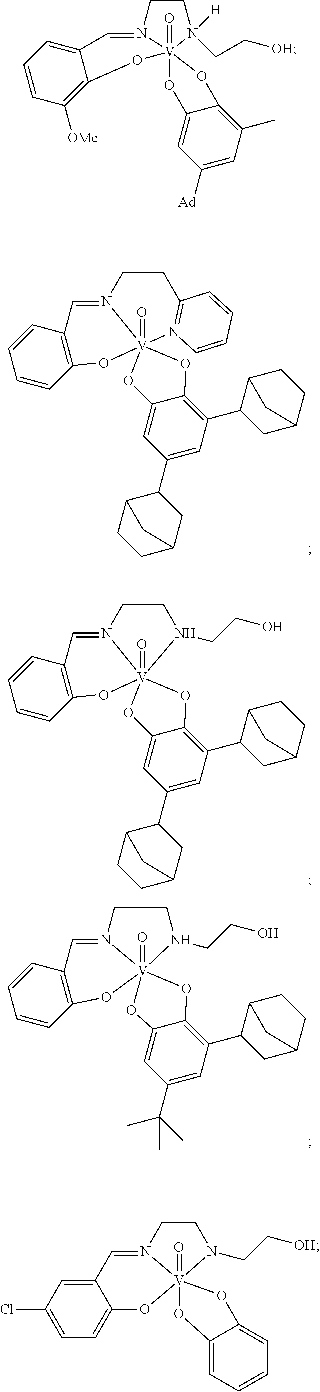

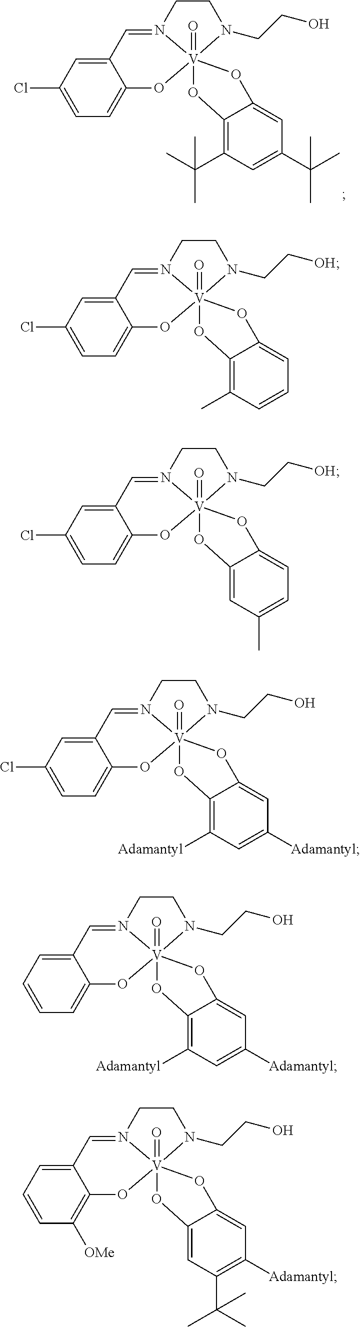

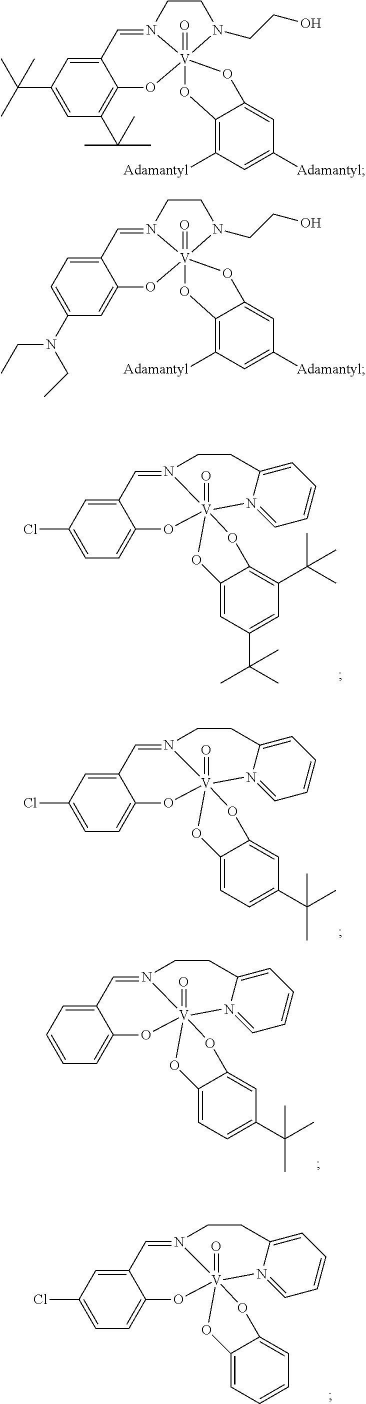

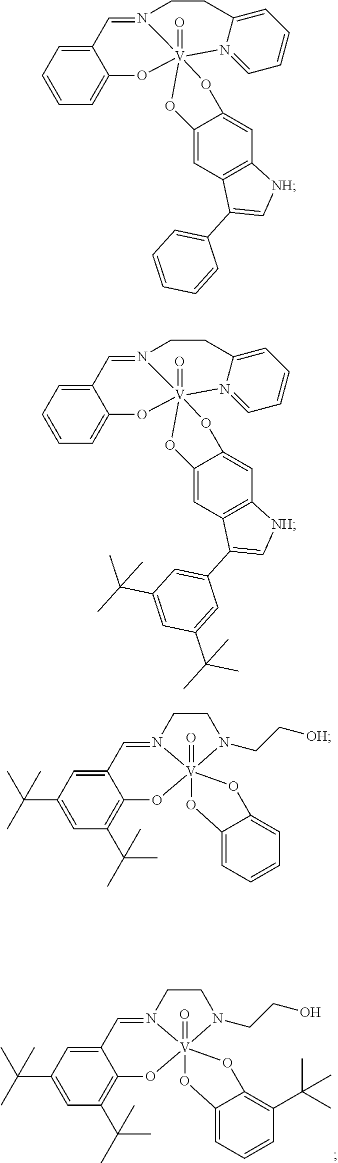

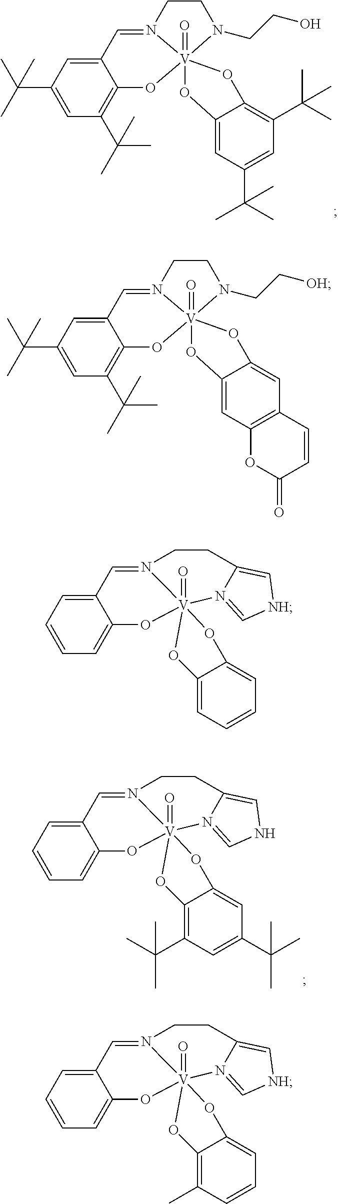

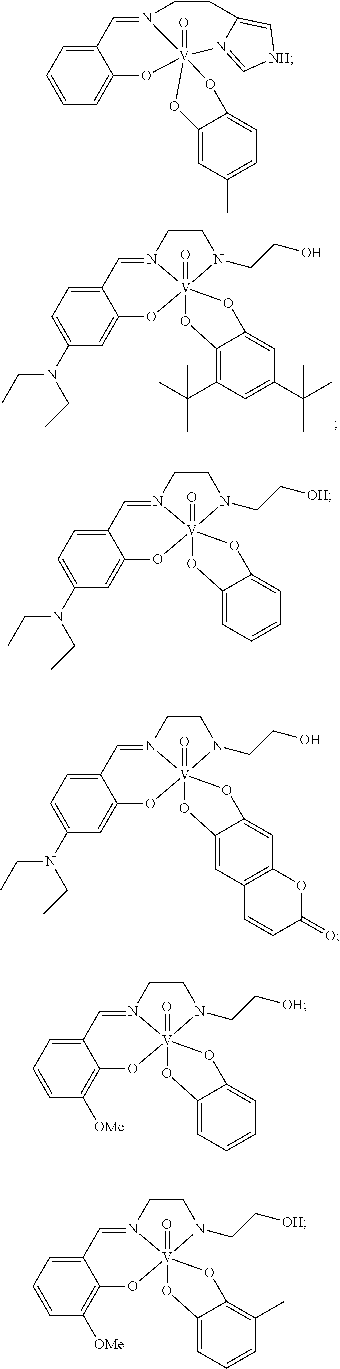

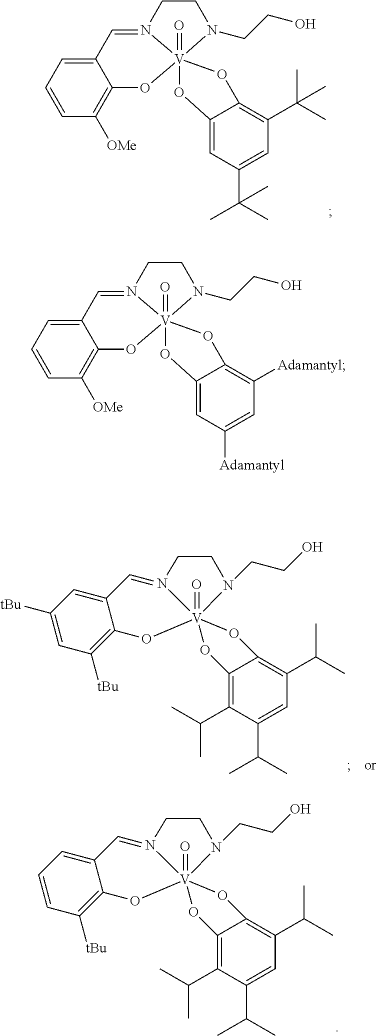

### Synthesis

The compounds described herein can be prepared using synthetic methodologies known in the art.

It will be appreciated by one skilled in the art that the processes described are not the exclusive means by which compounds provided herein may be synthesized and that a broad repertoire of synthetic organic, inorganic, and organometallic reactions is available to be potentially employed in synthesizing compounds provided herein. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H, ¹³C, ⁵¹V, etc.), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including crystallization, high performance liquid chromatography (HPLC), and normal phase silica chromatography.

### Methods of Use

The vanadium complexes described herein can be used for any suitable purpose. In some embodiments, the vanadium complexes described herein can be used to treat a subject having, suffering from, or at risk of cancer.

As used herein, the term “subject,” refers to any animal, including mammals. For example, the term “subject” includes, but is not limited to, mice, rats, other rodents, rabbits, dogs, cats, swine, cattle, sheep, horses, primates, and humans. In some embodiments, the subject is a human. The phrase “treating a subject having a cancer” can include achieving, partially or substantially, one or more of the following: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer (such as tissue or serum components).

Any suitable type of cancer can be treated by administering an effective amount of a vanadium complex or a nanoparticle including a vanadium complex to a subject in need thereof. Cancers that can be treated or prevented by administering an effective amount of a vanadium complex or a nanoparticle including a vanadium complex to a subject in need thereof include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, anal carcinoma, esophageal cancer, gastric cancer, hepatocellular cancer, bladder cancer, endometrial cancer, pancreatic cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small-cell lung cancer, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease. In certain embodiments, the cancer can comprise a glioma.

In certain embodiments, the vanadium compounds described herein can be administered locally to a tumor, for example, by intratumoral injection.

An “effective amount” is the quantity of compound in which a beneficial clinical outcome is achieved when the compound is administered to a subject. For example, when a vanadium compound is administered to a subject with a cancer, a “beneficial clinical outcome” includes a reduction in tumor mass, a reduction in metastasis, a reduction in the severity of the symptoms associated with the cancer or an increase in the longevity of the subject compared with the absence of the treatment.

The precise amount of compound administered to a subject will depend on the type and severity of the disease or condition and on the characteristics of the subject, such as general health, age, sex, body weight and tolerance to drugs. It may also depend on the degree, severity and type of cancer. The skilled artisan will be able to determine appropriate dosages depending on these and other factors. Effective amounts of the disclosed compounds may range between about 1 mg/mm² per day and about 10 grams/mm² per day. If co-administered with another anti-cancer agent for the treatment of cancer, an “effective amount” of the second anti-cancer agent will depend on the type of drug used. Suitable dosages are known for approved anti-cancer agents and can be adjusted by the skilled artisan according to the condition of the subject, the type of cancer being treated, the vanadium (V) compound being used, the nature of the pharmaceutical compositions, and the route of administration.

### Combination Therapies

In In some embodiments, the methods provided herein further comprise administering one or more additional therapeutic agents to the subject. In some embodiments, each of the one or more additional therapeutic agents is independently selected from the group consisting of a steroid, an anti-allergic agent, an anti-microbial agent, an anti-inflammatory agent, and a chemotherapeutic agent.

Example steroids include, but are not limited to, corticosteroids such as cortisone, dexamethasone, hydrocortisone, methylprednisolone, prednisolone, and prednisone.

Example anti-microbial agents include, but are not limited to, aminoglycosides (e.g., gentamicin, neomycin, and streptomycin), penicillins (e.g., amoxicillin and ampicillin), and macrolides (e.g., erythromycin).

Example anti-inflammatory agents include, but are not limited to, aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

Example chemotherapeutics include, but are not limited to, proteosome inhibitors (e.g., bortezomib), thalidomide, revlimid, and DNA-damaging agents such as melphalan, doxorubicin, cyclophosphamide, vincristine, etoposide, carmustine, and the like. For example, one or more of the following agents may be used in combination with the compounds provided herein and are presented as a non-limiting list: a cytostatic agent, cisplatin, taxol, etoposide, irinotecan, topotecan, paclitaxel, docetaxel, epothilones, tamoxifen, 5-fluorouracil, temozolomide, cyclophosphamide, gefitinib, erlotinib hydrochloride, imatinib mesylate, gemcitabine, uracil mustard, chlormethine, ifosfamide, chlorambucil, pipobroman, triethylenemelamine, triethylenethiophosphoramine, busulfan, lomustine, streptozocin, dacarbazine, floxuridine, cytarabine, 6-mercaptopurine, 6-thioguanine, fludarabine phosphate, oxaliplatin, folinic acid, pentostatin, vinblastine, vindesine, bleomycin, dactinomycin, daunorubicin, doxorubicin, epirubicin, idarubicin, mithramycin, deoxycoformycin, mitomycin-C, L-asparaginase, teniposide, 17α-ethinylestradiol, diethylstilbestrol, testosterone, prednisone, fluoxymesterone, dromostanolone propionate, testolactone, megestrol acetate, methyltestosterone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, vinorelbine, anastrazole, letrozole, capecitabine, reloxafine, hexamethylmelamine, bevacizumab, bexxar, velcade, zevalin, trisenox, xeloda, porfimer, erbitux, thiotepa, altretamine, trastuzumab, fulvestrant, exemestane, ifosfamide, rituximab, alemtuzumab, clofarabine, cladribine, aphidicolin, sunitinib, dasatinib, tezacitabine, triapine, trimidox, amidox, bendamustine, and ofatumumab.

### Pharmaceutical Compositions

When employed as pharmaceuticals, the compounds provided herein can be administered in the form of pharmaceutical compositions. These compositions can be prepared as described herein or elsewhere, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, (e.g., intrathecal or intraventricular, administration). Parenteral administration can be in the form of a single bolus dose, or may be, for example, by a continuous perfusion pump. In some embodiments, the compounds provided herein, or a pharmaceutically acceptable salt thereof, are suitable for parenteral administration. In some embodiments, the compounds provided herein are suitable for intravenous administration. In some embodiments, the compounds provided herein are suitable for oral administration. In some embodiments, the compounds provided herein are suitable for topical administration.

Pharmaceutical compositions and formulations for topical administration may include, but are not limited to, transdermal patches, ointments, lotions, creams, gels, drops, suppositories, sprays, liquids and powders. Conventional pharmaceutical carriers, aqueous, powder or oily bases, thickeners and the like may be necessary or desirable. In some embodiments, the pharmaceutical compositions provided herein are suitable for parenteral administration. In some embodiments, the pharmaceutical compositions provided herein are suitable for intravenous administration. In some embodiments, the pharmaceutical compositions provided herein are suitable for oral administration. In some embodiments, the pharmaceutical compositions provided herein are suitable for topical administration.

Also provided are pharmaceutical compositions which contain, as the active ingredient, a compound provided herein in combination with one or more pharmaceutically acceptable carriers (e.g. excipients). In making the pharmaceutical compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be, for example, in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof.

The active compound can be effective over a wide dosage range and is generally administered in an effective amount. It will be understood, however, that the amount of the compound actually administered will usually be determined by a physician, according to the relevant circumstances, including the condition to be treated, the chosen route of administration, the actual compound administered, the age, weight, and response of the individual subject, the severity of the subject's symptoms, and the like.

The compositions provided herein can be administered one from one or more times per day to one or more times per week; including once every other day. The skilled artisan will appreciate that certain factors can influence the dosage and timing required to effectively treat a subject, including, but not limited to, the severity of the disease or disorder, previous treatments, the general health and/or age of the subject, and other diseases present. Moreover, treatment of a subject with a therapeutically effective amount of a compound described herein can include a single treatment or a series of treatments.

Dosage, toxicity and therapeutic efficacy of the compounds provided herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds exhibiting high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1. Potential Applications of Vanadium-Based Anticancer Drugs for Intratumoral Injections

Overview

The administration of highly cytotoxic or immunomodulating drugs directly into a tumor is a method used in the clinic for late stages of cancers and in clinical trials for platinum-based drugs. A hydrophobic non-innocent Schiff base V(V) complex with a sterically hindered catecholate ligand was taken up rapidly into cancer cells and caused cell death. The synthesis was non-trivial at large scales and high purities. This class of complexes is sufficiently stable to survive briefly under physiological conditions before hydrolysis and/or redox reactions. Degradation reactions occur very rapidly for complexes with less sterically hindered catacholates.

Introduction

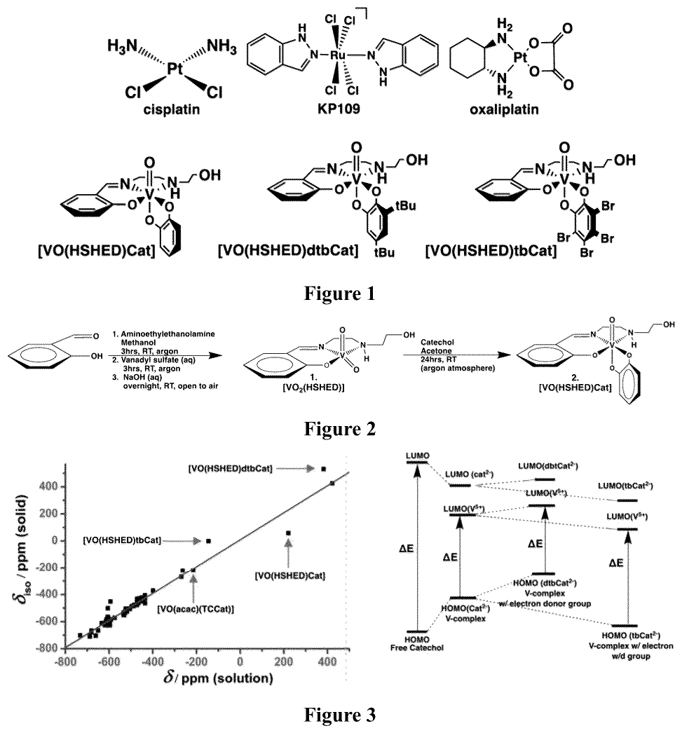

The first chemotherapeutic metal-complex, cisplatin, was approved by the FDA in 1978 and since that time, other cytotoxic metal-based chemotherapeutics have been approved, are in clinical trials, or under development. See FIG. 1. A major objective in the development of effective chemotherapeutics is increasing efficacy while reducing toxicity to healthy tissue to acceptable levels. After the approval of cisplatin, early investigations turned to the development of other metal-based drugs, including ruthenium- and titanium-based drugs. However, these drugs were cancelled during clinical trials due to nephrotoxicity, which limited dosage to ineffective levels. One strategy to reduce toxicity relies on designing metal-complexes that degrade after intratumoral injection and before encountering healthy tissue.

A major obstacle facing chemotherapeutic metal-complexes is effective movement across cell membranes. It is well established that the cell membrane is distinctly hydrophobic and interacts favorably with hydrophobic drugs. Hydrophobic non-innocent vanadium(V) complexes are one example of such a class of potential drugs that may be suitable for intratumoral injection due to their rapid uptake. These vanadium(V) complexes are designed to break down before diffusion or transport outside the tumor and into contact with healthy tissue. The breakdown products of these potential drugs are inoffensive to the human body and may have beneficial properties. In this way, such a compound acts to support healthy tissue during an otherwise toxic event. Currently, one intratumoral pharmaceutical, an oncolytic virus (T-VEC), is in clinical use for melanoma which cannot be surgically removed. Furthermore, seven platinum-based anticancer preparations are in clinical trials.

### Synthesis and Properties of Hydrophobic Non-Innocent Vanadium(V) Complexes

Metal-complexes containing redox active ligands are named non-innocent metal complexes and are widely used in catalytic reactions, sensors in biological systems, drug delivery, and water remediation. The term “non-innocent metal complex” refers to a complex where, when a redox reaction has occurred, it is not certain if the resulting charge is localized on the metal ion, or on the coordinated ligand. We first became interested in these compounds after observing their unusual electronic, and later cytotoxic, properties. With that in mind, we continued our investigations into the biological activities of these compounds, and have since synthesized, and tested, a number of these derivatives. See FIG. 1 and FIG. 2.

Among the vanadium(V) complexes we have synthesized are [VO(HSHED)cat], and the more sterically hindered derivative [VO(HSHED)dtb]. Regardless of the exact steric, electronic, or structural nature of a [VO(HSHED)cat] derivative, the synthetic scheme of these vanadium(V) complexes begins with the condensation product of the Schiff base ligand from 2-((aminomethyl)amino)ethan-1-ol and salicaldehyde under an inert atmosphere. See FIG. 2. Then, an aqueous solution of vanadyl sulfate was added to the Schiff base scaffold, followed by aqueous sodium hydroxide to give solid [VO2(HSHED)]. To an acetone solution of this solid, catechol was added to give a purple crystalline powder. FIG. 2.

Due to the profound effect of the catechol ligands upon the electronic properties of these compounds, these reactions and their corresponding products are quite distinctive and can be monitored by both ⁵¹V NMR spectroscopy and UV-vis spectrophotometry. Both methods are significantly influenced by the electronic properties of the vanadium(V) as they reflect the size of the HOMO-LUMO gap. FIG. 3. For instance, when the catechol substituent is decorated with electron-donating functional groups, the HOMO-LUMO gap is lowered and the ⁵¹V NMR is pushed downfield.

When functionalized with electron-withdrawing functional groups, the HOMO-LUMO gap is widened and the ⁵¹V NMR signal moves upfield. Similarly, the color of this class of compounds varies from the bright yellow vanadium scaffold to a non-innocent vanadium-complex that ranges from dark purple to green. Furthermore, the HOMO-LUMO gap impacts redox reactions of these complexes. While [VO(HSHED)cat] is a cytotoxic complex, it also rapidly hydrolyzes. With the substitution of two t-butyl (dtb) functional groups onto the catechol ligand, [VO(HSHED)(dtbCat)] demonstrated significantly prolonged lifetime in both water and cell culture medium as compared to [VO(HSHED)Cat]. Functionalization of catechol with electron donating di-tert-butyl groups raises both HOMO and LUMO, with a net decrease in HOMO-LUMO gap. In contrast, the tetra-brominated analogue, [VO(HSHED)tbCat], decreases both HOMO and LUMO, with a net increase in HOMO-LUMO gap. FIG. 3.

Biological Studies

To investigate if there is a relationship between hydrophobicity and V(V) complex cytotoxicity, we conducted experiments with [VO(HSHED)Cat] and [VO(HSHED)dtbCat] in a simple monolayer model membrane system. Using microemulsions (reverse micelles), we generated a self-assembled system containing H₂O/dioctyl sulfosuccinate sodium salt/organic solvent, where the organic solvent was either iso-octane or cyclohexane. Using ⁵¹V NMR and UV-vis spectroscopies to monitor the complex stability, we demonstrated that the more hydrophobic and sterically hindered [VO(HSHED)dtbCat] was stabilized by this monolayer and was slower to hydrolyze than the parent [VO(HSHED)Cat] complex. The greater cellular penetration of [VO(HSHED)dtbCat] would favor more potent anticancer properties and this was observed for human bone cancer cells (SW1353).

Cellular experiments were designed to cross-test several cancer cell lines against intact and hydrolyzed complex. Fresh [VO(HSHED)dtbCat] was tested alongside its breakdown products (aged [VO(HSHED)dtbCat]). Also tested were fresh and aged cisplatin, as reference compounds. These substances were all tested against cancerous brain (T98 g cells), breast (MDA-MB-231), pancreatic (PANC-1), lung (A549), and normal connective (HFF-1) tissues. In all cell lines, fresh [VO(HSHED)dtbCat] had the lowest IC₅₀ (highest activity) of the substances tested. Furthermore, in T98 g cells [VO(HSHED)dtbCat] was an order of magnitude more cytotoxic than its breakdown products or cisplatin. FIG. 4.

The IC₅₀ values of [VO(HSHED)dtbCat], fresh (intact) and aged (decomposed), showed promise as a candidate for intratumoral injection into brain cancers. Fresh [VO(HSHED)dtbCat] may be more effective at killing cancerous cells than cisplatin, however, the decomposition products were comparatively safe and harmless. In fact, some of these products have demonstrated neuroprotective and neurostimulating properties and may serve to support healthy tissue through both cancer and chemotherapy, however further studies are needed to understand how complex reactivity, degradation, and speciation within a biological system affects activity.

Conclusion

We describe [VO(HSHED)(dtbCat)] as a potential intratumoral drug, which exhibits rapid cellular uptake, enhanced reactivity, and non-toxic decomposition products. While some hydrophobic, sterically hindered vanadium(V) complexes perform quite well in in vitro experiments against cancer cell lines, it remains unclear if complexes with even greater still degrees of hydrophobicity and steric-hindrance will perform proportionally better. We are currently testing other complexes which we have designed with these properties. We have suggested that not only these non-innocent vanadium complexes, but also other complexes that have been investigated in clinical trials but abandoned because they did not exhibit sufficient stability to be administered by conventional methods could be excellent candidates for intratumoral administration.

### Example 2. Exploring Growth of Mycobacterium smegmatis Treated with Anticarcinogenic Vanadium Compounds

Overview

A major problem with patient treatments using anticancer compounds is accompanying bacterial infections, which makes more information on how such compounds impact bacterial growth desirable. In this Example, we investigated the growth effects of an anticancerous nontoxic Schiff base oxidovanadium(V) complex (N-(salicylideneaminato)-N0-(2-hydroxyethyl)ethane-1,2-diamine) coordinated to the 3,5-di-tert-butylcatecholato ligand on a representative bacterium, Mycobacterium smegmatis (M. smeg). We prepared the Schiff base V-complexes using the general methods described above and selected a few complexes to develop a V-complex series. Biological studies of M. smeg growth inhibition were complemented by spectroscopic studies using UV-Vis spectrophotometry and NMR spectroscopy to determine which complexes were intact under biologically relevant conditions. We specifically chose to examine (1) the growth effects of Schiff base oxidovanadium complexes coordinated to a catechol, (2) the growth effects of respective free catecholates on M. smeg, and (3) to identify complexes where the metal coordination complex was more potent than the ligand alone under biological conditions. Results from these studies showed that the observed effects of Schiff base V-catecholate complex are a combination of catechol properties including toxicity, hydrophobicity, and sterics.

Introduction

The power of metal complexes as anticancer agents, with the best examples being Pt-based compounds such as cisplatin, carboplatin, and oxaliplatin, has been demonstrated in clinical settings over several decades. In addition to Pt-based compounds, a number of other metal-based compounds have been reported to have anticancer properties including Ru-containing anticancer compounds, Cu-containing anticancer compounds, vanadium-containing compounds, and other transition metal-based compounds. Although the compounds that make it to the clinic are highly effective, patients treated with anticancer agents are very susceptible to bacterial infections. Microbial infections can be very serious; some have been reported to modulate host cell transformation and hence promote the production of carcinogenic metabolites participating in inflammation responses, to disrupt cell metabolism, and to modify genomic or epigenetic characteristics. Reducing the potential problems prompting patient evaluation and intravenous administration of broad-spectrum antibiotics is critically important and may represent a practice difference for the patient. Thus, information on the effects of anticarcinogenic compounds on various bacterial strains are of general interest.

When evaluating effects of metal complexes on bacterial cells, it is, however, important to consider that these compounds are coordination complexes and many of these compounds do not remain intact during study due to their inherent hydrolytic instability in complex biological media. In addition, the effects of the metal complex versus its ligand can be important, particularly in cases where the ligand has potent effects on the cells being studied. Here, we use a recently identified anticancer compound, a Schiff base vanadium-based catecholate complex (oxidovanadium(IV) complexed to N-(salicylideneaminato)-N0-(2-hydroxyethyl)ethane-1,2-diamine 3,5-di-tertbutylcatecholato ligand), which is significantly more potent than cisplatin in several cancer lines including glioma multiforme, an aggressive brain cancer (T98 g), lung cancer cells (A549), and pancreas cancer cells (PANC-1). Because this vanadium compound is uniquely more stable than generally observed for Schiff base V(V) catecholato complexes, this class of compounds can be used for targeted injections directly into tumors.

In this Example, we also investigate a number of related vanadium complexes (V-complexes) containing non-innocent catecholato ligands to determine whether there are identifiable relationships between effects of the metal complex on bacterial growth and complex stability in bacterial growth media. For those complexes that do not remain intact, biological effects of the resulting products including the non-innocent ligand is of considerable interest.

V-complexes, their ligands, and free metal ions have been evaluated in different bacteria, fungi, and pathogens. Although vanadate was reported to inhibit growth of Mycobacteria smegmatis (M. smeg) some time ago, we have found that cell growth of M. smeg is much more sensitive to the large compact decavanadate anion (V₁₀₋anion) than the smaller vanadate monomer (HVO₄²⁻, V₁ anion). Furthermore, replacing one of the V-atoms in the V₁₀ cluster toform a monosubstituted polyoxidovanadate affects growth inhibition with the order being V₁₀>V₉Pt>V₉Mo. The sensitivity of M. smeg to monosubstituted polyanions further motivated studies here examining cell growth effects for a number of related intact and hydrolyzed coordination complexes with non-innocent ligands in this representational bacterial system.

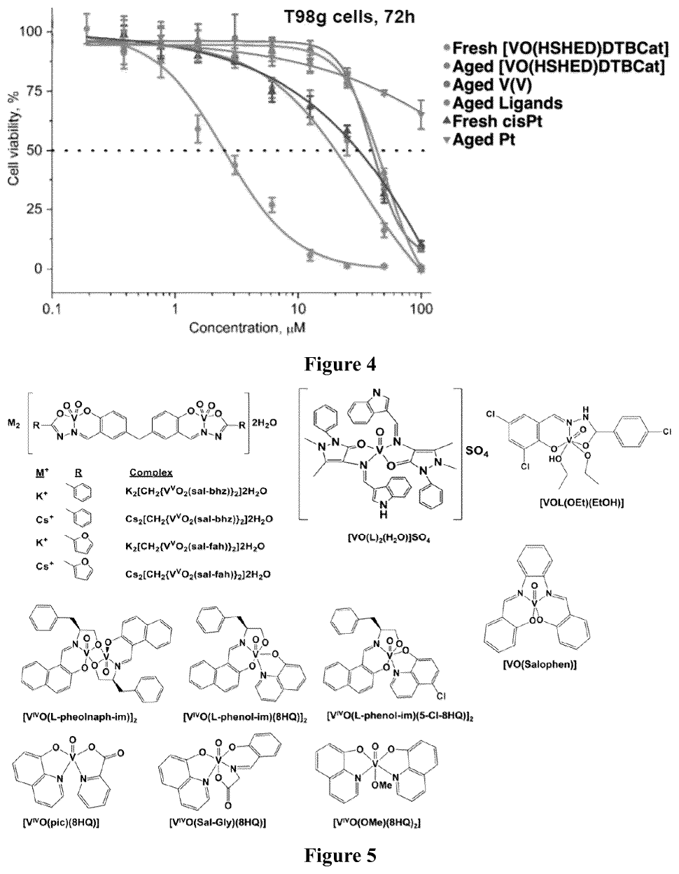

The structure of the classes of coordination complexes where both complexes and ligands have previously been investigated in bacteria are listed in FIG. 5. The compounds reported include complexes with hydrazones, Schiff bases, quinolones, and related studies with phenanthrolines and Vitamin E-hydroxylamino-Triazine ligands. Several phenanthroline complexes have been studied extensively in cancer cells; because the ligand has been shown to have variable effects depending on time of treatment, it serves as an excellent comparison for these studies carried out in bacteria. Compounds are sensitive to the oxidation state of the metal ion and the ligand as well as the nature of the coordination complexes. It is, however, important to note that both the biological and chemical systems investigated made direct comparisons difficult. Generally, complexes formed by hydrazones and Schiff bases were found to be more potent than their ligands, while compounds formed from quinolones were less potent than their respective ligands. Recently, we identified a non-toxic Schiff base oxidovanadium(V) complex (N-(salicylideneaminato)-N′-(2-hydroxyethyl)ethane-1,2-diamine) containing a hydrophobic and sterically hindered catecholate ligand (3,5-di-tert-butylcatecholato ligand) which is effective against several cancer cell types including glioma multiforme, an aggressive brain cancer (T98 g), lung cancer cells (A549), pancreas cancer cells (PANC-1), cultured human bone cancer cells, breast cancer (MDA-MB-231), and human foreskin fibroblasts (HFF-1). Several catecholate ligands have been reported to be toxic and, as a result, either more or less potent than their parent vanadium complexes. In contrast to the parent catecholate, the sterically hindered catechol 3,5-di-tert-butylcatechol in a complex is less toxic in female Swiss mice. To obtain more information on the effects of intact complexes versus ligands, we selected a group of complexes with varying stability that contained a ligand such as a catechol with the potential to inhibit M. smeg growth.

Results

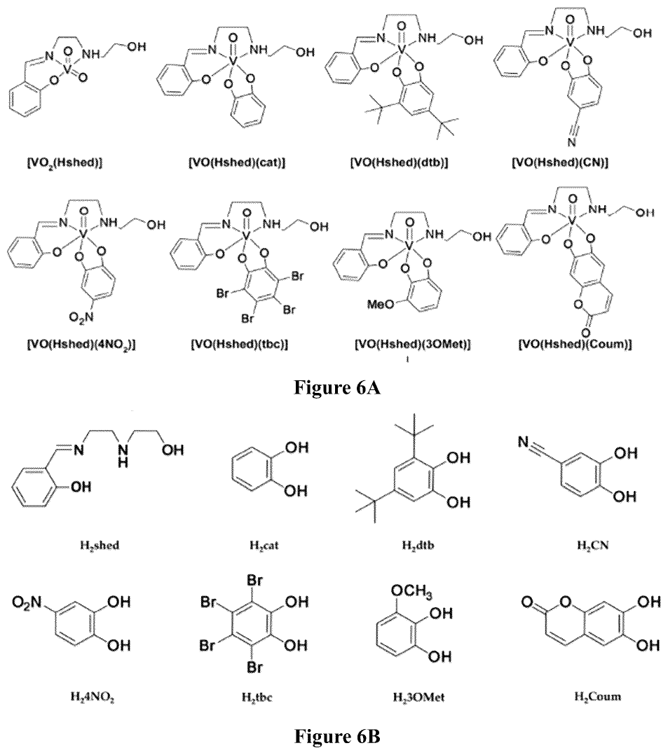

Compound Design. We first investigated the effects of a series of Schiff base vanadium(V) catecholato compounds related to the oxidovanadium(V) complex (N-(salicylideneaminato)-N′-(2-hydroxyethyl)ethane-1,2-diamine) which exhibits potent anticancer properties and is shown in FIGS. 6A-6B. The varying effects of these compounds on M. smeg growth of each vanadium complexes were compared to the effects of its free ligands. The complexes selected for investigation contained non-innocent ligands or formed complexes with different electronic properties. We selected the V-scaffold [VO2(Hshed)], the most potent anti-cancer agent [VO(Hshed)(dtb)], and control compounds with different electronic properties of the parent [VO(Hshed)(cat)], and two compounds with electronic withdrawing ligands [VO(Hshed)(tbc)] and [VO(Hshed)(4NO₂)]. In addition, we designed here three new complexes, the hydrophobic [VO(Hshed)(Coum)], and a complex with an electron withdrawing ligand —CN, [VO(Hshed)(CN)], and a complex with an electron donating ligand OMet, [VO(Hshed)(3OMet)]. In selecting this series of compounds, we expected varying degrees of complex stability in H₂O and in 7H9 media. The stability and speciation of the complexes was investigated using NMR and UV-Vis spectroscopy.

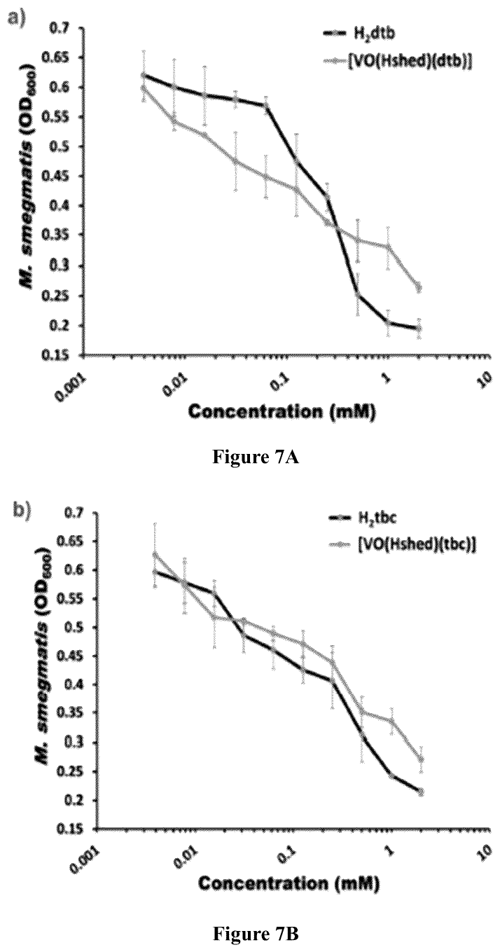

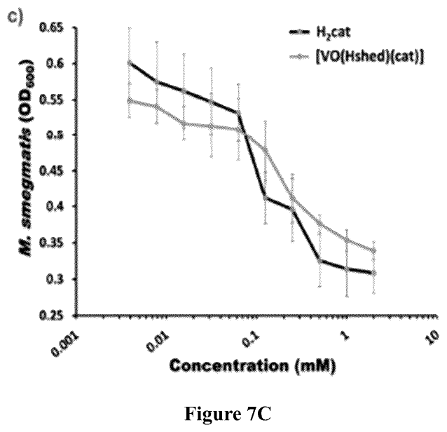

Growth Inhibition of Vanadium Complexes and Free Ligands Determined Using the Minimum Inhibitory Concentration. The biological activity of V-complexes and free ligands was investigated in terms of their respective antibacterial effects on the growth of M. smeg by measuring IC₅₀ values. The growth inhibition data of M. smeg treated with three different V-complexes and free catechol ligands are shown in FIGS. 7A-7C including (FIG. 7A) [VO(Hshed)(dtb)] and H₂dtb, (FIG. 7B) [VO(Hshed)(tbc)] and H₂tbc, and (FIG. 7C) [VO(Hshed)(cat)] and H₂cat. The IC₅₀ values and the growth inhibition curves for the effects of the additional V-complexes, their free catechol, and the Schiff base V-Hshed scaffold on the bacterial growth were also determined. We used DMSO to dissolve hydrophobic compounds into solution for preparation of stock solutions. For studies with M. smeg, the stock solution was diluted with cell medium to prepare a solution containing 10% DMSO which was then diluted via serial dilution to lower V-complex concentrations containing less DMSO. To clarify the role of DMSO in cell studies, cell growth inhibition up to 20% DMSO was evaluated and was significant. The effects of DMSO alone on the growth of M. smeg was also evaluated. When cells were treated with 10% DMSO alone, the highest DMSO concentration used in our studies of V-compound effects on M. smeg growth, growth inhibition was reduced to values that were still significant but markedly lower than seen for 20% DMSO. For compounds that were present in serially diluted medium and contained less than 10% DMSO, there were insignificant effects of DMSO on M. smeg growth indicating that the IC₅₀ values for all the complexes and catechols tested were due to compound effects and not to the presence of DMSO (Table 1).

TABLE 1
Antibacterial activity (IC₅₀) of Schiff base vanadium
complexes, the Schiff base [VO2Hshed] scaffold,
and free catechols against M. smeg at 0.250 mM.

| | | The Effect of Complexes |
|---|---|---|
| Tested Compounds | IC₅₀ (μM) | Compared to the Free Catechol |
| [VO(Hshed)(dtb)] | 119 ± 0.09 | 2x more potent in terms |
| H₂dtb | 241 ± 0.07 | of concentration |
| [VO(Hshed)(Coum)] | 370 ± 0.13 | 1.4x more potent in terms |
| H₂Coum | 506 ± 0.28 | of concentration |
| [VO(Hshed)(4NO₂)] | 472 ± 0.02 | 1.3x more potent in terms |
| H₂4NO₂ | 610 ± 0.05 | of concentration |
| [VO(Hshed)(tbc)] | 449 ± 0.22 | Same effect in terms |
| H₂tbc | 416 ± 0.19 | of concentration |
| [VO(Hshed)(cat)] | 518 ± 0.29 | 5x less potent in terms |
| H₂cat | 109 ± 0.05 | of concentration |
| [VO(Hshed)(3OMet)] | 555 ± 0.23 | 2x less potent in terms |
| H₂3OMet | 262 ± 0.15 | of concentration |
| [VO(Hshed)(CN)] | 608 ± 0.07 | 1.4x less potent in terms |
| H₂CN | 447 ± 0.04 | of concentration |
| [V(O)₂(Hshed)] | 516 ± 0.31 | Schiff base V-complex scaffold |

The IC₅₀ values of V-complexes and their free ligands are shown in Table 1. Although all of the V-complexes and their free ligands were comparatively weak growth inhibitors of M smeg, differences in growth inhibition were observed between the V-complex and its free ligand. The IC₅₀ of [VO(Hshed)(dtb)], [VO(Hshed)(Coum)], and [VO(Hshed)(4NO₂)] indicated that these compounds were more potent in inhibiting bacterial growth with an IC₅₀ of 119, 370, and 472 μM, respectively. In complexes, their ligands showed 2-, 1.4-, and 1.3-fold more growth inhibition than did the free ligands. [VO(Hshed)(cat)], [VO(Hshed)(3OMet)], and [VO(Hshed)(CN)] showed less inhibitory activity against the bacterial growth with an IC₅₀ of 518, 555, and 608 μM, respectively, when compared with their free ligands. Finally, the IC₅₀ of [VO(Hshed)(tbc)] had a similar activity, an IC₅₀ of 449 μM, when compared with its free ligand with an IC₅₀ of 416 μM.

Solution Chemistry and Stability of Vanadium-Catechol Complexes in Reference Solutions and in Growth Media as Monitored by UV-Vis Spectroscopy. To further investigate whether the observed effects of compounds or their ligands on M. smeg growth in cell culture were related to complex stability, spectroscopic studies were undertaken. We examined the V-compounds in three different solutions. (1) Simple H₂O-DMSO containing no other additives, (2) 7H9-DMSO media with nutrients for M. smeg growth but without other additives, and (3) 7H9 media in which M. smeg had grown and the media contained bovine serum albumin (BSA), oleic acid, and dextrose and from which cells had then been removed (referred to as “supernatant”). UV-Vis studies and subsequent NMR spectroscopic studies would show which compounds remained intact and for how long. The stability of the V-compounds was not expected to be the same in these three solutions nor was the response of each compound to these conditions likely to be the same.

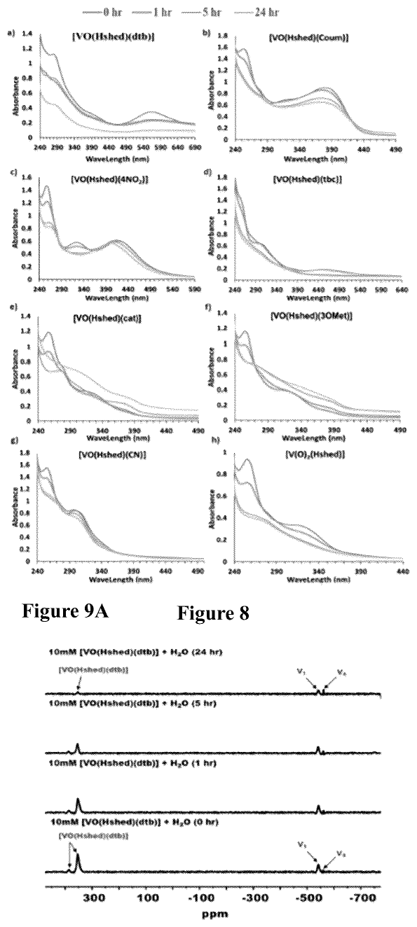

First, the V-catechol complexes with different ligands were analyzed using UV-Vis spectroscopy to explore the speciation and the stability of these complexes in both in H₂O:DMSO and in 7H9:DMSO at 37° C. Here, 7H9 medium was used in M. smeg growth studies which made its effects on compound speciation and stability of particular interest. The samples were initially dissolved in DMSO due to the low solubility of the compounds and their weak growth inhibition. Hence, the stabilities of the compounds described differ here from studies of compound stability reported in cancer cell lines studies where the compound concentration and temperature were lower and there was less DMSO present in the original stock solution. To conserve space, the spectra of the complexes shown in FIG. 8 were recorded in 7H9:DMSO at 37° C., while the UV-Vis spectra of complexes in H2O:DMSO and in supernatant:DMSO were collected but are not shown. Because of the dilution of the original compound stock solution to 0.250 mM V-compound with H₂O or 7H9 media, the DMSO content decreased from 50% in the stock solution to about 6% for the UV-Vis studies.

UV-Vis spectroscopy can be used to show time-dependent changes in complex structure and hence complex stability. The spectra of complexes and of free ligands at time zero (t=0) in H₂O:DMSO and in 7H9:DMSO were recorded as reference for the spectra in 7H9:DMSO and are summarized in Table 2. For comparison, the stability of the Schiff base oxido-vanadium(V) complex scaffold, [VO2(Hshed)], which provided the basic framework for all the complexes in this series, was also evaluated. The main absorption peaks were observed at wavelengths at 255 and 325 nm in aqueous solution and were similarly observed in 7H9 media. The addition of catechol ligands, as expected, added additional signals to the complexes with shifts that depended on the electronic properties of the catechol.

TABLE 2
UV-Vis parameters observed from the Schiff base V-catecholate complexes
and free catecholate ligands at t = 0 h with 0.250 mM V-compound at 37°
C. a in the mixed solutions (94:6 H₂O:DMSO or 94:6 7H9 medium:DMSO).

| | Complex (H₂O/DMSO) | Complex (7H9/DMSO) |
|---|---|---|
| | Absorbance (nm)/ [symbol] | Absorbance (nm)/ [symbol] |
| Complex | (×10³ M⁻¹) | (×10³ M⁻¹) |
| [VO(Hshed)(dtb)] | (280, 1.161, 4.6); | (280, 1.123, 4.5), |
| | (565, 0.396, 1.6) | (565, 0.347, 1.4) |
| [VO(Hshed)(Coum)] | (255, 1.344, 5.3); | (255, 1.562, 6.2); |
| | (275, 0.727, 2.9); | (280, 0.836, 3.3); |
| | (370, 0.870, 3.5) | (325, 0.678, 2.7); |
| | | (380, 0.901, 3.6) |
| [VO(Hshed)(4NO₂)] | (255, 1.299, 5.2); | (260, 1.455, 5.8); |
| | (325, 0.533, 2.1); | (325, 0.583, 2.3); |
| | (405, 0.552, 2.2) | (420, 0.568, 2.3) |
| [VO(Hshed)(tbc)] | (255, 0.854, 3.4); | (305, 0.613, 2.5) |
| | (310, 0.284, 1.1) | |
| [VO(Hshed)(cat)] | (255, 0.951, 3.8); | (255, 1.183, 4.7); |
| | (280, 0.579, 2.3); | (280, 0.743, 3.0); |
| | (375, 0.117, 0.47) | (325, 0.349, 1.4); |
| | | (380, 0.138, 0.55) |
| [VO(Hshed)(3OMet)] | (255, 0.831, 3.3); | (255, 1.162, 4.6); |
| | (280, 0.408, 1.6) | (325, 0.441, 1.8); |
| | | (385, 0.156, 0.62) |
| ([VO(Hshed)(CN)] | (255, 1.473, 5.9); | (255, 1.55, 6.2); |
| | (300, 0.666, 2.7) | (300, 0.789, 3.2) |
| [V(O)₂(Hshed)] | (255, 0.682, 2.7); | (255, 0.937, 3.7); |
| | (325, 0.200, 0.80) | (325, 0.312, 1.2) |
| [H₂dtb] ligand | (280, 0.218, 0.90) | (280, 0.513, 2.1) |
| [H₂Coum] ligand | (255, 0.371, 1.5); | (295, 0.639, 2.6); |
| | (300, 0.524, 2.1); | (350, 0.939, 3.8) |
| | (345, 0.972, 3.9) | |
| [H₂4NO₂] ligand | (310, 0.652, 2.6); | (265, 0.726, 2.9); |
| | (350, 0.784, 3.1) | (320, 0.575, 2.3); |
| | | (430, 0.767, 3.1) |
| [H₂tbc] ligand | (275, 0.241, 1.0); | (255, 0.941, 3.8); |
| | (300, 0.203, 0.81) | (300, 0.574, 2.3) |
| [H₂cat] ligand | (275, 0.364, 1.5) | (275, 0.642, 2.6) |
| [H₂3OMet] ligand | (270, 0.137, 0.50) | (270, 0.417, 1.7) |
| [H₂CN] ligand | (250, 1.172, 4.7); | (250, 1.293, 5.2); |
| | (290, 0.453, 1.8) | (290, 0.792, 3.2) |

^{a} Prepared from 10 mM V-complex stock solutions (50:50 H₂O:DMSO) and 50:50 7H9:DMSO. [symbol]
indicates data missing or illegible when filed

The UV-Vis spectra of [VO(Hshed)(dtb)] exhibited two absorption bands at 280 nm (sh) and 565 nm (m) in water and two major signals at 280 and 565 nm in 7H9:DMSO. Both the signal for the free H₂dtb catechol ligand at 280 nm and the signal at 565 nm decreased at the 1 h time point and were gone at the 5 h timepoint. This indicated that the compound was present initially in 7H9 media but, as shown in FIG. 8, began to hydrolyze. However, there are some differences in the spectra. For [VO(Hshed)(4NO₂)], [VO(Hshed)(tbc)], and [VO(Hshed)(CN)], there is a signal around 300-330 nm suggesting some isomer formation for these V-compounds. The data at the zero-time point were summarized for all the complexes (nm, absorbance, and extinction coefficient) and are listed in Table 2.

The UV-Vis spectra of [VO(Hshed)Coum] had main absorption peaks at wavelengths of 255 nm (m), 280 nm (sh), and 385 nm (m) in aqueous solution and, in 7H9 media, an extra peak of 325 nm (sh). This additional peak in 7H9 media indicated the formation of an additional species. The decrease of the two major species as a function of time suggested that this complex is present in solution for some time before hydrolyzing. [VO(Hshed)(4NO₂)] exhibited three major signals at 255, 325, and 405 nm. During the first hour the signal at 255 and 325 nm decreased, whereas the signal at 405 nm remained at the same intensity with a small change in the location of its maximum.

The spectra for the other complexes examined indicated that the complexes remained intact briefly in 7H9 media (FIG. 8) or H₂O:DMSO. However, between 1 and 5 h some remained intact for the first hour of the experiment when present at 6% DMSO concentrations and higher temperature, which showed enhanced stabilities compared to previous studies. In addition, when compounds were examined in 7H9 media, a 280 nm peak appeared that was due to formation of free catechol.

Previously, M. smeg was found to excrete a material that was able to catalyze the hydrolysis of V₁₀. For this reason, we also examined the effects of the V-compounds in the supernatant of the 7H9 media in which M. smeg had grown. Analogous experiments were conducted in this Example to determine if there was a difference from the results for media alone listed in Table 3. In this series, 0.250 mM V compounds were added to the supernatant obtained from 7H9 media after M. smeg growth. Data showing the stability of compounds after incubation in supernatant was also collected. These data show that there was no significant change in speciation in supernatant obtained after M. smeg culture when compared to data obtained using fresh media as shown in FIG. 8. However, the signal at 280 nm now indicates the presence of protein, primarily in the form of bovine serum albumin, although some formation of free catechol cannot be ruled out since this also yields an absorbance at 280 nm.

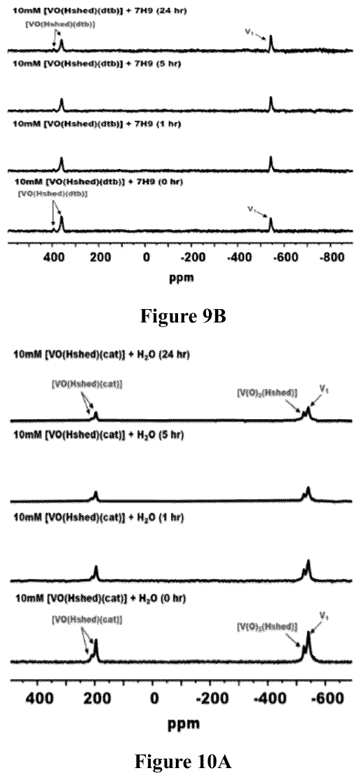

Solution Chemistry and Stability of Vanadium-Catechol Complexes in Reference Solution and in Cell Growth Media. The stability of vanadium complexes was also investigated at different times using ⁵¹V NMR spectroscopy in DMSO:H₂O, DMSO:7H9 medium, and supernatants obtained following M. smeg culture. The representative ⁵¹V NMR spectra of 10 mM [VO(Hshed)(dtb)], [VO(Hshed)(cat)], and [V(O)₂(Hshed)] in the mixture solution of H₂O, DMSO (50:50 H₂O:DMSO), and in 7H9 (50:50, H₂O:media) are shown in FIGS. 9A-9B, 10A-10B, and 11A-11B, respectively. The NMR spectra of 10 mM [VO(Hshed)(dtb)], [VO(Hshed)(cat)], and [V(O)₂(Hshed)] in the mixture with supernatants were also measured. The remaining data for the additional complexes are listed in Table 3.

TABLE 3
Species observed for 10 mM Schiff base vanadium-catecholate
complexes in the mixture solution (50:50 H₂O:DMSO)
and 50:50 7H9 growth medium:DMSO at t = 0 h.

| [symbol] | [symbol] | [symbol] | [symbol] | [symbol] | [symbol] |
|---|---|---|---|---|---|
| [symbol] | [symbol] | — | — | [symbol] | [symbol] |
| [symbol] | — | [symbol] | — | [symbol] | — |
| [symbol] | [symbol] | — | [symbol] | [symbol] | |
| [symbol] | — | — | — | [symbol] | — |
| [symbol] | [symbol] | — | [symbol] | [symbol] | — |
| [symbol] | — | [symbol] | [symbol] | [symbol] | — |
| [symbol] | [symbol] | | [symbol] | [symbol] | [symbol] |
| [symbol] | — | [symbol] | — | [symbol] | — |
| [symbol] | — | — | [symbol] | [symbol] | [symbol] |
| [symbol] | — | — | [symbol] | [symbol] | [symbol] |
| [symbol] | [symbol] | — | — | [symbol] | |
| [symbol] | — | — | [symbol] | [symbol] | [symbol] |
| [symbol] | [symbol] | — | [symbol] | [symbol] | |
| [symbol] | | [symbol] | — | [symbol] | — |
| [symbol] | [symbol] | — | [symbol] | [symbol] | — |
| [symbol] | — | — | — | [symbol] | — |

[symbol]

indicates data missing or illegible when filed

⁵¹V NMR spectra were obtained using 10 mM of [VO(Hshed)(dtb)] in 50:50 H₂O:DMSO. Subsequently, spectra were recorded to determine how much complex remained intact at different time points when the complex was in 50:50 H₂O:DMSO or in 50:50 7H9:DMSO media (FIGS. 9A-9B). The spectra of [VO(Hshed)(dtb)] at time=0 (t=0) showed two peak signals assigned to [VO(Hshed)(dtb)] at δ=387 and 353 ppm, in addition to the presence of the other two signals assigned to V₁ at δ=−541 ppm and vanadate tetramer (V₄O₁₂⁴⁻) V₄ at δ=−557 ppm. The percentage of each species measured at the initial time point is summarized in Table 3.

Over a 24 h time period, the decrease in the [VO(Hshed)(dtb)] complex in H₂O was monitored by ⁵¹V NMR spectroscopy and an increase in V₁, and V₄ was observed (FIG. 9A). The ⁵¹V NMR spectra of [VO(Hshed)(dtb)] in 7H9 was also recorded as a function of time. The spectrum recorded at the initial time (t=0 h) showed two complex signals at δ (ppm)=394 and 359 (m), in addition to one signal attributed to V₁ at δ=−544 ppm. Since only small changes were observed in the spectra from 0 h, the complex is stable in the 7H9 medium (FIG. 9B). The observation of the [VO(Hshed)(dtb)] complex in the growth assay is consistent with at least some of the observed growth effects that can be attributed to the intact [VO(Hshed)(dtb)] complex.

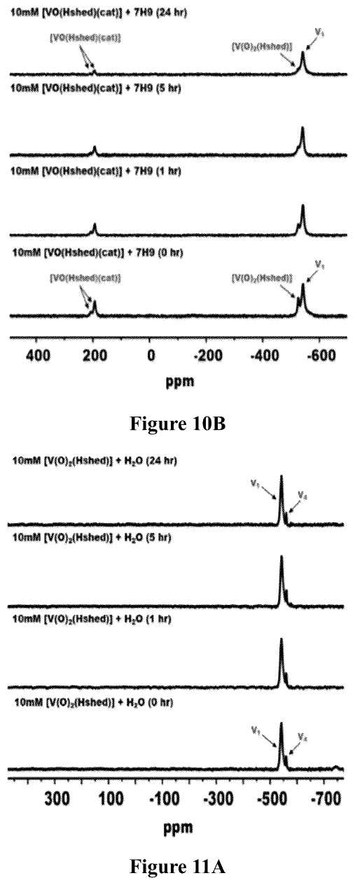

Monitoring the [VO(Hshed)(cat)] complex over a 24 h time period showed a decrease of the complex as a function of time in H₂O (FIG. 10A). The spectrum recorded at t=0 h showed two complex signals at δ (ppm)=196 and 206 (m) in addition to two signals attributed to [VO₂(Hshed)] at δ=−524 ppm and V₁ at δ=−544 ppm. Similarly, the ⁵¹V NMR spectra of [VO(Hshed)(cat)] in 7H9 were carried out as a function of time and the two signals for the complex were also found to slowly hydrolyze in 7H9 medium (FIG. 10B) to form [VO₂(Hshed)] and V₁. These spectra showed that [VO(Hshed)(cat)] complex existed in the 7H9 growth medium, suggesting that some of the observed growth inhibition can be attributed to the intact [VO(Hshed)(cat)] complex.

Studies were carried out with the other complexes and these results are summarized in Table 3. For the [VO(Hshed)(Coum)] complex, a significant amount of intact complex and [VO₂(Hshed)] was observed in H₂O:DMSO solution in addition to V₁ hydrolysis product. However, all of the complex was hydrolyzed in 7H9 media before the ⁵¹V NMR spectrum was recorded. For the [VO(Hshed)(4NO₂)] complex, a trace level of complex and an intermediate species are observed in addition to a 1:5 ratio of [VO2(Hshed)] scaffold and V₁ hydrolysis product in H₂O:DMSO. However, all complexes were hydrolyzed in 7H9 medium:DMSO before the ⁵¹V NMR spectrum was recorded. For the [VO(Hshed)(CN)], a 1:3 ratio of [VO2(Hshed)] scaffold and V₁ hydrolysis product were observed in H₂O:DMSO. However, all complexes were hydrolyzed in 7H9 medium:DMSO solution before the ⁵¹V NMR spectrum was recorded. For the [VO(Hshed)(tbc)] complex, the spectra were very noisy and only trace levels of signals were observed in addition to the major peak for V₁. Since this compound was investigated at the same concentrations as the other complexes, the noisy spectrum is presumably reflecting that this compound not only hydrolyzed but underwent redox chemistry at the 37° C. temperature in both H₂O:DMSO and the 7H9 medium:DMSO solutions. Combined, these results show that some of these compounds are more stable than reported previously at lower temperatures and DMSO concentrations, but that several of the compounds remain unstable in 7H9 growth medium:DMSO and only hydrolyzed compounds are observed by the time the ⁵¹V NMR spectra were recorded.

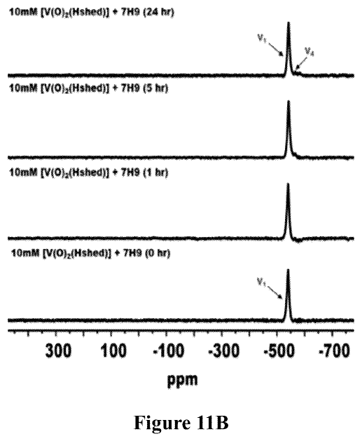

In FIGS. 11A-11B, NMR spectra are shown for the [VO(Hshed)] scaffold over a 24 h time period. These results indicate that the scaffold complex was unstable and immediately hydrolyzed to form V₁ within 1 h in both H₂O:DMSO and 7H9:DMSO.

Select studies were carried out with the [VO(Hshed)(3OMet)] complex and the [VO(Hshed)(CN)] complex. Both hydrolyzed rapidly with little intact complex left in the solution by the time the spectra were run. This information is summarized in Table 3. These data show higher stability than reported previously in pure aqueous solution or in media used to grow cancer cells.

### DISCUSSION

The results shown in Table 1 demonstrate that complexes with reported anticancer activity have limited growth effects on M. smeg. However, as shown in Table 1, some complexes are more biologically active than their catecholate ligand, while, for other complexes, the free catecholates are more potent growth inhibitors of M. smeg. Therefore, we were successful in demonstrating that within a class of compounds, biological activity can be based on subtle variations in the ligand coordinated to the vanadium.

As described above, most of the reported investigations have focused on compounds where the observed V-complex is more potent than the free ligand or metal ion. However, the quinolones and phenanthroline complexes deviate from these general observations because the free ligand is the most effective bioactive component of the complex. It should be noted, however, that most of these V-quinolone complexes are difficult to evaluate because the complexes are hydrophobic and, because of their hydrophobicity, their biological activities were not evaluated under the same conditions used in aqueous assays. A careful analysis has been reported for the effects of a range of different vanadium-complexes with phenanthroline ligands in different cancer cell lines. These studies show that bioprocessing occurs after administration of these complexes and that their hydrolysis products are important for complex activity. Importantly, time-dependent responses were also observed over 72 h and low concentrations of the ligand were found to be more effective than the complex. Speciation is particularly important when the ligand is, by itself, a potent agent. It is also important that interactions with cellular components, including proteins, be considered. Such interactions can affect delivery and uptake of both the ligand and the complex and, because of time-dependent effects, potentially produce more potent species with longer incubations. These studies also underscore the importance of complex bioprocessing and the need for studies exploring effects of speciation as well as investigation of effects of both ligands and complexes by metabolites as well as a range of proteins. Hence, we were interested in evaluating a system in which speciation and the biological effects could be determined for both the complex and free ligand.

In this Example, we characterized the effects of the Schiff base V-complex scaffold as well as the catecholate ligand examined in M. smeg growth assays. Limited effects of the Schiff base V-complex scaffold were observed, while the catecholate ligand showed more varied growth inhibitory activity. Due to the limited water solubility of these complexes, the speciation studies were undertaken in the presence of higher concentrations of DMSO which was used to solubilize the compound for addition to the cell culture at the high concentrations in this Example. Because the DMSO concentration was higher than those used previously, the complexes were more stable than reported previously except for the [VO(Hshed)(tbc)] complex that seems to be redox active when dissolved. Temperature may also contribute to compound stability; accompanying speciation studies conducted here used the same temperature from cell studies, whereas previous studies were conducted at ambient temperature. These subtle differences in conditions may explain changes in the observed stabilities of the compounds and are a reminder of the importance of investigating the activity of the ligands used in the complexes under consideration of conditions similar to corresponding biological studies.

The growth effects of the complexes were according to the following order: [VO(Hshed)(dtb)]>[VO(Hshed)(Coum)]>[VO(Hshed)(tbc)]>[VO(Hshed)(4NO₂)]>[VO(Hshed)(cat)]=[V(O)₂(Hshed)]>[VO(Hshed)(3OMet)]>[VO(Hshed)(CN)]. We anticipate that the ligand potency as a growth inhibitor is important to the overall properties of the complex as a growth inhibitor and thus determined the order of the ligand potencies. The order is H₂cat>H₂dtb>H₂3OMet>H₂tbc>H₂CN>H₂Coum>H₂4NO₂. Surprisingly, we found that H2cat was the most growth inhibiting catechol followed by H₂dtb and H₂3OMet. When examining the effects of the Schiff base V-complexes, only the [VO(Hshed)(dtb)] complex was found to be a potent growth inhibitor, while [VO(Hshed)(cat)] and [VO(Hshed)(3OMet)] were among the least potent complexes. These result suggests that the potency of the ligand is not one of the factors that is critical for effects by V-complexes. Hence, not only was the effect of the free catechol overcome by complex formation, but different properties were found to be important to the biological effects of these Schiff base V-complexes. It is possible that the electronic properties of the catechols are important. However, the anticipated electronic effects of substituents were H₂3OMet>H₂dtb>H2cat>H2tbc>H₂CN>H₂4NO₂, while the effects of the H2Coum ligand were less predictable. Electronic effects alone cannot explain the observed effects, although they may be contributing, at least in part, to the overall activity of the complex. We have investigated the electronic effects of the complexes elsewhere but in general found that the complexes that readily undergo redox chemistry are not very stable. These studies confirm the expectation that the electronic effects on the catechol ligand were not the sole factor explaining the effects of the Schiff base V-complexes or showing a pattern explaining the properties of these complexes.

Complex hydrophobicity and the stability of the Schiff base V-complex may be important to complex activity. Hence, it is important to know whether the complex remains intact when assessing its observed activities in growth effects. In this Example, we chose a system in which the activities of the complexes, although weak, were compared and differences could be identified. The two most stable and hydrophobic complexes were the [VO(Hshed)(dtb)] and [VO(Hshed)(Coum)] complexes and these complexes were also found to be the most effective inhibitors of M. smeg growth. This result suggests that stability and hydrophobicity of the complexes should be important factors when predicting compound effects on cell growth.

Perhaps the most surprising result in this series of work here is the fact that the H₂cat ligand is the catechol exhibiting the highest growth inhibitory activity. Both the H₂dtb ligand and the H₂Coum ligand are hydrophobic and hence are readily taken up by cells. The H₂dtb ligand is the most sterically hindered catechol. Sterics are likely to be important for complex formation because the H₂dtb ligand protects the V-atom from hydrolysis, as suggested by the complexes' greater stability. From these results, we conclude that both hydrophobicity and steric hindrance are important factors for observed growth inhibition effects on M. smeg but that the catecholato ligand toxicity is not necessarily related to this growth effect, particularly if the complex is, at least in part, stable as was observed under the conditions of these studies.

Experimental Procedures
Materials

Cell Culture Materials. The Middlebrook 7H9 Broth medium (Ref. no. 271310; Difco™)) was obtained from BD Biosciences (San Jose, CA, USA) and autoclaved before use. The medium was supplemented with oleic acid (0.6% v/v; Sigma-Aldrich (St. Louis, MO, USA), 5 mg mL⁻¹ albumin (VWR), 2 mg mL⁻¹ dextrose (Sigma-Aldrich), and 10% Tyloxapol (Chem-Impex Int'l Inc (Wood Dale, IL, USA)). Mycobacterium smegmatis mc² 155 (M. smeg) has been maintained in our laboratory for some time.

Chemicals for Synthesis and Spectroscopic Studies. Catechol ligands were purchased from Sigma-Aldrich. Dimethyl sulfoxide (DMSO, 99.9%) was purchased from Sigma-Aldrich and deuterium oxide (D₂O, 99.9%) was purchased from Cambridge Isotope Laboratories (Tewksbury, MA, USA). Reagents were used as received without further purification.

Eight Schiff base vanadium-catecholato coordination complexes were selected and prepared using procedures known in the art. DMSO was used for solubilizing the compounds for NMR spectroscopic studies and cell culture studies. Infrared spectra were obtained using a Bruker Tensor II FT-IR spectrometer (Billerica, MA, USA) in attenuated total reflectance mode with a 4 cm⁻¹ resolution.
Methods

Cell Culture and Growth Conditions. M. smeg was cultured in Middlebrook 7H9 medium (Ref. no. 271310; Difco) supplemented with 10% OAD (oleic acid, albumin, dextrose), 0.2% (v/v) glycerol, and 10% Tyloxapol, and incubated with shaking for 24 h at 37° C. M. smeg was grown to a mid-logarithmic growth phase assessed via spectrophotometry and occurred at an optical density of 0.6 at 600 nm (OD₆₀₀). In some experiments where M. smeg was treated with vanadium complexes, the cell culture supernatant was collected for ⁵¹V NMR studies.

Minimum Inhibitory Concentration (MIC) Measurements. The inhibitory activity of the tested compounds was determined by measuring the lowest concentration that caused bacterial growth inhibition of 50% (IC₅₀). M. smeg was grown to the mid-log phase of 0.6-0.8 at OD₆₀₀. The final concentrations of the compounds used in these studies were obtained using serial dilutions to a final concentration range of 2 to 0.0039 mM and placed with M. smeg in 7H9 media into a 96-well plate. Plates were incubated for 24 h at 37° C. A Bio-Rad Benchmark Reader (Bio-Rad Laboratories: Hercules, CA, USA) was used to check the cell viability at OD₆₀₀ at appropriate times. IC₅₀ was determined to be the lowest concentration of complexes that inhibited M. smeg growth by 50%.

Statistical Analysis. For each treatment, data were represented as the mean and standard deviation of the IC₅₀ from triplicate measurements. The IC₅₀ was calculated using GraFit 5 data analysis software (version 5.0.13).
Chemistry

Synthesis of Schiff Base Vanadium-Catecholato Coordination Complexes. The complexes were prepared from [V(O)₂(Hshed)] through a condensation reaction. See FIG. 6A for the structures of the compounds and their abbreviations. After preparation of the [VO2(Hshed)] precursor, the catechol was added to generate the complexes used in this Example: [VO(Hshed)(cat)]; [VO(Hshed)(dtb)]; [VO(Hshed)(4NO₂)]; [VO(Hshed)(tbc)]; [VO(Hshed)(CN)]; [VO(Hshed)(3OMet)]; and [VO(Hshed)(Coum)].

Synthesis of [VO(Hshed)(CN)]. To a 250 mL round bottom Schlenk flask, acetone (100 mL) was added, which was then degassed with argon. [VO2(Hshed)] (0.290 g, 1.00 mmol) was added to the degassed acetone followed by 3,4-dihydroxybenzonitrile (0.135 g, 1.00 mmol). A deep purple solution resulted after approximately 20 s. The solution was stirred overnight under argon. The reaction mixture was vacuum filtered and then concentrated to dryness under reduced pressure at room temperature. The purple residue was dissolved in a minimal amount of acetone and then n-hexane (100 mL) was added. The solution was allowed to stand in a −20° C. freezer overnight. The dark microcrystalline precipitate was vacuum filtered, washed with cold n-hexane (<0° C., 2×25 mL), and dried under vacuum for 3 days to yield 0.341 g (0.838 mmol, 84%) purple solid. ⁵¹V NMR (in CD₃CN): −138 ppm, −113 ppm (minor isomer). ¹H NMR (400 MHz, CD₃CN): δ 8.74 (s, 1H), 7.64-7.62 (dd, 1H), 7.59-7.54 (dt, 1H), 7.17-7.13 (m, 1H), 7.00-6.96 (dt, 1H), 6.91-6.88 (m, 1H) 6.83-6.80 (d, 1H), 4.23-4.18 (dd, 1H), 4.10-4.03 (dt, 1H), 3.79-3.72 (m, 1H), 3.69-3.63 (m, 1H), 3.59-3.49 (m, 2H), 3.39-3.31 (m, 1H), 3.04-2.90 (m, 2H), 2.81-2.78 (t, 1H). IR (ATR, 4 cm⁻¹): 3049 (O—H), 2218 (C≡N), 1712 (Aromatic), 953 (V═O). Calc for C₁₈H₁₈N₃O₅V: C, 53.08; H, 4.45; N, 10.32. Found: C, 52.52; H, 4.33; N, 9.96. UV-Vis peaks (in DMSO): 280 and 305 nm.

Synthesis of [VO(Hshed)(3OMet)]. To a 250 mL round bottom Schlenk flask, acetone (100 mL) was added, which was then degassed with argon. [VO2(Hshed)] (0.290 g, 1.00 mmol) was added to the degassed acetone followed by 3-methoxy catechol (0.140 g, 1.00 mmol). A deep purple solution resulted after approximately 20 s. The solution was stirred overnight under argon. The reaction mixture was vacuum filtered and then concentrated to dryness under reduced pressure at room temperature. The purple residue was dissolved in a minimal amount of acetone and then n-hexane (100 mL) was added. The solution was allowed to stand in a −20° C. freezer overnight. The dark microcrystalline precipitate was vacuum filtered, washed with cold n-hexane (<0° C., 2×25 mL), and dried under vacuum for 3 days to yield 0.280 g (0.680 mmol, 68%) purple solid. ⁵¹V NMR in CD₃CN: 288 ppm. ¹H NMR (400 MHz, CD₃CN): δ 8.63 (s, 1H), 7.51-7.48 (t, 1H), 6.87-6.83 (t, 1H), 6.76-6.66 (m, 2H), 6.34-6.32 (d, 1H), 6.07-6.05 (d, 1H), 5.99-5.97 (d, 1H), 3.90-3.87 (m, 3H), 3.84-3.76 (m, 2H), 3.66-3.61 (m, 2H), 3.49-3.39 (m, 1H), 3.38-3.31 (m, 1H), 2.99-2.91 (m, 1H), 2.84-2.81 (t, 1H). IR (ATR, 4 cm⁻¹): 1636 (Aromatic), 1304, 1267, 1108 (C—O), 954 (V═O). Calc for C₁₈H₂₁N₂O₆V: C, 52.43; H, 5.13; N, 6.79. Found: C, 52.26; H, 5.17; N 6.30. UV-Vis peaks (in DMSO): 280, 335, 390, and 565 nm.

Synthesis of [VO(Hshed)(Coum)]. The following reaction was carried out under argon atmosphere, typically using a Schlenk line to set up the reaction, followed by an argon balloon during the reaction. The H₂Coum (0.196 g, 1.10 mmol, recrystallized from benzene) and solid VO₂(Hshed) (0.290 g, 1.0 mmol) were added to a 100 mL round bottom Schlenk flask followed by absolute ethanol (50 mL). The conversion of the yellow VO₂(Hshed) to the purple/black VO(Hshed)(Coum) occurred over 4 h (colors changed from light yellow to brown to dark purple). However, the mixture was allowed to stir covered overnight under argon after which time the dark purple reaction mixture was cooled in a dry ice/acetone bath for 1 min and then was vacuum filtered, leaving a purple black solid on the filter paper. The solid was washed twice with cold absolute ethanol (<0° C., 25 mL) and then dried under vacuum for 3 days to yield 0.339 g (0.753 mmol, 75% yield) purple/black solid. The filtrate was concentrated, suspended in cold EtOH, and filtered but yielded no additional product. ⁵¹V NMR in CD₃CN: −46 ppm, −10 ppm (minor isomer). ¹H NMR (400 MHz, CD₃CN): δ 8.71 (s, 1H), 7.69-7.67 (d, 1H), 7.61-7.58 (dd, 1H), 7.56-7.51 (dt, 1H), 6.96-6.92 (dt, 1H), 6.80-6.77 (d, 1H), 6.65 (s, 1H), 6.24 (s, 1H), 5.98-5.95 (s, 1H), 4.49-4.44 (m, 1H), 4.23-4.17 (dd, 1H), 4.10-4.02 (dt, 1H), 3.82-3.74 (m, 1H), 3.70-3.63 (m, 1H), 3.60-3.48 (m, 2H), 3.39-3.31 (m, 1H), 3.04-2.91 (m, 1H), 2.81-2.78 (t, 1H). IR (ATR, 4 cm⁻¹): 1719 (C═O), 1636 (Aromatic), 1289, 1151 (C—O), 955 (V═O). Calc for C₂₀H₁₉N₂O₇V: C, 53.19; H, 4.07; N, 6.06. Found: C, 53.34; H, 4.25; N, 6.22. UV-Vis peaks (in DMSO): 280, 360, and 520 nm.

Preparation of Stock Solution for NMR Spectroscopic Studies. A 20.0 mM stock solution of the respective V-complex was prepared in DMSO by dissolving 0.0580 g, 0.0764 g, 0.0989 g, and 0.0825 g of [V(O)₂(Hshed)], [VO(Hshed)(cat)], [VO(Hshed)(dtb)], or [VO(Hshed)(3OMet)], respectively, in 10 mL DMSO. The solution of each V-complex was diluted to form a solution with highest final concentration of 10.0 mM in 50:50 DMSO:H₂O or 50:50 DMSO:7H9 media.

Preparation of Stock Solutions for Cell Culture Experiments. A 20 mM stock solution of each respective V-complex and its free ligands was prepared in DMSO and diluted with 7H9 media to a concentration of 2.0 mM for biological studies.

UV-Visible Spectra. Absorbance spectra of V-complexes were measured at 0, 1, 5, and 24 h using a UV-Visible spectrophotometer (BioTek Synergy™ HTX Multi-Mode Microplate Reader: Winooski, VT, USA) in the 200 to 800 nm wavelength range. Samples were prepared in solutions of H₂O:DMSO, 7H9 growth medium:DMSO, or the supernatant fraction obtained from M. smeg cell culture.

Nuclear Magnetic Resonance (NMR) Measurements. ⁵¹V NMR using a Bruker 400 MHz spectrometer was used to observe species present at different time points in DMSO:H₂O samples and DMSO:7H9 medium. Studies of the bacterial supernatant from M. smeg culture were attempted as well. Speciation of V-complexes were measured through the integration of the vanadium peaks in the NMR spectra. The stability of the V-complexes and precursor complex [V(O)₂(Hshed)] was also checked using ⁵¹V NMR spectroscopy.

Analysis of Species in Stock Solutions and Media as a Function of Time. The species present were analyzed based on spectroscopic data in aqueous-DMSO solutions and in growth media solutions before and after M. smeg growth as described above. The 20.0 mM stock solutions of V-complexes were prepared in DMSO and diluted to the final concentration of 10.0 mM in DI H₂O (50:50, DMSO:H₂O) or to the final concentration of 10.0 mM in 7H9 medium (50:50, DMSO:7H9). Samples were incubated at 37° C. during the stability studies. The V-complexes in double-distilled H₂O and in 7H9 medium (pH 6.65 prior to adding the DMSO solution) were measured at different time points (0, 1, 5, and 24 h) using UV-Vis spectroscopy and ⁵¹V NMR spectroscopy. The UV-Vis spectra were recorded using DMSO:H₂O (50:50) as a reference and for fresh media and for supernatant previously obtained from M. smeg culture. For studies using cell culture supernatant, at each time point (0, 1, 5, and 24 h), a 1 mL aliquot of supernatant was collected from the 5 mL sample for ⁵¹V NMR analysis. The 7H9 media samples measured as a function of time are shown in FIG. 8. The NMR spectra were analyzed by integrating the ⁵¹V NMR spectra and assuming that all the V-compounds were present as V(V) species. The species present in the spectra were calculated based on the mole fraction of the observed signals and the known concentration of V-complexes added to the solutions.

Conclusion

The growth effects of an anticancerous non-toxic Schiff base oxidovanadium(V) complex coordinated to 3,5-di-tert-butylcatecholato were assessed for comparison to the growth effects a series of subtly different non-toxic compounds using Mycobacterium smegmatis (M. smeg). We specifically chose to examine (1) the growth effects of Schiff base oxidovanadium complexes coordinated to a catechol and (2) the growth effects of the complexes' respective free catecholates for comparison and to determine (3) when the metal coordination complex is more potent than ligand alone. Schiff base oxidovanadium catecholate compounds are coordination complexes and many of these compounds undergo hydrolysis. To obtain more information on the effects of intact complexes versus ligands, we selected a class of complexes with varying stability that contained a ligand with the potential to inhibit growth of M. smeg. We then investigated both the species present under biological conditions and the effects of the intact complex and the hydrolyzed complex as well as the free ligand. The order of growth effects of ligands differed from that of the complexes and complex stability. This did not appear to be critical for growth inhibition because the most inhibitory catechol ligands were not found to form comparably inhibitory complexes. Considering speciation of the V-complex in cell growth medium, we concluded that both hydrophobicity and steric hindrance were important factors for the observed growth inhibition of M. smeg as well as the anticancer properties of the various V-complexes.

### Example 3. Advantageous Reactivity of Unstable Metal Complexes: Potential Applications of Metal-Based Anticancer Drugs for Intratumoral Injections

Overview

Injections of highly cytotoxic or immunomodulating drugs directly into the inoperable tumor is a procedure that is increasingly applied in the clinic and uses established Pt-based drugs. It is advantageous for less stable anticancer metal complexes that fail administration by the standard intravenous route. Such hydrophobic metal-containing complexes are rapidly taken up into cancer cells and cause cell death, while the release of their relatively non-toxic decomposition products into the blood has low systemic toxicity and, in some cases, may even be beneficial. This concept can be used to administer V(V) complexes with hydrophobic organic ligands. The potential beneficial effects include antidiabetic, neuroprotective and tissue-regenerating activities for V(V/IV). Utilizing organic ligands with limited stability under biological conditions, such as Schiff bases, further enhances the tuning of the reactivities of the metal complexes under the conditions of intratumoral injections. However, nanocarrier formulations can also be used for the delivery of unstable metal complexes into the tumor.

Introduction

The treatment of inoperable cancers, particularly those of the brain, head and neck, lung or pancreas, by direct injection of cytotoxic and/or immunomodulating drugs into the tumor is currently transitioning from experimental procedures to mainstream clinical practice. Detailed clinical guidelines for intratumoral injections (ITI) have been outlined, and hundreds of clinical trials are either underway or have been completed. The treatment of unresectable metastatic melanoma by ITI of an oncolytic virus (T-VEC) has been approved by the Food and Drug Administration (FDA) for human clinical use. A related technique, convection enhanced delivery (CED), which is based on intracranial injections of chemotherapeutic drugs to overcome the blood-brain barrier, continues to be extensively trialed for the treatment of malignant gliomas. Another related technique, pressurized intraperitoneal aerosolized chemotherapy (PIPAC), is under development for the treatment of metastatic cancers of the digestive system. One of the main aims of these techniques is to maximize the concentrations of cytotoxic drugs within the tumor and to minimize their concentrations in the blood, which reduces the systemic toxicity of the treatment. While ITI, CED and PIPAC treatments are generally regarded as palliative rather than curative, they can be applied in combination with systemic chemotherapy to reduce the spread of metastases and significantly prolong the life of cancer patients. Classical Pt(II)-based anticancer drugs (cisplatin, carboplatin and oxaliplatin) are increasingly used in ITI, CED and PIPAC formulations both in pre-clinical studies and in human clinical trials.

Extensive changes in the speciation of most metal-based drugs typically occur after their administration, due to the abundance of potential biomolecular ligands and reducing (or less commonly, oxidizing) agents in biological fluids. One possible solution for this problem is the design of substitutionally inert (mostly organometallic) complexes where the metal ion acts either as a scaffold to build a three-dimensional organic structure for selective binding to protein targets or as a catalytic center for intracellular redox reactions. Another approach is to use kinetically inert Pt(IV) or Co(III) prodrugs, which can be converted to their more labile Pt(II) or Co(II) counterparts in the reducing the environment of solid tumors. This approach is often proposed for the targeted delivery of biologically active organic molecules that are bound to such metal ions. However, their administration by intravenous injection can result in the reduction of the metal ion by Fe(II) in red blood cells with premature release of the active components.

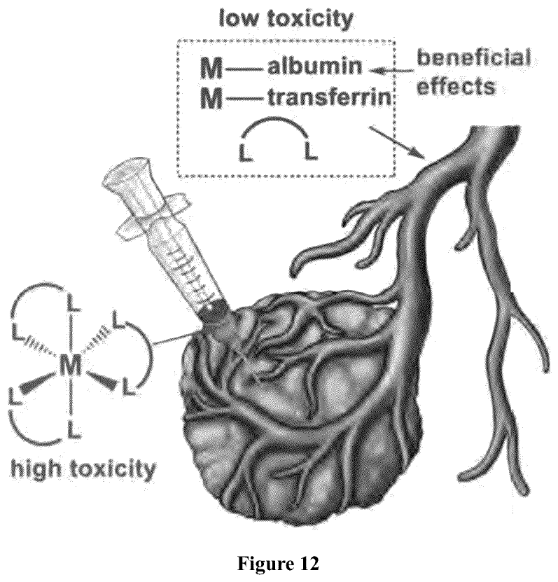

We propose the use of reactive metal complexes that have some stability but limited lifetimes in biological media. Such complexes are ideal agents for ITI and related delivery techniques of anticancer drugs. In this case, the binding of hydrophobic organic ligands to a toxic metal ion assists its efficient uptake into tumor cells and results in high cytotoxicity, while the decomposition products that are released into the blood stream consist of relatively non-toxic ligands and metal-protein complexes (FIG. 12). This approach is expected to exhibit low systemic toxicity, similar to photodynamic therapy or boron neutron capture therapy, where highly cytotoxic but short-lived agents are generated locally in the tumor tissue. Similar principles are also applied to organic anticancer prodrugs that hydrolyze in biological media with the formation of highly cytotoxic but short-lived active species. Importantly, the decomposition products of some metal anticancer drugs are likely to have beneficial biological effects, as in the case of many V(V) complexes with hydrophobic organic ligands. In this review, we briefly discuss the potential of vanadium metal complexes with anticancer properties for use in intratumoral applications.

Vanadium(V) Complexes

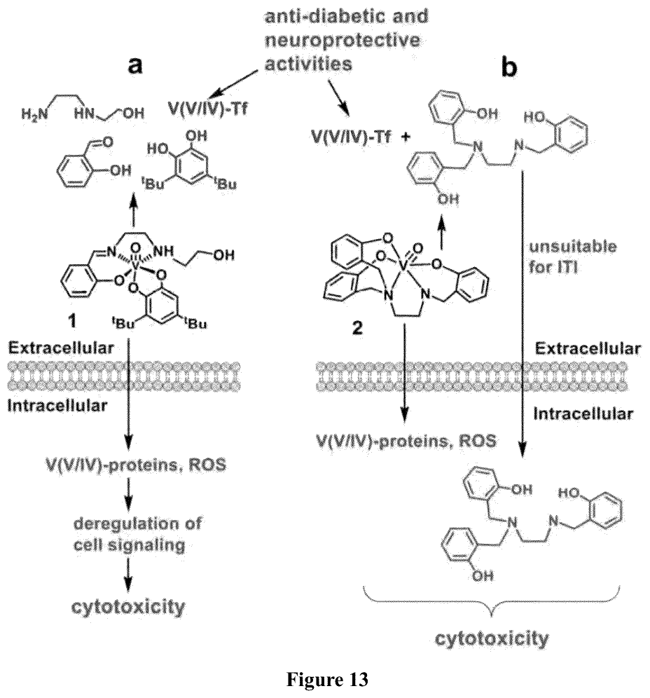

Many V(V/IV) complexes with varying structures exhibit anticancer activity. The concept of using relatively unstable metal complexes for ITI, where the complexes had some stability and exerted high reactivity, was developed for a noninnocent oxidovanadium(V) complex with a tridentate Schiff base and a redox-active di-3,5-tert-butylcatecholato ligand (1 in FIG. 13). Despite the vanadate-phosphate analogy, the nature of V-O bond in 1 and in other V(V/IV) complexes with organic ligands is closer to a triple than a double bond (2.5<n≤3, FIG. 13) due to the presence of one a and two π bonds, and the bond is thus presented as a triple bond. Due to the hydrophobic nature of the ligands and sufficient stability of the coordination complex in biological media, 1 is efficiently taken up by cancer cell monolayers and causes high cytotoxicity (IC₅₀˜ 1-4 μM in 72 h treatments). Complex 1 is ˜10-fold more toxic than cisplatin under the same conditions. This effect is likely to be caused by changes in cell signaling that could originate from direct interactions of the cell membrane with V-complexes, inhibition of protein phosphatases by V-derivatives, as well as from V(V) reactions with cellular reductants that generate reactive oxygen species (ROS); see FIG. 13. In parallel, rapid decomposition of 1 in cell culture medium occurs (halflife, ˜30 s at 37° C.), which involves hydrolysis of the Schiff base ligand, the release of oxidovanadium(V) species and their binding to serum proteins, predominantly transferrin (Tf, FIG. 13). This decomposition leads to a decrease in cytotoxicity by an order of magnitude, due to the low cellular uptake of V-Tf adducts and low cytotoxicity of the ligand fragments. Furthermore, V-Tf adducts are likely to be involved in the beneficial biological activities of V, such as antidiabetic, neuroprotective, and neurostimulatory effects. The latter activities, together with the favorable cytotoxicity ratio of fresh and decomposed 1 in human glioma multiforme (T98 g) cells, led to the suggestion that 1 can be used in the ITI formulations for this aggressive form of brain cancer. This suggestion is supported by the low acute oral toxicity of 1 in mice. Neuroprotective and neurostimulatory activities of the decomposition products of 1 may help to fight the neurological and cognitive disorders that commonly occur from cancer itself, or from standard chemotherapy.

For comparison, the parent analog of 1 without tert-butyl substituents in the catechol ligand (the simple catechol) decomposes completely within a few seconds in the cell culture medium and is not taken by the cells to a significant extent. Further developments in this field will involve tuning the hydrophobicity and aqueous stability of mixed-ligand V(V) complexes. This will enable optimization of their cellular uptake and decomposition rates and cytotoxic activities for the use in ITI and related techniques.

Like 1, V(V) complexes with reduced Schiff base (salan-type) ligands, such as 2 in FIG. 13, are efficiently taken into cultured human cancer cells and are highly cytotoxic. Unlike for 1, the cytotoxicity of 2 is predominantly due to the release of hydrolytically stable ligands, extracellularly and/or intracellularly (FIG. 13). Similar ligand-based cytotoxicity mechanisms have been proposed for V(V/IV) complexes with typical hydrophobic and cytotoxic chelating ligands, such as 1,10-phenanthroline or 8-hydroxyquinoline. The release of stable and highly cytotoxic ligands into the blood stream is likely to lead to significant systemic toxicity that complicates the use of 2 and other V(V) complexes with stable cytotoxic ligands in ITI (FIG. 13). However, salantype ligands in V(V) complexes can also be relatively non-toxic, which emphasizes the need for comparative biological activity studies of metal complexes and the corresponding free ligands.

Schiff bases, particularly those derived from salicylaldehyde and diamines (salentype ligands) are a staple of coordination chemistry. Numerous metal complexes of these ligands have undergone biological activity assays, but none have entered advanced preclinical development. Although the hydrolysis of Schiff bases to the original aldehyde and amine components in neutral aqueous solutions is known, its implications for biological activities of metal Schiff base complexes have not been widely recognized. For instance, the formation of aldehyde and amine precursors of the Schiff base ligand during the dissolution and subsequent decomposition of 1 in water (FIG. 13) has been demonstrated by ¹H NMR spectroscopy. The reactivity of the complex and ligand cleavage and V(V) release (FIG. 13) is responsible for the short lifetime of 1 under biologically relevant conditions, which forms the basis of the proposed use of 1 in ITI.

Drug Formulations for ITI

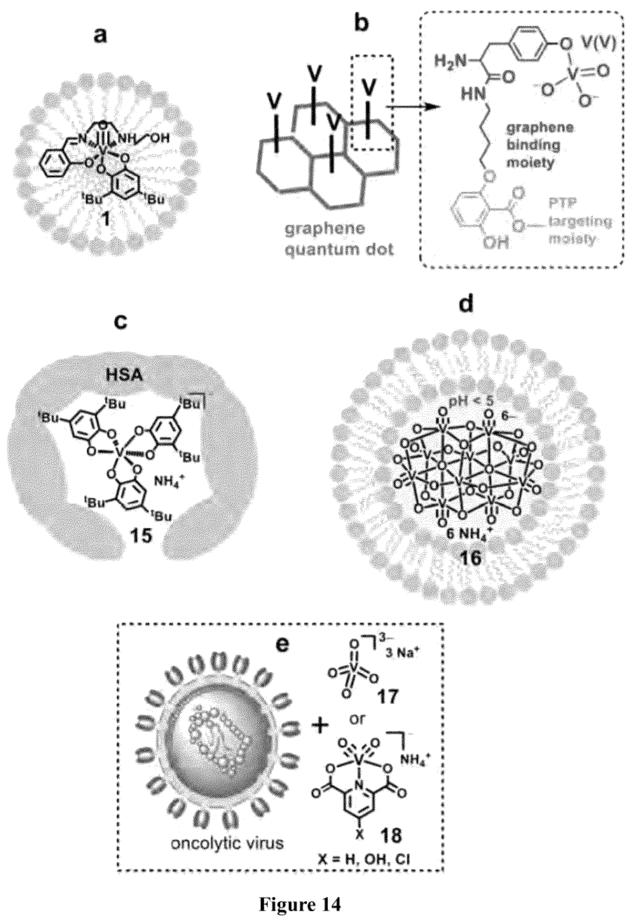

Producing stable, injectable formulations of poorly water soluble and/or watersensitive metal-based drugs is a significant challenge. Many of the proposed ITI formulations of cytotoxic drugs, including Pt(II) complexes, involve polymeric matrices that are designed for the slow release of the drug, but these are less applicable to unstable metal complexes that have to be delivered rapidly. Some of the possible solutions that can be applied to unstable and reactive V(V) complexes include micellar systems (FIG. 14, panel (a)), graphene quantum dots (FIG. 14, panel (b)), human serum albumin (HSA) adducts (FIG. 14, panel (c)), liposomal systems (FIG. 14, panel (d)) and oncolytic virus-metal complex suspensions (FIG. 14, panel (e)).

A simple approach that is compatible with ITI involves the encapsulation of hydrophobic complexes, such as 1, within micelles that are formed by a mixture of polyethylene glycol and fatty acids or triglycerides. The binding of inorganic vanadate to small peptides that are incorporated into cell-permeable graphene quantum dots can also be used for the precise delivery of V(V) to its cellular targets, such as a labile protein tyrosine phosphatase 1B (PTP1B) inhibitor, which was stabilized by the graphene framework. This delivery system can produce pronounced antidiabetic activity in mice. Such technology also enabled the targeting of the compound using protein tyrosine phosphatases (protein tyrosine phosphatase 1B and T-cell protein phosphatase). A similar approach could potentially be designed for the delivery of unstable anticancer metal complexes to tumors via ITI techniques.

Another way to increase the aqueous solubility and stability of hydrophobic metal complexes, such as the V(V) tris-3,5-di-tert-butylcatecholato complex 15, is to enclose them in hydrophobic pockets of human serum albumin (HSA). The use of HSA as a carrier of anticancer drugs can assist in their retention in tumors and a formulation using a HSA adduct of a Pt(IV) complex has entered human clinical trials. In a related approach, the binding of inorganic V(V) and V(IV) salts to HAS through a covalently attached chelating ligand (EDTA) led to their efficient cellular uptake through caveolae-mediated endocytosis and high antiproliferative activity in cultured cancer cells. This approach can potentially be used for the development of metalligand-HSA conjugates with an optimized lifetime for ITI applications.

Liposomal formulations of immunomodulating drugs can also be applied for use with ITI. Water-soluble complexes, such as ammonium decavanadate 16, or other polyoxometalates, can be encapsulated within unilamellar liposomes. The pH value within the liposomes can be regulated to increase the stability of such complexes. This approach may open the way for the wider use of unique biological activities of polyoxometalates that are different from those of mononuclear complexes. Liposomal formulations have also been developed to enhance the stability of hydrophobic V(V) complexes in biological media.

Another way to harness the effect of V complexes on cellular signal transduction is their use in enhancing the effects of oncolytic viruses. Co-administration of a virus with inorganic vanadate (17) or selected V complexes can enhance their uptake and cytotoxicity in cultured cancer cells and reduced tumor sizes. Viral infection and cytotoxicity in cancer cells was further enhanced by using more lipophilic V(V) complexes with dipicolinate ligands (18), although such complexes are short-lived in aqueous solutions. These findings are of immediate interest for the use in ITI of oncolytic viruses, which is the only ITI application currently approved for clinical use.

Conclusion

Metal-based anticancer drugs often have low stability in biological media, and this is one of the main obstacles to their wider use in clinical practice. One can take advantage of this instability and consequently reactivity and use these compounds in ITI applications. This concept is based on the results of in vitro stability studies and cell culture assays using a mixed-ligand V(V) complex showing significantly enhanced activity over cisplatin, 1 (FIG. 13). This highlights the other vanadium-based anticancer drugs that could potentially be suitable candidates for ITI injections.

In cell culture models, V(V) complexes with hydrophobic organic ligands were far superior to cisplatin in causing cancer cell death, particularly in short term treatments that are relevant to ITI. The use of these biologically active but relatively unstable V(V) complexes can be further enhanced by the development of suitable drug formulations that stabilize the compounds further (as discussed above). This is particularly relevant for their use in ITI and CED for the treatment of malignant gliomas. Based on the low acute toxicity of 1 in healthy mice, the use of stabilized formulations of 1 and other hydrophobic metal complexes for intratumoral injections in cancers offers promise.

Successful ITI has a cellular uptake of metal drugs that is faster than the extracellular complex decomposition. Since the proposed ITI approach is dependent on the kinetic competition between cellular uptake and extracellular decomposition, and this is characteristic for transition metal complexes, these complexes are ideal for such ITI applications. Generally, any cytotoxic metal complex can be considered for the use in ITI if it decomposes in an extracellular medium at a comparable rate with its cellular uptake and the decomposition products show lower toxicity compared with the initial complex. The latter consideration is crucial to exclude the possibility that the cytotoxicity of the metal complex is due to the release of stable and biologically active ligands either inside or outside of the cell, such as 2 in FIG. 13. Under the conditions of ITI, the release of such ligands into the blood stream (FIG. 12) is likely to lead to high systemic toxicity. Therefore, metal complexes of the ligands that have limited lifetimes in neutral aqueous solutions, such as Schiff bases, can be particularly suitable for the use in ITI. More research is needed to follow early kinetic studies on the decomposition of such ligands and their complexes under biologically relevant conditions as well as methods that will stabilize these systems and facilitate the administration of these complexes.

An important consideration in the use of metal complexes as anticancer drugs for ITI is the potential beneficial activity of their decomposition products, which is unlikely to occur for non-metal-based drugs. Some of the most promising examples include the antidiabetic, tissue regeneration and neurostimulatory activities of V(V/IV) complexes. The multiple modes of biological activity of many metal ions, dependent on their concentration and speciation in biological compartments highlight the unique potential for metal complexes in medicinal applications, which is far from being fully realized at this time.

### Example 4. Vanadium Chloro-Substituted Schiff Base Catecholate Complexes are Reducible, Lipophilic, Water Stable, and Have Anticancer Activities

Overview

A hydrophobic Schiff base catecholate vanadium complex was recently discovered to have anticancer properties superior to cisplatin and suited for intratumoral administration. This [VO(HSHED)(DTB)] complex, where HSHED is N-(salicylideneaminato)-N′-(2-hydroxyethyl)-1,2-ethanediamine and the non-innocent catecholato ligand is di-t-butylcatecholato (DTB), has higher stability compared to simpler catecholato complexes. Three new chloro-substituted Schiff base complexes of vanadium(V) with substituted catecholates as co-ligands were synthesized for comparison with their non-chlorinated Schiff base vanadium complexes, and their properties were characterized. Up to four geometric isomers for each complex were identified in organic solvents using ⁵¹V and ¹H NMR spectroscopies. Spectroscopy was used to characterize the structure of the major isomer in solution and to demonstrate that the observed isomers are exchanged in solution. All three chloro-substituted Schiff base vanadium(V) complexes with substituted catecholates were also characterized by UV-vis spectroscopy, mass spectrometry, and electrochemistry. Upon testing in human glioblastoma multiforme (T98 g) cells as an in vitro model of brain gliomas, the most sterically hindered, hydrophobic, and stable compound [t1/2 (298 K)=15 min in cell medium] was better than the two other complexes (IC₅₀=4.1±0.5 μM DTB, 34±7 μM 3-MeCat, and 19±2 μM Cat). Furthermore, upon aging, the complexes formed less toxic decomposition products (IC50=9±1 μM DTB, 18±3 μM 3-MeCat, 24 and 8.1±0.6 μM Cat). The vanadium complexes with the chloro-substituted Schiff base were more hydrophobic, more hydrolytically stable, more easily reduced compared to their corresponding parent counterparts, and the most sterically hindered complex of this series is only the second non-innocent vanadium Schiff base complex with a potent in vitro anticancer activity that is an order of magnitude more potent than cisplatin under the same conditions.

Background

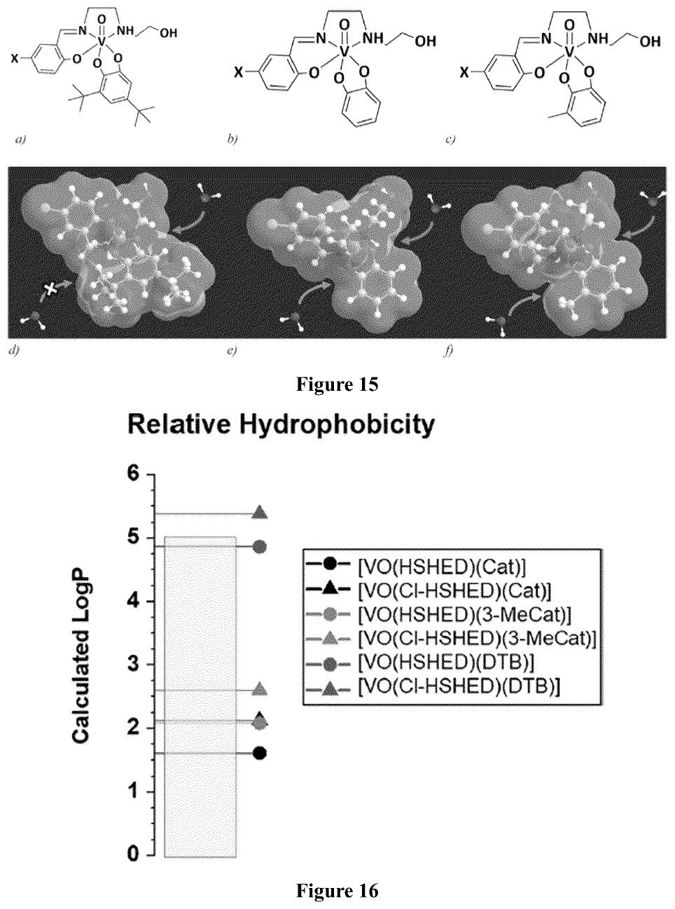

Aggressive cancers that include some brain and pancreatic cancers are notably difficult to treat and continue to present a challenge for life scientists to develop new anticancer strategies and therapies including intratumoral injections. In the following Example, we report a series of hydrophobic vanadium complexes and characterize their chemical and anticancer properties but with different redox properties than the lead complex, as shown in FIG. 15. Vanadium complexes act through many mechanisms in biology, which in broad strokes can be described by impacting signal transduction, interacting with regulatory protein phosphatases and kinases, transporting proteins as well as affecting the redox states of cells presumably through reactive oxygen species (ROS). Since cancer cells are known to support a more reducing environment, the design of a new class of Schiff base V-catecholates described in this work is to explore a series of complexes with different redox potentials. These non-innocent Schiff base V-catecholate complexes are oxidation state V vanadium complexes stabilized by a ternary ligand and coordinated to a redox active catecholato ligand. Non-innocent complexes are redox-active complexes that contain a ligand, which can undergo a redox reaction in addition to the metal, and thus making it difficult to determine whether the redox reaction took place on the metal, or on the ligand, or was substantially delocalized over both. The extent of such delocalization can be probed by a combination of XAS studies on XANES to determine the metal oxidation state and bond lengths combined with DFT calculations. The complex design originated from one vanadium Schiff base complex with the di-t-butylcatecholato (DTB) ligand (FIG. 15(a), X═H) that was more cytotoxic compared to cisplatin by 1 order of magnitude in T98 g cells (glioma multiforme, an aggressive brain cancer). It is also more active than cisplatin against models for other hard-to-treat cancers, A549 (lung), PANC-1 (pancreatic), and SW1353 (bone chondrosarcoma) cells. In contrast, this compound was not toxic in normal cells, such as normal human foreskin fibroblasts (HTT-1), or in animal studies in mice.

Members of the class of non-innocent Schiff base V-catecholates were designed to have sufficient stability and hydrophobicity to enter cells rapidly before decomposition in biological media. The essential design feature requires the compound to remain intact for uptake to occur but not too long to induce more systemic toxicity. After injecting directly into a tumor, the complexes will enter the tumor cells rapidly and exert their action at which point the decomposition products can diffuse away from the tumor and into healthy cells where they exhibit low cytotoxicity and potentially beneficial neuroprotective and other beneficial effects. Hundreds of clinical trials are ongoing using these intratumoral drug-delivery methods with one drug, T-Vec already in the clinics for treatment of late-stage cancers. Seven of these ongoing clinical trials involve platinum-based drugs, so the best of the non-innocent V-complexes are an order of magnitude more potent than cisplatin fuel the focus of this manuscript on the development on a new class of non-innocent chloro-substituted Schiff base V-catecholates. Complexes with superior antiproliferative effects compared to cisplatin have the potential to be suitable for an intratumoral injection (FIG. 15) and related administration methods and are important targets for future therapeutic agent development. Schiff bases are formed as condensation products between an aldehyde and an amine to generate versatile ligands with large structural diversity. Complexation with metal ions can lead to a wide range of metal complexes used in catalysis as materials and as bioactive complexes. These ligands offer several approaches to tailor a complex by changing the coordinating moieties of the Schiff base, substitution on the aromatic rings, and changes in the hydrophobicity of the ligands. The non-innocent vanadium-Schiff base complexes represent a much smaller class of V-Schiff base complexes containing a redox active ligand. The Schiff base scaffold can form a vanadium(IV) complex but with catecholate co-ligands, the Schiff base ligand serves to stabilize the vanadium(V) oxidation state.

Vanadium tris-catecholates are generally known to contain vanadium in oxidation state III or IV except for the vanadium tris-catecholato complex formed from 3,5-di-t-butyl catechol where the vanadium is in oxidation state V. X-ray absorption spectroscopy, in particular, demonstrated that the redox chemistry was mainly at the metal center rather than that on the catecholato ligand. The non-innocent vanadium(V) Schiff base complexes of interest are shown in FIG. 15 and have the vanadium stabilized by a ternary Schiff base-ligand. When a hydrophobic sterically hindered catecholato ligand is used, the complexes hydrolytic stabilities have been significantly increased. The electrochemical properties and electronic structure of these VO(HSHED) and related catecholate systems were investigated; however, little is known about how their solution redox chemistry impact their antiproliferative activities. Since biological properties of other vanadium(V)-complexes are known to involve ROS, we designed the series of new chloro-substituted Schiff base V-complexes to explore whether complexes that more readily undergo redox chemistry have improved anticancer effects. Hence, the new chloro-substituted non-innocent vanadium(V) Schiff base complexes were developed, and their chemistry and anticancer properties are described.

In this Example, we present three new complexes ([VO—(HSHED)(R₂Cat)] (R₂═H,H; H,Me; or ^{t}Bu, ^{t}Bu) (HSHED=N-(salicylideneaminato)-N-(2-hydroxyethyl)-1,2-ethanediamine, Cl-HSHED=4-chloro-N-(salicylideneaminato)-N′-(2-hydroxyethyl)-1,2-ethanediamine, Cat=pyrocatecholato, 3-MeCat=3-methylcatecholato, and DTB=3,5-di-t-butylcatecholato, FIG. 15a-c; X═Cl) designed to have different redox potentials, hydrophobicity (FIG. 15d-f, and waterstability compared to the compound, as shown in FIG. 15(a) when X═H. Specifically, the complexes were designed to test the following two hypotheses that (1) both hydrophobicity and stability are important to the anticancer properties of the complexes, as well as (2) redox chemistry is important because the mode of action may involve ROS. Furthermore, this Example will provide additional examples of non-innocent Schiff base V-catecholate complexes with different redox potentials and document their anticancer properties.

Materials and Methods General Materials. 5-Chlorosalicylaldehyde, N-(2-hydroxyethyl)-1,2-ethanediamine, vanadyl sulfate hydrate, catechol, 3,5-di-t-butylcatechol, and 3-methylcatechol were purchased from Sigma-Aldrich. Chemicals were used as is. Silver nitrate, ferrocene, and tetra-n-butylammonium perchlorate (TBAP) were purchased from Merk Millipore for the electrochemistry experiments. Ultrapure Ar (AR UHP300) from Airgas was used for degassing solutions. Deuterated PBS was prepared following a known protocol, replacing the water with deuterated water. The pH values of buffered solutions were adjusted with DCl and NaOD. The pre-sterilized media and sterile plasticware used in cell culture studies were purchased from Thermo Fisher Scientific Australia. General Methods and Instrumentation. The complexes were also characterized by UV-vis spectroscopy (AvaLight UV-vis/NIR Light Source and AvaSpex-UL S2048 Fiber-Optic Spectrometer), MS (Bruker amaZon SL spectrometer), and elemental analysis (ALS Environmental, Tucson, AZ). The hydrophobicity was calculated using Chemicalize software developed by ChemAxon and downloaded in May 2020.40 General Syntheses. [V(O)₂(HSHED)] and the non-halogenated analogues [VO(HSHED)(Cat)] and [VO(HSHED)(DTB)] were prepared using known procedures.

Briefly, the vanadium(V) complexes were prepared in situ after condensation of the chloro-substituted Schiff base from 5-chlorosalicylaldehyde and N-(2-hydroxyethyl)-1,2-ethanediamine with vanadyl sulfate because the Schiff base ligand was not stable and found to decompose upon storage.

[VO(HSHED)(3-MeCat)]. To a 250 mL round bottom Schlenk flask was added acetone (100 mL), which was then degassed with Ar. [V(O)₂(HSHED)] (0.29 g, 1.0 mmol) was added to the degassed acetone, followed by 3-methylcatechol (0.12 g, 1.0 mmol). A deep purple solution resulted after 3 h. The reaction was vacuum filtered and then concentrated to dryness under reduced pressure at room temperature. The purple residue was dissolved in a minimal amount of acetone, and then, n-hexane (100 mL) was added. The solution was allowed to stand in a −20° C. freezer overnight. The dark microcrystalline precipitate was vacuum filtered, washed with cold n-hexane (<0° C., 2×25 mL), and dried under vacuum for 3 d to yield 0.33 g (79%) as a purple solid. X-ray quality crystals were obtained by slow evaporation of a methanol solution. δ⁵¹V NMR (101 MHz, CD₃CN): 287 ppm (major), 324 ppm (minor). ¹H NMR (400 MHz, CD₃CN): δ 8.61 (s, 1H), 7.49 (m, 2H), 6.84 (t, 1H), 6.76 (m, 1H), 6.32 (d, 1H), 6.07 (d, 1H), 5.97 (d, 1H), 4.17 (m, 2H), 4.05 (m, 2H), 3.90 (s, 2H), 3.79 (m, 2H), 3.65 (m, 2H), 3.36 (m, 2H), 2.82, (t, 1H). λₘₐₓ (DMSO) 546 nm. Elemental Anal. Calcd for (VC₁₈H₂₁N₂O₅): C, 54.55; H, 5.34; N, 7.07. Found: C, 54.55; H, 5.41; N, 6.60. [V(O)₂(Cl-HSHED)]. 5-Chlorosalicylaldehyde (2.94 g, 18.8 mmol) and N-(2-hydroxyethyl)-1,2-ethanediamine (1.96 g, 18.8 mmol) were added to degassed methanol (75 mL), and the mixture was allowed to react for 1 h under Ar. Vanadyl sulfate hydrate (3.74 g, 18.8 mmol), dissolved in 50 mL of degassed water, was added to this solution, and after 3 h stirring under Ar, the solution was dark red/brown in appearance. Then, solid NaOH (1.12 g, 37.6 mmol) was added to the mixture, the solution was opened to the air, and the reaction mixture was stirred overnight. The light-yellow reaction mixture was cooled in a dry ice/acetone bath for ˜1 min, vacuum filtered, washed with cold EtOH (60 mL, <0° C.), washed with cold diethyl ether (60 mL, <0° C.), and then dried under vacuum for 3 d to yield 5.25 g (86% yield) as a light-yellow powder. δ⁵¹V NMR (101 MHz, CD₃CN): −531 ppm. ¹H NMR (400 MHz, CD₃CN): δ 8.63 (s, 1H), 7.45 (s, 1H), 7.40 (d, 1H), 6.81 (d, 1H), 4.44 (m, 1H), 4.16 (m, 1H), 4.01 (m, 1H), 3.84 (m, 1H), 3.34 (m, 2H), 3.28 (d, 1H), 3.01 (m, 1H), 2.74 (m, 1H). λₘₐₓ (DMSO) 335 nm. HRMS (ESI) calcd for C₁₁H₁₄ClN₂O₄V [MNa]+, 346.9979; found, 346.99739.

[VO(Cl-HSHED)(Cat)]. To a 250 mL round bottom Schlenk flask was added acetone (100 mL), which was then degassed with Ar. [V(O)₂(Cl-HSHED)] (0.33 g, 1.0 mmol) was added to the degassed acetone, followed by catechol (0.11 g, 1.0 mmol). A deep purple solution resulted in approximately 5 s. The solution was stirred overnight under Ar. The reaction mixture was vacuum filtered and then concentrated to dryness under reduced pressure at room temperature. The purple residue was dissolved in a minimal amount of acetone, and then, n-hexane (100 mL) was added. The solution stood in a −20° C. freezer overnight. The dark microcrystalline precipitate was vacuum filtered, washed with cold n-hexane (<0° C., 2 225×25 mL), and dried under vacuum for 3 d to yield 0.34 g (80%) as a purple solid. δ⁵¹V NMR (101 MHz, CD₃CN): 282 ppm (major), 322 ppm (minor). ¹H NMR (400 MHz, CD₃CN): δ 8.55 (s, 1H), 7.50 (d, 1H), 7.40 (dd, 1H), 6.75 (s, 1H), 6.73 (s, 1H), 6.52 (d, 1H), 6.33 (d, 2H), 4.25 (m, 1H), 4.08 (m, 3H), 3.77 (m, 1H), 3.61 (m, 1H), 3.44 (m, 1H), 3.32 (m, 1H), 2.94 (qd, 1H), 2.79 (t, 1H). λmax (DMSO) 531 nm. Elemental Anal. Calcd for (VC₁₇H₁₈N₂O₅Cl): C, 49.00; H, 4.35; N, 6.72. Found: C, 48.78; H, 4.10; N, 6.47. HRMS (ESI) calcd for C₁₇H₁₉ClN₂O₅V [MH]+, 417.04167; found, 417.04116.

[VO(Cl-HSHED)(3-MeCat)]. [VO(Cl-HSHED)(3-MeCat)] was synthesized following the [VO(Cl-HSHED)(Cat)] procedure to yield 0.34 g (79%) as a purple solid. δ⁵¹V NMR (101 MHz, 237 CD₃CN): 331 ppm (major), 362 ppm (minor), 369 ppm (minor), 409 ppm (minor). ¹H NMR (400 MHz, CD3CN): δ 8.56 (s, 1H), 7.51 (d, 1H), 7.41 (dd, 1H), 6.75 (d, 1H), 6.63 (m, 1H) 6.21 (d, 1H), 4.05 (m, 2H), 3.77 (m, 1H), 3.59 (m, 2H), 3.41 (m, 1H), 3.30 (m, 1H), 2.91 (m, 1H), 2.20 (s, 3H). λₘₐₓ (DMSO) 549 nm. HRMS (ESI) calcd for C₁₈H₂₁ClN₂O₅V [MH]+, 431.05732; found, 431.05702. 244

[VO(Cl-HSHED)(DTB)]. [VO(Cl-HSHED)(DTB)] was synthesized following the [VO(Cl-HSHED)(Cat)] procedure to yield 0.37 g (70%) as a purple solid. δ⁵¹V NMR (101 MHz, CD₃CN): 427 ppm (major), 467 ppm (minor). ¹H NMR (400 MHz, CD₃CN): δ 8.51 (s, 1H), 7.45 (d, 1H), 7.35 (dd, 2H), 6.67 (d, 1H), 6.35 (s, 1H), 6.29 (s, 1H), 4.03 (m, 2H), 3.80 (m, 1H), 3.51 (m, 2H), 3.41 (m, 1H) 3.33 (m, 1H), 2.95 (dd, 1H), 2.48 (m, 1H), 1.40 (s, 9H). λmax (DMSO) 552 nm. HRMS (ESI) calcd for C₂₅H₃₄ClN₂O₅V [MH]+, 528.15904; found, 528.15892. 253

NMR Spectroscopy. Complexes were characterized using ⁵¹V NMR spectroscopy recorded on a Bruker model AVANCE Neo400 spectrometer equipped with a BBFO smart probe and an automated tuning module operating at 101 MHz. The ⁵¹V NMR spectra were acquired with a spectral window of 86,200 Hz, 2048 scans, a 900 pulse, an acquisition time of 0.08 s, and a 0.01 s relaxation delay. 1D ⁵¹V NMR studies were referenced against [V(O)₂(HSHED)] at −529 ppm as a standard and reported in reference to VOCl₃ (0 ppm). 1D and 2D ¹H NMR studies were carried out in organic solvents using a Bruker NEO400 spectrometer operating at 400 MHz at an ambient temperature. Chemical shift values (δ) are reported in ppm and referenced against TMS using the internal solvent peaks in ¹H NMR spectra (DMSO-d₆, δ at 2.50 ppm; CDCl₃, δ at 7.26 ppm; d3-acetonitrile, δ at 1.94 ppm; C₆D₆, δ at 7.16 ppm) as internal standards. 1D ⁵¹V NMR studies were recorded on a Bruker NEO400 spectrometer at 105.2 MHz at an ambient temperature. All complexes were dissolved in either CD₃CN or DMSO-d6 at 10 mM concentrations for spectral comparison. Spectroscopic studies were carried out using solutions of isolated complexes, and both ¹H and ⁵¹V NMR spectra were recorded on the same samples. 1D samples were run within 5 h of preparation; no significant differences were observed in spectra recorded within 24 h.

¹H-¹H 2D COSY and NOESY NMR spectra in organic solutions were run overnight and recorded within 12 h of sample preparation. 2D NMR spectroscopic studies in organic solutions were carried out on a Bruker NEO400 spectrometer at 400 MHz at 26° C. A routine COSY pulse sequence provided by a Bruker software was used. A standard NOESY pulse sequence was used consisting of either 200 or 256 transients with 16 scans in the f1 domain using a 500 ms mixing time, 45° pulse angle, and a 1.5 s relaxation delay. The NMR was locked onto either DMSO-d6 or d3-CH₃CN and referenced to the internal solvent peak. The resulting spectrum was processed using MestReNova NMR software (version 12.0.1).

Mass Spectrometry. Low-resolution electrospray ionization mass spectrometry (ESI-MS) data were collected on a Bruker amaZon SL spectrometer, using the following parameters: nebulizer pressure, 27.3 psi; spray voltage, 4.5 kV; capillary temperature, 453 K; N2 flow rate, 4 L per min; m/z range, 100-1000 (alternating positive-ion and negative-ion modes). The compound stability parameter was set at 15% for all V(V) complexes, which provided mild ionization conditions at the expense of lower sensitivity. Smart parameter settings were fixed to the exact mass for each compound. Analyzed solutions (˜10 μM V in acetonitrile) were injected using a syringe pump (flow rate, 8 μL per min). Acquired spectra were the averages of 100-200 scans (scan time, 100 ms). Simulations of the mass spectra were performed using IsoPro 3.0 software (M. Senko, IsoPro 3.0, Sunnyvale, CA, USA, 1998). High-resolution positive-ion ESI-MS was performed on a Thermo Velos Pro Orbitrap mass spectrometer via syringe infusion (flow rate, 8 μL per min). Resolving power was set to 200,000 at 200 m/z. The instrument was externally calibrated before analysis and internally calibrated using dioctyl phthalate as a lock mass.

Electrochemistry. Electrochemistry was conducted using a WaveDriver 40 DC Bipotentiostat/Galvanostat and a Low Volume Three Electrode Cell Basic Kit (AFO1CKT1006) purchased from Pine Research Instrumentation. Software used during data acquisition was AfterMath v 1.5.9807 with the iR compensation option turned off, while eL-Chem Viewer and Microsoft Excel were used for post-acquisition processing.

Cyclic voltammograms (CVs) were measured in acetonitrile [2.0 mM V(V)] with an internal standard (ferrocene) in the presence of 0.1 M TBAP. Catecholates are well known to exhibit complex redox chemistry, which includes more than one redox step. The CVs were recorded from 1 to −1.5 to 1 V versus a non-aqueous Ag+/Ag reference electrode in triplicate at 100 mV s⁻¹.

Stock solutions of the electrolyte and analyte were prepared on the day of the experiment by first creating a 100 mM stock solution of TBAP dissolved in the solvent. The reference electrode was prepared by being filled with ˜2 mL of 10 mM silver nitrate and 100 mM TBAP in the solvent used for the electrochemical experiment. Analysis solutions consisted of one vial of 100 mM TBAP in the solvent, three 5.0 mL of samples of 2.0 mM analyte, 2.0 mM ferrocene, and 100 mM TBAP. Some samples were run without ferrocene in order to obtain voltammograms that were clear from potential interference of the Fc+/0 redox chemistry on the redox chemistry of the vanadium complex.

All electrochemistry experiments were undertaken using the classic three-electrode setup. All electrodes were purchased from BASi Research products. The working electrode was a glassy carbon electrode with a 3.0 mm diameter (2.997-2.972 mm) and an area of approximately 7 mm² (catalog no. MF-2012). The counter electrode was a platinum wire with a gold-plated connector mounted in a CTFE cylinder (catalog no. MW-1032). A non-aqueous silver(I)/silver/(Ag+/Ag) reference electrode consisted of a silver wire of 99.9% purity with a 0.5 mm diameter, is 30 cm long, and is contained within a double-junction reference electrode chamber with porous CoralPor tips, that are ⅛ in. long (catalog no. MF-2062). All solvents used for electrochemistry were dried on a Solv-Tek alumina drying column under argon to minimize the water content.

Cell Culture and Growth Conditions. The well-established human glioblastoma multiforme cell line (T98 g) and human foreskin fibroblast (HFF-1) cell lines were purchased from American Type Culture Collection (ATCC, cat. no. CRL-1690 and SCRC-1041). Due to their limited life span in culture, HFF-1 cells were used at passages four to six. The cells were cultured in Advanced DMEM (Thermo Fisher Scientific cat. no. 12491-015), supplemented with L-glutamine (2.0 mM), antibiotic-antimycotic mixture (100 U mL⁻¹ penicillin, 100 mg mL⁻¹ streptomycin, and 0.25 mg mL⁻¹ amphotericin B), and fetal calf serum (FCS; heat-inactivated; 2% vol). For proliferation and cytotoxicity experiments, cells were seeded in 96-well plates at an initial density of 1.5×10³ viable cells per well in 100 UL medium and left to attach overnight.

Freshly prepared stock solutions of V(V) complexes (10 mM in DMSO) were used for cell assays. These solutions were further diluted so that all the cell treatments, including controls, contained 1.0% (vol) of DMSO, which did not significantly affect the cell growth during the assays. Stock solutions of the treatment complexes were diluted with fully supplemented cell culture media to the required final concentrations, and the resultant media were either added to the cells within 1 min (fresh solutions) or left in cell culture incubator (310 K, 5% CO₂) for 24 h prior to the cell treatments (aged solutions).

Each treatment included six replicate wells with cells and two background wells without cells that contained the same components. After the addition of treatment complexes, the plates were incubated for 72 h at 310 K and 5% CO₂, then MTT reagent [1-(4,5-dimethylthiazol-2-yl)-3,5-diphenylformazan, Sigma M5655] was added (50 μL per well of freshly prepared 2.0 mg/mL solution in complete medium), and incubation was continued for 4-6 h. After that, the medium was removed, the blue formazan crystals were dissolved in 0.10 mL per well of DMSO, and the absorbance at 600 nm was measured using a Victor V3 plate reader. Typically, the treatment complexes were applied in a series of nine two-fold dilutions, starting from (100±20) μM V, plus the vehicle control. Exact V concentrations in the assays were verified by ICP-MS measurements using samples of cell culture media and used in the calculations of the IC50 values. Fitting of the experimental data and calculations of the IC50 values were performed using Origin 6.1 software (Microcal Origin, 1999). For all the cell assays, consistent results were obtained in at least two independent experiments using different passages of cells and varying stock solutions of the treatment complexes.

Results and Discussion

Design of New Vanadium Schiff Base Complexes. Some Schiff base vanadium(V) catecholate complexes have been synthesized and characterized. As discussed above, we have showed that increased hydrophobicity and steric bulk of the catecholato ligand improved cytotoxic properties of vanadium complexes with the di t-butyl catechol complex being the most potent. Here, we report solution characterization and anticancer properties of three new derivatives in a new class of Schiff base V-catecholate complexes with the addition of a single electron-withdrawing group (EWG) group on the Schiff base scaffold. These complexes were developed to modify parent complex stability, hydrophobicity, and redox properties. However, as we discovered with multinuclear NMR spectroscopic studies in several organic solvents, these complexes form rapidly interconverting isomers in solution.

Syntheses of Chloro Schiff Base V(V) Catecholato Complexes. The Schiff base V(V) precursor complex, [V(O)₂(Cl-HSHED)], was synthesized using a similar procedure as for the [V(O)₂(HSHED)] precursor. The Schiff base was first formed from a condensation reaction between chlorosalicyaldehyde and ethylenediamine which was then reacted in situ with vanadyl sulfate to form the vanadium scaffold, which was isolated as a solid after the addition of 2 equiv of NaOH. Isolation of the Schiff base ligand was found to be less optimal because the ligand did not store well and subsequently led to less pure and lower yielding V-complexes. In contrast, the solid vanadium Schiff base scaffold complex stored well, and it was reacted with a catechol ligand to form the desired vanadium catecholato complex in higher yields. In the catecholato complex synthesis, there was an immediate color change upon addition of the catechol for the chloro-substituted system, but for the parent catechol system, it took a few minutes. Prior protocols reduced the volume of the organic solvent before the solution was let stand overnight at −20° C. Here, we reduced the solution to dryness by rotary evaporation, re-dissolved the solid in minimal acetone, and then added 100 mL of hexane to the solution before allowing the mixture to stand overnight at −20° C. with yields increasing from 40 to 80%. The higher yields were attributed to the increased reaction times and changes in the crystallization conditions. The [V(O)₂(Cl-HSHED)] complex was a paler yellow than [V(O)₂(HSHED)], and the halogenated-Schiff base/catecholato complexes were deep purple like the complexes with the unsubstituted Schiff base. These Cl-HSHED complexes were characterized using UV-vis spectrophotometry, multinuclear NMR spectroscopy, electrochemistry, and MS-ESI spectrometry, as described in detail below.

Lipophilicity and P-Value. The lipophilicity of the V-compounds can be measured as a partition between a lipophilic organic phase, generally octanol, and a polar aqueous phase. The partitioning P-value is an important measure of the cell uptake and useful information in addition to the antiproliferative activity of these compounds. The problem with such measurements is that they are based on compounds that are hydrolytically stable, and hence, this experiment is difficult to conduct on compounds that hydrolyze within a few minutes and such experiments would be meaningless. We therefore carried out the shake-flask method studies, monitoring the partitioning using UV-visible spectroscopy only on the V-complexes with sterically hindered catecholates, [VO(HSHED)(DTB)] and [VO(Cl-HSHED)-(DTB)]. The results were at ˜100 μM; the parent complex separates into 4.6% in the water phase and 95.4% in the octanol phase, giving P=20 and log P 1.3. However, it should be noted that it is difficult to measure the low concentration of the complex in aqueous solutions, and that the observed ratios are a lower limit of the data.

To get a better understanding of the effects of the lipophilicity of the compounds that are readily hydrolyzed, we turned to computational methods which have been found to be successful in the analysis of a wide range of organic compounds. We used the website Chemicalize to estimate the log P values for the V(V) structures, as shown in FIG. 16. Since this program was developed for organic molecules, its use requires the assumption that a covalent bond can approximate a coordinate bond description in these metal complexes. Since all the complexes contain the same coordinate bonds, this method should be able to estimate the relative differences in these metal complexes. As anticipated, the larger the alkyl group (CH₃ and ^{t}Bu) were on the catecholato ligand, the more hydrophobic were the complexes. The addition of the 4-chloro-substituted Schiff base ligand increased the hydrophobicity of the [VO(Cl-HSHED)-(Cat)] to be comparable to that of [VO(HSHED)(3-MeCat)] as the chloro and the methyl groups are similar in size and effects in an aromatic system. Accordingly, the chloronated Schiff base V-catecholato complexes were estimated to be slightly more hydrophobic than their unsubstituted analogues. As shown in FIG. 16, the hydrophobicity estimated range from 1 to 4.5 (the yellow area) is based on Lipinski's rule of five. Furthermore, comparing these estimations with the experimental values obtained for [VO(HSHED)(DTB)] and [VO(Cl-HSHED)(DTB)], we find that the patterns observed with Chemicalize is as observed experimentally using the Shake-flask method. However, the calculated values are larger than the experimental values but within the range for most common drugs.

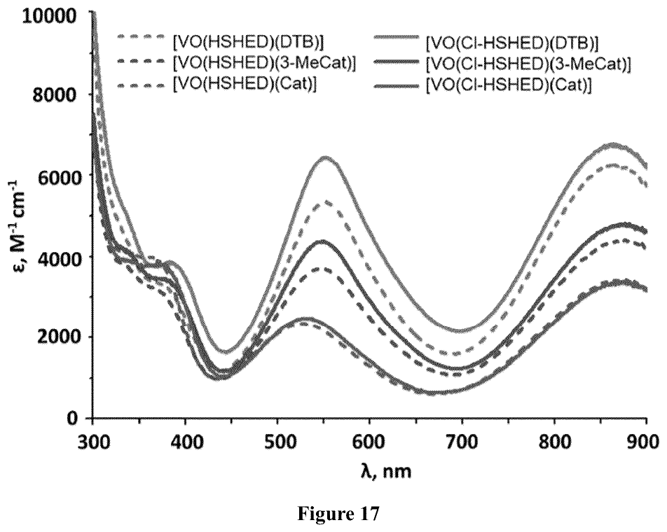

Characterization of Vanadium Schiff Base Catecholate Complexes in DMSO. The UV-vis spectra of the halogenated complexes and the parent complexes (0.10 mM, DMSO) exhibited maxima at approximately 390, 550, and 850 nm (FIG. 17) with λmax and εmax, as listed in Table 4. The [VO(Cl-HSHED)(DTB)] and [VO(Cl-HSHED)(3-MeCat)] complexes had higher extinction coefficients than the parent complexes. For the Cl-HSHED and the HSHED complex with the unsubstituted catechol, no difference in the λmax was observed. These data reflect the stabilizing effects the halogen substituent imparts on the alkyl-substituted catecholate complexes and the corresponding observed differences in the HOMO and LUMO orbitals of these complexes responsible for the observed absorption properties of the molecules.

TABLE 4
Band Positions and Corresponding Extinction Coefficients of the [VO(HSHED)]
and [VO(Cl-HSHED)] Compound Series.

| | [symbol] | | | [symbol] |
|---|---|---|---|---|
| compound | (nm) | [symbol] (M⁻¹ cm⁻¹) | [symbol] | (M⁻¹ cm⁻¹) |
| [VO(Cl-HSHED)(DTB)] | 552 | 6.5 × 10 [symbol] | 863 | 6.8 × 10 [symbol] |
| [VO(Cl-HSHED)(3-MeCat)] | 549 | 4.4 × 10 [symbol] | 873 | 4.8 × 10 [symbol] |
| [VO(Cl-HSHED)(Cat)] | 531 | 2.5 × 10 [symbol] | 873 | 3.4 × 10 [symbol] |
| [VO(HSHED)(DTB)] | 551 | 5.3 × 10 [symbol] | 861 | 6.2 × 10 [symbol] |
| [VO(HSHED)(3-MeCat)] | 546 | 3.7 × 10 [symbol] | 876 | 4.4 × 10 [symbol] |
| [VO(HSHED)(Cat)] | 528 | 2.3 × 10 [symbol] | 873 | 3.4 × 10 [symbol] |

[symbol]

indicates data missing or illegible when filed

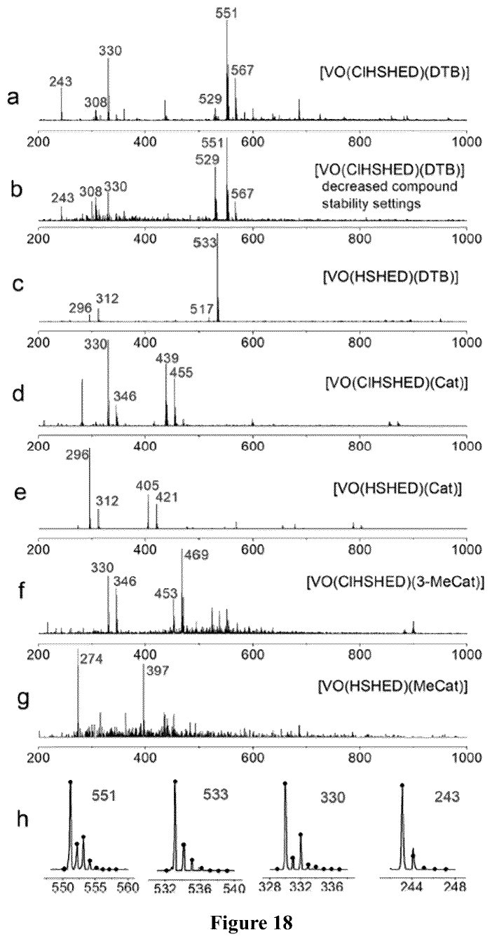

Characterization of [VO(X—HSHED)(DTB)], [VO(X—HSHED)(Cat)], and [VO(X—HSHED)(3-MeCat)] by ESI-MS. Mass-spectrometric characterization of the new V(V) complexes was particularly important because the elemental analysis proved problematic presumably due to the highly hygroscopic nature and air sensitivity of the isolated solids. Low-resolution ESI-MS data (positive-ion mode) for the newly synthesized Cl-HSHED complexes and their parent HSHED complexes are shown in FIG. 18, and assignments of the main signals are given in Table 5. Prominent signals of all the intact complexes were observed in the form of their Na⁺ or K⁺ adducts (Table 5), and the identities of these signals were confirmed by high-resolution mass spectrometry, where the same complexes were observed as H⁺ adducts. These results confirm the composition of intact [VO(Cl-HSHED)(DTB)], [VO(Cl-HSHED)(Cat)], and [VO(Cl-HSHED)(3-MeCat)] and the formation of the new halogenated Schiff base (Cl-HSHED) V(V) complexes.

Extensive decomposition of all the complexes was observed under ESI-MS conditions (FIG. 18 and Table 5). Relative intensities of signals due to decomposition products decreased when milder ionization conditions were employed (FIG. 18(b) vs 18(a)), which showed that decomposition of the original V(V) complexes occurred in the gas phase in addition to decomposition in solutions. Analysis of the decomposition products (Table 5) showed that redox chemistry of V(V)-Schiff base-catecholato complexes is taking place. Oxidation of catechols is known to occur in solution and is also observed in the gas phase now. Oxidation of catechols may be observed under some biological conditions in the presence of oxygen and metal complexes.

TABLE 5
Assignment of ESI-MS Signals for the Complexes
and their Decomposition Products.

| [symbol] | [ [symbol] ] [symbol] | [ [symbol] ] [symbol] | [ [symbol] ] [symbol] | [ [symbol] ] [symbol] | [ [symbol] ] [symbol] | [ [symbol] ] [symbol] |
|---|---|---|---|---|---|---|
| [VO(Cl-HSHED)( [symbol] )] [symbol] | [symbol] | [symbol] | [symbol] | [symbol] | [symbol] | |
| [VO(Cl-HSHED)( [symbol] )] [symbol] | [symbol] | [symbol] | [symbol] | [symbol] | [symbol] | |
| [VO(Cl-HSHED)( [symbol] )] [symbol] | [symbol] | | | | | [symbol] |
| [symbol] | | [symbol] | [symbol] | [symbol] | [symbol] | |
| [symbol] | [symbol] | [symbol] | [symbol] | [symbol] | [symbol] | |
| [symbol] | [symbol] | | | | | [symbol] |
| [symbol] | [symbol] | | | | | |

[symbol]

[symbol]

indicates data missing or illegible when filed

Characterization by ⁵¹V and ¹H NMR Spectroscopies. ⁵¹V NMR spectroscopy was used to demonstrate that interconverting isomers formed in solution at ambient temperature. Specifically, ⁵¹V NMR spectra of 1.0 mM solutions of the Cl-HSHED/catecholato complexes in CD₃CN and in DMSO-d6 showed more than one signal. We verified that these multiple signals were not due to impurities or hydrolysis products by concentration and time-dependent studies in different solvents.

In addition, no ⁵¹V NMR signal assigned to the vanadium precursor complexes, [V(O)₂(HSHED)] and [V(O)₂(Cl-HSHED)], was present in CD₃CN at −530 and −531 ppm, respectively. Similarly, the ¹H NMR signals of these precursors were also absent in the corresponding spectra of the catecholato complexes, though similar to the ⁵¹V NMR, we note that the signals are broad possibly also showing a reduction of V(V) to V(IV).

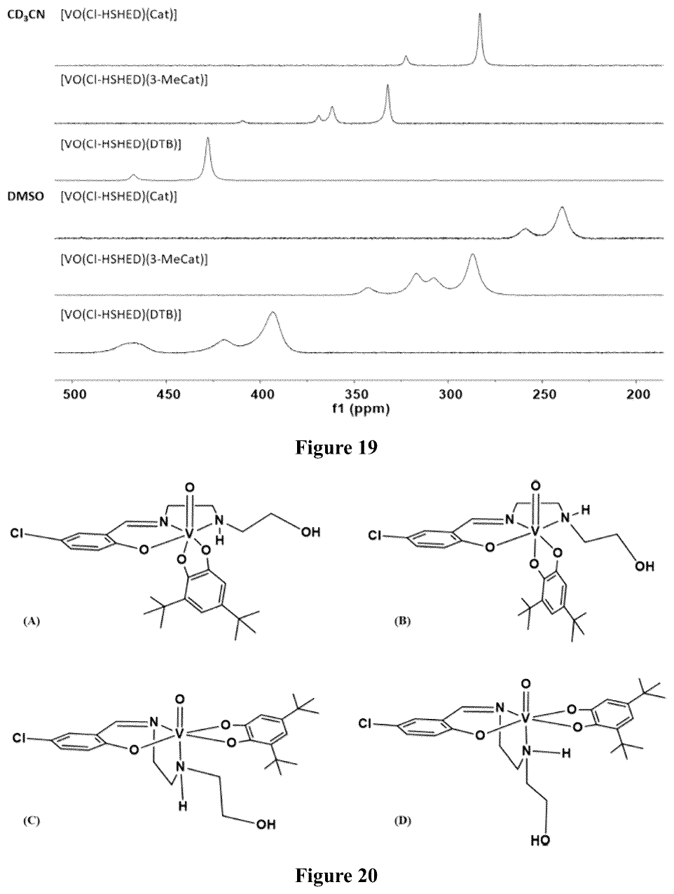

The multiple signals for each compound, as shown in FIG. 19, varied in the ratio as the solvent is changed as described below and consistent with the various peaks representing different isomers. The combination of the Schiff base and the catechol have the potential to form multiple racemic and diastereomeric complexes, and in FIG. 20, we show four of eight possible geometric isomers. Spectra for [VO(Cl-HSHED)(Cat)] in CD₃CN (FIG. 19) showed a sharp prominent peak at 283 ppm and a minor peak at 323 ppm. When dissolved in DMSO-d6, the peaks were shifted to 238 and 259 ppm, respectively; a shift of about 50 ppm and the line widths increased consistent with a more rapid exchange in DMSO-d6. For the catecholate complex, in CD₃CN, the ratio of major-to-minor peaks was 5:1, and in DMSO-d6, the ratio was 3:1. The change in the isomer ratio is indicative that the signals are due to exchanging isomers. In the case of [VO(Cl-HSHED)(DTB)] in CD₃CN, a difference of about 200 ppm with a significant peak at 428 ppm and a minor peak at 468 ppm was observed. In DMSO-d6 [VO(Cl-HSHED)(DTB)], and three signals are observed, a major peak at 394 ppm, and two other minor overlapping peaks of comparable intensity at 420 and 469 ppm. The changes in chemical shifts and the different number of isomers formed for [VO(Cl-HSHED)-(DTB)] suggest that the complexes are labile in solution. Furthermore, the differences in line widths in the two solvents are consistent with the kinetics of the isomer exchange in two different solvents, which confirmed that these signals are due to isomers and not impurities or hydrolysis products. Finally, similar to the complex with the asymmetric DTB ligand, [VO(Cl-HSHED)(DTB)], that with the asymmetric 3-MeCat ligand, [VO(Cl-HSHED)(3-MeCat)], showed four signals in the ⁵¹V NMR spectra in both solvents which are attributed to four isomers. The ¹H NMR spectra confirmed the interpretation that isomers are observed for the catecholate complex, the DTB complex, and the 3-MeCat complexes in solution.

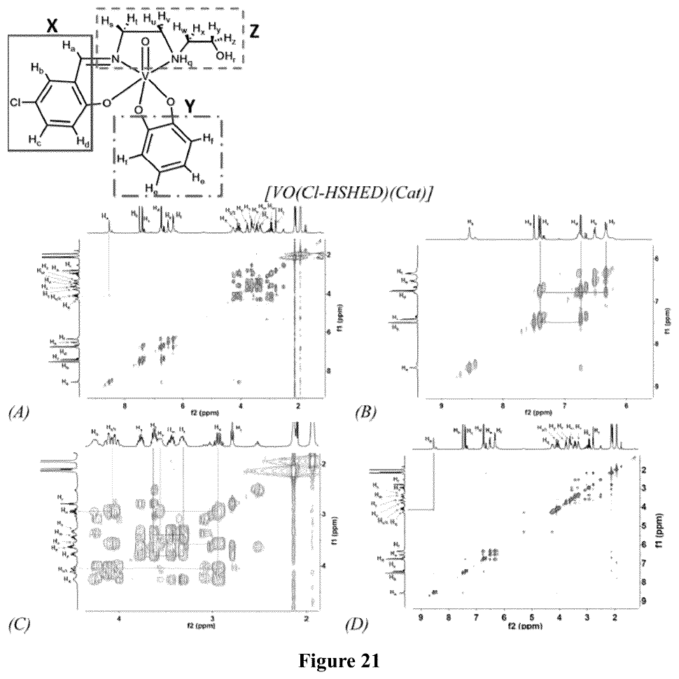

The 2D NMR spectra were recorded in CD₃CN and DMSO-d6, and selected spectra in CD₃CN are shown in FIGS. 21-23. To assign the protons in the conformation of [VO(Cl-HSHED)(Cat)], ¹H-¹H 2D COSY and the ¹H-¹H 2D NOESY were run in d3-CD₃CN and DMSO-d6. The NMR spectra contained groups of larger signals and groups of smaller signals, as anticipated for a compound with both major and minor isomers in solution but we focused on the assignment of the major isomer by identifying the three spin systems (X, Y, and Z) in the compound. FIG. 21 shows the labeled [VO(Cl-HSHED)-(Cat)]. Based on integrations in the ¹H based on the cross peaks in the COSY and the fact that He and Hd had coupling constants, NMR spectra, and correlations in the COSY (FIG. 7(b)), we assigned Hₐ as the proton on the imide, and H_{b}, H_{c}, and H_{d} are defined as the splitting system X (the Schiff base). These assignments were of 8.9 Hz. This left the assignments of the signals for H_{f} and Hₑ on splitting system Y, which showed that both protons had a coupling constant of 8.1 Hz. The slightly larger integration of the H_{f} signal is due to overlapping signals from the minor isomer.

The ¹H-¹H COSY NMR spectra of the aliphatic region of [VO(Cl-HSHED)(Cat)](FIG. 21(c)) displayed cross-peaks between H_{q} and H_{v/w/x}, which corresponded to the proton on the imine and one of the protons on the 1,2-ethanediamine chelate ring adjacent to the imide (H_{y}) and the Hₑ and Hₓ on the ethanol arm in spin system Z. Furthermore, the coupling between the triplet at 2.8 ppm (Hᵣ) and H_{y}/z was assigned to the proton on the oxygen (Hᵣ) and the adjacent downfield protons H_{y} and Hz that corresponded to the CH₂ adjacent to the OH group. Since H_{y} sees H_{z}, H_{w}, and Hₓ, the ethanol arm spin system is defined. The fact that both H_{w} and Hₓ interact with the NH (H_{q}) confirms that these protons are adjacent to the amine nitrogen in the same splitting system.

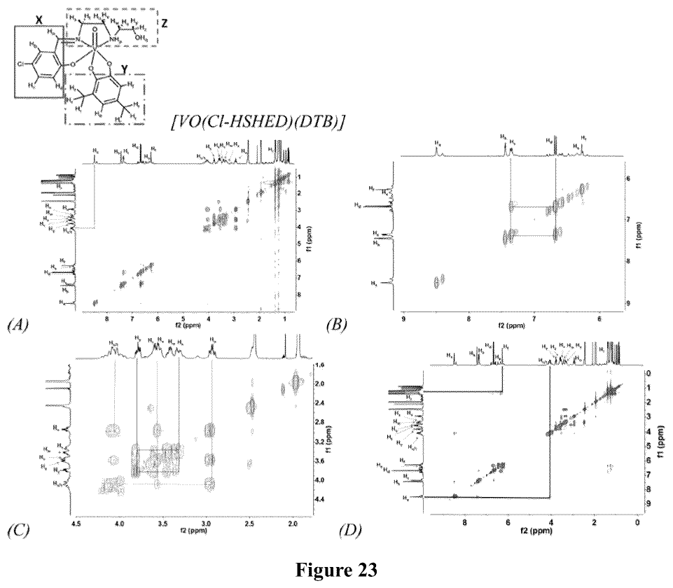

NOESY NMR spectra were collected to gather information on through-space interactions through spin-lattice relaxation within the 3D structure. Observation of off-diagonal cross-peaks between signals, as shown in FIG. 23(b), enabled reports on which protons are within the 5 Å. In the NOESY of [VO(Cl-HSHED)(Cat)] (FIG. 21(d)), Hₐ saw H_{s/t} which showed that the 1-2-ethanediamine chelate ring saw the imine nitrogen.

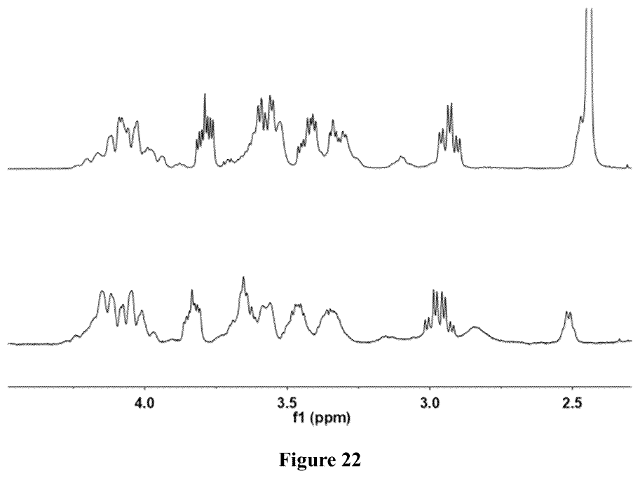

In the case of [VO(Cl-HSHED)(DTB)], the spectra showed less clear coupling patterns (FIGS. 22 and 23). Since coupling was observed with both the OH proton and the NH proton, ¹H NMR spectra were recorded with these groups deuterated with the intent to clarify the observed coupling patterns (FIG. 22). Deuteration simplified the coupling pattern, and the spin system was divided into two spin systems; in the partial spectrum, as shown in FIG. 22, from 2.0 to 4.5 ppm of [VO(Cl-HSHED)(DTB)] with and without the D₂O spike showing simplification of the doublet quartet (near 3.0 ppm) and the multiplet from 4.0 to 4.2 ppm. Not only did the D₂O remove the exchangeable OH and NH protons but also the integrations and the resolution of the remaining coupling patterns were simplified which enabled a more reliable interpretation of the spectra.

For [VO(Cl-HSHED)(DTB)], based on integrations in the ¹H NMR spectra and correlations in the COSY (FIG. 23(a)-(c)), Hₐ was assigned as the proton on the imine (8.48 ppm) on the spin system X. H_{b} was assigned as the doublet at 7.45 ppm and H_{c} as the doublet of doublets at 7.36 ppm. The extra smaller peaks in the NMR such as the peak at 8.39 ppm arose from another isomer in solution. Hd was assigned as the doublet at 6.68 ppm and completed the spin system X (the Schiff base aromatic ring). The He and H_{f} protons were assigned to the peaks at 6.34 and 6.26 ppm on the spin system Y (the catechol). Cross-peaks observed between Hₐ and H_{s/t} were similar to those of [VO(Cl-HSHED)(Cat)] at 4.10 and 4.02 ppm in the ¹H-¹H 2D COSY and NOESY, which enabled the aromatic part of the spectrum to be linked to the solution assignments for the aliphatic region on the spin system Z. Specifically, the NOESY NMR spectrum collected from [VO(Cl-HSHED)(DTB)] enabled the determination of the proximity between protons in the two aromatic regions, between aromatic and aliphatic regions, and between five-membered aliphatic ring and ethanol group. For instance, a NOE cross peak between H_{d} and Hᵣ, showed a close vicinity between the Schiff base aromatic group and the t-Bu protons. Cross-peaks between Hr and Hy/z showed a close proximity between these two aliphatic protons and showed that one t-Bu group was near the CH₂OH group.

In summary, as shown in FIG. 20, the most likely isomers are structures A and B for [VO(Cl-HSHED)(DTB)] observed in a CD3CN solution. The isomer interchange is consistent with the catechol dissociating in a stepwise manner; first, the axial Cat-O-bond will break. Next the cat ligand will rotate, followed by a bond reformation to form an equatorial Cat-O-bond. This process would correspond to conversion of isomer A into an isomer not shown. The NOESY and COSY spectra of [VO(Cl-HSHED)(DTB)] are consistent with the 1D ⁵¹V NMR and the ¹H NMR spectra, in that the DTB catechol is labile. This gives rise to multiple isomers in solution, as shown by the NMR spectra in FIG. 19. The focus on the spectrum, as illustrated in FIG. 20(A), is structure elucidation of the [VO(Cl-HSHED)(Cat)] complex where the t-Bu groups are replaced by H-atoms.

Finally, for the [VO(Cl-HSHED)(3-MeCat)] complex, both ¹H-¹H 2D COSY and a NOESY were collected. As in the cases of [VO(Cl-HSHED)(Cat)] and [VO(Cl-HSHED)(DTB)], major and minor isomers were visible in the ⁵¹V NMR spectra. Four isomers were observed in the ⁵¹V NMR spectra for the [VO(Cl-HSHED)(3-MeCat)] complex, three of which were also observed in the ¹H NMR spectra, making this system more difficult to interpret. The NMR spectrum was simplified by spiking the sample with a drop of D₂O to remove coupling across the heteroatoms. In summary, these spectra are consistent with the interpretations that multiple isomers are being formed in solution, and that for this complex, the minor isomers were more stable and had higher relative concentrations with respect to the major isomer than for the [VO(Cl-HSHED)(Cat)] and [VO(Cl-HSHED)(DTB)] complexes.

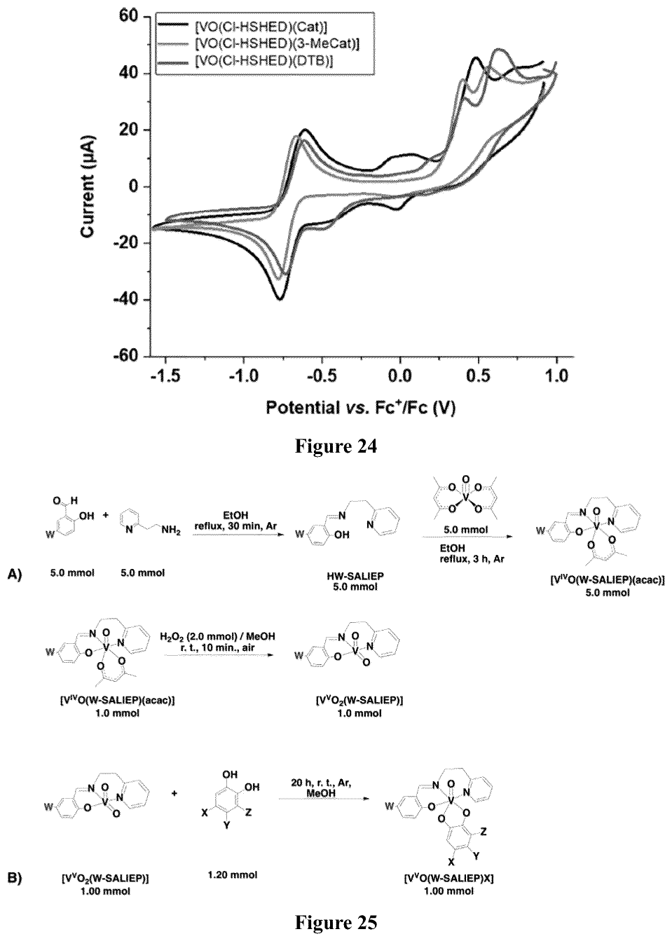

Electrochemistry of Non-Innocent Ligand [VO(Cl-705 HSHED)] Complexes in CH₃CN. The redox chemistry for these non-innocent vanadium complexes was particularly interesting because the reduction could happen at either the ligand or the vanadium center. The values are reported versus Fc+/Fc and also reported against the decamethylferrocenium/decamethylferrocene redox couple (Me₁₀Fc+/Me₁₀Fc). While Fc+/Fc is the IUPAC redox standard and more prevalent in the literature, the Me₁₀Fc+/Me₁₀F standard is less solvent dependent and, hence, more useful to translate the results in organic solution to aqueous solution where the vanadium complexes were too unstable to conduct the redox chemistry of relevance to the biological studies. CVs are measured in acetonitrile, which provides a large solvent window with excellent solubility of the vanadium complexes with 0.1 M TBAP as the supporting electrolyte and ferrocene as the internal standard. Solutions of 2.0 mM vanadium complexes, 2.0 mM ferrocene, and 100 mM TBAP CVs were recorded at 100 mV s⁻¹ (FIG. 24). Spectroelectrochemical data were also obtained to further characterize these complexes.

Two quasi-reversible waves were observed, one involving the catechol between 0.0 and 1.0 V and the other showing the V(V/IV) couple at ˜−0.7 to −0.8 V versus Fc+/Fc. The voltammograms of the free catechol ligand show ligand oxidations in a similar region as for the complexes. The E_{1/2} values are listed in Table 6. For these complexes, the observed chemically reversible redox couple in the −0.7 to −0.8 V region was consistent with reduction at the vanadium center and not the catechol. These values correspond to −0.17 to −0.30 versus Me₁₀Fc+/0 acetonitrile, or close to 0 V in water versus the NHE, ignoring the solvent dependence of the V(V/IV) couple, which is less affected in comparing values in different solvents using measurements referenced versus Me₁₀Fc+/0 than Fc+/0. Thus, the reduction potentials of the V(V) complexes are such that they are easily reduced by intracellular biological reductants.

TABLE 6
E_{1/2} Potentials and Other Electrochemical Data for the V(V/IV) Couples of the Cl-HSHED
and HSHED Catecholate Complexes Averaged with Standard Deviations from Three Samples.

| | | | D [symbol] × 10 [symbol] | | | |
|---|---|---|---|---|---|---|
| complex | E_{1/2} vs Fc⁺/Fc (V) | Δ [symbol] (V) | (cm [symbol] s⁻¹) | I [symbol] (μA) | I [symbol] (μA) | I [symbol] /I [symbol] (μA) |
| [VO(Cl-HSHED)(Cat)] | −0.679 ± 0.002 | 0.17 ± 0.001 | 6 ± 2 | 31 ± 4 | −31 ± 5 | 1.06 ± 0.09 |
| [VO(Cl-HSHED)(3-MeCat)] | −0.709 ± 0.005 | 0.145 ± 0.003 | 5 ± 2 | 29 ± 4 | −28 ± 5 | 1.02 ± 0.03 |
| [VO(Cl-HSHED)(DTB)] | −0.782 ± 0.004 | 0.14 ± 0.03 | 5 ± 2 | 26 ± 2 | −27 ± 4 | 0.98 ± 0.08 |
| [VO(HSHED)(Cat)] | −0.698 ± 0.001 | 0.092 ± 0.003 | 21 ± 4 | 62 ± 12 | −63 ± 12 | 1.00 ± 0.01 |
| [VO(HSHED)(3-MeCat)] | −0.727 ± 0.001 | 0.080 ± 0.003 | 10 ± 3 | 38 ± 5 | −38 ± 6 | 0.99 ± 0.05 |
| [VO(HSHED)(DTB)] | −0.802 ± 0.002 | 0.159 ± 0.001 | 7 ± 1 | 34 ± 2 | −36.55 ± 0.04 | 1.93 ± 0.04 |

^{a}CVs ran at 100 mV s⁻¹ in CH₃CN. [symbol]
indicates data missing or illegible when filed

As the substituent varied, so did the E_{1/2} potential of the [VO(Cl-HSHED)] series and the [VO(HSHED)] series of complexes (Table 6). The most negative E_{1/2} values reported for the complexes (most difficult to reduce) had the more electron-donating catechol (FIG. 24, Table 6). This showed that [VO(Cl-HSHED)(Cat)] was more readily reduced compared to the other Cl-HSHED and HSHED complexes. The vanadium center accepted electrons more readily for the complex with the catechol that was less electron-donating (Cat) than 3-Me (3-MeCat) and 3,5-di-t-Bu (DTB)-substituted catechols. A similar pattern was observed for the parent complexes.

The electronic structure of the vanadium is altered based on the electron-donating nature of the catecholate substituents, which was in turn directly observed in the V(V/IV) redox process in the CV, as was previously reported in the ligand-to-metal charge-transfer bands in the absorption spectroscopy of similar mono-oxido vanadium complexes. When the catechol is deprotonated and coordinated to the vanadium, it is more readily oxidized than the free catechol. In addition, the electrochemical measurements showed differences between the complexes with the halogenated Schiff base ligand and those of the corresponding complex with the parent Schiff base, with the latter being somewhat more difficult to reduce.

This smaller Schiff base substituent effect compared to those on the catecholato ligands arose because of the change in the electronic structure, with the addition of an EWG Cl-atom on the vanadium scaffold. The substitution of the EWG Cl-atom involved effects in the σ framework of the vanadium complexes, as well as the backbonding in the π-framework, which depended on the nature of the catechol as there are two oxidation processes, one for the complex as well as for the ligand. The V(V/IV) couples were chemically reversible (Ipa/Ipc values between 0.90 and 1.05, Table 6) but electrochemically quasi-reversible in CH₃CN as the peak-to-peak separations were large. These effects were not due to the lack of iR compensation in these measurements because the ΔEp values were much larger than those for the Fc+/0 couple under the same conditions. The diffusion coefficients were measured from the peak currents in the CVs at three different scan rates. A pattern emerges that more substituted catechol complexes diffuse slower for both series of complexes. As expected, similar diffusion values were observed for [VO(Cl-HSHED)(DTB)] and [VO(HSHED)(DTB)]. The diffusion rates are one of the factors that affect how rapidly and the extent by which the complexes enter the cells by passive diffusion. Other factors include hydrophobicity and stability under biologically relevant conditions. For similar hydrophobicity, the lower the diffusion rate, the slower the complex enters into the cell. This is an important factor to consider for these labile complexes. Similarities between diffusion constants between the two DTB complexes mean that any differences in the uptake are due to changes in hydrophobicity and stability in the media.

The observed large values of ΔEp for the V(V/IV) couples compared to the Fc+/0 couple under the same conditions were consistent with the presence of the geometric isomers that would have somewhat different redox potentials, as the isomers in the ⁵¹V NMR spectra have quite different chemical shifts and, hence, different electron densities at the V(V) centers. The equilibrium of different isomers for the V(V) and V(IV) oxidation states on the electrochemical timescales would shift between the two oxidation states because of changes in electronic and steric interactions (bond length changes). Therefore, the redox process was a weighted average of the reduction potentials of the V(V) isomers, whereas the oxidation process was the weighted average of the oxidation potentials of the V(IV) isomers, which complicated further interpretation of the electrochemical data with respect to one isomer.

Antiproliferative Activities of Vanadium Complexes. We conducted experiments from freshly prepared solutions in which the Schiff base vanadium-(V)-catechol complexes were intact at least initially. Activities were also examined with solutions that have complexes that had been decomposed for 24 h, at which time the complex had reacted completely with the components of the media. At that time, the decomposed complex was added to the assay medium, resulting in the measurement of the activity of the completely decomposed complex. These experiments enabled a comparison of the activity of the intact complex and the products that formed on its decomposition.

The IC50 values were measured for T98 g (human 826 glioblastoma) cells in a 72 h assay of both fresh solutions of vanadium complexes and 24 h aged solutions in culture media. No intact complexes were detected after the incubation with cell culture medium after 24 h at 310 K. The most favorable activity ratio of fresh and aged was observed for [VO(HSHED)(DTB)] solutions and [VO(Cl-HSHED)(DTB)] solutions. Consistent with the earlier results, fresh solutions of [VO(HSHED)-(DTB)] showed higher activity (IC50=1.9±0.2 μM in 72 h assay, Table 7) in T98 g cells, while aged solutions of this compound were ˜10-fold less active under the same conditions (Table 7). All the complexes decomposed within minutes after having been added to cell culture buffer, as was observed using UV-vis spectroscopy (see the t_{1/2} values in Table 7). The activities of fresh solutions of [VO(HSHED)(DTB)] and [VO(Cl-HSHED)(DTB)] in T98 g were about an order of magnitude higher than those of fresh solutions of Na₃VO₄ or a standard anticancer drug, cisplatin, in the same cell line (Table 7).

TABLE 7
Antiproliferative Activities in T98g Cells and Stabilities under Cell Culture
Conditions for [VO(HSHED)] and [VO(Cl-HSHED)] Complexese

| | IC₅₀ μM | | | | |
|---|---|---|---|---|---|
| | T98g | T98g | HFF-1 | t_{1/2} | |
| compound | (fresh) [symbol] | (aged) [symbol] | (fresh) [symbol] | (295 K) [symbol] | |
| [VO(Cl-HSHED)(DTB)] | 4.1 ± 0.3 | 9 ± 1 | 1.1 ± 0.1 | ~15 | min |
| [VO(Cl-HSHED)(3-MeCat)] | 34 ± 7 | 18 ± 3 | 22 ± 2 | ~15 | s |
| [VO(Cl-HSHED)(Cat)] | 19 ± 2 | 8.1 ± 0.6 | 8.7 ± 2 | ~15 | s |
| [V(O)₂(Cl-HSHED)] | 37 ± 4 | 67 ± 7 | ND^{d} | ND^{d} | |
| [VO(HSHED)(DTB)] | 1.9 ± 0.2 | 21 ± 1 | 1.9 ± 0.4 | ~5 | min |
| [VO(HSHED)(3-MeCat)] | 46 ± 9 | 20 ± 3 | 26 ± 7 | <15 | s |
| [VO(HSHED)(Cat)] | 19 ± 7 | 22 ± 4 | 6.7 ± 0.9 | <15 | s |

| | | | | |
|---|---|---|---|---|
| [V(O)₂(HSHED)] | 44 ± 4 | 70 ± 8 | ND [symbol] | ND [symbol] |
| Na₃VO₄ | 26 ± 4 | 24 ± 4 | 23 ± 3 | ND^{d} |
| cisplatine [symbol] | 33 ± 1 | >50 | 45 ± 4 | |

^{a} Half-life times of the complexes in fully supplemented cell culture medium (measured by UV-vis spectroscopy at 295K, [V] = 100 μM).
^{b} IC50 values in T98g cells or HFF-1 (72 h treatments) when the complexes were mixed with cell culture medium at ~1 min (295K) before the cell treatment.
^{c} IC50 values in T98g cells (72 h treatments) when the compounds were pre-incubated with cell culture medium for 24 h at 310K, 5% CO₂ before addition to the cells.
^{d}Not determined due to the absence of prominent absorbance bands in the visible range.
^{e} Human foreskin fibroblasts. [symbol]
indicates data missing or illegible when filed

Generally, the complexes with the Cl-HSHED ligand were slightly more stable in cell culture medium compared with those with the HSHED ligand, and the complexes with the DTB ligand were more stable than those with Cat and 3-MeCat ligands (Table 7). Despite a longer lifetime of [VO(Cl-HSHED)(DTB)] in cell culture medium compared with [VO(HSHED)(DTB)] (Table 7), the Cl-substituted compound was slightly less active (IC50=4.1±0.5 μM for the fresh complex in 72 h assay), and there was only ˜2-fold difference in the activities of fresh and decomposed [VO(Cl-HSHED)(DTB)] (Table 7). These results showed that the Cl atom in the Schiff base ligand increased hydrolytic stability and ease of reduction but slightly decreased the biological activity of V(V) complexes (see Table 6 and FIG. 24). This demonstrates that stability alone is not the only factor that affects the biological activity. Further work is needed to understand the effects of the redox activity by investigating complexes that are less easily reduced compared to the control.

Since cancer cells exist in the presence of normal cells, it is important that the compounds being developed for treatment of cancer cells in the presence of normal cells, the compounds and the components (ligands and metal) show lower toxicity against the normal cells. Studies with cells, in which the proliferative cell effects are investigated, are often accompanied with cells in a normal cell line. The cytotoxicities of V(V) complexes in a human brain cancer cell line, T98 g, were compared with those in a human non-cancer (foreskin fibroblasts, HFF-1) cell line. Cytotoxicities of fresh compounds in 72 h assays were similar or higher in HFF-1 cells compared with T98 g cells, judging from the IC50 values listed in Table 7. These results are consistent with the toxicity of anticancer drugs to rapidly growing non-cancer cells, including fibroblasts, which is the main source of side effects of cancer chemotherapy. On the other hand, analysis of concentration-viability profiles has shown that treatments of non-cancer HFF-1 cells with low concentrations (0.4-5 μM V) of fresh V(V) complexes for 72 h have led to increased cell viability (120-140%) compared with no-V controls. This effect was most pronounced for the two V(V) complexes with DTB ligands ([VO(HSHED)-(DTB)] and ([VO(Cl-HSHED)(DTB)]), to a lesser extent, in [VO(HSHED)(3-MeCat)].

Previously, we demonstrated that the free ligands showed negligible cytotoxicity in the absence of V (IC50>50 μM). We found that the greatest antiproliferative activities of the ligands were due to the catechols compared to the Schiff base scaffold. In the case of the normal human foreskin fibroblast cell line HFF-1, and furthermore, we reported that they show medium cytotoxicity at 14.7±0.5 μM for the [VO(HSHED)-(DTB)] (Table 7). The 10-fold higher cytotoxicity in the current study toward HFF-1 cells, 1.9±0.4 μM, presumably reflects the fact that cytotoxicities of HFF-1 cells are more sensitive to the details of the experiment, such as number of cells, the doubling rates, the number of passages, and other factors. The increase in cell viability at low V concentrations was not observed for the cancer T98 g cell line but was reported previously for bone fibroblasts (osteoblasts) and can contribute to the known tissue-regenerating properties of V compounds. Data emphasize that the activities of anticancer drugs in various cell lines cannot be compared based on the IC50 values alone.

Indeed, studies in other cells such as Mycobacterium smegmatis documented that the Schiff base scaffold and the catechols are at least 10-fold less toxic than the complex in M. smegmatis. These studies showed a 100-fold lower toxicity in M. smegmatis than that observed in the T98 g cells. Importantly, the most toxic catechol was the parent catechol, and the sterically hindered catechols were much less toxic. This conclusion was confirmed in animal studies, in mice, that showed the complex was less toxic than vanadate and the DTB ligand.

Typical IC50 values for the parent and Cl-substituted complexes that rapidly decomposed (˜20-40 μM in 72 h assays, Table 7, those not containing the DTB ligand) were close to those of their decomposition products (˜20 μM) and for Na₃VO₄ (˜40 μM) in T98 g cells. This was consistent with the interpretation that the complexes had decomposed in the medium before they had the opportunity to enter the cells by passive diffusion to any significant extent, and that V(V/IV) decomposition products were responsible for their antiproliferative activities. In the case of [VO(Cl-HSHED)(Cat)], the decomposed complex was ˜2-fold more active than the fresh solution (Table 7). This may be due to the redox reactions of the catechol ligand and V(V) in cell culture medium under an ambient atmosphere, generating a range of cytotoxic species, including V(V) peroxido complexes and semiquinone radicals.

Relatively low antiproliferative activities (IC50˜40 μM in 72 h assay) were observed for fresh solutions of the Schiff base scaffold complexes {both [V(O)₂(HSHED)] and [V(O)₂(Cl-HSHED)] in Table 7}. These complexes were both less potent after pre-incubation with cell culture medium for 24 h at 310 K (IC50˜70 μM in 72 h assay, Table 7). In contrast, antiproliferative activity of a pre-incubated solution of Na₃VO₄ has similar potency to that of a fresh solution. These results, together with UV-vis spectroscopy data, support the interpretation that the HSHED or Cl-HSHED ligands remain at least partially bound to V(V) for a sufficient time to enable some intact complex to enter cells for the DTB complexes during the cell assays but the complexes with the other catecholato ligands decompose too rapidly to enter cells intact. These results are consistent with the interpretation that the observed greater toxicity of aged solutions of the [VO(Cl-HSHED)(Cat)], [VO(Cl-HSHED)-(3-MeCat)], and [VO(HSHED)(3-MeCat)] complexes are due to the free catechol (or substituted methyl substituted catechol) reactions in the culture medium suggested above.

In summary, the three new complexes designed were more hydrophobic, more hydrolytically stable, and more redox active than their HSHED analogues. The patterns observed for the [VO(Cl-HSHED)] series of complexes were similar to those for the [VO(HSHED)] complexes. Most importantly, the [VO(Cl-HSHED)(DTB)] and the [VO(HSHED)(DTB)] complexes were by far the antiproliferative complexes in their respective series and an order of magnitude more potent than cisplatin under the same conditions. This observation confirms the fact that the most sterically hindered and hydrophobic complex in both series were most effective. However, the electronic effects are more difficult to evaluate because the electronic changes were accompanied by changes in hydrophobicity and sterics/stability. For the DTB complexes, the Cl-substitution made the complex more stable, hydrophobic, and more easily reduced. The higher stability and greater lipophilicity of the complex with the Cl-substituted Schiff base scaffold should change its intracellular distribution in a way that influences antiproliferative activities beneficially. This suggested that future complex designs should focus on complexes less easily reduced than the parent complexes since the ease of intracellular reduction may have been responsible for the reduced antiproliferative efficacy.

Conclusion

Developing new complexes and comparing their potency as anticancer agents will be important for structure-activity relationships for future complex development. Access to additional complexes will allow future studies on the anticancer mode of action of non-innocent Schiff base V-catecholate complexes. The newly synthesized [VO(Cl-HSHED)(Cat-X)] complexes where the catecholate is either Cat, 3-MeCat, or DTB were characterized by UV-vis, multinuclear and multidimensional NMR, MS-ESI, and electrochemistry. The three new complexes designed and tested demonstrated that the sterically hindered, more hydrophobic, and less easily reduced non-innocent Schiff base V-catecholato complex is the most potent antiproliferative complex, with the lowest IC50 for the fresh solutions and a higher IC50 value for aged solutions.

In summary, the studies of the three new complexes with Cl-substituted Schiff base scaffold and substituted catecholates have provided an additional example of an anticancer agent that is superior to cisplatin in the T98 g cell line. The observed activity ratio of fresh and decomposed solutions of the best of the new complexes, [VO(Cl-HSHED)(DTB)], showed similar improved activity compared to cisplatin as the parent [VO(HSHED)(DTB)] complex in the T98 g cell line that is an in vitro model for difficult to treat gliomas. This antiproliferative activity is sensitive to the stability and hydrophobicity of the complex in biological media in both the parent and Cl-substituted Schiff base V-complexes. Our research has enhanced our knowledge and shown that the increased redox properties of the Cl-substituted complexes has not been favorable with regard to the complexes antiproliferative effects, but steric effects that increased hydrolytic stability of the complexes are important in optimizing the activity in both the HSHED and Cl-HSHED series.

Where does this leave us with regard to structure-reactivity relationships? In this Example, three new non-innocent vanadium Schiff base complexes were introduced, and the addition of the Cl-group improved stability, increased hydrophobicity, and increased the ease of reduction of the complexes. Importantly, our studies have introduced a second complex, [VO(Cl-HSHED)(DTB)], with potential for intratumoral injection. Such complexes should enter cancer cells by passive diffusion, leading to rapid cancer cell death, while the non-toxic V(V/IV) decomposition products that diffuse away from the tumor cause no toxic effects to the healthy cells. One important aspect of the current work is that it has provided detailed characterization of the three new complexes introduced and combined with previous complexes allowed us to carry out structure-activity analysis. We conclude that the limited stability and hydrophobicity of V(V)-Schiff base-catecholato complexes under cell culture conditions remain crucial factors for the observed antiproliferative activity in vitro. The ˜10-fold decrease in the observed IC50 values and antiproliferative activities of [VO(HSHED)(DTB)] and [VO(Cl-HSHED)(DTB)] in cancer cell lines after their decomposition in cell culture medium (24 h at 310 K) remains an advantage for the potential use in intratumoral injections. We hypothesize that rapid cellular uptake and rapid reactions within cancer cells in tumor tissue are likely to have beneficial effects in vivo. However, since the introduction of the Cl-group did not lead to the significant increase in antiproliferative effects compared to the parent complex, the combined introduced changes in properties in the new series of complexes have not at all been beneficial. Since increased stability and hydrophobicity have been reported to increase antiproliferative activity, the ease of reduction of these complexes may have counteracted the increased stability and hydrophobicity of the new complexes, and/or the increased stability and changes in hydrophobicity may have affected intracellular biodistributions, yielding complexes with somewhat less activity. Combined analysis led us to continue to identify stability and hydrophobicity as key factors to be targeted in the future design of these complexes and that decreased ease of reduction may improve anticancer effects of complexes.

### Example 5. Vanadium(V) Pyridine-Containing Schiff Base Catecholate Complexes are Novel Lipophilic, Redox-Active and Selectively Cytotoxic in Glioblastoma (T98 g) Cells

Overview

Two series of complexes with pyridine-containing Schiff bases, [VVO(SALIEP)L] and [VVO(Cl-SALIEP)L] (SALIEP=N-(salicylideneaminato)-2-(2-aminoethylpyridine; Cl-SALIEP=N-(5-chlorosalicylideneaminato)-2-(2-aminoethylpyridine, L=catecholato(2-) ligand) were synthesized. Characterization by ¹H and ⁵¹V NMR and UV-Vis spectroscopies confirmed that: 1) most complexes form two major geometric isomers in solution, and [VVO(SALIEP)(DTB)] (DTB=di-tert-butylcatecholato(2-)) forms two isomers that equilibrate in solution; and 2) tert-butyl substituents was necessary to stabilize the reduced V(IV) species (EPR spectroscopy and cyclic voltammetry). The pyridine moiety within the Schiff base ligands significantly changed their chemical properties with unsubstituted catecholate ligands compared with the parent HSHED (N-(salicylideneaminato)-N′-(2-hydroxyethyl)-1,2-ethanediamine) Schiff base complexes. Immediate reduction to V(IV) occurred for the unsubstituted-catecholato V(V) complexes on dissolution in DMSO. By contrast, the pyridine moiety within the Schiff base significantly improved the hydrolytic stability of [VVO(SALIEP)(DTB)] compared with [VVO(HSHED)(DTB)]. [VVO(SALIEP)(DTB)] had moderate stability in cell culture media. There was significant cellular uptake of the intact complex by T98 g (human glioblastoma) cells and very good antiproliferative activity (IC50 6.7±0.9 μM, 72 h), which was ˜five-fold higher compared with the non-cancerous human cell line, HFF-1 (IC50 34±10 μM). This made it a potential drug candidate for the treatment of advanced gliomas by intracranial injections.

Introduction

Aggressive cancers that include brain and pancreatic cancers are difficult to treat and continue to pose a challenge to develop antitumor therapies. Glioblastoma is by far the most common and aggressive form of glioma (brain tumor) with an average incidence of three cases per 100,000 individuals. Glioblastoma (previously known as glioblastoma multiforme, GBM) is characterized by a highly proliferative population of cells that invade surrounding tissue and that frequently recur after surgical intervention and chemotherapy. Traditional methods to treat glioblastoma include surgical intervention and/or chemotherapy. Due to the very high rate of recurring, new treatment methods for glioblastoma are of high interest. One of the main challenges for glioblastoma drug development is the design of drug candidates that can cross the blood brain barrier (BBB), which normally requires them to be small and lipophilic. A promising approach to circumvent the impediment of the BBB is to use intratumoral injections of highly cytotoxic or immunomodulating drugs directly into the inoperable tumor, which represents the next generation of glioblastoma treatments. Such intratumoral injections are currently in Phase I and II human clinical trials for other drugs including platinum-based drugs, such as cisplatin and oxaliplatin, and injections of T-Vec, drugs used to treat advanced melanoma and a range of other cancers. Vanadium salts and a coordination complex, bismaltolatooxovanadium(IV) was considered for the treatment of diabetes and underwent Phase I and II clinical trials before the studies were discontinued and bismaltolatooxovanadium(IV) went off-patent in 2011. The reported efficacy of vanadium compounds against different types of cancer suggests that vanadium compounds may be more successful against cancer rather than diabetes. Furthermore, the reported adverse side effects of V anticancer compounds are better tolerated. Indeed recently, intratumoral injections of the combination of vanadyl sulfate and Newcastle disease virus have been shown to be effective in murine cancer models. This data, combined with the superior activity of the non-innocent vanadum(V) catecholates complexes over cisplatin in cell culture, provide a potential alternative treatment against glioblastoma where there is little hope.

Recently, intratumoral injections of cytotoxic, non-innocent vanadium(V) catecholates with hydrophobic substituents have been proposed as a potential mode of administration of these drugs for difficult-to-treat cancers. Vanadium is a well-known antidiabetic, antimutagenic, antituberculosis, and antiviral and studied first-row transition metal with reported anticancer properties. The beneficial properties are attributed to vanadate, a four-coordinate species and a structural and electronic phosphate analog, which can regulate phosphatases, kinases and mammalian signal transduction. Vanadium(V) catecholate complexes have rather unique properties compared to the reported vanadium Schiff base complexes. Their distinct feature is the short hydrolytic stability under physiological conditions, which enables them to remain intact during cellular uptake in vitro and the potential to cause localized systemic toxicity in cancerous tissues. By contrast, upon hydrolysis the decomposition products that diffuse away would not cause systemic toxicity and indeed can be neuroprotective. The lack of systemic toxicity of the hydrolysis products has been confirmed in an in vivo animal model. The design of V(V)-based drugs has been inspired by the early work by the Pecoraro group in which the structures and spectroscopic signatures of the catecholates of different parent Schiff base and substituted systems, and the stability and biological activity of the decomposition product [VO(dtb)₃]⁻ (dtb=3,5-di-tert-butylcatecholato(2-)) studied by the Lay group. In the catecholate complexes, Schiff base ligands are used to stabilize the vanadium center in the vanadium(V) oxidation state. The lead analog [VO(HSHED)(DTB)] (HSHED═N-(salicylideneaminato)-N′-(2-hydroxyethyl)ethane-1,2-diamine) was 12-fold more cytotoxic than cisplatin in T98 g (glioblastoma) cells. It is also highly cytotoxic in A549 (lung), PANC-1 (pancreatic) and SW1353 (bone chondrosarcoma) cell lines, and non-toxic in mice. The stability and superior cytotoxicity of the lead drug candidate are attributed to the hydrophobic tert-butyl substituents on the catecholate ligand, which increased cell permeability and provided steric effects that increased the overall stability to hydrolysis and V sequestration by transferrin, which inhibits V uptake and cytotoxicity. Additionally, the HSHED Schiff base hydrolyzes into non-toxic decomposition side products under physiological conditions. Another promising agent for intratumoral injections, [VO(Cl-HSHED)(DTB)] (Cl-HSHED=N-(5-chlorosalicylideneaminato)-N′-(2-hydroxyethyl)ethane-1,2-diamine), has been reported recently. The addition of a chloro substituent on the Schiff base of parent complex increases hydrophobicity and tripled the half-life for hydrolysis under physiological conditions, making the [VO(Cl-HSHED)(DTB)] complex another promising agent for intratumoral injections. The

[VO(HSHED)(DTB)] and [VO(Cl-HSHED)(DTB)] complexes have complex dynamic isomer speciation with up to four geometric isomers in solution. This makes it difficult to determine which isomer(s) is(are) responsible for the high cytotoxicity and have the most influence on hydrolytic stability. Thus, we have investigated vanadium(V) catecholates with aromatic Schiff bases to limit the potential number of geometric isomers to two depending on the coordination of the catecholate ligand (assuming equatorial coordination of the Schiff base ligand). In this Example, we report two series of vanadium(V) catecholate complexes with pyridine-containing Schiff bases, [VO(SALIEP)L] and [VO(Cl-SALIEP)L] (FIG. 25). The compound design was derived from the previously reported [VO(HSHED)(DTB)] and [VO(Cl-HSHED)(DTB)] analogs (FIG. 25). Interestingly, a structurally similar Schiff base complex with a methylene linker abbreviated [VO(SALIMP)(DTB)] where SALIMP stands for N-(salicylideneaminato)-2-(2-aminoethylpyridine) can also be prepared. We have chosen the complexes with pyridine-containing Schiff bases to investigate how the lack of H-bonding on the Schiff base affects hydrolytic stability and cytotoxicity of the complexes. The pyridine moiety also improves permeability through membranes and increases metabolic stability of many pharmaceuticals. The structure of the [VO₂(SALIEP)] precursor was first reported by the Pessoa group. This dioxido complex has catalytic properties in the hydroamination of styrene and vinyl pyridine with amines (aniline and diethylamine) where the anti-Markovnikov hydroamination product is favored. The [VO₂(SALIEP)] complex, however, has not been tested in any biological systems or used for the preparation of any biologically relevant vanadium complexes.

Vanadium(V) catecholate complexes are redox-active, and the electrochemical properties of the [VO(HSHED)L] and [VO(Cl-HSHED)L] (L=substituted or unsubstituted catecholato(2-)) complexes have been studied. Solid tumors generally have highly reducing environments due to the presence of intracellular reductants, mainly glutathione and ascorbate and hypoxic regions within the tumor. Thus, studies on the redox chemistry of vanadium(V) catecholates is of interest, particularly since the cytotoxicity of the complexes can result, in part, from intracellular ROS activity. Indeed, many vanadium compounds, generally vanadium(IV) compounds, undergo Fenton type of chemistry with the formation of the ROS species, whereas vanadium(V) complexes must first convert to vanadium(IV) complexes before such chemistry takes place. Little is known about the effects of the Schiff base structure and the substitution pattern on the catecholate ligand on the observed redox properties, and establishing those relationships is also of interest. The VOHSHED and VOCl—HSHED complexes show reversible couples in organic solvents. The stability of the reduced V(IV) species is attributed to the bulky hydrophobic substituents on the catecholate ligand and the stability of the five-membered ring core between NN coordination sites of the Schiff base, the ethylene arm and V center. This may not be the case for the [VO(SALIEP)L] and [VO(Cl-SALIEP)L] complexes, since the pyridine ring on the Schiff base results in a six-membered core between NN coordination sites of the Schiff base, the ethylene arm and V center. In this Example, we explore the impact of these different structural motifs on the observed redox chemistry of vanadium(V) catecholates.

Herein, we report the syntheses and spectroscopic characterization of two vanadium(V) catecholate series abbreviated [VO(SALIEP)L] and [VO(Cl-SALIEP)L] (L=catecholato(2-), 4-tert-butylcatecholato(2-) and di-3,5-tert-butylcatecholato(2-). Specifically, we examined whether: 1) the increased hydrophobicity due to the pyridine ring improved cellular uptake and cytotoxicity; 2) the removal of the H bonding on the aromatic Schiff base affected the redox chemistry and the hydrolytic stability; and 3) different structural motifs impacted redox chemistry of the complexes. Antiproliferative properties of the [VO(SALIEP)L] and [VO(Cl-SALIEP)L] complexes were also studied in T98 g (human glioblastoma) and HFF-1 (human normal skin fibroblasts) cell lines. We established the relationships between the structure, spectroscopic properties and observed cytotoxicity, and compared them with the [VO(HSHED)L] and [VO(Cl-HSHED)L] analogs, as well as cisplatin.

Results and Discussion

Design of new vanadium Schiff base complexes. The complex design was inspired by the promising anti-cancer activity of the [VO(HSHED)(DTB)] analog, which was found to be 12-fold more potent than cisplatin. Cisplatin and oxaliplatin are still used in the clinic, and hence serve as excellent reference compounds to which new potential therapeutics are compared to. Indeed, vanadium compounds can be just as potent or superior to some Pt-compounds for different types of cancer. The synthesis of [VO(HSHED)(DTB)] was first reported by the Pecoraro group in 1992 along with the catecholates containing other Schiff bases. The Pessoa group then reported the synthesis and characterization of [V^{V}O₂(SALIEP)] in 2009 as a catalyst for hydroamination of olefins where the anti-Markovnikov product was favored. Vanadium(V) pyridine-containing Schiff base catecholates, however, have not been reported previously. We explored pyridine-containing Schiff base complexes due to their catalytic properties which, consequentially, resulted in different reactivities that the corresponding HSHED complexes.

Syntheses of the complexes. The [V^{V}O₂(SALIEP)] precursor was synthesized by using a modified procedure where the reaction solvent was changed from acetonitrile to methanol (FIG. 25). The Schiff base was first formed from a condensation reaction between salicylaldehyde and 2-(2-aminoethyl)pyridine in ethanol for 30 min, followed by the addition of the ethanolic solution of [VO(acac)₂]. The isolated [VO(SALIEP)(acac)] complex was oxidized by 30% aqueous H₂O₂ in air for 10 min, yielding the [VO₂(SALIEP)] precursor in a 80% yield. The catecholato complexes were synthesized using 1.0 equivalent of [VO₂(SALIEP)] and 1.2 equivalents of the catecholate ligand. The start of the reaction was evident by an immediate color change from mustard-yellow to deep purple. The reaction mixture was allowed to stir for 20 hr at room temperature (20° C.) under Ar. The yields of the isolated complexes were calculated after drying the complexes under high vacuum and are tabulated in Table 8. The [VO₂(Cl-SALIEP)] precursor and the [VO(Cl-SALIEP)L] series were synthesized using similar methods using 5-chlorosalicylaldehyde for the Schiff base condensation reaction (FIG. 25). The yields (Table 8) of all isolated complexes were after drying the complexes under high vacuum and can potentially be further improved by synthesis optimization.

Unlike other reported V(V) catecholate series, the pyridine series had different isolation methods, as the complexes readily precipitated from solution upon reaction completion. The complexes were isolated using vacuum filtration and washed with cold methanol, and their yields are tabulated below (Table 8). The yields of both parent and chloro-substituted Schiff base analogs were comparable, which showed that the presence of the chloro substituent on the Schiff base did not affect the coordination of the catecholate ligand to the dioxido precursors.

TABLE 8
Percent yields and appearances of the isolated complexes.

| Complex | % Yield | Appearance |
|---|---|---|
| [VO₂(SALIEP)] | 80% (this work | Dark mustard |
| | and previous work) | yellow solid |
| [VO(SALIEP)(Cat] | 61% | Deep purple |
| | | solid |
| [VO(SALIEP)(4TB)] | 25% | Purple solid |
| [VO(SALIEP)(DTB)] | 31% | Deep purple |
| | | solid |
| [VO₂(Cl-SALIEP)] | 75% | Dark mustard |
| | | yellow solid |
| [VO(Cl-SALIEP)(Cat)] | 44% | Purple solid |
| [VO(Cl-SALIEP)(4TB)] | 57% | Deep purple |
| | | solid |
| [VO(Cl-SALIEP)(DTB)] | 22% | Deep purple |
| | | solid |

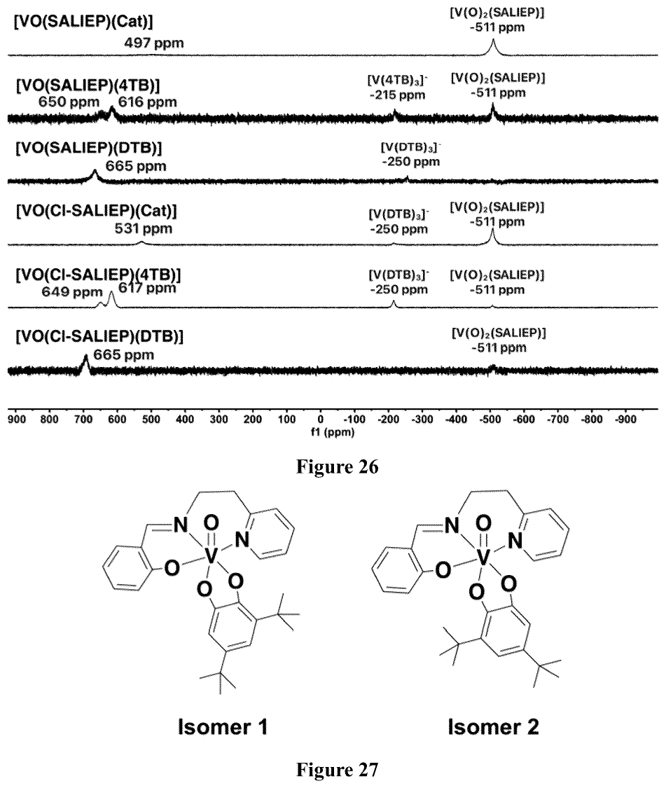

⁵¹V NMR Spectroscopic Characterization. The [VO(SALIEP)L] and [VO(Cl-SALIEP)L] complexes were characterized by ⁵¹V NMR spectroscopy in 10.0 mM solutions of d3-MeCN and d6-DMSO (FIG. 26). The recorded spectra of the complexes were compared to those of the vanadium(V) precursors, [VO₂(SALIEP)] and [VO₂(Cl-SALIEP)]. The [VO(SALIEP)(Cat)] complex exhibits a weak signal at 497 ppm in d6-DMSO. The complex rapidly decomposes to the [VO₂(SALIEP)] precursor, which is evident by the appearance of the peak at −511 ppm. This peak is much broader (half-height width of −25 ppm) compared to that from the isolated complex (˜12 ppm) in the same solvent, which is indicative of paramagnetic broadening by the presence of V(IV). The [VO(SALIEP)(4 TB)] complex exhibits two peaks at 616 and 650 ppm in a 1:1.5 ratio by ⁵¹V NMR integration which correspond to two geometric isomers of the complex. The complex decomposes into [VO(4 TB)₃]⁻ decomposition product and the [VO₂(SALIEP)] precursor which is evident by the appearance of the peaks at −216 and −511 ppm, respectively. V(V) catecholates form reactive tris-catecholato intermediates which exhibit ⁵¹V NMR peaks in the −200 ppm region, such as the vanadium(V) tris-3,5-di-tertbutylcatecholato complex ([V(DTB)₃)]⁻) at −250 ppm. The [VO(SALIEP)(DTB)] complex is the most stable complex of the [VO(SALIEP)L] series, which is evident by the highest intensity broad peak at 665 ppm and a minor peak at −250 ppm. The broadness of the [VO(SALIEP)(DTB)] peak indicates potential formation of two geometric isomers with similar chemical shifts in ⁵¹V NMR spectrum that differ with regard to the orientation of the two tert-butyl groups on the catecholate ligand (FIG. 27). One of the TB groups is spatially closer to the pyridine ring in one isomer and in the other isomer, one of the TB groups is spatially closer to the salicylidene portion of the Schiff base.

The ⁵¹V NMR data of the [VO(SALIEP)L] series demonstrate that the ligand substitution affects the observed ⁵¹V NMR shift. DTB groups are electron-donating groups that result in an increase in the HOMO-LUMO gap and, consequentially, an upfield shift for the vanadium(V) catecholate complex. Thus, the [VO(SALIEP)(DTB)] complex has the most upfield shift in the ⁵¹V NMR spectrum at 665 ppm, although the [VO(SALIEP)(4 TB)] only has a slight downfield shift in the ⁵¹V NMR spectrum. This is distinctly different from the [VO(SALIEP)(Cat)] complex that does not have any substituents on the catecholate ligand and, thus, has a more downfield chemical shift at 497 ppm due to the smaller HOMO-LUMO gap.

The ⁵¹V NMR data of the [VO(Cl-SALIEP)L] showed that they are more stable to decomposition, compared to the parent analogs. The [VO(Cl-SALIEP)(Cat)] complex, for example, exhibits a peak at 531 ppm which has a much higher intensity to that of the [VO(SALIEP)(Cat)] complex. The complex, however, partially decomposes into the [VO₂(C₁-SALIEP)] precursor. The [VO(Cl-SALIEP)(4 TB)] complex exhibits two peaks at 649 and 617 ppm in a 1:1.5 ratio, which correspond to two geometric isomers of the complex. The complex partially decomposes into [VO(4 TB)₃]- and the [VO₂(Cl-SALIEP)] precursor, which is evident by the appearance of the peaks at −216 and −510 ppm, respectively. The [VO(Cl-SALIEP)(DTB)] complex exhibits a major isomer peak at 665 ppm and partially decomposes into the [VO₂(Cl-SALIEP)] precursor. The ⁵¹V NMR data show that the electron-donating tert-butyl substituents result in an increased HOMO-LUMO gap and more upfield chemical shifts of the [VO(Cl-SALIEP)(4 TB)] and [VO(Cl-SALIEP)(DTB)], respectively. Interestingly, the electron-donating nature of the Cl substituent on the π system caused an increased stability of the [VO(Cl-SALIEP)(Cat)] complex compared to its parent analog. All complexes, however, are much less stable compared to the [VO(HSHED)L] and [VO(Cl-HSHED)L] series, since all spectra in d6-DMSO were collected within 5 minutes of sample preparation.

Most [VO(SALIEP)L] and [VO(Cl-SALIEP)L] complexes, except for the [VO(SALIEP)(DTB)] and [VO(Cl-SALIEP)(DTB)], immediately decomposed into their corresponding precursors in d3-MeCN, which indicated that both stability and isomer distribution of the complexes were solvent-dependent. The decreased stability of the complexes in d3-MeCN was consistent with weak hydrogen bonding ability of acetonitrile which, consequentially, reduces the stability of the V(V) complexes. The stability of [VO(SALIEP)(DTB)] and [VO(Cl-SALIEP)(DTB)], however, is unaffected by the highly hygroscopic nature of d3-MeCN. The solvent environment also did not affect the chemical shifts of the [VO₂(SALIEP)] and [VO₂(Cl-SALIEP)] precursors which could be explained by similar dielectric constants of MeCN and DMSO (35.9 and 47.1, respectively).

Most ⁵¹V NMR spectra had a relatively high signal-to-noise ratio, which indicated partial reduction of the complexes to V(IV). The observations could be explained by the fact that the six-membered core connecting NN coordination sites of the Schiff base, which causes the ethylene connector of the Schiff base and the vanadium to canter significantly. The reduction was also solvent-dependent; for example, the observed reduction in acetonitrile, was consistent with the ability of this weak hydrogen bonding solvent to reduce the electron density at the metal center in Cr(V) species by hydrogen bonding to coordinated oxygen donors. Similar effects would facilitate the reduction for V(V) to V(IV), as was observed in Cr(V/IV) redox potentials, and described in the effects of specific solvation in the classical and semi-classical theories on the thermodynamics and kinetics of electron transfer described. Such distortions would decrease the stability of the V(V) complex and increase the likelihood of reduction to V(IV), compared to the VOHSHED complexes that have a five-membered core structure. In addition, the pyridine group also has weak p-acceptor properties that tends to stabilize lower oxidation states, such as V(IV). The observation that both [VO(SALIEP)(Cat)] and [VO(Cl-SALIEP)(Cat)] were immediately reduced to V(IV) in d3-MeCN but were relatively stable in d6-DMSO could also be explained, in part, by the sensitivity of the analogs to trace moisture in the organic solvent. The weak hydrogen bonding ability of d3-MeCN, thus, resulted in the higher residual water content and decreased the overall stability of both [VO(SALIEP)L] and [VO(Cl-SALIEP)L] complexes. Overall, the ⁵¹V NMR data showed the predominance of one geometric isomer for most complexes and their rather unstable nature in organic solvent compared to the reported [VO(HSHED)L] and [VO(Cl-HSHED)L] analogs.

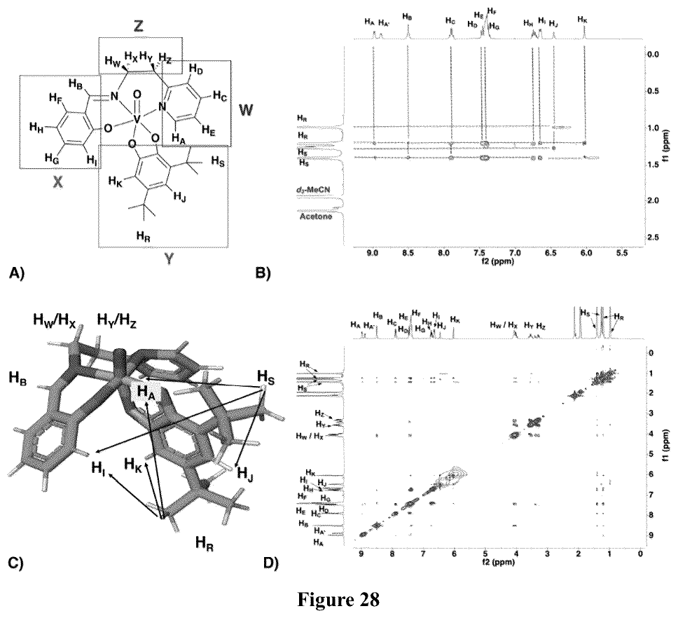

2D NMR Characterization. 2D NMR experiments were undertaken to determine isomer distribution of the parent DTB complex, [VO(SALIEP)(DTB)], and the 3D structure of its major isomer due to the tendency of V(V) catecholates to form multiple geometric isomers in solution. Unlike previously reported HSHED and Cl-HSHED Schiff bases, the SALIEP Schiff base consists of two rigid π systems (salicilydene and pyridine; FIG. 28(A)), which limit the number of potential geometric isomers in solution to two (on the assumption that the Schiff base is constrained to the equatorial plane), depending on the coordination of the asymmetric DTB ligand.

The [VO(SALIEP)(DTB)] complex has four distinctly different nuclear spin systems, abbreviated W-Z where W corresponds to the pyridine ring, X—to the Schiff base, Y—to the catecholate ring and Z—to the ethyl arm (FIG. 28(A)). Since W, X, and Y spin systems exclusively consist of the aromatic protons, their chemical shifts were determined using ¹H-¹H gCOSY NMR. The deduced assignments are shown in FIG. 28(A). The most upfield peak at 8.98 ppm was assigned to the H in the 2 position on the pyridine ring (HA), in addition to the peak at 8.88 ppm, which corresponded to the H_{A′} proton of the minor isomer. According to the ¹H NMR integration, H_{A} and H_{A′} protons are in the 1.3:1 ratio which indicates the major to minor isomer distribution. The singlet at 8.50 ppm was assigned to the imine proton (H_{B}). The protons on the ethylene arm (H_{Y} and H_{Z}) are nonequivalent, which was evidenced by the presence of the multiplets at 3.54 and 3.30 ppm, respectively. The presence of two geometric isomers in solution was also confirmed by the presence of two peaks corresponding to each tert-butyl group, abbreviated H_{R} (1.21 and 0.98 ppm) and Hs (1.40 and 1.27 ppm). The 1H integration of those peaks confirmed that the isomers exist in approximately a 2:1 ratio in solution. Thus, the tertbutylcatechol ligand can be coordinated in two different orientations where 1) one of the tert-butyl groups is spatially closer to the pyridine ring and 2) one of the tert-butyl groups is spatially closer to the salicylidene portion of the Schiff base.

The ¹H-¹H NOESY NMR experiments enabled the major geometric isomer of [VO(SALIEP)(DTB)] to be determined based on the crosstalk between protons. The data showed that both DTB groups coupled with both salicylidene and pyridine spin systems (FIG. 28(B)-(D)). The interproton measurements between H_{R}-H_{A} and H_{S}-H_{A} confirmed that the major isomer contains a tert-butyl group Hs spatially closer to H_{A} of the pyridine ring. Additionally, both DTB groups had medium to weak cross-peaks with H_{c}, H_{D}, and H_{E} protons on the pyridine ring, and H_{H} and H_{I} protons on the salicylidene. H_{R} was the only DTB group to have a crosstalk with the pyridine proton H_{A}, which indicated the closer proximity of the H_{R} protons on the DTB group to the pyridine ring. The interproton distance measurement from the 3D model is 2 Å, which confirmed the presence of the cross-peak with the medium intensity. Both H_{R} and Hs have a weak cross-peak with H_{B}. The interproton distance measurements from the 3D model confirmed a stronger H_{B}-H_{S} cross-peak due to a shorter interproton distance (7.3 Å, 8.1 Å for H_{B}-H_{R}). Crosstalk was also observed between the imine proton H_{B} and two nonequivalent protons on the ethyl arm, H_{W} and H_{X}, confirming the proximity of the X and Z spin systems. To summarize, the 2D NMR data show that the major isomer of [VO(SALIEP)(DTB)] has the TB group spatially closer to the pyridine ring, although two geometric isomers are stable and equilibrate in solution.

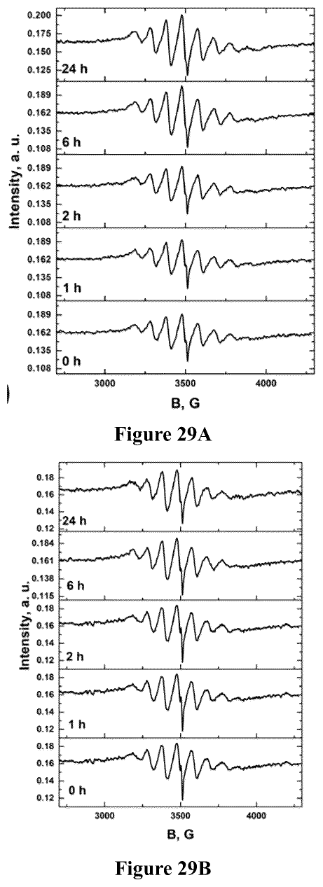

EPR Spectroscopy. EPR studies confirmed that the complexes with unsubstituted catecholate ligands existed predominantly as the reduced V(IV) species in solution. Initially, EPR spectra in 10.0 mM DMSO solutions were collected to demonstrate the formation of V(IV) species and to enable the gᵢₛₒ and Aᵢₛₒ values of the complexes to be determined ([VO(SALIEP)(Cat)]: g(1)=1.955, g(2)=1.987, A(1)=369.8 Hz, A(2)=85.0 Hz and [VO(Cl-SALIEP)(Cat)]: g(1)=1.951, g(2)=1.987, A(1)=369.7 Hz, A(2)=83.2 Hz; FIG. 29(A)-(B)). Additional experiments were performed to demonstrate the formation of the semiquinone ligands in DMSO solutions, by the use of the metal ionstabilization technique. A solution of Zn(OAc)₂ was used because it: 1) trapped the resulting semiquinone radicals; and 2) the zinc salt dissolved in several organic solvents, including DMSO and MeCN. The data showed an increased concentration of [VIVO(SALIEP)(Cat)] starting at 0 h, as evidenced by the increasing intensities of the peaks (FIG. 29(A)). Additionally, formation of semiquinone radicals was observed at all time points, which confirmed the hypotheses that the formation of semiquinone radicals occurred upon oxidation of the catecholates by V(V). We also performed EPR experiments using a 3,400-3,600 G window to confirm the formation of semiquinone radicals over time. The EPR spectra of the [VIVO(Cl-SALIEP)(Cat)] complex showed a different trend where the amount of the reduced species remained approximately the same but slightly increased at t=6 h (FIG. 29(B)). The formation of the semiquinone radicals was also observed at all time points, which confirmed that the chloro analogs also form semiquinone radicals upon reduction in solution.

This is the first EPR spectroscopic study with V(IV) mixed-Schiff base/catecholato ligand complexes. Our data showed that the six-membered core which result from the N—N coordination sites of the Schiff base, the methylene arm of the Schiff base and the vanadium to canter significantly and decrease the stability of the V(V) precursor with respect to V(IV). Hence, this results in the increased ease of reduction, compared to the VOHSHED analogs. This is because the [VO(HSHED)L] complexes have a five-membered ring core resulting from the N—N coordination sites of the Schiff base, the ethylene arm of the Schiff base and the vanadium center which, consequentially, significantly increased their stability and decreased the ease of reduction to the corresponding V(IV) species. The EPR spectroscopic data confirmed that both [VO(SALIEP)(Cat)] and [VO(Cl-SALIEP)(Cat)] predominantly existed as the reduced V(IV) species, when the product of the reaction of the [VO2(X-SALIEP)] with catechol was dissolved in the organic solvents, DMSO and MeCN. This observation was consistent with our hypothesis that the weak ⁵¹V NMR signals of the V(V) complexes in these organic solvents was due to substantial reduction to V(IV) and the broadening of the ⁵¹V NMR peaks. The higher signal-to-noise ratio in ¹H NMR spectra confirmed the reduction of both [VO(SALIEP)(Cat)] and [VO(Cl-SALIEP)(Cat)] complexes to the respective V(IV) species. This showed that this series of V(V) complexes with pyridine-containing Schiff bases substantially reduced to the corresponding V(IV) species, particularly in the absence of bulky hydrophobic substituents on the catecholate ligand.

Cyclic Voltammetry. Given the reduction to V(IV) observed in the EPR spectroscopic results, the redox chemistry of pyridine Schiff base-containing catecholates was particularly interesting given that the reduction can happen at either the vanadium center, or the catecholate ligand, or be delocalized over both. The extent of the redox delocalization can be probed by XAS studies using XANES to determine the metal oxidation state and bond lengths combined with DFT calculations and in the [V(DTB)₃]ⁿ⁻ (n=1, 2, 3) series, this was predominantly at the vanadium center. Dry acetonitrile was chosen as a solvent due to its wide potential window (−3.1 to +1.5 V) and good solubility of the vanadium complexes with 0.1 M tetrabutylammonium perchlorate (TBAP) as the supporting electrolyte. The E_{1/2} values were reported against an external ferrocene standard (Fc+/Fc) in acetonitrile. Solutions of 2.0 mM vanadium complexes in 0.1 M TBAP acetonitrile solution were prepared and their cyclic voltammograms (CVs) were recorded at 100 mV s⁻¹ at the 3 mm diameter glassy carbon electrode.

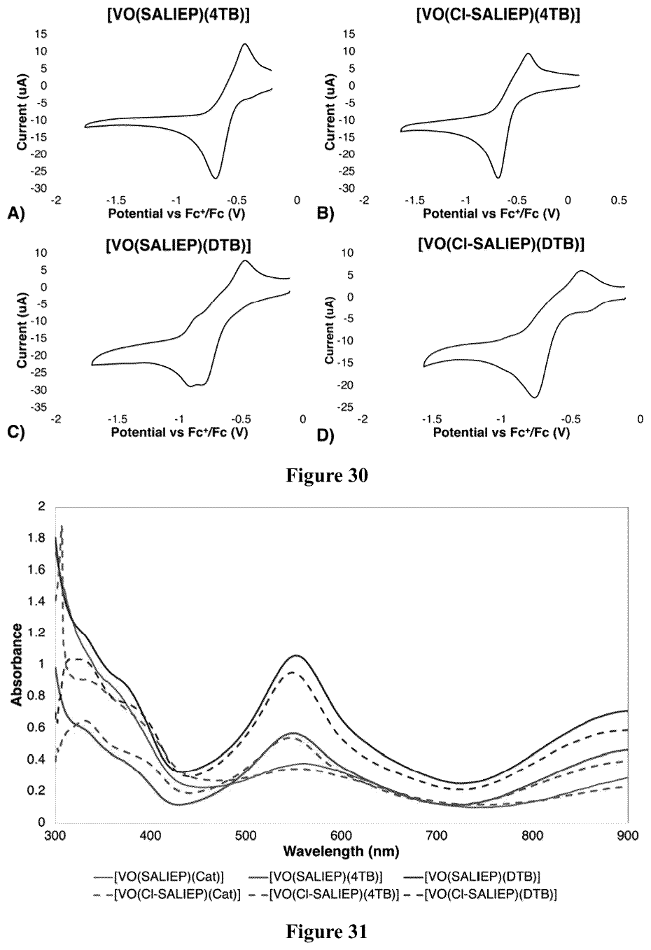

The potentials of these redox process were consistent with V centered redox reactions and not those on the catecholate ligand (FIG. 30), which typically occurred at much more negative potentials, unlike those of the catecholate ligands. Like the HSHED complexes, the complexes with 4 TB ligand had quasi-reversible V(V/IV) electrochemical responses at E₁/2 values of −0.548 V and −0.527 V vs Fc+/Fc with large ΔEp values of −0.279 V and −0.334 V for the SALIEP and Cl-SALIEP complexes, respectively (FIG. 30). As was the case for the HSHED complexes, the large ΔEp values were not due to uncompensated resistance since the ΔEp values of [VO(HSHED)(DTB)] (−0.158 V) for the Fc+/Fc couple were much less. Thus, as for the case of the HSHED complexes, the large ΔEp values for the V(V/IV) couples were attributed to different geometric isomers being more thermodynamically stable in the V(IV) oxidation state than in the V(V) oxidation state with the mixture of V(IV) complexes rapidly equilibrating on the electrochemical timescale at 100 mV s⁻¹. This interpretation is supported by the results of the cyclic voltammetry of the DTB complexes, which exhibit separate redox responses for different geometric isomers (FIG. 30 and Table 9). Clearly, the steric bulk of the DTB ligand and the rigidity and steric effects of the SALIEP ligands slow down the geometric isomerization in both oxidation states to enable the observation of separate redox processes for the different geometric isomers (Table 9 and FIG. 30). For, the [VO(SALIEP)(4 TB)] complex, there is a weak initial reduction peak at −0.524 V vs Fc⁺/Fc, which is attributed to a minor isomer, which indicates that the rate of geometric isomerism in the V(V) oxidation state is slow on the electrochemical timescale, but not in the V(IV) oxidation state. The CVs over five cycles confirmed that none of the observed peaks were contributed by decomposition side products.

TABLE 9
Half-wave potentials of V(V) complexes. The CV data were
collected in 2.0 mM dry acetonitrile solutions at ambient
temperature (~20-22° C.) at a scan rate of 100 mV
s⁻¹ at a 3-mm diameter glassy carbon electrode.

| | E_{1/2} (1) | E_{1/2} (2) | E_{1/2} (3) | |
|---|---|---|---|---|
| | vs Fc [symbol] /Fc | vs Fc [symbol] /Fc | vs Fc [symbol] /Fc | |
| Compound | (V) | (V) | (V) | Ref. |
| [VO(SALIEP)(4TB)] | −0.548 | N/A | N/A | This work |
| [VO(SALIEP)(DTB)] | −0.680 | −0.827 | −0.890 | This work |
| [VO(Cl-SALIEP)(4TB)] | −0.527 | N/A | N/A | This work |
| [VO(Cl-SALIEP)(DTB)] | −0.590 | −0.848 | −0.936 | This work |
| [VO(HSHED)(Cat)] | −0.698 | N/A | N/A | Murakami |
| | | | | et. al. |
| | | | | 2022 [ [symbol] ] |
| [VO(HSHED)(DTB)] | −0.802 | N/A | N/A | Murakami |
| | | | | et. al. |
| | | | | 2022 [ [symbol] ] |
| [VO(Cl-HSHED)(Cat)] | −0.679 | N/A | N/A | Murakami |
| | | | | et. al. |
| | | | | 2022 [ [symbol] ] |
| [VO(Cl-HSHED)(DTB)] | −0.782 | N/A | N/A | Murakami |
| | | | | et. al. |
| | | | | 2022 [ [symbol] ] |

[symbol]

indicates data missing or illegible when filed

The substitution pattern on the catecholate ligand affected the observed E_{1/2} values. Both [VO(SALIEP)L] and [VO(Cl-SALIEP)L] complexes with sterically hindered DTB ligands had the most negative E_{1/2} values (Table 9) and, hence, were the most difficult to reduce. The CVs for those complexes indicated that two V(V) isomers were in equilibrium with one V(IV) reduced species. Additionally, the [VO(Cl-SALIEP)(DTB)] complex had the most negative E_{1/2} values (−0.936 V and −0.848 V, respectively) due to the back-donating nature of the Cl group to the framework. The complexes with 4 TB ligands reduced more easily to V(IV), which is evidenced by more positive E_{1/2} values (Table 9). The electrondonating effect of the Cl group had little effect on the redox properties of the [VO(SALIEP)(4 TB)] and [VO(Cl-SALIEP)(4 TB)] complexes, since their E_{1/2} values were similar, but this may have been complicated by the effects of geometric isomerism. The CVs little to no redox activity was observed for the [VO(SALIEP)(Cat)] and [VO(Cl-SALIEP)(Cat)] complexes, meaning that the complexes immediately and irreversibly reduced to V(IV). The findings were confirmed by the ⁵¹V NMR of the complexes in acetonitrile, as those spectra exhibited no V(V) peaks, and EPR studies in DMSO which demonstrated an instant reduction to the respective V(IV) complexes (FIG. 29(A)-(B)). Thus, the [VO(SALIEP)(Cat)] and [VO(Cl-SALIEP)(Cat)] predominantly exist as V(IV) species.

With increased hydrophobicity of the complexes, they were less easily reduced due to steric and electronic effects of the different ligand sets. In particular, the presence of the pyridine ring in the VOSALIEP complexes is the main contributor to increased hydrophobicity and changes in half-wave potential values, compared to the VOHSHED complexes. Since the cellular environment is reducing and has high concentrations of glutathione, most abundant intracellular reductant, future electrochemical characterization will be carried out in the presence of glutathione to probe how it affects the redox properties of the complexes under conditions appropriate for the biological assays and physiological conditions.

UV-Vis Characterization. The [VO(SALIEP)L] and [VO(Cl-SALIEP)L] complexes were characterized by UV-Vis spectroscopy to probe how structural changes of both the Schiff base and the catecholate ligand contributed to the changes of the HOMO-LUMO gap. The UV-Vis spectra were collected in dry acetonitrile because all complexes are sensitive to reduction in the presence of trace moisture in the organic solvents. The UV-Vis spectra of all complexes (0.10 mM, dry MeCN) exhibited maxima at around 335 nm, 550 nm and 899 nm (FIG. 31) with λₘₐₓ and εₘₐₓ values listed in Table 11. The maxima around 500 nm and 900 nm were ligand-to-metal charge transfer (LCMT) bands, which confirmed the presence of the catecholate ligand coordinated to the vanadium center and obscured the d-d transitions, which are an order of magnitude less intense (FIG. 31). The maxima around 899 nm corresponded to the lower energy LMCT transition bands of the vanadium. Generally, the presence of hydrophobic substituents increased the molar absorptivity of the complexes. These data reflected that the halogen substituent decreased the stability of the alkyl substituted catecholate complexes and, thus, increased the gap between the HOMO-LUMO orbitals. The changes in maxima and decreased intensity of the bands for the Cat complexes are consistent with their substantial reduction to V(IV) in solution. In addition, the decreased molar absorptivity and changes in the Fmax values are consistent with partial reduction to V(IV), particularly for the Cat and 4 TB complexes, as was observed in the NMR and EPR spectroscopic results.

TABLE 11
Band Positions and Corresponding Molar Extinction Coefficients of the
[VOSALIEP)] and [VO(Cl-SALIEP)] compound series.

| | λₘₐₓ, | | λₘₐₓ, | |
|---|---|---|---|---|
| Compound | nm | ε (M⁻¹ cm⁻¹) | nm | ε (M⁻¹ cm⁻¹) |
| [VO(SALIEP)(Cat)] | 561 | 3.8 × 10³ | 899 | 2.8 × 10³ |
| (VO(SALIEP)(4TB)] | 551 | 5.6 × 10³ | 899 | 4.6 × 10³ |
| [VO(SALIEP)(DTB)] | 551 | 1.1 × 10³ | 899 | 7.1 × 10³ |
| [VO(Cl-SALIEP)(Cat)] | 560 | 3.4 × 10³ | 899 | 2.3 × 10³ |
| [VO(Cl-SALIEP)(4TB)] | 548 | 5.4 × 10³ | 899 | 3.9 × 10³ |
| [VO(Cl-SALIEP)(DTB)] | 549 | 9.5 × 10³ | 899 | 5.9 × 10³ |

* The UV-Vis spectra recorded in 0.1 mM solutions of dry acetonitrile.

Lipophilicity and P-value. Lipophilicity, or log P, is a partition between a lipophilic organic phase, generally n-octanol, and a polar aqueous phase. While log P values contribute to an understanding of the cell uptake of the complexes and, hence, provide useful information with regard to the antiproliferative activities of these complexes, such measurements are only accurate with hydrolytically stable compounds. In the case of the [VO(SALIEP)L] complexes, the experiment is difficult to conduct because most of these complexes hydrolyze within a few minutes, as evidenced by the hydrolytic stability data. Because such experiments would produce results that are not accurate, we used Chemicalize software to estimate the log P values (Table 11), to understand the effects of lipophilicity of the complexes on their uptake by the glioblastoma cells. Since this program was developed for organic molecules, we use these log P values to identy a pattern among a series of similar molecules that all contain a V-N coordinate bond. However, since any systematic errors will be similar throughout the series of compounds, this method enabled estimates of relative lipophilicity amongst these closely related metal complexes. The calculations showed that increasing the number of tert-butyl substituents increased lipophilicity of the complexes. Additionally, the presence of the chloro group on the Schiff base increased lipophilicity due to the electron-donating effects of the chloro group on the p aromatic system. The log P values of the [VO(SALIEP)L] complexes were also compared to those of the [VO(HSHED)L] analogs. The [VO(SALIEP)L] complexes were significantly more hydrophobic due to the presence of the pyridine ring on the Schiff base. The log P values of the [VO(SALIEP)L] complexes did not follow under the Lipinski's rule of five, as most of their log P values exceeded 4.5. The log P calculations, thus, predicted that the complexes would be slightly less active than the reported [VO(HSHED)L] and [VO(Cl-HSHED)L] series.

Stabilities and In Vitro Anti-Proliferative Activities of V(V) Complexes. As for [VV((Cl)HSHED)L], [VV(Cl)SALIEP)L] complexes decomposed within seconds or minutes under the conditions matching those of cell culture assays (100 mM V, 310 K, fully supplemented cell culture medium with added 10 mM HEPES buffer to maintain the pH value at 7.4 in the absence of 5% CO₂). The decomposition kinetics were followed by the disappearance of characteristic absorbance bands in the visible and near-IR range (FIG. 30). The presence of DTB ligands greatly increased the stability of V(V) complexes under biologically relevant conditions. Stabilities of V(V) complexes containing DTB ligands decreased in the series [VVO(Cl-SALIEP)(DTB)]>[VVO(SALIEP)(DTB)]>[VVO(HSHED)(DTB)]; the corresponding half-life-time (t_{1/2}) values are listed in Table 11. Decomposition of all three complexes resulted in the formation of a new absorbance band at ˜650 nm, which was assigned to the [VV(DTB)₃]-intermediate. Mixed-ligand V(V) complexes with 4 TB and CAT ligands decomposed immediately after the addition of their stock solutions in DMSO to cell culture medium (t_{1/2}<5 s, Table 12). Decomposition products of these complexes showed an absorbance band at −390 nm, which is similar to that observed for [VVO₂(SALIEP)] and [VVO₂(Cl-SALIEP)] complexes. Kinetic studies of decomposition of the other V complexes listed in Table 11 were not performed due the absence of characteristic absorbance bands in the visible range. Low-resolution ESI-MS data (˜10 mM V in MeOH solutions) showed that all of the studied V(V/IV)—(Cl)SALIEP complexes decomposed with the formation of free (Cl)SALIEPH ligands, which is consistent with the generation of [VV(DTB)₃]⁻ as one of the products.

Of all the studied V—(Cl)SALIEP complexes, [VV(O)(SALIEP)(DTB)] showed the highest anti-proliferative activity of freshly diluted solutions in T98 g cells (IC50=6.7±0.9 mM at 72 h treatment, condition A in Table 12). The activity decreased ˜two-fold after the decomposition of the complex for 30 min at 310 K and 5% CO₂ (condition B in Table 12). The activities of all the other V(V/IV) complexes with (Cl)SALIEP ligands were similar or lower than those of the modelled decomposition products, such as Na3VO4, DTBH2 or their combination (all pre-incubated with the medium for 30 min, Table 12). Notably, [VV(O)(SALIEP)(DTB)] and [VV(O)(Cl-SALIEP)(DTB)] complexes showed lower antiproliferative activities and 5-10-fold lower cellular V content compared with [VVO(HSHED)(DTB)] (Table 12), despite being more stable in cell culture medium. These results confirm that the in vitro antiproliferative activities of V(V)-Schiff base-catecholato complexes with cancer cells are determined by a combination of factors, including stabilities in extracellular media, redox properties and penetration rates through the cell membranes. On the other hand, cellular V contents in T98 g cells after the 30 min treatments with 100 mM [VV(O)(SALIEP)(DTB)] and [VV(O)(Cl-SALIEP)(DTB)] were 15-30-fold higher than that for the cells treated with Na₃VO₄ under the same conditions, and ˜three-fold higher than for the cells treated with [VIVO(SALIEP)(acac)] (Table 12). These data are consistent with the interpretation that the former two complexes are at least partially taken by cells intact via passive diffusion through the cell membranes. The lower uptake of the XSALIEP complexes compared with their HSHED counterparts is consistent with the increased lipophilicity moving them out of the range of optimal drug uptake and efficacy.

However, on the positive side for therapeutic applications, unlike for [VVO(HSHED)(DTB)], V(V) complexes with (C₁)SALIEP ligands were consistently less toxic to non-cancer HFF-1 (human skin fibroblast) cells compared with T98 g (human glioblastoma) cells (Table 12). In particular, freshly diluted solutions of [VVO(SALIEP)(DTB)] were five-fold less toxic in HFF-1 compared with T98 g cells. In addition, subtoxic concentrations of V(V)—(Cl)SALIEP complexes, as well as of Na₃VO₄, increased the viability of HFF-1, but not of T98 g, cells by 120-140% compared with the vehicle control. Similar selective stimulating effect of V(V)—(Cl)HSHED complexes on non-cancer HFF-1 cells has been recently reported. Stimulation of growth of cultured cells by low concentrations of V(V/IV) complexes is likely due to their phosphatase-inhibiting properties and can contribute to the potential beneficial effects of decomposition products of cytotoxic V(V) complexes used in cancer treatment.

TABLE 12
Stabilities in cell culture medium, anti-proliferative activities and cellular
uptakes of [VO(SALIEP)(L)], [VO(Cl-SALIEP)(L)] complexes and reference compounds

| | | | IC₅₀ | IC₅₀ | Uptake |
|---|---|---|---|---|---|
| Compound | t_{1/2},^{a} | | (T98g)^{b} | (HFF-1)^{b} | (T98g)^{c} |
| [V^{V}O₂(SALIEP)] | ND | | 50 ± 4 (A) | ND^{d} | ND |
| [V^{IV}O(SALIEP)(acac)] | ND | | 50 ± 4 (A) | ND | (1.7 ± 0.3) × 10⁸ |
| [V^{V}O(SALIEP)(Cat)] | <5 | s | 39 ± 4 (A) | ND | ND |
| [V^{V}O(SALIEP)(4TB)] | <5 | s | 28 ± 3 (A) | ND | ND |
| [V^{V}(SALIEP)(DTB)] | ~100 | s | 6.7 ± 0.9 (A) | 34 ± 10 (A) | (9 ± 2) × 10⁸ |
| | | | 11.3 ± 0.8 (B) | 32 ± 8 (B) | |
| [V^{V}O₂(Cl-SALIEP)] | ND | | 45 ± 4 (A) | ND | ND |
| [V^{IV}O(Cl-SALIEP)(acac)] | ND | | 34 ± 6 (A) | ND | ND |
| [V^{V}O(Cl-SALIEP)(Cat)] | <5 | s | 33 ± 5 (A) | ND | ND |
| [V^{V}O(Cl-SALIEP)(4TB)] | <5 | s | 30 ± 3 (A) | ND | ND |
| [V^{V}O(Cl-SALIEP)(DTB)] | ~180 | s | 18 ± 3 (A) | 47 ± 5 (A) | (5.3 ± 0.6) × 10⁸ |
| | | | 33 ± 12 (A) | 47 ± 4 (B) | |
| [V^{V}O(HSHED)(DTB)]^{e} | ~50 | s | 1.9 ± 0.2 (A) | 1.9 ± 0.4 (A) | (4.9 ± 0.9) × 10⁹ |

| | | | | |
|---|---|---|---|---|
| Na₃VO₄ + DTBH₂ | ND | 9 ± 1 (B) | 28 ± 3 (B) | ND |
| Na₃VO₄ | ND | 18 ± 3 (B) | 35 ± 3 (B) | (3.3 ± 0.8) × 10⁷ |
| DTBH₂ | ND | 15 ± 3 (B) | 24 ± 2 (B) | ND |

^{a}Half-life time of the complex in fully supplemented cell culture medium at 310K, determined by UV-vis spectroscopy.
^{b}Typical IC50 values (mM; means and standard deviations of six replicate wells) in T98g (human glioblastoma cells) and HFF-1 (non-cancer human fibroblasts). Designations of assay conditions: in (A), freshly diluted compounds were applied to cells for 72 h; and in (B), compounds were pre-incubated with cell culture medium for 30 min at 310K and 5% CO₂, then applied to cells for 72 h. The corresponding concentration-viability curves were determined. Uptake of V (atoms per cell; means and standard deviations of four replicate wells) by T98g cells after 30 min treatments with 100 mM of freshly diluted V complexes.
^{d}ND = not determined.
^{e}HSHED = N-(salicylideneaminato)-N′-(2-hydroxyethyl)-1,2-ethanediamine.

Conclusion

In this Example, we designed and synthesized novel non-innocent pyridine containing Schiff base vanadium(V) catecholates and carried out their characterization by a variety of spectroscopic (NMR, EPR, UV-Vis) and electrochemical methods. The presence of the pyridine ring on the Schiff base resulted in an increase of hydrophobicity, as evidenced by the increase of the log P values. Additionally, the increase in hydrophobicity and steric effects of the Schiff base ligand made the thermodynamics of reduction of the complexes to V(IV) less favorable, according to the cyclic voltammetry data. By contrast, the ⁵¹V NMR and EPR spectroscopic studies, showed that the presence of the pyridine ring increased the rates of reduction to V(IV), as pyridine tends to stabilize the species in lower oxidation states. The EPR studies have shown that the complexes with unsubstituted catecholate ligands are most likely to reduce to V(IV) and form semiquinone radicals in the organic solvent.

The presence of bulky aliphatic substituents increases the stability of the V(V) catecholate complexes in both organic solvent and under the assay conditions. The UV-Vis hydrolytic stability study has demonstrated that both [VVO(SALIEP)(DTB)] and [VVO(Cl-SALIEP)(DTB)] are stable for 5 min. under the assay conditions, followed by the decomposition to multiple species including the [VO(DTB)₃]-intermediate as one of the V(V) products. One of the newly synthesized mixed-ligand V(V) complexes, [VVO(SALIEP)(DTB)], has shown moderate stability in cell culture media, significant cellular uptake of the intact complex by T98 g (human glioblastoma) cells and high antiproliferative activity (IC50<10 mM at 72 h treatment) in this cell line, which was ˜5-fold higher than that for a non-cancerous human cell line, HFF-1.

The increased hydrophobicity of the [VVO(SALIEP)(DTB)] complex, compared to the lead [VO(HSHED)(DTB)] analog, resulted in the lower uptake by T98 g cells and also introduced increased selectivity for T98 g cells. Decomposition of [VVO(SALIEP)(DTB)] in cell culture medium decreased its anti-proliferative activity in T98 g cells ˜two-fold, while the activities of the fresh complex and its decomposition products in HFF-1 cells were equally low. These properties make [VVO(SALIEP)(DTB)] a potential drug candidate for the treatment of advanced glioblastomas by intracranial injections.

The structure-activity relationships have demonstrated that the presence of the pyridine ring on the Schiff base results in the selective uptake of the complexes by T98 g cells compared to the non-cancerous HFF cells. Additionally, pyridine significantly improves hydrolytic stability of the DTB analogs, as evidenced by the UV-Vis data. The complex redox chemistry of the [VO(SALIEP)X] complexes, however, requires further spectroscopic and cyclic voltammetry studies in the presence of intracellular reductants, such as ascorbate and glutathione. Those studies would help us understand the effects of intracellular reductants on the redox properties and redox reaction mechanism of the [VO(SALIEP)X] complexes. The knowledge of the chemical properties, cytotoxicity and metal uptake of the [VO(HSHED)L] and the [VO(SALIEP)L] series will be crucial to optimizing the chemical properties of potential candidates for intratumoral injections to treat glioblastoma.

In summary, this Example presents V(V) catecholates that form one major isomer and show selectivity towards glioblastoma (T98 g) cells. These complexes are very hydrophobic and, as a result, less readily taken up by glioblastoma cells. However, due to lower levels of intracellular uptake, they are just as efficacious as previously reported [VO(HSHED)L] and [VO(Cl-HSHED)L] analogs.

Materials and Methods

General Materials and Methods. Salicylaldehyde (98%), 5-chlorosalicylaldehyde (98%), [VO(acac)₂] (acac=acetylacetonatonato(1-)) (98%) and aqueous H₂O₂ (30%) were purchased from Sigma Aldrich and used as received. 2-(2-Aminoethyl)pyridine (98%) was purchased from Oakwood Chemical and used as received. Catechol (>99.0%), 4-tert-butylcatechol (≥99.0%) and di-3,5-tert-butyl-catechol (98%) were purchased from Sigma Aldrich and recrystallized from toluene, pentane and petroleum ether, respectively, prior to use. The purity of recrystallized ligands was confirmed by ¹H NMR spectroscopy in d6-DMSO. d3-Acetonitrile (≥99.8 atom % D) and d6-DMSO (≥99.9 atom % D) were purchased from Cambridge Isotope Laboratories and used as received. Silver nitrate, ferrocene and tetra-n-butylammonium perchlorate (TBAP) were purchased from Merck Millipore for electrochemistry experiments and used as received. All syntheses were carried out under an Ar atmosphere unless noted otherwise.

Pre-sterilized media and sterile plasticware used in cell culture were purchased from Thermo Fisher Scientific Australia. High purity Na₃VO₄ (99.98%, Cat. No. 450243) and DTBH2 (98% Car. No. D45800) for cell experiments were purchased from Merck. The well-established human cancer cell lines: T98 g (glioblastoma multiforme, Cat. No. CRL-1690; and HFF-1 (normal human foreskin fibroblasts, Cat. No. SCRC-1041) were purchased from ATCC and used at passages four to six. The cells were cultured in Advanced DMEM (Thermo Fisher Scientific Cat. No. 12491-015), supplemented with L-glutamine (2.0 mM), antibiotic-antimycotic mixture (100 U mL⁻¹ penicillin, 100 mg mL⁻¹ streptomycin and 0.25 mg mL⁻¹ amphotericin B) and foetal calf serum (FCS; Thermo Fisher Scientific Cat. No. 10100147, heatinactivated; 2% vol). For proliferation and cytotoxicity experiments, cells were seeded in 96-well plates at an initial density of (1-2)×10³ viable cells per well in 0.10 mL medium and left to attach overnight.

Synthesis. The [V(O)₂(SALIEP)] precursor was synthesized using the procedure described below.

Step 1. Synthesis of 2-[[[2-(2-pyridinyl)ethyl]imino]methyl]phenol (HSALIEP). To 10 mL of absolute EtOH, salicylaldehyde (0.611 g, 5.00 mmol) and 2-(2-aminoethyl)pyridine (0.611 g, 5.00 mmol), were added and then stirred under reflux for about 30 min. The resulting Schiff base was used immediately in the second step to avoid noticeable degradation.

Step 2. Preparation of [VIVO(SALIEP)(acac)] To 25 mL of absolute EtOH, [VIVO(acac)₂] (1.33 g, 5.00 mmol) was added and stirred at 60° C. to enable the complex to dissolve. Subsequently, this solution was added to the ethanolic Schiff base solution from Step 1, and the resulting reaction mixture was stirred under reflux for 3 h. The resulting red solid was filtered off, washed with 50 mL cold (0° C.) diethyl ether and dried in vacuo. Yield 60%. FT-IR: 3100 (sp² C—H stretch), 2916 (sp³ C—H stretch), 1590-1416 (aromatic C═C and C═N and imine C═N stretches), 1383 (sp³ CH bend), 1342 (aliphatic C—N stretch), 929 (V═O stretch) cm⁻¹.

Step 3. Preparation of [V^{V}(O)₂(SALIEP)] [VIVO(SALIEP)(acac)] (0.391 g, 1.00 mmol) was dissolved in 20 mL of methanol and after the addition of aqueous 30% H₂O₂ (0.2 mL, 2.00 mmol), the solution was exposed to aerial oxidization for 10 min. The resulting yellow solid was filtered, washed with 50 mL cold (0° C.) diethyl ether and dried in vacuo. Yield 59%. NMR: ¹H (d₆-DMSO): 8.52 (d, 1H), 8.34 (s, 1H), 7.66 (t, 1H), 7.46 (t, 1H), 7.33 (d, 1H), 7.22 (t, 1H), 7.18 (d, 1H), 4.04 (t, 2H), 3.32 (t, 2H). ¹H (d₃-MeCN): 8.90 (d, 1H), 8.69 (s, 1H), 7.99 (t, 1H), 7.49 (t, 2H), 7.43 (t, 1H), 6.87 (m, 2H), 4.03 (t, 2H), 3.39 (t, 1H), 3.29 (d, 1H). ⁵¹V NMR (d₆-DMSO): −507, −539 ppm; (d₃-MeCN): −511 ppm. FT-IR: 3060 (sp² C—H stretch), 1620-1470 (aromatic C═C and C═N and imine C═N stretches), 1310 (aliphatic C—N stretch), 917 (V═O stretch), 761 (sp² C—H bend, 1,2-disubstituted) cm⁻¹. HRMS (ESI) calc. for C₁₉H₂₀N₂O₄V [M]⁺=391.08572, found 391.08557.

[VO(SALIEP)(Cat)] To 25.0 mL of degassed methanol, [VV(O)₂(SALIEP)] (0.308 g, 1.00 mmol) was added, followed by the addition of catechol (0.132 g, 1.20 mmol). The reaction mixture was stirred for 20 h at room temperature (−20° C.) under Ar. The resulting product was vacuum filtered, washed with cold (0° C.) diethyl ether (30 mL) and dried under high vacuum for 24 h. Yield: 58%. NMR: ¹H (d₃-MeCN, 400 MHz): 8.80 (d, 1H), 8.54 (s, 1H), 7.91 (t, 1H), 7.47 (d, 1H), 7.44 (s, 1H), 7.43 (s, 1H), 7.41 (dd, 1H), 6.78 (t, 1H), 6.69 (d, 2H), 6.45 (d, 1H), 6.04 (d, 1H); ⁵¹V (d₃-MeCN, 105.2 MHz): −508 ppm (broad, [V(O)₂(SALIEP)]), ⁵¹V (d₆-DMSO, 105.2 MHz): −497 ppm, −511 ppm ([V(O)₂(SALIEP)] decomposition product). EPR ([VIVO(SALIEP)(Cat)]: g(1)=1.955, g(2)=1.987, A(1)=369.8 Hz, A(2)=85.0 Hz. FT-IR: 3060 (sp² C—H stretch), 1620-1450 (aromatic C═C and C═N and imine C═N stretches), 1310-1210 (aliphatic C—N stretch), 1150 (C—O stretch), 965 (V═O stretch) cm⁻¹. UV-Vis: kmax (0.10 mM in MeCN)=561 nm, F (M−1 cm-1)=3.8×10³. HRMS (ESI) calc. for C₂₀H₁₇N₂O₄V [M+H]⁺=401.07007, found 401.07015.

[VO(SALIEP)(4 TB)] To 50.0 mL of degassed methanol, [VV(O)₂(SALIEP)] (0.308 g, 1.00 mmol) was added, followed by the addition of 4-tert-butyl-catechol (0.199 g, 1.20 mmol). The reaction mixture was stirred for 20 h at room temperature under Ar. The resulting product was vacuum filtered, washed with cold (0° C.) diethyl ether (30 mL) and dried under high vacuum for 24 h. Yield: 39%. NMR: ¹H (d₃-MeCN, 400 MHz): 8.85 (d, 1H), 8.51 (s, 1H), 7.90 (t, 1H), 7.52 (d, 1H), 7.43 (t, 1H), 7.41 (t, 1H), 6.78 (t, 1H), 6.69 (d, 1H), 6.67 (d, 1H), 6.54 (d, 1H), 6.40 (d, 1H), 6.10 (s, 1H), 4.01 (dd, 2H), 3.52 (dd, 2H) 1.21 (s, 9H); ⁵¹V (d₃-MeCN, 105.2 MHz): −511 ppm ([V(O)₂(SALIEP)] decomposition product), ⁵¹V (d₆-DMSO, 105.2 MHz): 650 ppm, 616 ppm, −216 ppm ([V(4 TB)₃]⁻ decomposition product), −511 ppm ([V(O)₂(SALIEP)] decomposition product). FT-IR: 3050 (sp² C—H stretch), 2950 (sp³ C—H stretch), 1620-1450 (aromatic C═C and C═N and imine C═N stretches), 1340 (aliphatic C—N stretch), 952 (V═O stretch) cm⁻¹. UV-Vis: λmax (0.10 mM in MeCN)=551 nm, ε (M⁻¹ cm⁻¹)=5.6×10³. HRMS (ESI) calc. for C₂₄H₂₅N₂O₄V [M+H]⁺=457.13267, found 457.13274.

[VO(SALIEP)(DTB)] To 50.0 mL of degassed methanol, [VV(O)₂(SALIEP)] (0.308 g, 1.00 mmol) was added, followed by the addition of di-tert-butyl-catechol (0.267 g, 1.20 mmol). The reaction mixture was let stirred for 20 h at room temperature under Ar. The resulting product was vacuum filtered, washed with cold (0° C.) diethyl ether (30 mL) and dried under high vacuum for 24 h. Yield: 58%. NMR: ¹H (d₃-MeCN, 400 MHz): 8.98 (d, 1H), 8.50 (d, 1H), 7.90 (q, 1H); 7.46 (t, 1H), 7.42 (d, 1H), 7.40 (d, 1H), 7.38 (d, 1H), 6.75 (m, 1H), 6.65 (m, 1H), 6.46 (s, 1H), 6.03 (s, 1H), 4.07 (m, 2H), 3.55 (m, 1H), 3.42 (m, 1H), 1.40 (s, 6H), 1.27 (s, 3H), 1.21 (s, 6H), 0.98 (s, 3H); ⁵¹V (d₃-MeCN, 105.2 MHz): −511 ppm ([V(O)₂(SALIEP)] decomposition product), ⁵¹V (d₆-DMSO, 105.2 MHz): 665 ppm, −250 ppm ([V(DTB)₃]-decomposition product). FT-IR: 3050 (sp² C—H stretch), 2950 (sp³ C—H stretch), 1620-1450 (aromatic C═C and and imine C═N stretches), 1320 (aliphatic C—N stretch), 940 (V═O stretch) cm⁻¹. UV-Vis: λmax (0.10 mM in MeCN)=555 nm, E (M⁻¹ cm⁻¹)=1.1×10⁴. HRMS (ESI) calc. for C₂₈H₃₃N₂O₄V [M+H]⁺=513.19527, found 513.19520.

[V^{V}(O)₂(Cl-SALIEP)] Step 1. Synthesis of 2-[[[2-(2-pyridinyl)ethyl]imino]methyl]chlorophenol (H-CISALIEP) To 10 mL of absolute EtOH, 5-chlorosalicylaldehyde (0.783 g, 5.00 mmol) and 2-(2-aminoethyl)pyridine (0.611 g, 5.00 mmol), were added and then stirred under reflux for about 30 min. The ligand was used immediately in the second step to avoid noticeable degradation.

Step 2. Preparation of [VIVO(Cl-SALIEP)(acac)] To 25 mL of absolute EtOH, [VIVO(acac)₂] (1.33 g, 5.00 mmol) was added and the mixture was stirred at 60° C. until the complex dissolved. Subsequently, this solution was added to the ethanolic Schiff base solution from Step 1, and the resulting reaction mixture was stirred under reflux for 3 h. The resulting shiny brown solid was filtered off, washed with 50 mL cold (0° C.) diethyl ether, and dried in vacuo. Yield 57%. FT-IR: 3050 (sp² C—H stretch), 2950-2920 (sp³ C—H stretch), 1590-1460 (aromatic stretch C═C and C═N and imin e C═N stretches), 1376 (sp³ C—H bend), 1308 (aliphatic C—N stretch), 1183 (C—O stretch), 929 (V═O stretch), 703 (C—Cl stretch) cm⁻¹. HRMS (ESI) calc. for C₁₉H₁₉ClN₂O₄V [M]⁺=425.04674, found 425.04649.

Step 3. Preparation of [V^{V}(O)₂(Cl-SALIEP). [VIVO(Cl-SALIEP)(acac)] (0.426 g, 1.00 mmol) was dissolved in 20 mL of methanol and after the addition of aqueous 30% H₂O₂ (0.2 mL, 2.00 mmol), the solution underwent aerial oxidation for 10 min. The resulting brown solid was filtered, washed with 50 mL of cold (0° C.) diethyl ether and dried in vacuo. Yield 75%. ⁵¹V NMR (d₃-MeCN, 10.0 mM): −512 ppm. FT-IR: 3060 (sp² C—H stretch), 1630-1490 (aromatic C═C and imine C═N stretches), 1300 (aliphatic C—N stretch), 927 (V═O stretch), 786 (C—Cl stretch) cm⁻¹. HRMS (ESI) calc. for C₁₄H₁₂ClN₂O₃V [M⁺H]⁺=343.00488, found 343.00487.

[VO(Cl-SALIEP)(Cat)] To 12.5 mL of degassed methanol, [VV(O)₂(Cl-SALIEP)](0.343 g, 1.00 mmol) was added, followed by the addition of catechol (0.132 g, 1.20 mmol). The reaction mixture was stirred for 20 h at room temperature under Ar. The resulting product was vacuum filtered, washed with cold (0° C.) diethyl ether (30 mL) and dried under high vacuum for 24 h. Yield: 44%. NMR: ¹H (d₃-MeCN, 400 MHz): 8.76 (d, 1H), 8.49 (s, 1H), 7.92 (t, 1H), 7.48 (d, 1H), 7.43 (s, 1H), 7.42 (s, 1H), 7.36 (dd, 1H), 6.69 (d, 2H), 6.43 (d, 1H), 6.03 (d, 1H); ⁵¹V (d₃-MeCN, 105.2 MHz): no signal, ⁵¹V (d₆-DMSO, 105.2 MHz): 531 ppm, −216 ppm ([V(Cat)₃]⁻ decomposition product), −510 ppm ([V(O)₂(Cl-SALIEP)] decomposition product). EPR ([VIVO(Cl-SALIEP)(Cat)]: g(1)=1.951, g(2)=1.987, A(1)=369.7 Hz, A(2)=83.2 Hz. FT-IR: 3100 (sp² C—H stretch), 2960 (sp³ C—H stretch), 1630-1460 (aromatic CC and imine C═N stretches), 1390 (sp³ C—H bend), 1380 (aliphatic C—N stretch), 921 (V═O stretch), 786 (C—Cl stretch) cm⁻¹. UVVis: λmax (0.10 mM in MeCN)=555 nm, E (M⁻¹ cm⁻¹)=3.4×10³. HRMS (ESI) calc. for C₂₀H₁₆ClN₂O₄V [M⁺H]⁺=457.01304, found 457.01323.

[VO(Cl-SALIEP)(4 TB)] To 12.5 mL of degassed methanol, [VV(O)₂(Cl-SALIEP)](0.171 g, 0.500 mmol) was added, followed by the addition of 4-tert-butylcatechol (0.100 g, 0.600 mmol). The reaction mixture was stirred for 20 h at room temperature under Ar. The resulting product was vacuum filtered, washed with cold (0° C.) diethyl ether (30 mL) and dried under high vacuum for 24 h. Yield: 57%. NMR: ¹H (d₃-MeCN, 400 MHz): 8.81 (d, 1H), 8.46 (s, 1H), 7.91 (t, 1H), 7.47 (t, 1H), 7.42 (d, 1H), 7.41 (s, 1H), 7.36 (d, 1H), 6.67 (d, 1H), 6.50 (s, 1H), 6.40 (d, 1H), 6.09 (s, 1H), 4.04 (dt, 2H), 3.40 (dt, 2H), 1.26 (s, 3H), 1.21 (s, 6H); 51V (d₃-MeCN, 105.2 MHz): −536 ppm ([V(O)₂(Cl-SALIEP)]). FT-IR: 3100 (sp² CH stretch), 2960 (sp³ C—H stretch), 1630-1460 (aromatic C═C and imine C═N stretches), 1390 (sp³ C—H bend), 1310 (aliphatic C—N stretch), 951 (V═O stretch), 786 (C—Cl stretch) cm⁻¹. UV-Vis: λmax (0.10 mM in MeCN)=551 nm, ε(M⁻¹ cm⁻¹)=5.4×10³. HRMS (ESI) calc. for C₂₄H₂₄ClN₂O₄V [M⁺H]⁺=491.09369, found 491.09322.

[VO(Cl-SALIEP)(DTB)]. To 50.0 mL of degassed methanol, [VV(O)₂(Cl-SALIEP)](0.343 g, 1.00 mmol) was added, followed by the addition of 3,5-di-tert-butylcatechol (0.267 g, 1.20 mmol). The reaction mixture was stirred for 20 h at room temperature under Ar. The resulting product was vacuum filtered, washed with cold (0° C.) diethyl ether (30 mL) and dried under high vacuum for 24 h. Yield: 54%. NMR: ¹H (d₃-MeCN, 400 MHz): 8.94 (d, 1H), 8.45 (d, 1H), 7.89 (q, 1H); 7.47 (t, 1H), 7.40 (s, 1H), 7.37 (dd, 1H), 7.33 (dd, 1H), 6.46 (s, 1H), 6.03 (s, 1H), 4.07 (m, 2H), 3.55 (m, 1H), 3.44 (m, 1H), 1.39 (s, 6H), 1.27 (s, 3H), 1.21 (s, 6H), 0.97 (s, 3H). ⁵¹V (d₃-MeCN, 105.2 MHz): −531 ppm ([V(O)₂(Cl-SALIEP)] decomposition product), ⁵¹V (d₆-DMSO, 105.2 MHz): 665 ppm, −510 ppm ([V(O)₂(Cl-SALIEP)] decomposition product). FT-IR: 3100 (sp² C—H stretch), 2960 (sp³ C—H stretch), 1630-1460 (aromatic C—C and imine C═N stretches), 1390 (sp³ C—H bend), 1380 (aromatic C—N stretch), 923-921 (V═O stretch), 786 (C—Cl stretch) cm⁻¹. UV-Vis: λmax (0.10 mM in MeCN)=551 nm, E (M⁻¹ cm⁻¹)=9.5×10³. HRMS (ESI) calc. for C₂₈H₃₂ClN₂O₄V [M⁺H]⁺=547.15629, found 547.15550.

1D NMR Experiments. The complexes were characterized using ¹H NMR spectroscopy recorded on a Bruker NEO400 equipped with an automated tuning module operating at 400 MHz at an ambient temperature and ⁵¹V NMR spectroscopy recorded on a Varian MR400 spectrometer equipped with an automated tuning module operating at 105.2 MHz at an ambient temperature. The ⁵¹V NMR spectra were acquired with a spectral window of 3000 ppm (−996 ppm to 1977 ppm), 4096 scans, a 450 pulse, an acquisition time of 0.08 s, and a 0.01 s relaxation delay. 1D ⁵¹V NMR studies were referenced against [V(O)₂(HSHED)] at −529 ppm as a standard and reported in reference to VOCl₃ (0 ppm). 1D and 2D ¹H NMR studies were performed with deuterated organic solvents using a Bruker NEO400 spectrometer operating at 400 MHz at an ambient temperature. Chemical shift values (6) are reported in ppm and referenced against TMS using the internal solvent peaks in ¹H NMR spectra (d₃-MeCN, 6 at 1.94 ppm; d6-DMSO, δ at 2.50 ppm) as internal standards. All complexes (10 mM) were dissolved in either d3-MeCN or d6-DMSO at 10 mM for spectral comparison. Spectroscopic studies were carried out using solutions of isolated complexes, and both ¹H and ⁵¹V NMR spectra were recorded on the same samples. Both ¹H and ⁵¹V NMR samples were run within 5 min. of preparation, and no significant differences were observed in ¹H NMR spectra recorded within 24 h.

2D NMR Experiments. ¹H-¹H gCOSY experiments were carried out on a Bruker NEO400 spectrometer using the following parameters: 8 scans, 13 ppm spectral window, a 2.0 s relaxation delay, a 0.19 s acquisition time and a 12 s pulse. ¹H-¹H NOESY experiments were carried out on a Bruker NEO400 spectrometer using the following parameters: 256 scans, 1.5 s relaxation delay, 500 ms mixing time. ¹H-¹H ROESY experiments were carried out on a Bruker NEO400 spectrometer using the following parameters: 256 scans, 2.0 s relaxation delay, 400 ms mixing time.

EPR Spectroscopy. Electron paramagnetic resonance (EPR) spectra were recorded at 298 K using a Bruker X-Band EPR Spectrometer (9.84 GHz). The X-band EPR spectra were recorded in 1 mm quartz capillary tubes that were placed in 4- or 5-mm quartz tubes at ambient temperature. The spectra were referenced to a DPPH external standard (g=2.0037). The spectra were collected using the following parameters: 16 scans, 1600 G sweep width, 22 dB attenuation, 10 modulation amplitude, 60 s sweep time, 60 s conversion time. The EPR spectra were collected at 0, 1, 2, 6 and 24 h. The giso and Aiso values were calculated using “garlic” simulation in EasySpin (version 5.2.35) open-source MatLab toolbox (version R2022b) that uses analytical diagonalization to account for second-order effects. The stacked EPR spectra were plotted using OriginLab software (2022 version, Northampton, MA, USA).

Sample preparation: [VO(SALIEP)(CAT)] and [VO(Cl-SALIEP)(CAT)] (10.0 mM) in 2.0 mL of DMSO dried over 3 Å activated molecular sieves for 2 d prior was used to collect the EPR spectra. To check for the presence of semi-quinone radicals, solutions of [VO(SALIEP)(CAT)] and [VO(Cl-SALIEP)(CAT)] (10.0 mM in 2.0 mL of dry DMSO to which 100 μL of 50.0 mM DMSO solution of Zn(OAc)₂ was added), EPR spectra were collected at 0, 1, 2, 6 and 24 h.

Cyclic Voltammetry. Cyclic voltammetry was undertaken using a WaveDriver 40 DC Bipotentiostat/Galvanostat and a Low Volume Three Electrode Cell Basic Kit (AFO1CKT1006) purchased from Pine Research Instrumentation (AfterMath v 1.5.9807 data acquisition). The working electrode was a glassy carbon electrode with a 3.0 mm diameter (2.997-2.972 mm) and an area of approximately 9 mm2, and polished using silica pad wetted with a small amount of DDI water. The software used automatically had the iR Compensation option turned off, while eL-Chem Viewer and Microsoft Excel were used for post-acquisition processing. All cyclic voltammograms were externally referenced to the ferrocenium/ferrocene (Fc+/0 couple. Normal cyclic voltammograms were recorded at a scan rate of 100 mV s¹ and two segments using the following parameters: ferrocene, initial potential 1.0 V, vertex potential −1.5 V, final potential 1.0 V; [VO(SALIEP)(4 TB)], initial potential −0.10 V, vertex potential −1.65 V, final potential −0.10 V; [VO(SALIEP)(DTB)], initial potential −0.45 V, vertex potential −1.65 V, final potential −0.45 V; [VO(Cl-SALIEP)(4 TB)], initial potential 0.25 V, vertex potential −1.5 V, final potential 0.25 V; and [VO(Cl-SALIEP)(DTB)], initial potential −0.55 V, vertex potential −1.85 V, final potential −0.55 V.

Sample preparation: Complexes (2.0 mM) or ferrocene (10 mM) were dissolved in 100 mM TBAP in dry acetonitrile solution. A 2.0 mM silver nitrite solution was used to fill a refillable Ag reference electrode. All samples were degassed using Ar for 10 min prior to cyclic voltammogram collection.

Electrospray mass spectrometry (ESI-MS). Low-resolution ESI-MS data were collected on a Bruker amazon SL spectrometer, using the following parameters: nebulizer pressure, 27.3 psi; spray voltage, 4.5 kV; capillary temperature, 453 K; N2 flow rate, 4 L min⁻¹; m/z range, 100-1000 (alternating positive- and negative-ion modes). Analyzed solutions (5.0 μL) were injected into a flow of MeOH (flow rate, 0.30 mL min⁻¹). Acquired spectra were the averages of 100-200 scans (scan time, 10 ms). Solutions for mass spectrometry were prepared by dissolving ˜0.1 mg of complexes in 0.50 mL MeOH (˜100 mM V) immediately before the experiments. Simulations of the mass spectra were performed using IsoPro software (version 3.0, M. Senko, Sunnyvale, CA, USA, 1998). High-resolution positive-ion ESI-MS was performed on a Bruker Solarix 2XR 7T Fourier transform ion cyclotron resonance mass spectrometer via syringe infusion at 120 μL hr⁻¹. The transient length was 2 M and acquired in 2-w mode and the Fourier transform was performed in adsorption mode. The instrument was externally calibrated from 300-2000 m/z prior to analysis, and the isotopic patterns were simulated using Bruker Compass Data Analysis 5.0 software.

UV-Vis Spectroscopy experiments. All complexes were characterized by UV-Vis spectroscopy in 0.10 mM solutions in dry acetonitrile. The hydrolytic stability studies of the DTB analogs were performed with 0.10 mM solutions in PBS 1× buffer (pH 7.4) at t=0 h, 15 min., 30 min., 45 min., 1 h, 2 h, 4 h, 24 h and 48 h. Due to the hydrophobic nature of the complexes, 10.0 mM stocks solutions in acetonitrile were prepared, and then dilute to the 0.10 mM with the buffer solution immediately before recording the spectra.

Cell culture, proliferation and V uptake assays. Anti-proliferative activities of V(V/IV) complexes and their model decomposition products were measured using the standard MTT assay. Freshly prepared stock solutions of V(V/IV) complexes, DTBH₂ (all 10 mM in DMSO) and Na₃VO₄ (10 mM in Milli-Q H₂O) were used for cell assays. These solutions were further diluted so that all the cell treatments, including controls, contained 1.0% (vol.) of DMSO (for the treatments that included Na₃VO₄ only, DMSO was added separately into cell culture medium). Stock solutions of the treatment compounds were diluted with fully supplemented cell culture media to the required final concentrations, and the resultant media were either added to the cells within 1 min (fresh solutions), or left in cell culture incubator (310 K, 5% CO₂) for 24 h prior to the cell treatments (aged solutions). Each treatment included six replicate wells and two background wells that contained the same components except the cells. After the addition of treatment compounds, the plates were incubated for 72 h at 310 K and 5% CO₂, then the treatment medium was removed and MTT reagent (1-(4,5-dimethylthiazol-2-yl)-3,5-diphenylformazan, Merck M5655) was added (1.0 mg mL⁻¹ solution in complete medium), and incubation was continued for 4-6 h. After that, the medium was removed, the blue formazan crystals were dissolved in 0.10 mL per well of DMSO, and the absorbance at 600 nm was measured using Victor V3 plate reader. Typically, the treatment compounds were applied in a series of nine two-fold dilutions, starting from 100 μM V, plus the vehicle control. Fitting of the experimental data and calculations of the IC50 values were performed using Origin Pro software (2022 version, OriginLab, Northampton, MA, USA).

For V uptake experiments, T98 g cells were grown to ˜80% confluence in 12-well plates (three replicates per treatment). Incubations with the treatment compounds (100 μM V; freshly added to fully supplemented medium) were performed for 30 min at 310 K, 5% CO₂. After that, V-containing media were removed, the cell layers were washed twice with phosphate buffered saline (PBS), detached with TrypLE solution (Thermo Fisher Scientific Cat. No. 12605028), pelleted, and washed again with PBS. Detachment and washing of cell pellets were used to remove V that was absorbed on the culture plates and on the cell surface. Cell numbers in each pellet were counted with a Countess automatic counter, typical numbers were (5±1)×10⁵ cells/well, cell viability was >95% (Trypan blue staining). Cell pellets were digested with 0.20 mL of 65% HNO₃ (trace pure, Merck 225711) for 3 d at 295 K, then diluted with Milli-Q water to 1.0 mL for ICPMS analysis. The analysis was performed with a Perkin-Elmer Nexion 350× spectrometer, using a standard V(IV) solution (Choice Analytical, Australia) and 193Ir peak as an internal standard. The measurements were performed in kinetic energy discrimination (KED) mode to eliminate the interference of [³⁵Cl¹⁶O]⁺ ions with the determination of ⁵¹V⁺. Cellular V concentrations were expressed in atoms V per cell. For all the cell assays, consistent results were obtained in at least two independent experiments, using different passages of cells and different stock solutions of the treatment compounds.

### Example 6. A Ligand-to-Metal-Charge-Transfer Route to Increasing Temperature Sensitivity for ⁵¹V NMR Thermometers

In this Example, we present a route of amplifying temperature sensitivity of the ⁵¹V chemical shift via ligand-to-metal charge transfer electronic structure design criteria. We demonstrate this design strategy can boost the temperature dependence of the ⁵¹V chemical shift by an order of magnitude.

Designing molecules with variable-temperature magnetic resonance characteristics is vital to non-invasive imaging of temperature—a key means of monitoring thermal procedures and deeper understanding of physiological temperature management. Among many magnetic resonance characteristics, the chemical shift of a nucleus is a spectroscopic signature, where the resonant frequency directly reports as a probe on the local temperature. Toward better, higher sensitivity thermometers, molecular design can control and improve temperature sensitivity.

Our group has recently reported several studies of controlling the temperature sensitivity of the ⁵⁹Co nuclear magnetic resonance chemical shift. This nucleus generally exhibits a highly temperature-sensitive NMR δ parameter of the ⁵⁹Co nucleous when found in Co(III) complexes (Δδ/ΔT>2 ppm/° C.) relative to other nuclei (e.g. ¹H, 0.01 ppm/° C. or 19F, 0.05 ppm/° C.). This parameter therefore exhibits a high potential for non-invasive imaging. In one case, we set a record for high temperature sensitivity of the chemical shift of all nuclei, up to 150 ppm/° C., in a spin-crossover ⁵⁹Co complex. Yet, in these cobalt complexes, the spectroscopic linewidths are large (ca. 150 ppm), in part because of the quadrupolar moment, which is intrinsically large for the ⁵⁹Co nucleus. A large linewidth is disadvantageous for imaging, because broadening lowers resolution.

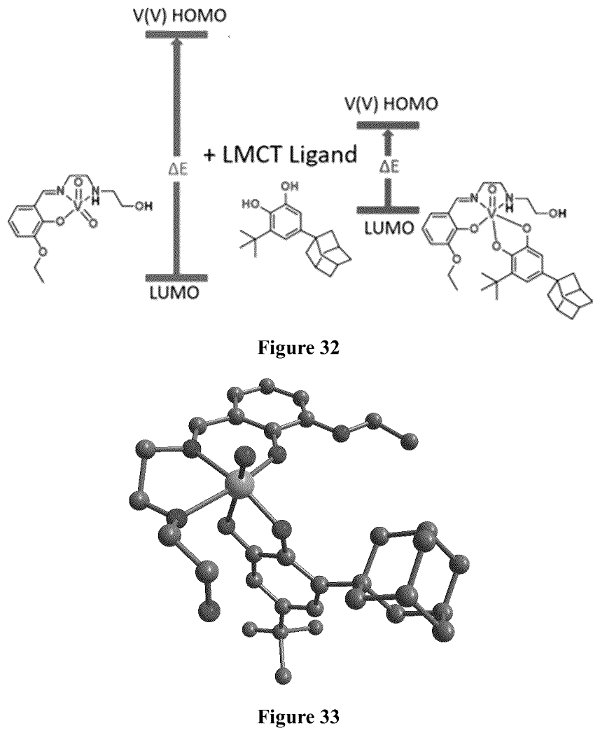

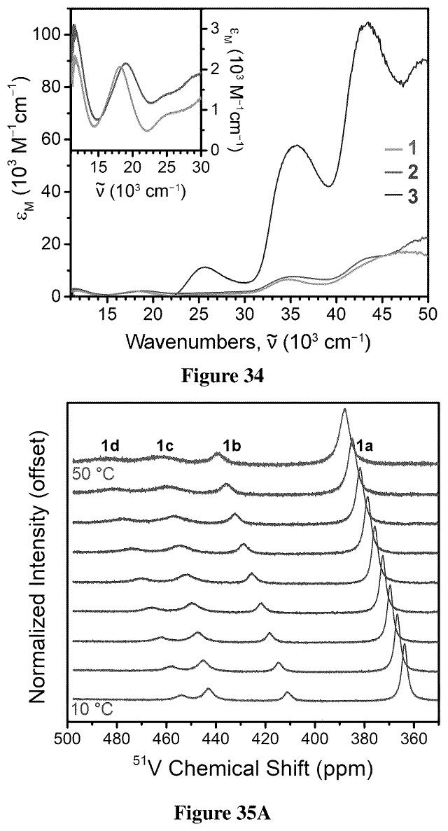

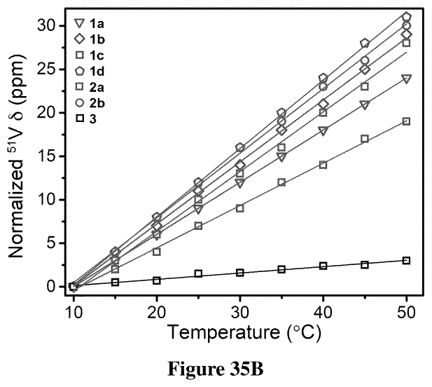

⁵¹V can function as an alternative promising nucleus for high resolution NMR thermometry because ⁵¹V possesses an intrinsically low quadrupolar moment. However, design strategies for temperature sensitivity for the ⁵¹V nucleous are currently unexplored. We note that ⁵¹V possesses no d electrons in the most common NMR-active oxidation state, V(V). Hence, the relevant energy gaps that direct a temperature dependent paramagnetic shift (via Ramsey's equation) would likely be ligand-to-metal charge transfer (LMCT) states. We hypothesize that explicit tuning of these states could provide the first design strategy for temperature sensitivity in ⁵¹V NMR spectroscopy (FIG. 32). Importantly, we hypothesized that non-innocent ligands would be desirable in this design, as said species would ensure high-energy frontier MOs for low-energy LMCTs.

To test the foregoing hypotheses, we herein report the structure, electronic spectroscopy, and variable-temperature ⁵¹V NMR spectroscopic properties of a series of three complexes: [VO(EtHshed)(tbad)] (1, tbad=5-(adamantan-1-yl)-3-(tert-butyl)benzene-1,2diol, EtHshed=(E)-2-ethoxy-6-(((2-((2-hydroxyethyl)amino)ethyl)imino)methyl)phenol), [VO(EtHshed)(cat)] (2, cat=catechol), and [VO2(EtHshed)] (3). In 1 and 2, the presence of the tbad and catecholate ligands insert filled, high-energy ligand orbitals that enforce the existence of a low-energy LMCT, which is absent for 3. Crucially, 1 and 2 also exhibit an order of magnitude higher Δδ/ΔT, ca. 0.7 ppm/° C., relative to 3 (0.08 ppm/° C.). These data support the interpretation that LMCT considerations are one viable path toward amplifying Δδ/ΔT for ⁵¹V NMR thermometers.

Syntheses of 1 and 2 is prepared from the pre-cursor Schif base complex 3. The 3 is prepared from the condensation reaction of 3-ethoxy salicylaldehyde and N-(2-hydroxyethyl) ethylenediamine followed by the addition of vanadyl sulfate in degassed methanol, as reported previously. Syntheses of 1 and 2 proceed by addition of tbad and catechol, respectively, in degassed methanol to the yellow, 3, as reported previously for other noninnocent vanadium-Schiff base complexes. Upon the addition of either tbad or catechol to 3, the colors of the reaction mixtures change from faint yellow to a deep, intense purple color.

The geometry of 1 contains a distorted octahedral vanadium atom in the crystalline state which presumably also are present in both 2 and 3. Note that 3 exists both as a monomer and dimer of two [VO2(Hshed)] units which likely disassociate in solution. In 3, the bond distance for the V-O bond is 1.616 (3) Å, which is typical for a V(V) oxo group. For 1, the bond distance of the V-O bond is 1.591 (7) Å, which is also in the expected range for V(V) oxo group. The C—O bond distances for catechol in 1 are 1.31 (1) Å and 1.328 (9) Å, which is shorter than the observed value of approximately 1.35 Å in free catechol. This bond is however longer than the expected value of 1.29 Å in a reduced semiquinone radical and much longer than the expected value of 1.22 Å in a reduced o-benzoquinone. These observations are consistent with all complexes being V(V) oxos bound to catecholate ligands.

In contrast to the yellow color of 3, the 1 and 2 complexes are dark purple and burgundy, respectively. The UV-Vis spectrum for 1 has three distinguishable peaks at 288 nm (6500 M⁻¹ cm⁻¹), 551 nm (2063 M⁻¹ cm⁻¹) and 862 nm (2375 M⁻¹ cm⁻¹). Complex 2 has four distinguishable peaks at 223 nm (15374 M⁻¹ cm⁻¹), 284 nm (7626 M⁻¹ cm⁻¹), 531 nm (2200 M⁻¹ cm¹), and 865 nm (2956 M⁻¹ cm⁻¹). The magnitude of the extinction coefficient for the three lower energy peaks coupled with the fact that V(V) has 0 d-electrons suggests that these peaks are low energy LMCT bands for 1 and 2. In contrast, the UV-Vis spectrum for 3 shows three distinguishable peaks at 230 nm (77667 M⁻¹ cm⁻¹), 281 nm (42333 M⁻¹ cm⁻¹), and 390 nm (8000 M⁻¹ cm⁻¹). The extinction coefficients for these peaks suggest that they could also be attributed to LMCT bands or LLCT bands. In either case, they are at a notably higher energy than the bands in 1 and 2, and likely stem from the absence of a catecholate-like ligand.

The ⁵¹V NMR spectra at ambient temperature for 1 and 2 contains several pis consistent with the presence of isomers in solution; the possibility that the signals are impurities were ruled out. The ⁵¹V NMR spectra of 1 gives peaks for four isomers in solution at 466 ppm (minor 1 (new 1d), 450 ppm (minor 2 1c), 422 ppm (minor 3 1b), and 373 ppm (major 1a). The asymmetry of the tbad ligand in this case is likely contributing to the observation of some of these to isomers. For 2, the ⁵¹V NMR spectra gives two isomeric peaks in solution at 204 ppm (major) and 258 ppm (minor). In this species, the catecholate ligand is symmetric, and we see half as many isomers as 1 further suggesting ligand asymmetry contributes to the isomer distribution. In contrast to both 1 and 2, the ⁵¹V NMR spectra for 3 shows only one peak, at −525 ppm. The chemical shifts of 1, 2, and 3 follow expectations for V(V) species with (and without) noninnocent ligands. Importantly, the ⁵¹V S values for 1-3 also trend with the observed energies of the LMCT transitions in the UV-Vis spectra a lower energy LMCT accompanies a complex with a more downfield ⁵¹V S.

Next, we analyzed the variable-temperature ⁵¹V NMR spectra of 1-3 in CH₃CN to determine how affected the temperature sensitivity of the ⁵¹V signals are relative to the signals in 3. Measurements were all made at ca. 105 MHz for ⁵¹V over a 10 to 50° C. window with a 400 MHz (¹H) NMR magnet. For 1 and 2, the temperature dependence of each isomer's peak can be clearly tracked. The major peak of 1 shows a Δδ/ΔT of 0.61 (1) ppm/° C. from 10-50° C. The other minor peaks, in order of increasing S, show temperature sensitivities of 0.70(1), 0.49(1), 0.61 (1) ppm/° C., respectively. The peaks for 1 also broaden with increasing temperature, showing linewidth changes from 3 to 5 ppm over the measured window consistent with isomer interconversions. For 2, ⁵¹V δ of the major peak shifts by 0.67 (1) ppm/° C. and one of the minor peak shifts by 0.79 (1) ppm/° C. from 10-50° C. Both isomers see a broadening in linewidth as the temperature increases from 3 ppm at 10° C. to 5 ppm at 50° C. In contrast, the vanadium shift 3, is virtually temperature insensitive: the ⁵¹V S changes only by 0.07 (1) ppm/° C. from 10-50° C. with insignificant change in linewidth.

The observation of a high sensitivity for ⁵¹V signals in 1 and 2, but not in 3, trends with the presence of a low-energy LMCT and the catecholate-type ligands. Hence, results highlight the importance of the presence of the noninnocent ligand in the ⁵¹V NMR thermometers with a high Δδ/ΔT. These values show the high-temperature sensitivity of the ⁵¹V S signals, e.g. VO(Oi-pr)₃ (0.61 ppm/° C.), but these values are lower than reported LV(CO)ₙ complexes, which can exhibit temperature sensitivities close to 1.2 ppm/° C. The current record Δδ/ΔT for a V(V) complex belongs to V(NO)₂(THF)₄Br with a shift of 1.23 ppm/° C., also higher than that observed for compound 1. These species all possess different oxidation states, V(—I), V(I) than ours V(V) and feature organometallic or other reactive ligand shells that are disadvantageous for bioimaging applications. Compounds 1 and 2 hold the record temperature sensitivities for oxidation state V complexes, and their ligand shells are compatible with biological applications.

A final point worth considering in these molecules concerns the ⁵¹V NMR linewidths. A linewidth, ν_{1/2}, of 3-5 ppm, as we found over the entire range of measurement of 1-3, is in general smaller than the typical linewidths for competitor ⁵⁹Co thermometer systems, which can have 10 s to 100 s of ppm linewidths. The ratio of the temperature sensitivity to the linewidth (Δδ/ΔT)/ν_{1/2} yields the resolution of the thermometry. For 1-3, these resolutions are ca. X (1), Y (2), and Z (3). These values are smaller than many of the state-of-the-art systems, despite the isomer exchange in addition to the biological compatibility of these species not shared by other complexes.

The ability to design higher temperature sensitivities from magnetic resonance responses is vital toward design of novel non-invasive thermometers. The foregoing data show the first design strategy for amplifying temperature sensitivity for the ⁵¹V NMR signals in V(V) complexes, affected by LMCT bands. As LMCT bands are tied to the complexes structures, their ability to penetrate cells and their signals are readily tunable they are potential probes. We anticipate tuning the Δδ/ΔT parameter and investigating how structural changes impact ⁵¹V temperature-dependent properties will identify a number of potential temperature probes.

### Example 7. Synthesis and Characterization of Vanadium(V) Complexes

Materials and Methods

General Materials. Salicylaldehyde (98%), 5-chlorosalicylaldehyde (98%), 3-methoxysalicylaldehyde (98.0%), vanadyl sulfate (98.0%) vanadyl acetylacetonate (98%), catechol (≥99.0%), 4-tert-butyl-catechol (≥97.0%), 3-methoxycatechol (99.0%), 3-methylcatechol (98.0%), tetrabromocatechol (98.0%), 4-nitrocatechol (98.0%), 4-nitrilecatechol (98.0%), 4-methylcatechol (98.0%), 6,7-dihydroxycoumarin (98.0%), 1-adamantanol (99.0%), 4-(diethylamino)-2-hydroxybenzaldehyde (99%), 3-ethoxysalicylaldehyde (99%), 2-hydroxynapthlene-1-carbaldehyde (99%), Exo-norborneol (98.0%) 2-(2-aminoethyl)pyridine (98%), ellagic acid (95%) and 5,6-di-hydroxyindoleand hydrogen peroxide solution (30%) were purchased from Sigma Aldrich and used as received. 2-(2-aminoethyl)pyridine (98%) was purchased from Oakwood Chemical and used as received. Catechol (≥99.0%), 4-tert-butyl-catechol (≥99.0%) and di-tert-butyl-catechol (98%) were purchased from Sigma Aldrich and recrystallized from toluene, pentane and petroleum ether, respectively, prior to use. Deuterated DMSO (d₆-DMSO), deuterated acetonitrile (d₃-MeCN), d₆-acetone, and deuterated chloroform (CDCl₃) were purchased from Cambridge Isotope and used as received.

General Methods. All syntheses were carried out using reflux under argon atmosphere unless noted otherwise. 1D and 2D ¹H NMR studies were carried out in organic solvents using a Bruker NEO400 spectrometer operating at 400 MHz at an ambient temperature. Chemical shift values (δ) are reported in ppm and referenced against TMS using the internal solvent peaks in ¹H NMR spectra (d₆-DMSO, δ at 2.50 ppm, CDCl₃, δ at 7.26 ppm) as internal standards. The complexes were also characterized using ⁵¹V NMR spectroscopy recorded on a Bruker NEO400 spectrometer equipped with an automated tuning module operating at 105.2 MHz at an ambient temperature. The ⁵¹V NMR spectra were acquired with a spectral window of 800 ppm, 4096 scans, a 90° pulse, an acquisition time of 0.08 s, and a 0.01 s relaxation delay as reported previously. 1D ⁵¹V NMR studies were referenced against [V(O)₂(HSHED)] at −529 ppm as a standard and reported in reference to VOCl₃ (0 ppm).

### Synthesis of Catechol Ligands

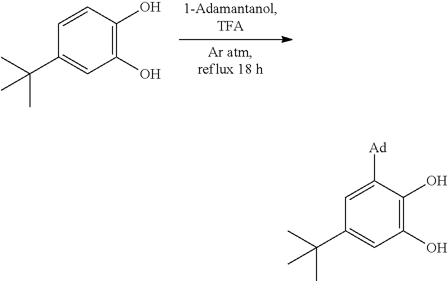

3-adamantyl-5-tert-butyl catechol (tbad). A 50 mL round bottom flask charged with a stir bar was flame dried and purged with Argon. 4-tert-Butylcatechol (1.66 g, 10.0 mmol) was added along with 1-adamantanol (3.06 g, 20.0 mmol) and trifluoroacetic acid (20 mL). The reaction mixture was brought to reflux and left to react under an Argon atmosphere for 18 hrs monitored by TLC. A milky-white precipitate was observed after 15 minutes. The white precipitate was filtered and washed with DI H₂O until the filtrate was neutral. The crude solid was purified by flash column chromatography (20:1 hexane/ethyl acetate) or was sublimated at 150° C. The white solid was dried on vacuum for 2 days yielding 2.34 g (78%). δ¹H NMR (CDCl₃, 400 MHz) δ 6.84 (d, J=2.3 Hz, 1H), 6.76 (d, J=2.3 Hz, 1H), 5.44 (s, 1H, OH), 4.67 (s, 1H, OH), 2.13 (m, 6H), 2.09 (m, 3H), 1.79 (m, 6H), 1.27 (s, 9H).

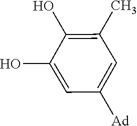

3-methyl-5-adamantyl catechol (3mad). A 10 mL round bottom flask charged with a stir bar was flame dried and purged with Argon. 3-methyl catechol (0.12 g, 1.0 mmol) was added along with 1-adamantanol (0.15 g, 1 mmol) and Trifluoroacetic acid (3 mL). The system was fitted with an Argon balloon and the mixture was left to react at room temperature for two days. A milky-tan precipitate was observed after two hrs. After two days the precipitate was filtered and washed with DI H₂O until the filtrate was neutral. The crude solid was purified by flash column chromatography (1000 mL of 230-400 mesh SiO₂, 70 mm column 30:1 hexane/ethyl acetate). The yellow solid obtained was dried under reduced pressure to yield 1.6 g (62%) as a while solid. δ¹H NMR (CDCl₃, 400 MHz) δ 6.74 (d, J=2.3 Hz, 1H), 6.69 (d, J=2.3 Hz, 1H), 5.16 (s, 1H), 4.98 (s, 1H), 2.25 (s, 3H), 2.14-2.03 (m, 3H), 1.85 (d, J=2.9 Hz, 6H), 1.66-1.81 (m, 6H).

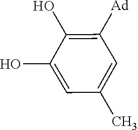

3-adamantyl-5-methyl catechol (5mad). A 10 mL round bottom flask charged with a stir bar was flame dried and purged with Argon. 4-methyl catechol (0.124 g, 1.0 mmol) was added along with 1-adamantanol (0.15 g, 1 mmol) and Trifluoroacetic acid (3 mL). The system was fitted with an Argon balloon and the mixture was left to react at room temperature for two days. A milky-tan precipitate was observed after two hrs. After two days the precipitate was filtered and washed with DI H₂O until the filtrate was neutral. The crude solid was purified by flash column chromatography (1000 mL of 230-400 mesh SiO₂, 70 mm column 30:1 hexane/ethyl acetate). The yellow solid obtained was dried under reduced pressure to yield 0.11 g (41%). δ¹H NMR (CDCl₃, 400 MHz) δ 6.61 (d, J=2.0 Hz, 1H), 6.55 (d, J=2.0 Hz 1H), 5.47 (s, 1H), 4.82 (s, 1H), 2.23 (s, 3H), 2.10 (m, 6H), 2.07 (m, 3H), 1.75-1.89 (m, 6H), 1.72-1.67 (m, 3H).

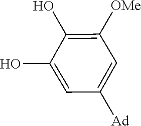

3-methoxy-5-adamantyl catechol (30mad). A 10 mL round bottom flask charged with a stir bar was flame dried and purged with Argon. 3-methoxy catechol (0.140 g, 1.0 mmol) was added along with 1-adamantanol (0.15 g, 1 mmol) and Trifluoroacetic acid (3 mL). The system was fitted with an Argon balloon and the mixture was left to react at room temperature for two days. The reaction turned red in color after 24 h and after 48 h the product was extracted with saturated sodium bicarbonate (20 mL) and ethyl acetate (30 mL). The organic layer was washed with saturated bicarb (15 mL) followed by DI water (15 mL) and brine (2×10 mL) then dried over magnesium sulfate. The crude solid was purified by flash column chromatography (1000 mL of 230-400 mesh SiO₂, 70 mm column 30:1 hexane/ethyl acetate) to yield 0.12 g (45%) as a pink solid. δ¹H NMR (CDCl₃, 400 MHz) δ 6.62 (d, J=2.0 Hz, 1H), 6.48 (d, J=2.0 Hz, 1H), 5.19 (d, J=6.5 Hz, 2H), 3.89 (s, 3H), 2.11-2.06 (m, 4H), 1.86 (d, J=2.9 Hz, 6H), 1.81-1.67 (m, 7H).

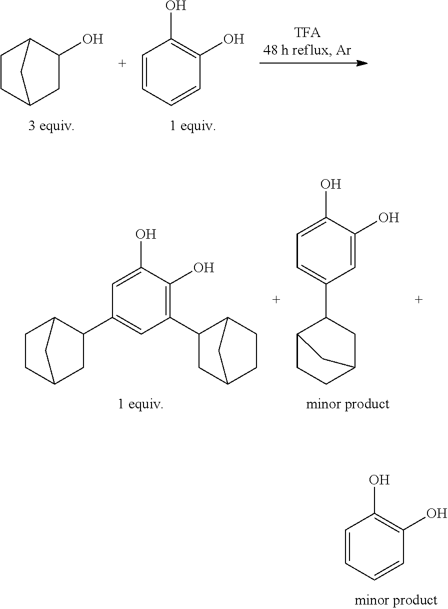

3,5-di-norbornane-catechol (DNC). To 5 mL of trifluoroacetic acid, catechol (0.028 g, 0.25 mmol, 1 equiv.) and exo-norborneol (0.084 g, 0.75 mmol, 3 equiv.) were added. The reaction mixture was allowed to reflux for 48 h at 80° C. under Ar and monitored by TLC (solvent: 80/20 PET ether/ethyl acetate). The reaction mixture was then quenched with ice cold water (20 mL) and then washed with saturated NaHCO₃ (50 mL). The reaction mixture was extracted with pentane (3×50 mL) and dried over sodium sulfate. The reaction mixture was concentrated in vacuo. ¹H NMR of the crude oil was recorded in d₆-DMSO. The product was isolated by flash column chromatography (solvent: 80/20 PET ether/ethyl acetate). Yield: 0.050 g (67%). δ¹H NMR (d₆-DMSO, 400 MHz)=6.76 (s, 1H), 6.54 (s, 1H), 2.88 (t, 2H), 1.65 (m, 4H), 1.54 (m, 8H), 1.50 (m, 4H), 1.43 (d, 2H), 1.42 (m, 2H), 1.34 (m, 4H), 1.12 (d, 2H).

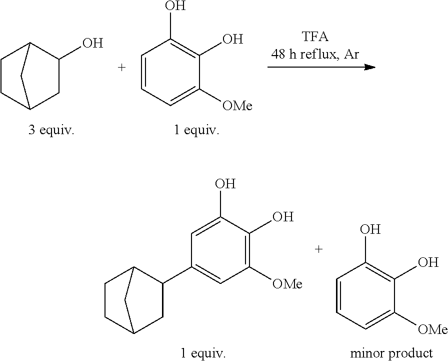 [SMILES: OC1CC2CCC1C2 ; COc1cccc(O)c1O ; COc1cc(C2CC3CCC2C3)cc(O)c1O ; COc1cccc(O)c1O]

3-methoxy-5-di-norbornane-catechol (OMeNC). To 5 mL of trifluoroacetic acid, 3-methoxy-catechol (0.035 g, 0.25 mmol, 1 equiv.) and exo-norborneol (0.084 g, 0.75 mmol, 3 equiv.) were added. The reaction mixture was then quenched with ice cold water (20 mL) and then washed with saturated NaHCO₃ (50 mL). The reaction mixture was extracted with pentane (3×50 mL) and dried over sodium sulfate. The reaction mixture was concentrated in vacuo. ¹H NMR of the crude oil was recorded in d₆-DMSO. The product was isolated by flash column chromatography (solvent: 80/20 PET ether/ethyl acetate). Yield: 0.020 g (34%). δ¹H NMR (d₆-DMSO, 400 MHz): 6.48 (s, 1H), 6.38 (s, 1H), 3.72 (s, 3H), 2.84 (q, 1H), 2.17 (m, 1H), 2.11 (m, 1H), 1.90 (m, 1H), 1.51 (m, 3H), 1.31 (m, 2H).

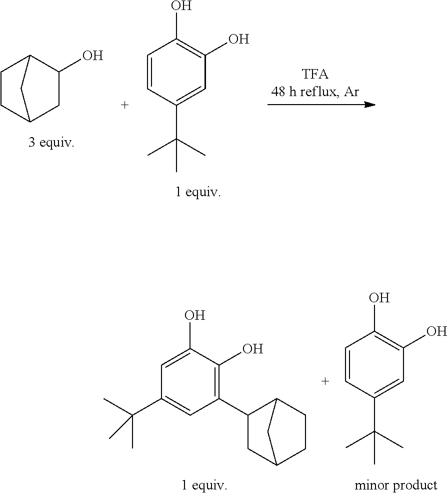

3-tert-butyl-5-di-norbornane-catechol (TBNC). To 5 mL of trifluoroacetic acid, 4-tert-butylcatechol (0.042 g, 0.25 mmol, 1 equiv.) and exo-norborneol (0.084 g, 0.75 mmol, 3 equiv.) were added. The reaction mixture was then quenched with ice cold water (20 mL) and then washed with saturated NaHCO₃ (50 mL). The reaction mixture was extracted with pentane (3×50 mL) and dried over sodium sulfate. The reaction mixture was concentrated in vacuo. ¹H NMR of the crude oil was recorded in d₆-DMSO. Yield: 0.049 g (75%). δ¹H NMR (d₆-DMSO, 400 MHz): 6.79 (dd, 1H), 6.76 (dd, 1H), 2.86 (m, 1H), 2.42 (s, 2H), 1.52 (m, 1H), 1.26 (s, 9H), 1.20 (m, 4H), 1.17 (m, 2H).

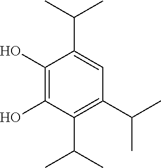 [SMILES: CC(C)c1cc(C(C)C)c(C(C)C)c(O)c1O]

Synthesis of 3,4,6-tri-isopropyl catechol. Catechol (2.02 g, 20.0 mmol) and 2-propanol (4.59 mL, 60.0 mmol) were stirred together under reflux until a temperature of 58° C. was reached. Sulfuric acid (4.26 mL, 80.0 mmol) was added dropwise and the reaction was allowed to reflux for 4 hours. The reaction mixture was neutralized with ice water (100 mL) and sodium bicarbonate (s), and the organic phase was extracted with ethyl acetate. The solvent was removed under evaporated pressure and the crude product was purified via column chromatography in 10% ethyl acetate: hexanes to yield 1.18 g (25%) of 3,4,6-tri-isopropyl catechol as a red solid. ¹H NMR: (400 MHz, Chloroform-d) δ 6.64 (s, 1H), 5.32 (s, 1H), 4.72 (s, 1H), 3.42-3.32 (m, 1H), 3.17 (hept, J=6.5 Hz, 1H), 3.05 (hept, J=6.9 Hz, 1H), 1.39 (dd, J=7.1, 0.8 Hz, 6H), 1.27 (dd, J=6.9, 0.8 Hz, 6H), 1.21 (dd, J=6.9, 0.8 Hz, 6H.

Vanadium Complexes

[VO(HSHED)(DHI)]. To a 1:1 mixture of degassed methanol and ethanol (5 mL total), the [VO₂(HSHED)] precursor (0.073 g, 0.25 mmol, 1 equiv.) and the 3,5-di-norbornane-catechol ligand (0.045 g, 0.30 mmol, 1.2 equiv.) were added. The reaction mixture was allowed to stir for 20 h at r.t. under argon. The resulting product was vacuum filtered, washed with cold methanol and dried under high vacuum. Yield: 0.026 g (25%). Characterization: δ¹H NMR (d₆-DMSO, 400 MHz): δ 8.89 (s, 1H), 7.55 (d, J=7.7 Hz, 1H), 7.44 (t, J=7.8 Hz, 1H), 6.87-6.75 (m, 2H), 5.53 (s, 1H), 4.86 (t, J=5.2 Hz, 1H), 3.79 (d, J=5.6 Hz, 2H), 3.40 (d, J=6.9 Hz, 2H), 3.17 (d, J=5.3 Hz, 1H), 2.89 (s, 1H). X. ⁵¹V NMR (d₆-DMSO, 105.2 MHz): −527 ppm. UV-vis λₘₐₓ: 375 nm.

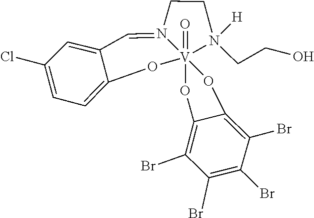

[VO(Cl-HSHED)(TB)]. To a 250 mL dry Schenk flask, ethyl acetate (100 mL) was added and then degassed with argon for 15 minutes. The [VO(Cl-HSHED)] precursor (0.325 g, 1.00 mmol) was added, followed by the addition of tetrabromocatechol (0.426 g, 1.00 mmol). The reaction mixture changed from light yellow to deep purple within 2 minutes and the solution was stirred for 24 hours at an ambient temperature under Ar. The reaction mixture was then vacuum filtered and concentrated to dryness in vacuo. The solid product was redissolved in the minimum amount of acetone (7.00 mL) and 100 ml of hexane. The mixture was stored at −20° C. overnight. The final product was vacuum filtered, washed with 2×25.0 mL cold hexane (<0° C.) and dried under high vacuum for 2 days. Yield: 0.572 g (78%). δ¹H NMR (d₃-MeCN, 400 MHz): 8.91 (s, 1H), 7.84 (d, 1H), 7.73 (d, 1H), 7.71 (d, 1H), 7.04 (d, 1H), 4.82 (m, 1H), 4.40 (m, 1H), 4.27 (m, 1H), 3.81 (m, 2H), 3.66 (m, 1H), 3.54 (m, 1H), 3.13 (m, 1H), 3.00 (m, 1H) δ⁵¹V NMR (d₃-MeCN, 101 MHz): 99 ppm. UV-vis λₘₐₓ: 524 nm, 821 nm

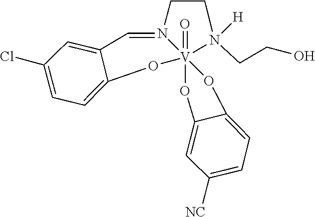

[VO(Cl-HSHED)(4Ni)]. To a 250 mL round bottom Schlenk flask, ethyl acetate (100 mL), which was then degassed with argon for 15 minutes. [VO(Cl-HSHED)] (0.325 g, 1.0 mmol) was added, followed by the addition of 4-nitrilecatechol (0.135 g, 1.0 mmol). The reaction mixture changed from light yellow to deep purple within 5 minutes and the solution was stirred for 24 h at an ambient temperature under Ar. The reaction mixture was then vacuum filtered and then concentrated to dryness under reduced pressure at room temperature. The purple residue was dissolved in a minimal amount of acetone and then n-hexane (100 mL) was added. The solution stored at −20° C. freezer overnight. The dark microcrystalline precipitate was vacuum filtered, washed with cold n-hexane (<0° C., 2×25 mL), and dried under vacuum for 2 days. Yield: 0.268 g (60%). ¹H (d₃-MeCN): 8.58 ppm (s, 1H); 7.53 ppm (m, 1H); 7.41 ppm (m, 1H); 7.05 ppm (m, 1H); 6.81 ppm, (m, 1H); 6.72 ppm (d, 1H); 4.45 ppm (s, 1H); 4.10-4.13 ppm (m, 1H); 3.96-4.01 ppm (m, 1H); 3.67 ppm (m, 1H); 3.56-3.59 ppm (m, 1H); 3.41 ppm (m, 1H); 3.27 ppm (m, 1H); 2.84-2.90 ppm (m, 1H); 2.71 (s, 1H)⁵¹V (d₃-MeCN) −73 ppm, −98 ppm.

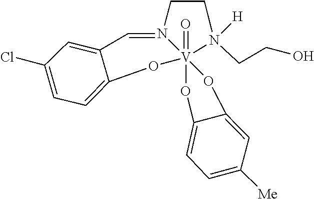

[VO(Cl-HSHED)(4Me)]. To a 250 mL round bottom Schlenk flask, ethyl acetate (100 mL), which was then degassed with argon for 15 minutes. [VO(Cl-HSHED)] (0.325 g, 1.00 mmol) was added, followed by the addition of 4-methylcatechol (0.124 g, 1.0 mmol). The reaction mixture changed from a light yellow to deep purple within 1 minute and the solution was stirred for 24 h at an ambient temperature under argon. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold n-hexane (<0° C., 2×25 mL), and dried under vacuum for 2 days. Yield: 0.294 g (68%). Characterization. δ¹H NMR (d₃-MeCN, 400 MHz): δ 8.54 (s, 1H), 7.49 (d, 1H), 7.40 (dd, 2H), 6.74 (d, 1H), 6.43 (m, 1H), 6.22 (s, 1H), 4.08 (m, 4H), 3.79 (dd, 1H), 3.64 (m, 1H), 3.55 (m, 1H), 3.44 (m, 1H), 3.33 (m, 1H), 2.95 (dd, 1H), 2.83 (s, 1H), 2.55 (m, 2H). δ⁵¹V NMR (d₃-MeCN, 101 MHz) 397 ppm (major), 431 ppm (minor), 454 ppm (minor). UV-vis λₘₐₓ: 546 nm, 868 nm.

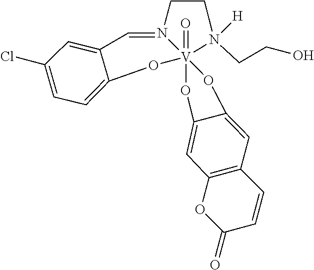

[VO(Cl-HSHED)(Coumarin)]: To a 250 mL round bottom Schlenk flask, ethyl acetate (100 mL), which was then degassed with argon for 15 minutes. [VO(Cl-HSHED)] (0.325 g, 1.0 mmol) was added followed by the addition of 6,7-dihydroxycoumarin (0.178 g, 1.0 mmol). The reaction mixture changed from a light yellow to deep purple within 5 minutes and the solution was stirred for 24 h at an ambient temperature under argon. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold n-hexane (<0° C., 2×25 mL), and dried under vacuum for 2 days. Yield: 0.256 g (53%). Characterization: δ¹H NMR (d₃-MeCN, 400 MHz) δ 8.67 (s, 1H), 7.70 (dd, 1H), 7.61 (d, 1H), 7.51 (m, 1H), 6.80 (d, 1H), 6.68 (d, 1H), 6.28 (s, 1H), 6.00 (d, 1H), 4.50 (m, 1H), 4.22 (m, 1H), 4.11 (m, 1H), 3.66 (m, 2H), 3.52 (m, 1H), 3.40 (m, 1H), 3.01 (m, 1H), 2.12 (s, 1H). δ⁵¹V NMR (d₃-MeCN, 101 MHz) 10 ppm (major), 40 ppm (minor). UV-vis λₘₐₓ: 359 nm, 524 nm, 881 nm

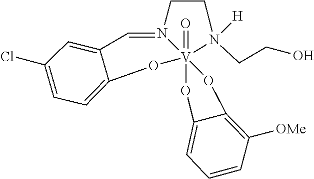

[VO(Cl-HSHED)(3-OMe)]. To a 250 mL round bottom Schlenk flask, ethyl acetate (100 mL), which was then degassed with argon for 15 minutes. [VO(Cl-HSHED)] (0.325 g, 1.0 mmol) was added, followed by the addition of 3-methoxy catechol (0.140 g, 1.0 mmol). The reaction mixture changed from a light yellow to deep purple within 1 minute and the solution was stirred for 24 h at an ambient temperature under argon. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold n-hexane (<0° C., 2×25 mL), and dried under vacuum for 3 days to yield purple solid. Yield: 0.238 g (53%). Characterization: δ¹H NMR (d₃-MeCN, 400 MHz) δ 8.54 (s, 1H), 7.49 (s, 1H), 7.40 (d, 1H), 6.74 (d, 3H), 6.31 (t, 1H), 6.05 (d, 1H) 5.96 (d, 1H), 4.08 (m, 3H), 3.83 (m, 4H), 3.63 (m, 1H), 3.39 (m, 2H), 2.98 (m, 1H), 2.81 (t, 1H). δ⁵¹V NMR (d₃-MeCN, 101 MHz) 334 ppm (major), 367 ppm (minor). UV-vis λₘₐₓ: 562 nm, 831 nm.

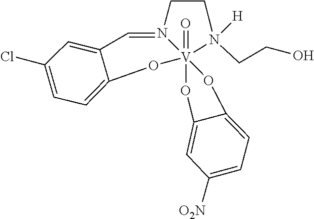

[VO(Cl-HSHED)(4NO)]. To a 250 mL round bottom Schlenk flask, ethyl acetate (100 mL), which was then degassed with argon for 15 minutes. [VO(Cl-HSHED)] (0.325 g, 1.0 mmol) was added, followed by the addition of 4-nitrocatechol (0.155 g, 1.0 mmol). The reaction mixture changed from a light yellow to deep purple within 3 minutes and the solution was stirred for 24 h at an ambient temperature under Ar. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold n-hexane (<0° C., 2×25 mL), and dried under vacuum for 3 days to yield a purple solid. Yield: 0.357 g (77%). Characterization: δ¹H NMR (d₃-MeCN, 400 MHz) δ 8.73 (s, 1H), 7.90 (d, 1H), 7.68 (dd, 1H), 7.45 (s, 1H), 6.85 (d, 1H), 6.41 (d, 1H), 4.60 (m, 1H), 4.25 (m, 1H), 4.12 (m, 1H), 3.72 (m, 1H), 3.60 (m, 2H), 3.01 (m, 1H). δ⁵¹V NMR (d₃-MeCN, 101 MHz) −160 ppm (major), −138 ppm (minor), −120 ppm (minor). UV-vis λₘₐₓ: 411 nm, 770 nm.

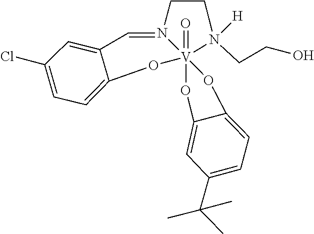

[VO(Cl-HSHED)(4 TB)]. To a 250 mL round bottom Schlenk flask, ethyl acetate100 mL), which was then degassed with argon for 15 minutes. [VO(Cl-HSHED)] (0.324 g, 1.0 mmol) was added, followed by the addition of 4-tertbutylcatechol (0.166 g, 1.0 mmol). The reaction mixture changed from a light yellow to deep purple within 1 minute and the solution was stirred for 24 h at an ambient temperature under Ar. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold n-hexane (<0° C., 2×25 mL), and dried under vacuum for 3 days to yield a purple solid. Yield: 0.356 g (75%). δ¹H (DMSO-d₆): 8.76 ppm (s, 1H), 7.62 ppm (s, 1H), 7.42 ppm (d, 1H), 6.76 ppm (d, 1H), 6.63-6.70 ppm (m, 1H), 6.01 ppm (d, 1H), 5.94 ppm (d, 1H), 4.76 ppm (s, 1H), 4.13-4.21 ppm (m, 1H), 3.95-4.00 ppm (m, 1H), 3.80 (s, 3H), 3.64-3.73 ppm (s, 3H), 3.44-3.55 ppm (m, 3H), 3.33 ppm (s, 6H), 3.27 ppm (s, 3H), 2.80-2.86 ppm (m, 1H), 2.37 ppm (s, 1H). δ⁵¹V (DMSO-d₆): 400 ppm, 386 ppm, 315 ppm, 299 ppm. UV-vis λₘₐₓ: 539 nm, 882 nm.

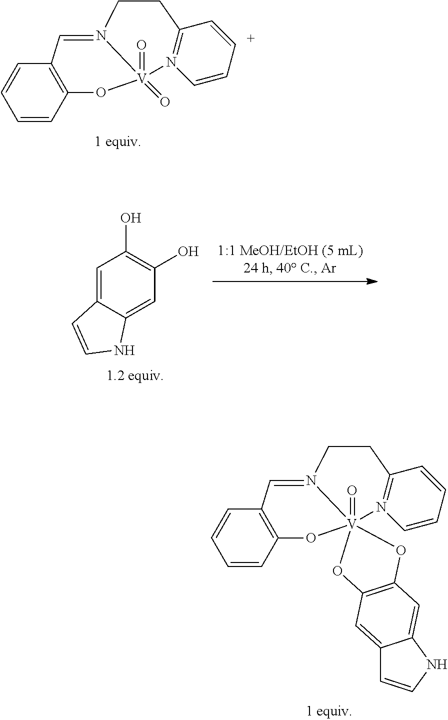

[VO(SALIEP)(DHI)]. To X mL of degassed methanol, the [VO₂(SALIEP)] precursor (0.145 g, 0.50 mmol, 1 equiv.) and the 3,5-di-norbornane-catechol ligand (0.179 g, 0.60 mmol, 1 equiv.) were added. The reaction mixture was allowed to stir for 20 h at r.t. under argon. The resulting product was vacuum filtered, washed with cold methanol and dried under high vacuum. Yield: 0.112 g (51%). δ¹H NMR (d₆-DMSO, 400 MHz): δ 8.52 (s, 1H), 8.27 (d, 1H), 8.16 (s, 1H), 7.67 (s, 1H), 7.46 (s, 1H), 7.29 (m, 2H), 7.22 (s, 1H), 7.15 (d, 1H), 6.92 (d, 1H), 6.79 (q, 1H), 6.52 (s, 1H), 5.75 (s, 1H), 4.09 (m, 2H), 3.44 (m, 2H). δ⁵¹V (d₆-DMSO, 105.2 MHz): −500, −519 ppm. UV-vis λₘₐₓ: 375 nm, 550 nm.

Schiff Base Scaffold with OCH₃-Substitution

The series of complexes with the —OCH₃ substituted scaffold was investigated. We believe that all the complexes are new, however, there is an x-ray of a related molecule. This series of compounds was synthesized but when using the general method, we had used to make the HSHED complex, we found that the yields were too low. In Schemes 1 and 2 we show optimization of 1) examine the solvents ad more than doubling the yield and 2) other minor changes such as stoichiometry and temperature; we find that we need excess of ligand, and that temp other than ambient temperature really did not improve the yield terrible much.

TABLE 13
Optimizing solvent in the [VO₂(3-OMeHshed)dtb].

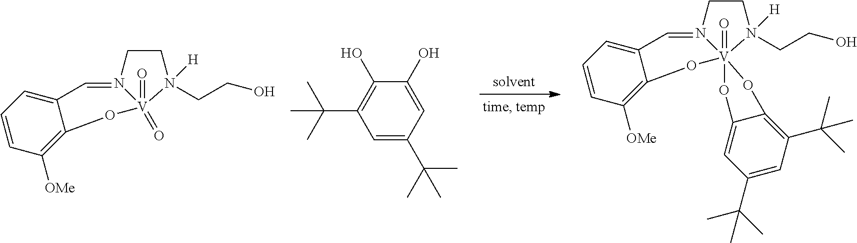

| | | | mmol | | | |
|---|---|---|---|---|---|---|
| Entry | Solvent | mmol cat. | V Precursor | Temp (º C.) | Time (hrs.) | Yield |
| 1 | EtOH | 1 | 1 | rt | 24 | 48% |
| 2 | Toluene | 1 | 1 | rt | 24 | 0% |
| 3 | DMSO | 1 | 1 | rt | 24 | <10% |
| 4 | DCM | 1 | 1 | rt | 24 | <10% |
| 5 | DMF | 1 | 1 | rt | 24 | 66% |
| 6 | EtOAc | 1 | 1 | rt | 24 | 80% |
| 7 | Et₂O | 1 | 1 | rt | 24 | 68%** |

**Reaction color indicates heavy impurity of starting material

TABLE 14
Optimizing acetone solvent and stoichiometry for synthesis of the [VO₂(3-
OMeHshed)dtb].

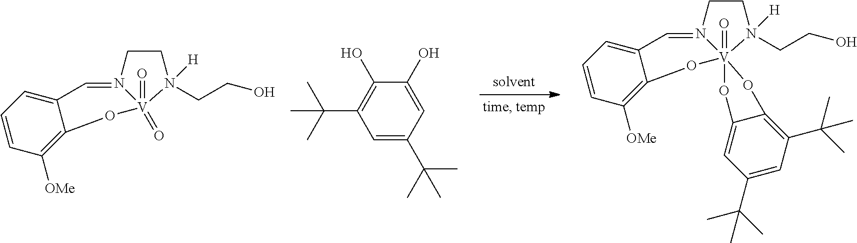

| | | | mmol | | | |
|---|---|---|---|---|---|---|
| Entry | Solvent | mmol cat. | V Precursor | Temp (º C.) | Time (hrs.) | Yield |
| 1 | Acetone | 1 | 1 | rt | 24 | 53% |
| 2 | Acetone | 1 | 1 | rt | 48 | 61% |
| 3 | Acetone | 1 | 1.2 | rt | 24 | 50% |
| 4 | Acetone | 1.2 | 1 | rt | 24 | 53% |
| 5 | Acetone | 1.5 | 1 | rt | 24 | 57% |
| 6 | Acetone | 1 | 1 | 0 | 24 | 70% |
| 7 | Acetone | 1 | 1 | −78 | 24 | 70% |
| 8 | MeOH | 1 | 1 | rt | 24 | 52% |
| 9 | MeCN | 1 | 1 | rt | 24 | 60% |
| 10 | DMP | 1 | 1 | rt | 24 | 0% |
| 11 | DME | 1 | 1 | rt | 24 | 0% |

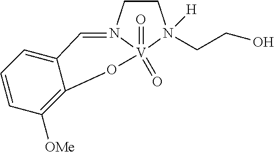

Synthesis of [VO2 (3-OMeHshed)]. To a 100 mL Schlenk flask, 40 mL of ACS reagent-grade methanol was added and degassed with argon for 20 minutes. This was followed by the addition of 2-hydroxy-3-methoxybenzaldehyde (1.43 g, 9.40 mmol) and N-(2-hydroxyethyl) ethylenediamine (0.979 g, 9.40 mmol). The reaction mixture was let to stir for an hour at ambient temperature under argon. Vanadyl sulfate (1.87 g, 11.5 mmol), dissolved in 25 mL degassed water, was added to the reaction mixture after 1 hour. The reaction mixture was let then to stir for 3 hours at ambient temperature under argon. After 3 hours, sodium hydroxide (0.752 g, 18.8 mmol) was added, the reaction mixture was opened to air and let to stir overnight. The product then was vacuum filtered, washed with 25 mL cold (0° C.) methanol and left to dry under high vacuum for 3 days. The product was a yellow-green powder. Yield: 0.296 g (91%). δ⁵¹V NMR (101 MHz, CD₃CN): −532 ppm. δ¹H NMR (400 MHz, CDCl₃): 8.93 ppm (s, 1H), 7.20 ppm (d, 1H), 7.14 ppm (d, 1H), 6.82 ppm (t, 1H), 4.13-4.04 ppm (m, 2H), 3.86 ppm (m, 2H), 3.80 ppm (s, 3H), 3.46-3.33 ppm (m, 1H), 3.32 ppm (m, 1H), 2.97-2.92 ppm (m, 1H), 2.66 ppm (m, 1H).

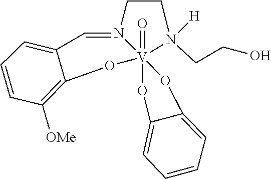

[VO(3-OMeHshed)(cat)]. To a 250 mL round bottom Schlenk flask, ethyl acetate (100 mL), which was then degassed with argon for 15 minutes. [VO(3-OMe-HSHED)] (0.325 g, 1.00 mmol) was added, followed by the addition catechol (0.110 g, 1.00 mmol). The reaction mixture changed from a light yellow to deep purple within 1 minute and the solution was stirred for 24 h at an ambient temperature under Ar. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold hexanes (<0° C., 2×25 mL), and dried under vacuum for 3 days to yield a purple solid. Yield: 0.247 g (60%). δ⁵¹V NMR (101 MHz, CD₃CN): 207 ppm (major), 256 ppm (minor). δ¹H NMR (400 MHz, CDCl₃): 8.53 ppm (s, 1H), 7.01 ppm (t, 1H), 6.72 ppm (m, 2H), 6.64 ppm (m, 2H), 6.43 ppm (d, 1H), 6.26 ppm (m, 2H), 4.14 ppm (m, 1H), 4.04-3.92 ppm (m, 2H), 3.67 ppm (s, 3H), 3.55 ppm (m, 1H), 3.45 ppm (m, 1H), 3.39-3.34 ppm (m, 1H), 3.27-3.20 ppm (m, 1H), 2.85-2.79 ppm (m, 1H), 2.76-2.73 ppm (m, 1H). UV-vis □ₘₐₓ: 525 nm, 873 nm

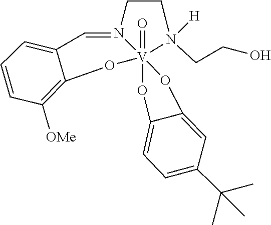

[VO(3-OmeHshed)(4-tbu)]. To a 250 mL round bottom Schlenk flask, ethyl acetate (100 mL), which was then degassed with argon for 15 minutes. [VO(3-Ome-HSHED)] (0.325 g, 1.00 mmol) was added, followed by the addition of 4-tert-butylcatechol (0.166 g, 1.00 mmol). The reaction mixture changed from a light yellow to deep purple within 1 minutes and the solution was stirred for 24 h at an ambient temperature under Ar. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold hexanes (<0° C., 2×25 mL), and dried under vacuum for 3 days to yield a purple solid. Yield: 0.365 g (78%). δ⁵¹V NMR (101 MHz, CD₃CN): 346 ppm (major), 374 ppm (minor), 392 ppm (minor). δ¹H NMR (400 MHz, CDCl₃): 8.48 ppm (s, 1H), 6.99 ppm (m, 3H), 6.87 ppm (t, 1H), 6.36-6.27 ppm (m, 2H), 6.27 ppm (m, 2H), 4.03-3.90 ppm (m, 4H), 3.66 ppm (s, 3H), 3.55 ppm (m, 1H), 3.47 ppm (m, 1H), 3.33 ppm (m, 1H), 3.23 ppm (m, 1H), 2.82 ppm (m, 1H), 2.73 ppm (m, 1H), 1.20 ppm (s, 3H), 1.17 ppm (s, 6H). UV-vis λₘₐₓ: 543 nm

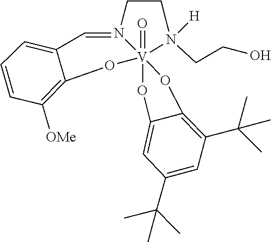

[VO(3-OmeHshed)(dtb)]. To a 250 mL round bottom Schlenk flask, ethyl acetate (100 mL), which was then degassed with argon for 15 minutes. [VO(3-Ome-HSHED)] (0.325 g, 1.00 mmol) was added, followed by the addition of 3,5-di-tert-butylcatechol (0.222 g, 1.00 mmol). The reaction mixture changed from a light yellow to deep purple within 3 minutes and the solution was stirred for 24 h at an ambient temperature under Ar. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold hexanes (<0° C., 2×25 mL), and dried under vacuum for 3 days to yield a purple solid. Yield: 0.420 g (80%). δ⁵¹V NMR (101 MHz, CD₃CN): 368 ppm (major); 423 ppm (minor), 456 ppm (minor), 478 ppm (minor). δ¹H NMR (400 MHz, CDCl₃): 8.46 ppm (s, 1H), 6.96 ppm (m, 3H), 6.64 ppm (t, 1H), 6.17 ppm (m, 1H), 4.02-3.89 ppm (m, 4H), 3.72 ppm (m, 1H), 3.63 ppm (s, 3H), 3.55 ppm (m, 1H), 3.44 ppm (m, 2H), 3.36 ppm (m, 1H), 3.24 ppm (m, 1H), 2.83-2.75 ppm (m, 2H), 1.32 ppm (s, 6H), 1.20 ppm (s, 3H), 1.17 ppm (s, 6H), 1.16 ppm (s, 3H). UV-vis λₘₐₓ: 553 nm, 869 nm

Schiff Base Scaffold with —OCH₂CH₃-Substitution

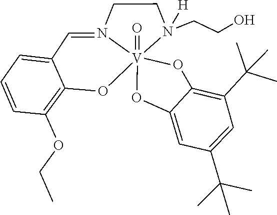

[VO(3-OEtHshed)(dtb)]. To a clean, dry 100 mL Schlenk flask, 25 mL of HPLC-grade methanol was added and degassed with nitrogen for 15 minutes. Following this, VO2(EtHshed) (0.334 g, 1.000 mmol) was added to the methanol followed by 3,5-di-tert-butyl catechol (0.222 g, 1.000 mmol). Immediately after the addition of the catechol, the reaction mixture was placed under nitrogen. Upon the addition of the catechol, the color slowly changed from yellow to brown to dark violet. The reaction mixture was allowed to stir overnight under nitrogen. The solution was then filtered in vacuo and the filtrate was then evaporated to complete dryness via rotary evaporator. To the resulting solid was then dissolved in minimal GC-Resolved grade acetone followed by the addition of ACS-grade hexanes (100 mL). The solution was then capped and stored at −20° C. overnight to precipitate out the desired purple solid. Yield: 63%. δ¹H NMR (CDCl₃, 400 MHz) δ 8.33 (s, 1H) 7.01 (d, 1H) 6.95 (d, 1H) 6.61 (t, 1H) 6.35 (s, 1H) 6.27 (s, 1H) 4.16 (m, 1H) 3.96 (m, 4H) 3.62 (m, 1H) 3.45 (m, 1H) 3.34 (s, 1H) 3.10 (m, 1H) 1.41 (s, 12H) 1.21 (m, 20H) 0.97 (t, 3H). δ⁵¹V NMR (d₃-MeCN, 101 MHz) δ 375 ppm (major) 427 ppm (minor) 456 ppm (minor) 485 ppm (minor).

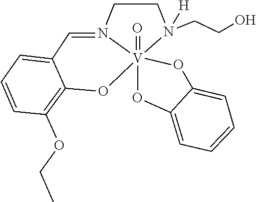

[VO(3-OEtHshed)(cat)]. To a clean, dry 100 mL Schlenk flask, 25 mL of HPLC-grade methanol was added and degassed with nitrogen for 15 minutes. Following this, VO₂(EtHshed) (0.167 g, 0.500 mmol) was added to the methanol followed by catechol (0.055 g, 0.500 mmol). Immediately after the addition of the catechol, the reaction mixture was placed under nitrogen. Upon the addition of the catechol, the color slowly changed from yellow to brown to dark violet. The reaction mixture was allowed to stir overnight under nitrogen. The solution was then filtered in vacuo and the filtrate was then evaporated to complete dryness via rotary evaporator. To the resulting solid was then dissolved in minimal GC-Resolved grade acetone followed by the addition of ACS-grade hexanes (100 mL). The solution was then capped and stored at −20° C. overnight to precipitate out the desired purple solid. Yield: 66%. δ¹H NMR (CDCl₃, 400 MHz) δ 8.42 (s, 1H) 7.14 (d, 1H) 7.07 (d, 1H) 7.01 (d, 1H) 6.70 (t, 1H) 6.58 (d, 1H) 6.47 (d, 1H) 6.38 (d, 1H) 4.21 (m, 1H) 4.02 (m, 4H) 3.86 (m, 1H) 3.68 (m, 1H) 3.50 (m, 1H) 3.34 (m, 1H) 3.07 (m, 1H) 1.66 (s, 2H), 1.23 (t, 3H). δ⁵¹V NMR (d₃-MeCN, 101 MHz) δ 204 ppm (major) 259 ppm (minor).

Schiff Base Scaffold with —N(CH₂CH₃)₂-Substitution

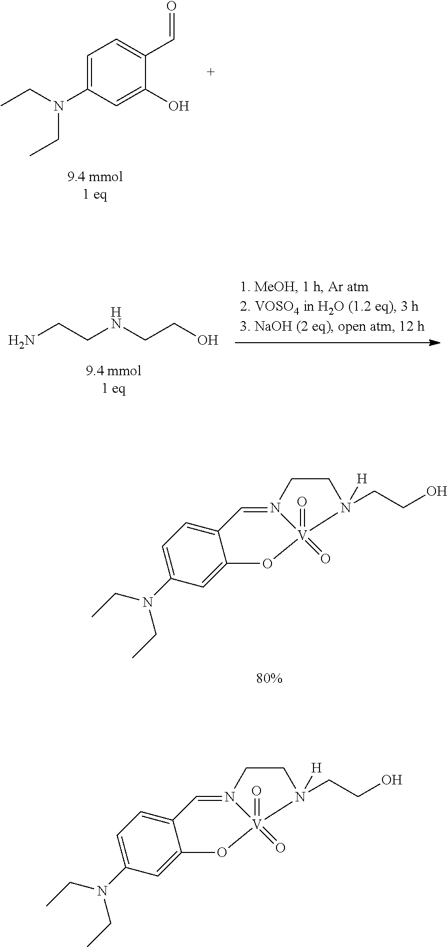

[VO2(dea-Hshed)]. To a 100 mL round bottom flask HPLC grade methanol (40 mL) was added and degassed with Argon for 20 minutes. 4-(diethylamino)-2-hydroxybenzaldehyde (1.82 g, 9.4 mmol) was then added followed by N-(2-hydroxyethyl)-1,2-ethanediamine (0.95 mL, 9.4 mmol) creating a light red solution. The reaction mixture was left to stir for 1 h under Argon. Vanadyl sulfate hydrate (1.87 g, 11.5 mmol) was dissolved in degassed water (25 mL) and added turning the reaction dark red. The resulting dark red mixture was left to stir at ambient temperature under Argon. After 3 hours, NaOH (0.75 g, 18.8 mmol) dissolved in a minimum amount of water was added and the reaction was allowed to stir overnight open to air. The yellow-brown mixture was put in an ice bath for 20 minutes then vacuum filtered and rinsed with with cold methanol (2×15 mL) to yield 2.4 g (80%) of a yellow solid. δ¹H NMR (d₆-DMSO, 400 MHz): 8.51 (s, 1H), 7.26 (d, J=8.9 Hz, 1H), 6.27 (dd, J=8.9, 2.4 Hz, 1H), 5.89 (d, J=2.4 Hz, 1H), 5.39 (s, 1H), 4.77 (t, J=5.3 Hz, 1H), 3.90 (q, J=3.9 Hz, 2H), 3.81 (q, J=5.4 Hz, 2H), 3.38 (q, J=7.1 Hz, 4H), 3.32 (s, 3H), 3.23-3.12 (m, 1H), 2.83 (dq, J=13.0, 5.9 Hz, 1H), 2.64-2.53 (m, 1H), 1.11 (t, J=7.0 Hz, 6H). δ⁵¹V NMR (d₆-DMSO, 105 MHz): −515 ppm.

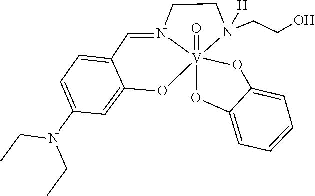

[VO(dea-Hshed)(cat)]. To a 100-mL round bottom Schlenk flask ACS grade acetone (50 mL) was added and degassed with Argon for 20 minutes. [VO2(dea-Hshed)] (0.18 g, 0.5 mmol) was added to the degassed acetone, followed by pyrocatechol (0.05 g, 0.5 mmol). A deep purple solution resulted after 30 min but stirred for 24 h. The reaction mixture was vacuum filtered and then concentrated to dryness under reduced pressure at room temperature. The purple residue was dissolved in a minimal amount of HPLC grade acetone and then n-hexane (50 mL) was added. The solution was stored at −20° C. for 2-3 days. The black microcrystalline precipitate was vacuum filtered, washed with cold hexanes (20 mL), and dried under vacuum for 3 days to yield 0.086 g (38%) of a black solid. δ¹H NMR (400 MHz, CDCl₃): 8.13 (s, 1H), 7.15 (d, J=8.9 Hz, 1H), 6.78 (s, 1H), 6.62 (d, J=8.1 Hz, 1H), 6.51 (d, J=8.1 Hz, 1H), 6.39 (s, 1H), 6.22 (d, J=9.0 Hz, 1H), 6.15 (s, 1H), 4.14 (d, 1H), 4.06 (s, 2H), 3.92 (d, J=13.1 Hz, 1H), 3.85 (s, 1H), 3.67 (s, 1H), 3.44 (s, 1H), 3.37 (q, J=7.5 Hz, 4H), 3.02 (d, 1H), 1.16 (t, J=7.1 Hz, 6H). δ⁵¹V NMR (CDCl₃, 105 MHz): 124 ppm. UV/Vis (DMSO), λₘₐₓ/nm: 540, 887.

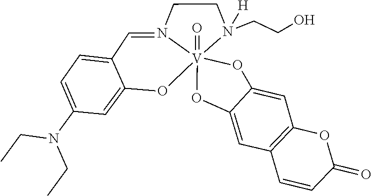

[VO(dea-Hshed)(coumarin)]. To a 100-mL round bottom Schlenk flask ACS grade acetone (50 mL) was added and degassed with Argon for 20 minutes. [VO2(dea-Hshed)] (0.18 g, 0.5 mmol) was added to the degassed acetone, followed by 6,7-dihydroxy coumarin (0.20 g, 0.50 mmol) A dark purple solution resulted after 30 min but stirred for 24 h. The reaction mixture was vacuum filtered and the filter product was collected. The filtrate was concentrated to dryness under reduced pressure at room temperature. The dark green residue was dissolved in a minimal amount of HPLC grade acetone and then n-hexane (50 mL) was added. The solution was stored in −20° C. freezer for 2-3 days. The dark green microcrystalline precipitate was vacuum filtered, washed with cold hexanes (20 mL), and dried under vacuum for 3 days to yield 0.18 g (69%) of a dark green solid. ¹H NMR (d₃-MeCN, 400 MHz) δ 8.36 (s, 1H), 7.68 (m, 1H), 7.34 (d, J=9.0 Hz, 1H), 6.64 (s, 1H), 6.39 (dd, J=9.0, 2.5 Hz, 1H), 6.23 (s, 1H), 5.99-5.89 (m, 2H), 4.31 (s, 1H), 4.04 (dt, J=11.4, 6.7 Hz, 1H), 3.82-3.69 (m, 1H), 3.68-3.58 (m, 1H), 3.49 (td, J=8.6, 4.1 Hz, 1H), 3.41 (q, J=7.1 Hz, 4H), 3.37-3.25 (m, 1H), 2.90 (qd, J=11.4, 4.9 Hz, 1H), 1.14 (t, J=7.0 Hz, 6H). δ⁵¹V NMR (d₃-MeCN, 105 MHz): −114 ppm (major), −73 ppm (minor). UV/Vis (DMSO), λₘₐₓ/nm: 847.

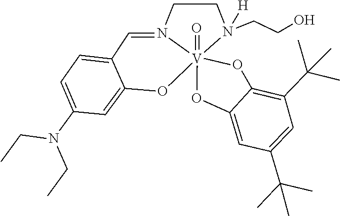

[VO(dea-Hshed)(dtb)]. To a 250-mL round bottom Schlenk flask ACS grade acetone (100 mL) was added and degassed with Argon for 20 minutes. [VO2(dea-Hshed)] (0.36 g, 1.0 mmol) was added to the degassed acetone, followed by 3,5-di-tert-butyl catechol (0.22 g, 1.0 mmol). A dark purple solution resulted after 30 min but stirred for 24 h. The reaction mixture was vacuum filtered and then concentrated to dryness under reduced pressure at room temperature. The purple residue was dissolved in a minimal amount of HPLC grade acetone and then n-hexane (100 mL) was added. The solution was stored at −20° C. for 2-3 days. The black microcrystalline precipitate was vacuum filtered, washed with cold hexanes (2×20 mL), and dried under vacuum for 3 days to yield 0.46 g (71%) of a black solid. δ¹H NMR (d₆-DMSO 400 MHz): 8.36 (s, 1H), 7.24 (d, J=8.8 Hz, 1H), 6.22 (m, 3H), 5.77 (s, 1H), 4.80 (s, 1H), 4.74 (t, 1H), 4.46 (m, 1H), 4.13-3.95 (m, 2H), 3.85 (m, 1H), 3.71 (m, 1H), 3.46 (m, 1H), 3.35 (q, J=7.6 Hz, 4H), 3.29-3.23 (m, 1H), 2.75 (qd, 1H), 2.47-2.23 (m, 1H), 1.37 (s, 9H), 1.22 (s, 9H), 1.09 (t, J=7.0 Hz, 6H). δ⁵¹V NMR (d₆-DMSO, 105 MHz): 279 ppm (major), 326 ppm (minor), 341 ppm (minor), 362 ppm (minor).

Schiff-Base Scaffold with Naphthalene Substitution

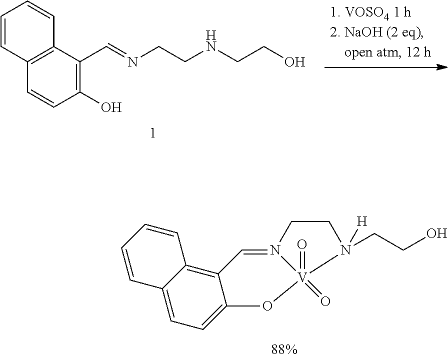

[VO2(nap-Hshed)]. To a 250 mL round bottom flask HPLC grade methanol (50 mL) was added and degassed with Argon for 20 minutes. The condensation product of 2-hydroxy-1-naphthaldehyde and N-(2-hydroxyethyl)-1,2-ethanediamine (1) (1.3 g, 5.0 mol) was added creating a yellow solution. Vanadyl sulfate hydrate (0.91 g, 5.0 mmol) was dissolved in degassed water (15 mL) and added turning the reaction mixture black. The resulting mixture was left to stir for 1 h then NaOH (0.40 g, 10 mmol) was dissolved in water (8.0 mL) and added slowly over 5 minutes. The reaction was left to stir open to air for 12 h producing a yellow precipitate. The reaction was placed in an ice bath for 20 minutes then the product was filtered and rinsed with cold methanol (30 mL). The solid was dried under vacuum for 3 days to yield 88% of a yellow solid. ¹H NMR (d₆-DMSO, 400 MHz): δ 9.76 (s, 1H), 8.39 (d, J=8.6 Hz, 1H), 8.00 (d, J=9.2 Hz, 1H), 7.89-7.82 (m, 1H), 7.59 (ddd, J=8.5, 6.9, 1.5 Hz, 1H), 7.37 (t, J=7.5 Hz, 1H), 7.05 (d, J=9.1 Hz, 1H), 5.67 (s, 1H), 4.83 (t, J=5.3 Hz, 1H), 4.32-4.23 (m, 1H), 4.11 (m, 1H), 3.83 (q, J=5.7 Hz, 2H), 3.43-3.34 (m, 2H), 2.88 (dt, J=11.9, 5.1 Hz, 1H), 2.68 (td, J=11.8, 4.8 Hz, 1H). δ⁵¹V NMR (d₆-DMSO, 1015 MHz): −524 ppm.

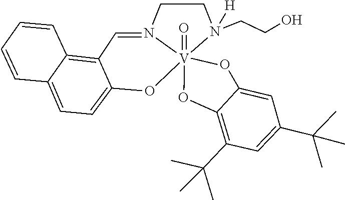

[VO(nap-Hshed)(dtb)]. To a 250-mL round bottom Schlenk flask ACS grade acetone (100 mL) was added and degassed with Argon for 20 minutes. [VO2(nap-Hshed)] (0.34 g, 1.0 mmol) was added to the degassed acetone, followed by 3,5-di-tert-butyl catechol (0.22 g, 1.0 mmol). A dark purple solution resulted after 1 hour but stirred for 24 h. The reaction mixture was vacuum filtered and then concentrated to dryness under reduced pressure at room temperature. The purple residue was dissolved in a minimal amount of HPLC grade acetone and then n-hexane (100 mL) was added. The solution was stored at −20° C. for 2-3 days. The dark purple microcrystalline precipitate was vacuum filtered, washed with cold hexanes (2×20 mL), and dried under vacuum for 3 days to yield 0.30 g (56%) of a purple solid. δ¹H NMR (d₆-DMSO, 400 MHz) δ 9.58 (s, 1H), 8.33 (d, J=8.5 Hz, 1H), 7.95 (d, J=9.1 Hz, 1H), 7.82 (d, J=8.1 Hz, 1H), 7.57 (t, J=7.8 Hz, 1H), 7.34 (t, J=7.5 Hz, 1H), 6.93 (d, J=9.2 Hz, 1H), 6.25 (m, 2H), 4.75 (m, 2H), 4.35 (d, J=13.5 Hz, 1H), 4.24 (s, 1H), 4.04 (t, J=13.2 Hz, 1H), 3.84-3.69 (m, 1H), 3.65-3.45 (m, 2H), 2.82 (m, 1H), 2.46-2.31 (m, 1H), 1.40 (s, 9H), 1.22 (s, 9H). δ⁵¹V NMR (d₆-DMSO, 105 MHz): 401 ppm (minor), 363 ppm (minor), 332 ppm (major). UV/Vis (DMSO), λₘₐₓ/nm: 411, 553, 859.

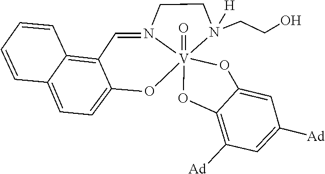

[VO(nap-Hshed)(dad)]. To a 250-mL round bottom Schlenk flask ACS grade acetone (100 mL) was added and degassed with Argon for 20 minutes. [VO2(nap-Hshed)] (0.34 g, 1.0 mmol) was added to the degassed acetone, followed by 3,5-di-adamantyl catechol (0.38 g, 1.0 mmol). A dark purple solution resulted after 1 hour but stirred for 24 h. The reaction mixture was vacuum filtered and then concentrated to dryness under reduced pressure at room temperature. The filter product was collected. The purple residue and filter product were dissolved in a minimal amount of HPLC grade acetone and then n-hexane (100 mL) was added. The solution was stored at −20° C. for 2-3 days. The dark purple microcrystalline precipitate was vacuum filtered, washed with cold hexanes (2×20 mL), and dried under vacuum for 3 days to yield 0.34 g (49%) of a purple solid. δ¹H NMR (CDCl₃, 400 MHz) 9.20 (s, 1H), 7.99 (d, J=8.3 Hz, 1H), 7.81 (d, J=9.2 Hz, 1H), 7.70 (d, J=8.0 Hz, 1H), 7.49 (t, J=7.7 Hz, 1H), 7.29 (t, J=7.7 Hz, 1H), 7.11 (d, J=9.3 Hz, 1H), 6.41-6.25 (m, 1H), 5.2-4.9 (m, 1H), 4.27 (m, 1H), 4.10 (m, 2H), 3.92 (s, 1H), 3.67 (m, 1H), 3.51 (s, 1H), 3.38 (s, 1H), 3.12 (s, 1H), 2.65 (s, 1H), 2.26-2.02 (m, 12H), 1.92-1.65 (m, 18H). δ⁵¹V NMR (CDCl₃, 105 MHz) 401 ppm (major), 502 ppm (minor), 512 ppm (major). UV/Vis (DMSO), λₘₐₓ/nm: 401, 549, 863.

### SALIEP Compounds, Including V^{V}O₂(SALIEP)-Catecholate, V^{V}O₂(Cl-SALIEP)-catecholate, and [VIVO(SALIEP)]-complexes

The [V^{V}O₂(SALIEP)] precursor was synthesized by using multistep procedure outlined below.

Step 1. Synthesis of 2-[[[2-(2-pyridinyl)ethyl]imino]methyl]phenol (HSALIEP). To 10 mL of absolute EtOH, salicylaldehyde (0.611 g, 5.00 mmol) and 2-(2-aminoethyl)pyridine (0.611 g, 5.00 mmol), were added and then stirred under reflux for about 30 min. The resulting Schiff base was used immediately in the 2^{nd} step to avoid noticeable degradation.

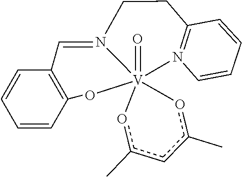

Step 2. Preparation of [VIVO(SALIEP)(acac)]. To 25 mL of absolute EtOH, [V^{IV}O(acac)₂] (1.33 g, 5.00 mmol) was added and stirred at 60° C. to let the complex dissolve. Subsequently, this solution was added to the ethanolic Schiff base solution from Step 1, and the resulting reaction mixture was stirred under reflux for 3 h. The resulting red solid was filtered off, washed with 50 mL cold (0° C.) ethyl ether and dried in vacuo. Yield 0.235 g (60%). FT-IR: 3100 (sp² C—H stretch), 2915.89 (sp³ C—H stretch), 1589.88-1415.88 (aromatic stretch), 1383.32 (sp³ C—H bend), 1342.14 (aromatic C—N stretch), 929.32 (V═O stretch) cm⁻¹.

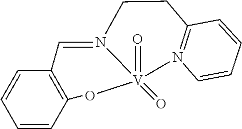

Step 3. Preparation of [VVO₂(SALIEP)]. The [V^{IV}O(SALIEP)(acac)] complex (0.391 g, 1.00 mmol) was dissolved in 20 mL of methanol and after the addition of aqueous 30% H₂O₂ (0.2 mL, 2.00 mmol), the solution was aerially oxidized for 10 minutes. The resulting yellow solid was filtered, washed with 50 mL cold (0° C.) ethyl ether and dried in vacuo. Yield: 0.182 g (59%). δ¹H NMR (d₆-DMSO, 400 MHz): 8.52 (d, 1H), 8.34 (s, 1H), 7.66 (t, 1H), 7.46 (t, 1H), 7.33 (d, 1H), 7.22 (t, 1H), 7.18 (d, 1H), 4.04 (t, 2H), 3.32 (t, 2H). ¹H (d₃-MeCN): 8.90 (d, 1H), 8.69 (s, 1H), 7.99 (t, 1H), 7.49 (t, 2H), 7.43 (t, 1H), 6.87 (m, 2H), 4.03 (t, 2H), 3.39 (t, 1H), 3.29 (d, 1H). 51 V NMR (d₆-DMSO, 101 MHz): −507, −539 ppm; (d₃-MeCN): −511 ppm. FT-IR: 3056.62 (sp² C—H stretch), 1623.28-1473.42 (aromatic stretch), 1304.52 (aromatic C—N stretch), 917.05 (V═O stretch), 760.59 (sp² C—H bend, 1,2-disubstituted) cm⁻¹.

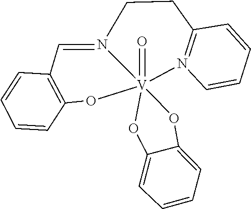

[VO(SALIEP)(cat)]. To 25.0 mL of degassed methanol, the [V^{V}O₂(SALIEP)] precursor (0.308 g, 1.00 mmol) was added, followed by the addition of catechol (0.132 g, 1.20 mmol). The reaction mixture was let to stir for 20 h at room temperature under argon. The resulting product was vacuum filtered, washed with cold (0° C.) ethyl ether (30 mL) and dried under high vacuum for 24 h. Yield: 0.232 g (58%). δ¹H NMR (d₃-MeCN, 400 MHz): 8.80 (d, 1H), 8.54 (s, 1H), 7.91 (t, 1H), 7.47 (d, 1H), 7.44 (s, 1H), 7.43 (s, 1H), 7.41 (dd, 1H), 6.78 (t, 1H), 6.69 (d, 2H), 6.45 (d, 1H), 6.04 (d, 1H); δ⁵¹V NMR (d₃-MeCN, 105.2 MHz): −508 ppm (broad). FT-IR: 3060.39 (sp² C—H stretch), 1617.57-1446.87 (aromatic stretch), 1308.34-1205.77 (aromatic C—N stretch), 1149.97 (C—O stretch), 965.26 (V═O stretch) cm⁻¹ λₘₐₓ(0.10 mM in MeCN)=561 nm.

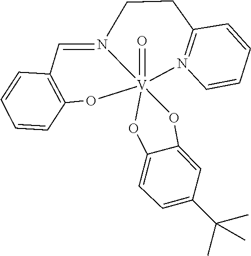

[VO(SALIEP)(4 TB)]. To 50.0 mL of degassed methanol, the [V^{V}O₂(SALIEP)] precursor (0.308 g, 1.00 mmol) was added, followed by the addition of 4-tert-butyl-catechol (0.199 g, 1.20 mmol). The reaction mixture was let to stir for 20 h at room temperature under argon. The resulting product was vacuum filtered, washed with cold (0° C.) ethyl ether (30 mL) and dried under high vacuum for 24 h. Yield: 0.178 g (39%). δ¹H NMR (d₃-MeCN, 400 MHz): 8.85 (d, 1H), 8.51 (s, 1H), 7.90 (t, 1H), 7.52 (d, 1H), 7.43 (t, 1H), 7.41 (t, 1H), 6.78 (t, 1H), 6.69 (d, 1H), 6.67 (d, 1H), 6.54 (d, 1H), 6.40 (d, 1H), 6.10 (s, 1H), 4.01 (dd, 2H), 3.52 (dd, 2H) 1.21 (s, 9H); δ⁵¹V NMR (d₃-MeCN, 105.2 MHz): −511 ppm. FT-IR: 3054.09 (sp² C—H stretch), 2950.68 (sp³ C—H stretch), 1621.31-1450.88 (aromatic stretch), 1339.13 (aromatic C—N stretch), 952.45 (V═O stretch) cm⁻¹. λₘₐₓ (0.10 mM in MeCN)=551 nm.

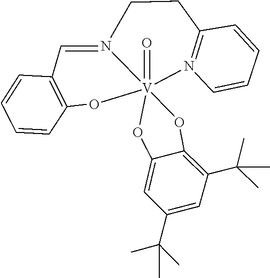

[VO(SALIEP)(DTB)] To 50.0 mL of degassed methanol, the [V^{V}O₂(SALIEP)] precursor (0.308 g, 1.00 mmol) was added, followed by the addition of di-tert-butyl-catechol (0.267 g, 1.20 mmol). The reaction mixture was let to stir for 20 h at room temperature under argon. The resulting product was vacuum filtered, washed with cold (0° C.) ethyl ether (30 mL) and dried under high vacuum for 24 h. Yield: 0.298 g (58%). δ¹H NMR (d₃-MeCN, 400 MHz): 8.98 (d, 1H), 8.50 (d, 1H), 7.90 (q, 1H); 7.46 (t, 1H), 7.42 (d, 1H), 7.40 (d, 1H), 7.38 (d, 1H), 6.75 (m, 1H), 6.65 (m, 1H), 6.46 (s, 1H), 6.03 (s, 1H), 4.07 (m, 2H), 3.55 (m, 1H), 3.42 (m, 1H), 1.40 (s, 6H), 1.27 (s, 3H), 1.21 (s, 6H), 0.98 (s, 3H); δ⁵¹V NMR (d₃-MeCN, 105.2 MHz): −511 ppm. FT-IR: 3054.09 (sp² C—H stretch), 2950.68 (sp³ C—H stretch), 1618.17-1445.58 (aromatic stretch), 1311.91 (aromatic C—N stretch), 939.49 (V═O stretch) cm⁻¹. λₘₐₓ (0.10 mM in MeCN)=555 nm.

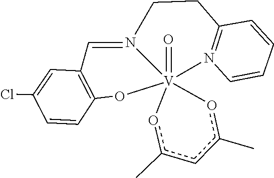

[V^{V}O₂(Cl-SALIEP)]

Step 1. Synthesis of 2-[[[2-(2-pyridinyl)ethyl]imino]methyl]chlorophenol (H-CISALIEP). To 10 mL of absolute EtOH, 5-chlorosalicylaldehyde (0.783 g, 5.00 mmol) and 2-(2-aminoethyl)pyridine (0.611 g, 5.00 mmol), were added and then stirred under reflux for about 30 min. They were used immediately in the 2^{nd} step to avoid noticeable degradation.

Step 2. Preparation of [VIVO(Cl-SALIEP)(acac)]. To 25 mL of absolute EtOH, [VIVO(acac)₂] (1.33 g, 5.00 mmol) was added and stirred at 60° C. to let the complex dissolve. Subsequently, this solution was added to the ethanolic Schiff base solution from Step 1, and the resulting reaction mixture was stirred under reflux for 3 h. The resulting shiny brown solid was filtered off, washed with 50 mL cold (0° C.) ethyl ether and dried in vacuo. Yield 0.243 g (57%). FT-IR: 3048.97 (sp² C—H stretch), 2952.85-2922.42 (sp³ C—H stretch), 1586.40-1462.76 (aromatic stretch), 1376.12 (sp³ C—H bend), 1308.44 (aromatic C—N stretch), 1182.93 (C—O stretch), 929.17 (V═O stretch), 702.97 (C—Cl stretch) cm⁻¹.

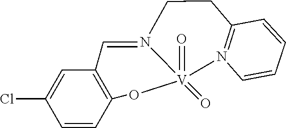

Step 3. Preparation of [VVO2 (Cl-SALIEP)]. The [VIVO(Cl-SALIEP)(acac)] complex (0.426 g, 1.00 mmol) was dissolved in 20 mL of methanol and after the addition of aqueous 30% H₂O₂ (0.2 mL, 2.00 mmol), the solution was aerially oxidized for 10 minutes. The resulting brown solid was filtered, washed with 50 mL cold (0° C.) ethyl ether and dried in vacuo. Yield 0.257 g (75%). δ⁵¹V NMR (d₃-MeCN, 10.0 mM): −512 ppm. FT-IR: 3057.05 (sp² C—H stretch), 1626.27-1485.3 (aromatic stretch), 1298.45 (aromatic C—N stretch), 926.53 (V═O stretch), 767.76 (C—Cl stretch) cm⁻¹.

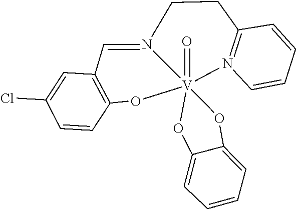

[VO(Cl-SALIEP)(cat)]. To 12.5 mL of degassed methanol, the [V^{V}O₂(Cl-SALIEP)] precursor (0.343 g, 1.00 mmol) was added, followed by the addition of catechol (0.132 g, 1.20 mmol). The reaction mixture was let to stir for 20 h at room temperature under argon. The resulting product was vacuum filtered, washed with cold (0° C.) ethyl ether (30 mL) and dried under high vacuum for 24 h. Yield: 0.191 g (44%). δ¹H NMR (d₃-MeCN, 400 MHz): 8.76 (d, 1H), 8.49 (s, 1H), 7.92 (t, 1H), 7.48 (d, 1H), 7.43 (s, 1H), 7.42 (s, 1H), 7.36 (dd, 1H), 6.69 (d, 2H), 6.43 (d, 1H), 6.03 (d, 1H); δ⁵¹V NMR (d₃-MeCN, 105.2 MHz): N/A. FT-IR: 3100 (sp² C—H stretch), 2958.06 (sp³ C—H stretch), 1624.75-1460.7 (aromatic stretch), 1388.27 (sp³ C—H bend), 1384.11 (aromatic C—N stretch), 921.30 (V═O stretch), 785.97 (C—Cl stretch) cm⁻¹. λₘₐₓ (0.10 mM in MeCN)=555 nm.

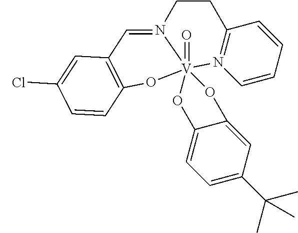

[VO(Cl-SALIEP)(4 TB)]. To 12.5 mL of degassed methanol, the [V^{V}O₂(Cl-SALIEP)] precursor (0.171 g, 0.500 mmol) was added, followed by the addition of 4-ter-butyl-catechol (0.100 g, 0.600 mmol). The reaction mixture was let to stir for 20 h at room temperature under argon. The resulting product was vacuum filtered, washed with cold (0° C.) ethyl ether (30 mL) and dried under high vacuum for 24 h. Yield: 0.280 g (57%). δ¹H NMR (d₃-MeCN, 400 MHz): 8.81 (d, 1H), 8.46 (s, 1H), 7.91 (t, 1H), 7.47 (t, 1H), 7.42 (d, 1H), 7.41 (s, 1H), 7.36 (d, 1H), 6.67 (d, 1H), 6.50 (s, 1H), 6.40 (d, 1H), 6.09 (s, 1H), 4.04 (dt, 2H), 3.40 (dt, 2H), 1.26 (s, 3H), 1.21 (s, 6H); δ⁵¹V NMR (d₃-MeCN, 105.2 MHz): −536 ppm. FT-IR: 3100 (sp² C—H stretch), 2958.06 (sp³ C—H stretch), 1624.75-1460.7 (aromatic stretch), 1388.27 (sp³ C—H bend), 1309 (aromatic C—N stretch), 951.30 (V═O stretch), 785.97 (C—Cl stretch) cm⁻¹. λₘₐₓ (0.10 mM in MeCN)=551 nm.

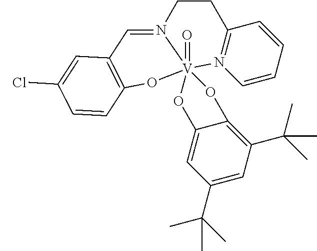

[VO(Cl-SALIEP)(DTB)] To 50.0 mL of degassed methanol, the [V^{V}O₂(Cl-SALIEP)] precursor (0.343 g, 1.00 mmol) was added, followed by the addition of di-tert-butyl-catechol (0.267 g, 1.20 mmol). The reaction mixture was let to stir for 20 h at room temperature under argon. The resulting product was vacuum filtered, washed with cold (0° C.) ethyl ether (30 mL) and dried under high vacuum for 24 h. Yield: 0.295 g (54%). δ¹H NMR (d₃-MeCN, 400 MHz): 8.94 (d, 1H), 8.45 (d, 1H), 7.89 (q, 1H); 7.47 (t, 1H), 7.40 (s, 1H), 7.37 (dd, 1H), 7.33 (dd, 1H), 6.46 (s, 1H), 6.03 (s, 1H), 4.07 (m, 2H), 3.55 (m, 1H), 3.44 (m, 1H), 1.39 (s, 6H), 1.27 (s, 3H), 1.21 (s, 6H), 0.97 (s, 3H). δ⁵¹V NMR (d₃-MeCN, 105.2 MHz): −531 ppm. FT-IR: 3100 (sp² C—H stretch), 2958.06 (sp³ C—H stretch), 1624.75-1460.7 (aromatic stretch), 1388.27 (sp³ C—H bend), 1384.03 (aromatic C—N stretch), 922.91-921.30 (V═O stretch), 785.97 (C—Cl stretch) cm¹. λₘₐₓ (0.10 mM in MeCN)=551 nm.

Non-Innocent Vanadium Complexes Containing Monosubstituted Tbu-Substituted Schiff Base Ligands

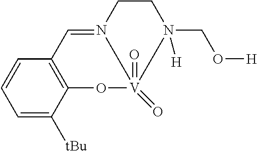

[VO₂(TB-HSHED)]. 3-tBu-salicylaldehyde (3.12 g, 18.8 mmol) was dissolved in degassed MeOH (50 ml), and added via a dropping funnel to a solution of N-(2-hydroxyethyl)ethylenediamine (1.9 ml, 18.8 mmoles) in degassed MeOH (50 ml), and the reaction allowed to stir for 2 hours. Vanadyl sulfate hydrate (3.74 g, 18.8 mmoles) was dissolved in degassed DDI water (50.0 ml) and added to the reaction via a dropping funnel. The reaction was allowed to stir for 4 hours before a solution of NaOH(aq) (1.51 g, 37.6 mmoles) in DI water (50 ml) was added, and the reaction allowed to stir open to air overnight. The reaction was then filtered, and the solid sonicated in cold pentane and filtered before being dried in vacuo for two days. Yield: 4.52 g (72%). ¹H NMR (CDCl₃, 400 MHz): δ 8.18 (s, 1H), 7.52-7.50 (d, 1H), 7.00-6.99 (d, 1H), 6.78-6.74 (t, 1H), 5.06 (s, 1H) 4.86 (t, 1H), 4.22-4.16 (t/d, 1H), 3.93-3.90 (d, 1H), 3.79-3.73 (d/d, 1H), 3.60 (bs, 1H), 3.50-3.40 (m, 2H), 3.25-3.22 (m, 1H), 3.12 (s, 1H), 2.87 (s, 1H), 2.87-2.83 (m, 1H), 1.41 (s, 9H) ppm. ⁵¹V NMR (CDCl₃, 400 MHz): δ −532.90 ppm.

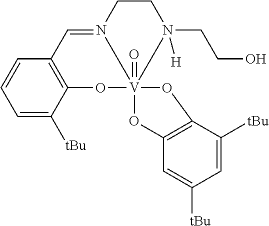

[VO(tbuHSHED)dtb]. 3,5-di-tert-butylcatechol (0.134 g g, 0.602 mmol) was added to a solution of [VO₂(tbuHSHED)] (0.200 g, 0.602 mmol) and stirred in CHCl₃ (25 ml) for 24 hrs under an argon atmosphere. Upon addition of the 3,5-di-tert-butylcatechol the reaction was observed to rapidly change color from a moderately dark but transparent yellow, to a dark, almost black, purple. The reaction was wrapped in tinfoil in an effort to prevent unwanted photodegradation. After 48 hrs, the solution was then cooled to −78° C., and filtered through a fritted filter. The solvent was removed by rotovap, and the residue dried in vacuo for 4 days. At this point the tacky purple-black crystalline solid was scrapped out of the round-bottomed flask, crushed as best as possible, and put back under vacuum at 30° C. for a week. The compound was again scraped out of the round-bottomed flask and pulverized before being placed under vacuum at 30° C. for another week. Yield 0.158 g (49%). ¹H NMR (CDCl₃, 400 MHz): δ 8.36 (s, 1H), 7.43-7.41 (d, 1H), 7.19-7.17 (d, 1H), 6.74-6.69 (m, 1H), 6.40-6.34 (d, 2H), 4.28 (bs, 1H), 4.19-4.11 (m, 3H), 4.00-3.96 (m, 2H), 3.67 (bs, 1H), 3.46-3.41 (d, 2H), 3.13 (m, 1H), 1.46 (s, 6H), 1.42 (s, 3H), 1.25-1.24 (d, 18H) ppm. ⁵¹V NMR (CDCl₃, 400 MHz): δ 419.27 (minor), 306.46 (major) ppm.

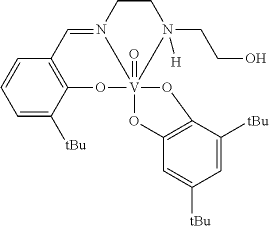

[VO(TB-HSHED)cat]. Catechol (0.078 g g, 0.722 mmol) was added to a solution of [VO₂(tbuHSHED)] (0.250 g, 0.722 mmol) and stirred in MeOH (25 ml) for 24 hrs under an argon atmosphere. Upon addition of the catechol the reaction was observed to rapidly change color from a moderately dark but transparent yellow, to a dark, almost black, purple color. The reaction was wrapped in tinfoil in an effort to prevent unwanted photodegradation. After 48 hrs, the solution was then cooled to −78° C., and filtered through a fritted filter. The solvent was removed by rotovap, and the residue dried in vacuo for 4 days. At this point the tacky purple-black crystalline solid was scrapped out of the round-bottomed flask, crushed as best as possible, and put back under vacuum at 30° C. for a week. The compound was again scraped out of the round-bottomed flask and pulverized before being placed under vacuum at 30° C. for another week. Yield 89 mg (29%). ¹H NMR (CDCl₃, 400 MHz): δ 8.38 (s, 1H), 7.48-7.46 (d, 1H), 6.78-6.76 (m, 3H), 6.64 (s, 2H), 6.48 (m, 1H), 4.50 (s, 1H), 4.18 (s, 1), 3.97-3.87 (m, 3), 3.74 (s, 1), 3.47-3.40 (d, 2), 3.08 (s, 1H), 1.25 (2, 9H). ⁵¹V NMR (CDCl₃, 400 MHz): δ 83.46 (major), 215.22 (minor) ppm.

Disubstituted tbu Schiff Bases

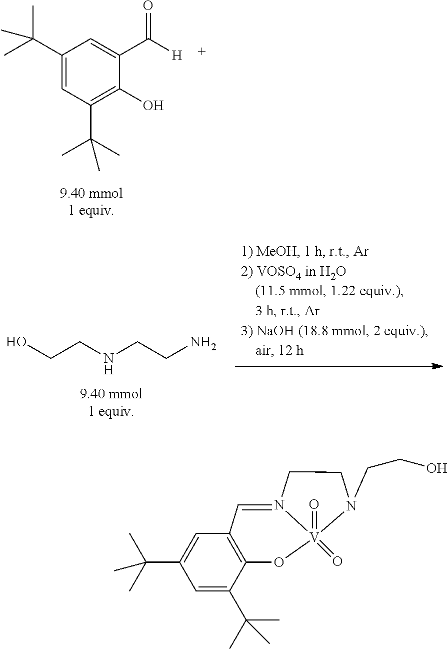

[VO2(dtbHSHED)]. To a 100 mL Schlenk flask, 40 mL of HPLC-grade methanol was added and degassed with argon for 30 minutes. This was followed by the addition of 3,5-di-tert-butylsalicylaldehyde (2.203 g, 9.400 mmol) and N-(2-hydroxyethyl) ethylenediamine (0.979 g, 9.40 mmol). The reaction mixture was let to stir for 1 h at ambient temperature under argon. Vanadyl sulfate (1.866 g, 11.45 mmol), dissolved in 25 mL of degassed water, was added to the reaction mixture after 1 h. The reaction mixture was let then to stir for 3 h at ambient temperature under argon. After 3 h, sodium hydroxide (0.752 g, 18.8 mmol) was added, and the reaction mixture was then opened to air and let to stir overnight. The product then was vacuum filtered, washed with 25 mL cold (0° C.) methanol and let to dry under high vacuum for 3 days. Yield: 6.51 g (86%). ¹H NMR (CDCl₃, 400 MHz): δ 8.26 (s, 1 h), 7.59 (s, 1 h), 7.02 (s, 1H), 5.26 (BS, 1H), 4.79-4.75 (t, 1H) 4.21-4.16 (t, 1H), 3.90-3.87 (m, 1H), 3.44 (s, 1H), 3.22 (s, 1H), 3.09 (s, 1H), 2.86-2.78 (m, 1H), 1.41 (s, 9H), 1.31 (s, 9H). ⁵¹V NMR (CDCl₃, 400 MHz): δ 540.29 ppm.

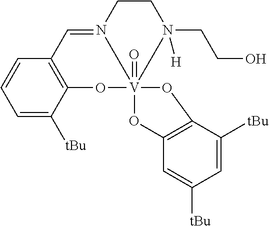

[VO(dtbHSHED)dtb]. 3,5-di-tert-butylcatechol (5.07 g g, 13.1 mmol) was added to a solution of [VO₂(dtbHSHED)] (2.89 g, 13.1 mmol) and stirred in CHCl₃ (1000 ml) for 48 hrs under an argon atmosphere. Upon addition of the 3,5-di-tert-butylcatechol the reaction was observed to rapidly change color from a moderately dark but transparent yellow, to a dark, almost black, purple. The reaction was wrapped in tinfoil in an effort to prevent unwanted photodegradation. After 48 hrs, the solution was then cooled to −78° C., and filtered through a fritted filter. The solvent was removed by rotovap, and the residue dried in vacuo for 4 days. At this point the tacky purple-black crystalline solid was scrapped out of the round-bottomed flask, crushed as best as possible, and put back under vacuum at 30° C. for a week. The compound was again scraped out of the round-bottomed flask and pulverized before being placed under vacuum at 30° C. for another week. Yield 5.26 g (69%). ¹H NMR (CDCl₃, 400 MHz): δ 8.34 (s, 1H), 7.48 (s, 1H), 6.84 (s, 1H), 6.37-6.33 (d, 2H), 4.29 (d, 2H), 4.17-4.09 (m, 2H), 3.97-3.93 (m, 2H), 3.65 (s, 1H), 3.41 (s, 2H), 3.11 (s, 1H), 1.44 (s, 6H), 1.40 (s, 3H), 1.28 (s, 9H), 1.28 (s, 9H), 1.25 (s, 9H), 1.22 (s, 9H). ⁵¹V NMR (CDCl₃, 400 MHz): δ 308.07 ppm.

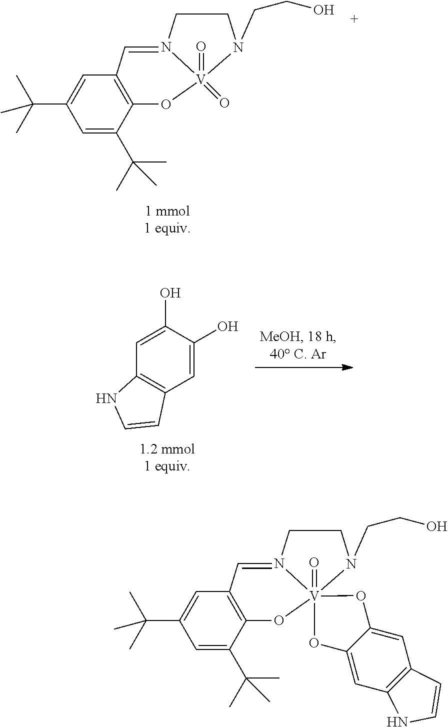

[VO(dtbHSHED)(DHI)]. To a 50 mL Schlenk flask, 25 mL of HPLC-grade methanol was added and degassed with argon for 30 minutes. This was followed by the addition of the [VO₂(DTB-HSHED)] precursor (0.401 g, 1.00 mmol) and 5,6-dihydroxyindole (0.179 g, 1.20 mmol). The reaction mixture was let to stir for 18 h at 40° C. under argon. The resulting product was vacuum filtered, washed with 25 mL cold (0° C.) methanol and let to dry under high vacuum for 3 days. Yield 0.134 g (25%). δ¹H NMR (d₆-DMSO, 400 MHz) δ 8.89 (s, 1H), 8.53 (s, 1H), 7.44 (d, J=2.5 Hz, 1H), 7.37 (d, J=2.5 Hz, 1H), 7.28 (d, J=15.8 Hz, 3H), 5.49 (s, 1H), 4.01 (s, 3H), 3.83 (t, J=5.7 Hz, 5H), 2.82 (t, J=6.2 Hz, 5H), 1.38 (s, 13H), 1.31 (s, 12H), 1.28 (s, 11H), 1.27 (s, 13H). δ⁵¹V NMR (d₆-DMSO, 105 MHz): −533 ppm. λₘₐₓ=550 nm.

Vanadium Complexes with Isopropyl-Substituted Catecholate Ligands

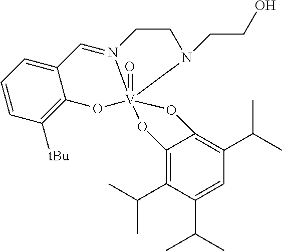

[VO(tBuHshed)(tipcat)]. 3,4,6-tri-isopropylcatechol (0.105 g, 0.443 mmol) was added to a solution of [VO2(tBuHshed)] (0.154 g, 0.443 mmol) and stirred in acetone (50 mL) for 24 hours under an argon atmosphere. The solvent was then removed under evaporated pressure. The crude substrate was redissolved in a minimal amount of acetone, diluted with pentane, cooled to −78° C., and vacuum filtered. The solvent was removed from the filtrate via evaporated pressure to yield [VO(tBuHshed)(tipcat)] in quantitative yield.

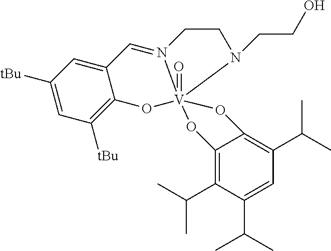

[VO(di-tBuHshed)(tipcat)]. 3,4,6-tri-isopropylcatechol (0.118 g, 0.500 mmol) was added to a solution of [VO2(tBuHshed)] (0.200 g, 0.500 mmol) and stirred in acetone (50 mL) for 24 hours under an argon atmosphere. The solvent was then removed under evaporated pressure. The crude substrate was redissolved in a minimal amount of acetone, diluted with pentane, and cooled to −78° C. The solvent was removed from the filtrate via evaporated pressure to yield [VO(di-tBuHshed)(tipcat)] in quantitative yield.

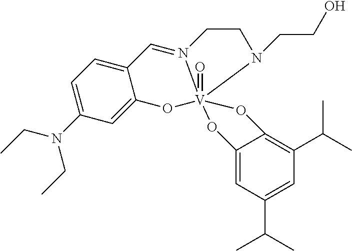

[VO(dea-Hshed)(dipcat)]. 3,5-di-isopropyl catechol (0.048 g, 0.250 mmol) was added to a solution of [VO2(dea-Hshed)] and stirred in degassed acetone (25 mL) under an argon atmosphere for 24 hours. The crude product was isolated by vacuum filtration and excess solvent was removed under evaporated pressure. The crude product was then dissolved in a minimal amount of acetone, diluted with pentane, and cooled to −78° C. The solvent was removed from the filtrate via evaporated pressure to yield 0.06 g (22%) of [VO(dea-Hshed)(dipcat)] as a purple solid.

Vanadium Complexes with Adamantyl-Substituted Catecholate Ligands

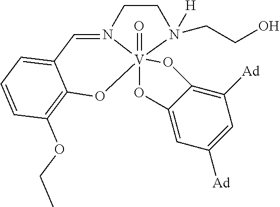

[VO(3-OEtHshed)(dad)]. To a clean, dry 100 mL Schlenk flask, 25 mL of HPLC-grade methanol was added and degassed with nitrogen for 15 minutes. Following this, VO₂(EtHshed) (0.167 g, 0.500 mmol) was added to the methanol followed by 3,5-di-(adamantyl) catechol (0.189 g, 0.500 mmol). Immediately after the addition of the catechol, the reaction mixture was placed under nitrogen. Upon the addition of the catechol, the color slowly changed from yellow to brown to dark violet. The reaction mixture was allowed to stir overnight under nitrogen. The solution was then filtered in vacuo and the filtrate was then evaporated to complete dryness via rotary evaporator. To the resulting solid was then dissolved in minimal GC-Resolved grade acetone followed by the addition of ACS-grade hexanes (100 mL). The solution was then capped and stored at −20° C. overnight to precipitate out the desired purple solid. Yield: 77%. δ¹H NMR (CDCl₃, 400 MHz) δ8.33 (s, 1H) 7.01 (d, 1H) 6.93 (d, 1H) 6.60 (t, 1H) 6.29 (s, 1H) 6.22 (s, 1H) 3.99 (m, 4H) 3.88 (m, 1H) 3.63 (m, 1H) 3.44 (m, 1H) 3.32 (m, 1H) 3.14 (m, 1H) 2.06 (d, 9H) 1.75 (m, 25H) 1.16 (t, 3H). δ⁵¹V NMR (d₃-MeCN, 105 MHz) δ 379 ppm (major) 427 ppm (minor) 453 ppm (minor) 471 ppm (minor).

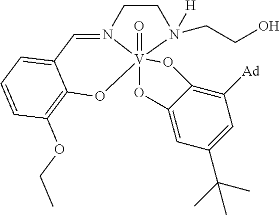

[VO(3-OEtHshed)(tbad)]. To a clean, dry 100 mL Schlenk flask, 25 mL of HPLC-grade methanol was added and degassed with nitrogen for 15 minutes. Following this, VO₂(EtHshed) (0.334 g, 1.000 mmol) was added to the methanol followed by 3-(tert-butyl)-5-(adamantyl) catechol (0.300 g, 1.000 mmol). Immediately after the addition of the catechol, the reaction mixture was placed under nitrogen. Upon the addition of the catechol, the color slowly changed from yellow to brown to dark violet. The reaction mixture was allowed to stir overnight under nitrogen. The solution was then filtered in vacuo and the filtrate was then evaporated to complete dryness via rotary evaporator. To the resulting solid was then dissolved in minimal GC-Resolv grade acetone followed by the addition of ACS-grade hexanes (100 mL). The solution was then capped and stored at −20° C. overnight to precipitate out the desired purple solid. Yield: 41%. δ¹H NMR (CDCl₃, 400 MHz) δ 8.33 (s, 1H) 7.01 (d, 1H) 6.94 (d, 1H) 6.60 (t, 1H) 6.34 (s, 1H) 6.22 (s, 1H) 3.98 (m, 4H) 3.88 (m, 2H) 3.63 (m, 1H) 3.44 (m, 1H) 3.32 (m, 1H) 3.14 (m, 1H) 2.09 (s, 6H) 1.81 (m, 8H) 1.58 (s, 3H) 1.21 (s, 9H) 0.86 (t, 3H). δ⁵¹V NMR (d₃-MeCN, 105 MHz) δ 372 ppm (major) 422 ppm (minor) 450 ppm (minor) 466 ppm (minor).

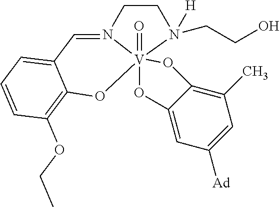

[VO(3-OEtHshed)(3mad)]. To a clean, dry 100 mL Schlenk flask, 25 mL of HPLC-grade methanol was added and degassed with nitrogen for 15 minutes. Following this, VO2(EtHshed) (0.167 g, 0.500 mmol) was added to the methanol followed by 3-(methyl)-5-(adamantyl) catechol (0.129 g, 0.500 mmol). Immediately after the addition of the catechol, the reaction mixture was placed under nitrogen. Upon the addition of the catechol, the color slowly changed from yellow to brown to dark violet. The reaction mixture was allowed to stir overnight under nitrogen. The solution was then filtered in vacuo and the filtrate was then evaporated to complete dryness via rotary evaporator. To the resulting solid was then dissolved in minimal GC-Resolved grade acetone followed by the addition of ACS-grade hexanes (100 mL). The solution was then capped and stored at −20° C. overnight to precipitate out the desired purple solid. Yield: 62%. δ¹H NMR (CDCl₃, 400 MHz) δ 8.35 (s, 1H) 7.04 (d, 1H) 6.97 (d, 1H) 6.63 (m, 1H) 6.29 (s, 1H) 6.20 (s, 1H) 4.17 (m, 1H) 4.02 (m, 4H) 3.86 (m, 1H) 3.64 (m, 1H) 3.45 (m, 1H) 3.33 (m, 1H) 3.08 (m, 1H) 2.03 (s, 3H) 1.72 (m, 16H) 1.21 (t, 3H). δ⁵¹V NMR (d₃-MeCN, 105 MHz) δ 398 ppm (major) 447 ppm (minor) 456 ppm (minor) 517 ppm (minor).

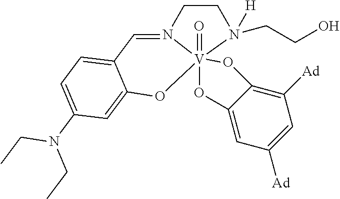

[VO(dea-Hshed)(dad)]: To a 100-mL round bottom Schlenk flask ACS grade acetone (50 mL) was added and degassed with Argon for 20 minutes. [VO2(dea-Hshed)] (0.18 g, 0.5 mmol) was added to the degassed acetone, followed by 3,5-diadamantyl catechol (0.19 g, 0.5 mmol). A dark purple solution resulted after 30 min but stirred for 24 h. The reaction mixture was vacuum filtered, and the filter product was collected. The filtrate was concentrated to dryness under reduced pressure at room temperature. The resulting dark purple residue and filter product were dissolved in a minimal amount of HPLC grade acetone and then n-hexane (50 mL) was added. The solution was stored at −20° C. for 2-3 days. The black microcrystalline precipitate was vacuum filtered, washed with cold hexanes (20 mL), and dried under vacuum for 3 days to yield 0.25 g (69%) of purple solid. δ¹H NMR (d₆-DMSO, 400 MHz): 8.35 (s, 1H), 7.24 (d, J=8.8 Hz, 1H), 6.24 (d, J=9.1 Hz, 1H), 6.18 (s, 1H), 6.13 (s, 1H), 5.78 (s, 1H), 4.79 (s, 1H), 4.73 (s, 1H), 4.39 (s, 1H), 4.13-3.93 (m, 2H), 3.85 (d, J=13.4 Hz, 1H), 3.71 (s, 1H), 3.54 (m, 1H), 3.45 (m, 1H), 3.35 (q, J=7.1 Hz, 4H), 2.74 (s, 1H), 2.02 (s, 6H), 1.81-1.67 (m, 24H), 1.09 (t, J=7.1 Hz, 6H). δ⁵¹V NMR (d₆-DMSO, 105 MHz): 346 ppm (major), 456 ppm (minor), 446 ppm (minor). UV/Vis (DMSO), λₘₐₓ/nm: 553, 889.

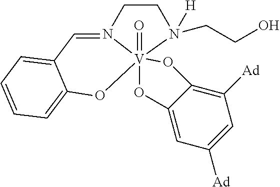

[VO(Hshed)(dad)]. To a 100-mL round bottom Schlenk flask ACS grade acetone (50 mL) was added and degassed with Argon for 20 minutes. [VO2(Hshed)] (0.15 g, 0.5 mmol) was added to the degassed acetone, followed by 3,5-diadamantyl catechol (0.19 g, 0.5 mmol). A deep purple solution resulted after 15 min but stirred for 24 h. The reaction mixture was vacuum filtered, and the filter product was collected. The filtrate was concentrated to dryness under reduced pressure at room temperature. The purple residue was dissolved in a minimal amount of HPLC grade acetone and then n-hexane (50 mL) was added. The solution was stored at −20° C. for 2-3 days. The purple microcrystalline precipitate was vacuum filtered, washed with cold hexanes (20 mL), and dried under vacuum for 3 days to yield 0.23 g (71%) of purple solid. δ¹H NMR (400 MHz, DMSO): 8.73 (s, 1H), 7.50 (d, J=7.7 Hz, 1H), 7.40 (t, J=7.7 Hz, 1H), 6.76 (t, J=7.3 Hz, 1H), 6.66 (d, J=8.4 Hz, 1H), 6.19 (s, 1H), 6.17-6.12 (m, 1H), 4.83 (m, 1H), 4.74 (t, J=5.4 Hz, 1H), 4.60 (s, 1H), 4.13 (d, J=12.5 Hz, 1H), 3.94 (dt, J=15.2, 7.6 Hz, 1H), 3.73 (m, 1H), 3.48 (q, J=6.0 Hz, 2H), 2.92-2.74 (m, 1H), 2.47-2.28 (m, 1H), 2.02 (m, 6H), 1.83-1.66 (m, 24H). δ⁵¹V NMR (d₆-DMSO, 105 MHz): 352 ppm (major), 386 ppm (minor), 409 ppm (minor). UV/Vis (DMSO), λₘₐₓ/nm: 550, 869.

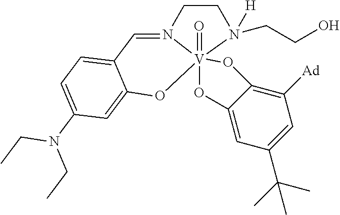

[VO(dea-Hshed)(tbad)]. To a 100-mL round bottom Schlenk flask ACS grade acetone (50 mL) was added and degassed with Argon for 20 minutes. [VO2(dea-Hshed)] (0.18 g, 0.5 mmol) was added to the degassed acetone, followed by 3-adamantyl-5-tBu-catechol (0.15 g, 0.5 mmol). A deep purple solution resulted after 30 min but stirred for 24 h. The reaction mixture was vacuum filtered, and the filter product was collected. The filtrate was concentrated to dryness under reduced pressure at room temperature. The purple residue was dissolved in a minimal amount of HPLC grade acetone and then n-hexane (50 mL) was added. The solution was stored at −20° C. for 2-3 days. The black microcrystalline precipitate was vacuum filtered, washed with cold hexanes (20 mL), and dried under vacuum for 3 days to yield 0.086 g (38%) of black solid. δ¹H NMR (d₆-DMSO, 400 MHz): 8.36 (s, 1H), 7.25 (d, J=8.9 Hz, 1H), 6.30-6.23 (m, 1H), 6.19 (m, 2H), 5.79 (s, 1H), 4.80 (s, 1H), 4.73 (t, J=5.5 Hz, 1H), 4.42 (s, 1H), 4.12-3.95 (m, 1H), 3.86 (d, J=11.8 Hz, 1H), 3.72 (q, J=5.5 Hz, 1H), 3.55 (m, 1H), 3.50-3.44 (m, 1H), 3.36 (q, J=7.1 Hz, 4H), 3.28 (m, 1H), 2.76 (s, 1H), 2.15-2.01 (m, 3H), 1.95-1.56 (m, 12H), 1.21 (s, 9H), 1.10 (t, J=6.9 Hz, 6H). δ⁵¹V NMR (d₆-DMSO, 105 MHz): 277 ppm (major), 344 ppm (minor), 327 ppm (minor). UV/Vis (DMSO), λₘₐₓ/nm: 582, 884.

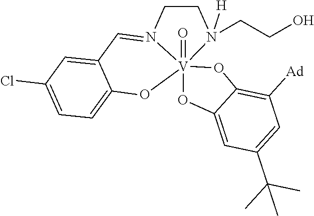

[VO(Cl-Hshed)(tbad)]. To a 250 mL round bottom flask ACS grade acetone (100 mL) was added and degassed with Argon for 20 minutes. [VO2(Cl-Hshed)] (0.325 g, 1.0 mmol) was added followed by 3-adamantyl-5-tBu-catechol (0.30 g, 1.0 mmol). A deep purple solution resulted after 10 min but stirred for 24 h. The reaction mixture was vacuum filtered, and the filter product was collected. The filtrate was concentrated to dryness under reduced pressure at room temperature. The resulting purple residue and the filter product were dissolved in a minimal amount of HPLC grade acetone and then n-hexane (100 mL) was added. The solution was stored at −20° C. for 2-3 days. The purple microcrystalline precipitate was vacuum filtered, washed with cold hexanes (2×20 mL), and dried under vacuum for 3 days to yield 0.182 g (30%) of purple solid. δ¹H NMR (d₆-DMSO, 400 MHz): 8.72 (s, 1H), 7.58 (s, 1H), 7.39 (d, J=9.3 Hz, 1H), 6.68 (d, J=9.0 Hz, 1H), 6.24-6.12 (m, 2H), 4.83 (s, 1H), 4.73 (s, 1H), 4.66 (s, 1H), 4.10 (s, 1H), 3.96 (d, J=13.1 Hz, 1H), 3.74 (s, 1H), 3.68-3.53 (m, 1H), 3.48 (s, 2H), 2.85 (s, 1H), 2.06 (m, 9H), 1.77 (s, 6H), 1.21 (s, 9H). δ⁵¹V NMR (d₆-DMSO, 105 MHz): 395 ppm (major). UV/Vis (DMSO), λₘₐₓ/nm: 550, 859.

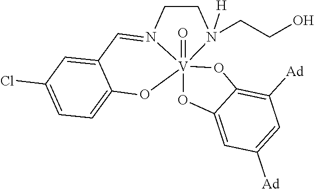

[VO(Cl-Hshed)(dad)]. To a 250 mL round bottom flask ACS grade acetone (100 mL) was added and degassed with Argon. [VO2(Cl-Hshed)] (0.325 g, 1.0 mmol) was added followed by 3,5-di-adamantyl-catechol (0.38 g, 1.0 mmol). A deep purple solution resulted after 10 min but stirred for 24 h. The reaction mixture was vacuum filtered, and the filter product was collected. The filtrate was concentrated to dryness under reduced pressure at room temperature. The resulting purple residue and the filter product were dissolved in a minimal amount of HPLC grade acetone and then n-hexane (100 mL) was added. The solution was stored at −20° C. for 2-3 days. The purple microcrystalline precipitate was vacuum filtered, washed with cold hexanes (2×20 mL), and dried under vacuum for 3 days to yield 0.24 g (35%) of purple solid. δ¹H NMR (CDCl₃):8.28 (s, 1H), 7.30 (d, J=12.0 Hz, 1H), 6.87 (d, J=8.8 Hz, 1H), 6.4-6.61 (m, 1H), 6.33 (s, 1H), 6.28-6.15 (m, 1H), 4.84-5.2 (m, 1H), 4.33-3.79 (m, 4H), 3.67 (s, 1H), 3.48 (s, 1H), 3.37 (s, 1H), 3.13 (s, 1H), 2.63 (s, 1H), 2.08 (m, 12H), 1.77 (m, 18H). δ⁵¹V NMR (CDCl₃, 105 MHz): 585 ppm, 553 ppm, 468 ppm. UV/Vis (DMSO), λₘₐₓ/nm: 551, 871.

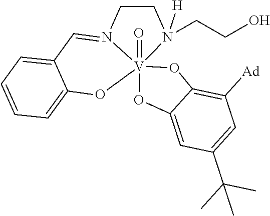

[VO(Hshed)(tbad)]. To a 100-mL round bottom Schlenk flask ACS grade acetone (50 mL) was added and degassed with Argon for 20 minutes. [VO2(Hshed)] (0.15 g, 0.5 mmol) was added, followed by 3-adamantyl-5-tBu-catechol (0.15 g, 0.5 mmol). A deep purple solution resulted after 15 min but stirred for 24 h. The reaction mixture was vacuum filtered, and the filter product was collected. The filtrate was concentrated to dryness under reduced pressure at room temperature. The resulting purple residue and the filter product were dissolved in a minimal amount of HPLC grade acetone and then n-hexane (50 mL) was added. The solution was stored at −20° C. for 2-3 days. The purple microcrystalline precipitate was vacuum filtered, washed with cold hexanes (20 mL), and dried under vacuum for 3 days to yield 0.09 g (31%) of purple solid. δ¹H NMR (d₆-DMSO, 400 MHz): 8.74 (s, 1H), 7.51 (d, J=7.8 Hz, 1H), 7.42 (d, J=7.9 Hz, 2H), 6.77 (t, J=7.3 Hz, 1H), 6.66 (d, J=8.5 Hz, 1H), 6.25-6.11 (m, 2H), 4.84 (s, 1H), 4.74 (s, 1H), 4.62 (s, 1H), 4.14 (d, J=13.5 Hz, 1H), 3.96 (m, 1H), 3.74 (m, 1H), 3.49 (m, 3H), 2.83 (m, 1H), 2.07 (s, 6H), 1.80 (m, 9H), 1.22 (s, 9H). δ⁵¹V NMR (d₆-DMSO, 105 MHz) δ 346 (major), 379 (minor), 398 (minor), 410 (minor).

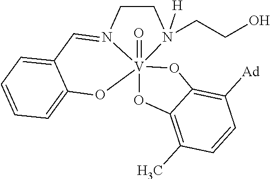

[VO(Hshed)(3mad)]. To a 100-mL round bottom Schlenk flask ACS grade acetone (50 mL) was added and degassed with Argon for 20 minutes. [VO2(Hshed)] (0.15 g, 0.5 mmol) was added, followed by 3-methyl-5-adamantyl-catechol (0.13 g, 0.5 mmol). A deep purple solution resulted after 15 min but stirred for 24 h. The reaction mixture was vacuum filtered, and the filter product was collected. The filtrate was concentrated to dryness under reduced pressure at room temperature. The resulting purple residue was dissolved in a minimal amount of HPLC grade acetone and then n-hexane (50 mL) was added. The solution was stored at −20° C. for 2-3 days. The purple microcrystalline precipitate was vacuum filtered, washed with cold hexanes (20 mL), and dried under vacuum for 3 days to yield 0.09 g (31%) of purple solid. δ¹H NMR (400 MHz, CDCl₃): 8.38 (s, 1H), 7.42 (t, J=8.0 Hz, 1H), 7.31 (d, J=8.0 Hz, 1H), 6.96 (d, J=8.5 Hz, 1H), 6.76 (d, J=7.5 Hz, 1H), 6.33 (s, 1H), 6.20 (s, 1H), 4.28-4.07 (m, 2H), 4.07-3.81 (m, 3H), 3.66 (s, 1H), 3.47 (s, 1H), 3.35 (s, 1H), 3.07 (m, 1H), 2.26 (s, 3H), 2.05 (m, 3H), 1.83-1.65 (m, 12H). δ⁵¹V NMR (CDCl3, 105 MHz): 518 ppm, 421 ppm.

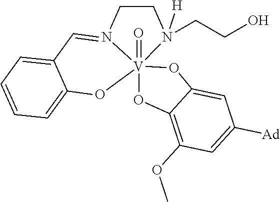

[VO(Hshed)(3omad)]. To a 100-mL round bottom Schlenk flask ACS grade acetone (50 mL) was added and degassed with Argon for 20 minutes. [VO2(Hshed)] (0.15 g, 0.5 mmol) was added, followed by 3-methoxy-5-adamantyl-catechol (0.14 g, 0.5 mmol). A deep purple solution resulted after 15 min but stirred for 24 h. The reaction mixture was vacuum filtered, and the filter product was collected. The filtrate was concentrated to dryness under reduced pressure at room temperature. The resulting purple residue was dissolved in a minimal amount of HPLC grade acetone and then n-hexane (50 mL) was added. The solution was stored at −20° C. for 2-3 days. The purple microcrystalline precipitate was vacuum filtered, washed with cold hexanes (20 mL), and dried under vacuum for 3 days to yield 0.1 g (35%) of purple solid. δ¹H NMR (CDCl₃, 400 MHz): 8.73 (s, 1H), 7.50 (dd, J=7.8, 1.8 Hz, 1H), 7.45-7.37 (m, 1H), 6.76 (t, J=7.3 Hz, 1H), 6.69 (d, J=8.2 Hz, 1H), 5.99-5.93 (m, 2H), 4.76 (t, J=5.4 Hz, 1H), 4.62 (s, 1H), 4.19-4.07 (m, 1H), 4.03-3.88 (m, 1H), 3.83 (s, 3H), 3.76-3.65 (m, 1H), 3.47 (dd, J=11.3, 5.5 Hz, 2H), 3.25 (s, 2H), 2.80 (d, J=12.2 Hz, 1H), 2.02 (s, 3H), 1.82-1.66 (m, 12H). δ⁵¹V NMR (105 MHz, CDCl₃): 389 ppm, 364 ppm. δ⁵¹V NMR (CDCl3, 105 MHz): 389 ppm, 364 ppm.

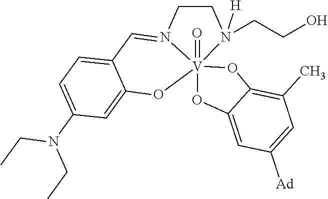

[VO(dea-Hshed)(3mad)]. To a 250-mL round bottom Schlenk flask ACS grade acetone (100 mL) was added and degassed with Argon for 20 minutes. [VO₂(dea-Hshed)] (0.36 g, 1.0 mmol) was added to the degassed acetone, followed by 3-methyl-5-adamantyl catechol (0.26 g, 1.0 mmol). A dark purple solution resulted after 30 min but stirred for 24 h. The reaction mixture was vacuum filtered and then concentrated to dryness under reduced pressure at room temperature. The purple residue was dissolved in a minimal amount of HPLC grade acetone and then n-hexane (100 mL) was added. The solution was stored at −20° C. for 2-3 days. The black microcrystalline precipitate was vacuum filtered, washed with cold hexanes (2×20 mL), and dried under vacuum for 3 days to yield 0.36 g (59%) of a black solid. δ¹H NMR (CDCl₃, 400 MHz): 8.07 (s, 1H), 7.10 (d, J=8.8 Hz, 1H), 6.34 (s, 1H), 6.18 (s, 2H), 6.15 (d, J=8.9 Hz, 2H), 4.84 (s, 1H), 4.11 (d, J=12.8 Hz, 2H), 3.86 (s, 2H), 3.60 (s, 1H), 3.36 (q, J=7.1 Hz, 4H), 3.29 (m, 1H), 3.02 (s, 1H), 2.62 (m, 1H), 2.28 (s, 3H), 2.04 (s, 3H), 1.79 (d, J=3.0 Hz, 6H), 1.72 (m, 6H), 1.16 (t, J=7.0 Hz, 6H). δ⁵¹V NMR (CDCl₃, 105 MHz): 453 ppm (minor), 413 ppm (minor), 323 ppm (major). UV/Vis (DMSO), λₘₐₓ/nm: 555, 892.

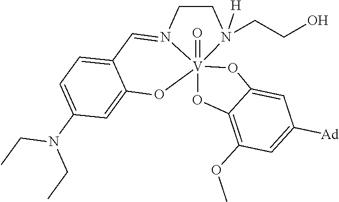

[VO(dea-Hshed)(3omad)]. To a 250-mL round bottom Schlenk flask ACS grade acetone (100 mL) was added and degassed with Argon for 20 minutes. [VO₂(dea-Hshed)] (0.36 g, 1.0 mmol) was added to the degassed acetone, followed by 3-methoxy-5-adamantyl catechol (0.27 g, 1.0 mmol). A dark purple solution resulted after 30 min but stirred for 24 h. The reaction mixture was vacuum filtered and then concentrated to dryness under reduced pressure at room temperature. The purple residue was dissolved in a minimal amount of HPLC grade acetone and then n-hexane (100 mL) was added. The solution was stored at −20° C. for 2-3 days. The black microcrystalline precipitate was vacuum filtered, washed with cold hexanes (2×20 mL), and dried under vacuum for 3 days to yield 0.24 g (39%) of a black solid. δ¹H NMR (400 MHz, d₆-Acetone): 8.28 (s, 1H), 7.20 (d, J=8.9 Hz, 1H), 6.23 (dd, J=8.9, 2.5 Hz, 1H), 5.99 (m, 2H), 5.89 (d, J=2.5 Hz, 1H), 4.21 (d, J=11.3 Hz, 1H), 4.09-4.00 (m, 2H), 3.92 (m, 2H), 3.86 (s, 3H), 3.73 (m, 1H), 3.62 (d, J=5.9 Hz, 1H), 3.56-3.45 (m, 1H), 3.40 (q, J=7.0 Hz, 4H), 3.39 (m, 1H), 2.56 (m, 1H), 1.91-1.68 (m, 15H), 1.14 (t, J=6.9 Hz, 6H). δ⁵¹V NMR (d₆-Acetone, 105 MHz): 446 ppm (minor), 396 ppm (minor), 350 ppm (minor), 323.70 ppm (major). UV/Vis (DMSO), λₘₐₓ/nm: 570, 834.

[VO(SALIEP)(DAD)]. To 12.5 mL of degassed methanol, the [VO₂(SALIEP)] precursor (0.145 g, 0.50 mmol, 1 equiv.) and the di-adamantyl-catechol ligand (0.227 g, 0.60 mmol, 1.2 equiv.) were added. The reaction mixture was allowed to stir for 20 h at r.t. under argon. The resulting product was vacuum filtered, washed with cold methanol and dried under high vacuum. Yield: 50%. δ¹H NMR (d₃-MeCN, 400.3 MHz): δ 8.98 (d, J=5.5 Hz, 1H), 8.50 (s, 1H), 7.89 (q, J=7.7 Hz, 1H), 7.47 (d, J=9.1 Hz, 1H), 7.40 (s, 2H), 6.73 (dt, J=13.9, 7.4 Hz, 1H), 6.68-6.61 (m, 1H), 6.03 (s, 1H), 3.62-3.51 (m, 1H), 3.29 (dd, J=16.3, 6.2 Hz, 1H), 2.13 (s, 3H), 1.94 (s, 5H), 1.40 (s, 5H), 1.27 (s, 4H), 1.21 (s, 5H), 0.98 (s, 3H). δ⁵¹V NMR (d₆-DMSO, 105.2 MHz): −511, −531 ppm. λₘₐₓ (0.05 mM in DMSO)=560 nm.

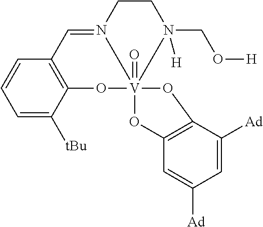

[VO(tbuHSHED)dad]. 3,5-di-adamantyl-catechol (46 mg, 0.132 mmol) was added to a solution of [VO₂(tbuHSHED)] (50 mg, 0.132 mmol) and stirred in acetone (15 mL) for 24 hrs under an argon atmosphere. Upon addition of the catechol the reaction was observed to rapidly change color from a moderately dark but transparent yellow, to a dark, almost black, purple color. The reaction was wrapped in tinfoil in an effort to prevent unwanted photodegradation. After 48 hrs, the solution was then cooled to −78° C., and filtered through a fritted filter. The solvent was removed by rotovap, and the residue dried in vacuo for 4 days. At this point the tacky purple-black crystalline solid was scrapped out of the round-bottomed flask, crushed as best as possible, and put back under vacuum at 30° C. for a week. The compound was again scraped out of the round-bottomed flask and pulverized before being placed under vacuum at 30° C. for another week. Yield 36.4 mg (40%).

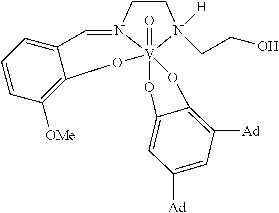

[VO(3-OmeHshed)(dad)]. To a 250 mL round bottom Schlenk flask, ethyl acetate (100 mL), which was then degassed with argon for 15 minutes. [VO(3-Ome-HSHED)] (0.325 g, 1.00 mmol) was added, followed by the addition of 3,5-diadamantylcatechol (0.379 g, 1.00 mmol). The reaction mixture changed from a light yellow to deep purple within 5 minutes and the solution was stirred for 24 h at an ambient temperature under Ar. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold hexanes (<0° C., 2×25 mL), and dried under vacuum for 3 days to yield a purple solid. Yield: 0.361 g (53%). δ⁵¹V NMR (101 MHz, CD₃CN): 380 ppm (major), 458 ppm (minor). δ¹H NMR (400 MHz, CDCl₃): 8.46 ppm (s, 1H), 6.96 ppm (t, 2H), 6.64 ppm (t, 1H), 6.57 ppm (s, 1H), 6.11 ppm (s, 1H), 4.01-3.92 ppm (m, 4H), 3.72 ppm (m, 1H), 3.64 ppm (s, 3H), 3.54 ppm (m, 1H), 3.44 ppm (m, 1H), 3.35 ppm (m, 2H), 2.82-2.74 ppm (m, 2H), 1.75-1.60 ppm (m, 18H). UV-vis λₘₐₓ: 552 nm, 882 nm

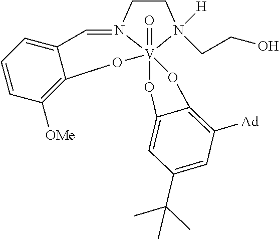

[VO(3-OmeHshed)(tbad)]. To a 250 mL round bottom Schlenk flask, ethyl acetate (100 mL), which was then degassed with argon for 15 minutes. [VO(3-Ome-HSHED)] (0.325 g, 1.00 mmol) was added, followed by the addition of 3-adamantyl-5-tert-butylcatechol (0.300 g, 1.00 mmol). The reaction mixture changed from a light yellow to deep purple within 5 minutes and the solution was stirred for 24 h at an ambient temperature under Ar. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold hexanes (<0° C., 2×25 mL), and dried under vacuum for 3 days to yield a purple solid. Yield: 0.331 g (55%). δ⁵¹V NMR (101 MHz, CD₃CN): 372 ppm (major), 418 ppm (minor), 452 ppm (minor), 461 ppm (minor). δ¹H NMR (400 MHz, CDCl₃): 8.46 ppm (s, 1H), 6.96 ppm (m, 2H), 6.66 ppm (t, 1H), 6.57 ppm (m, 1H), 6.18 ppm (s, 1H), 4.01-3.92 ppm (m, 4H), 3.72 ppm (m, 1H), 3.64 ppm (s, 3H), 3.54 ppm (m, 2H), 3.46 ppm (m, 1H), 3.36 ppm (m, 1H), 3.23 ppm (m, 1H), 2.82-2.70 ppm (m, 2H), 1.80 ppm (s, 18H), 1.19 ppm (s, 3H), 1.17 ppm (s, 6H). UV-vis λₘₐₓ: 552 nm, 878 nm

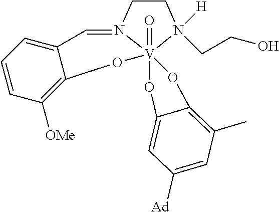

[VO(3-OmeHshed)(3mad)]. To a 250 mL round bottom Schlenk flask, ethyl acetate (100 mL), which was then degassed with argon for 15 minutes. [VO(3-Ome-HSHED)] (0.325 g, 1.00 mmol) was added, followed by the addition of 3-methyl-5-adamantylcatechol (0.258 g, 1.0 mmol). The reaction mixture changed from a light yellow to deep purple within 5 minutes and the solution was stirred for 24 h at an ambient temperature under Ar. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The dark microcrystalline precipitate was vacuum filtered, washed with cold hexanes (<0° C., 2×25 mL), and dried under vacuum for 3 days to yield a purple solid. Yield: 0.280 g (50%). δ⁵¹V NMR (101 MHz, CD₃CN): 401 ppm (major), 441 ppm (minor), 464 ppm (minor), 461 ppm (minor). δ¹H NMR (400 MHz, CDCl₃): 8.48 ppm (s, 1H), 6.98 ppm (m, 2H), 6.65 ppm (t, 1H), 6.17 ppm (s, 1H), 6.07 ppm (s, 1H), 3.98-3.89 ppm (m, 4H), 3.71 ppm (m, 1H), 3.66 ppm (s, 3H), 3.56 ppm (m, 2H), 3.45 ppm (m, 1H), 3.33 ppm (m, 1H), 3.21 ppm (m, 1H), 2.72 ppm (m, 1H), 1.95 ppm (s, 3H), 1.74 ppm (s, 6H), 1.67 ppm (m, 8H). UV-vis λₘₐₓ: 555 nm, 877 nm.

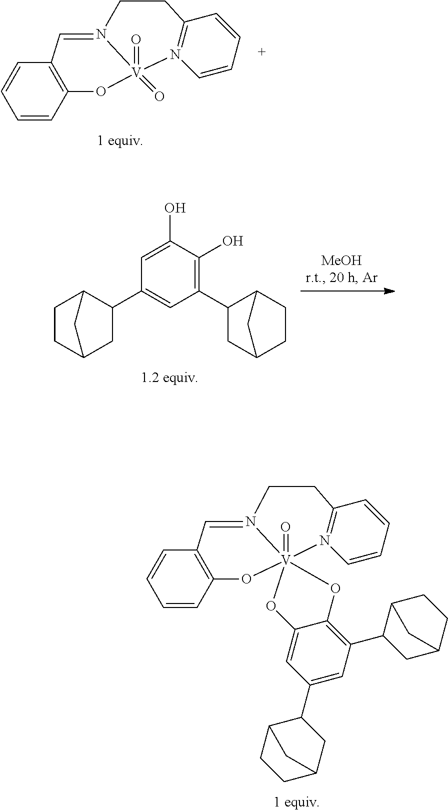

[VO(SALIEP)(DNC)]. To 12.5 mL of degassed methanol, the [VO₂(SALIEP)] precursor (0.154 g, 0.50 mmol, 1 equiv.) and the 3,5-di-norbornane-catechol ligand (0.179 g, 0.60 mmol, 1.2 equiv.) were added. The reaction mixture was allowed to stir for 20 h at r.t. under argon. The resulting product was vacuum filtered, washed with cold methanol and dried under high vacuum. Yield: 0.074 g (25%). δ¹H NMR (d₃-MeCN, 400.3 MHz): 8.52 (d, 1H), 8.34 (s, 1H), 7.66 (t, 1H), 7.46 (t, 1H), 7.33 (d, 1H), 7.22 (t, 1H), 7.18 (d, 1H), 4.04 (t, 2H), 3.32 (t, 2H). ¹H (d₃-MeCN): 8.90 (d, 1H), 8.69 (s, 1H), 7.99 (t, 1H), 7.49 (t, 2H), 7.43 (t, 1H), 6.87 (m, 2H), 6.76 (s, 1H), 6.54 (s, 1H), 4.03 (t, 2H), 3.39 (t, 1H), 3.29 (d, 1H). 2.88 (t, 2H), 1.65 (m, 4H), 1.54 (m, 8H), 1.50 (m, 4H), 1.43 (d, 2H), 1.42 (m, 2H), 1.34 (m, 4H), 1.12 (d, 2H). δ⁵¹V NMR (d₃-MeCN, 105.2 MHz): −553 ppm. λₘₐₓ (0.05 mM in DMSO)=561 nm.

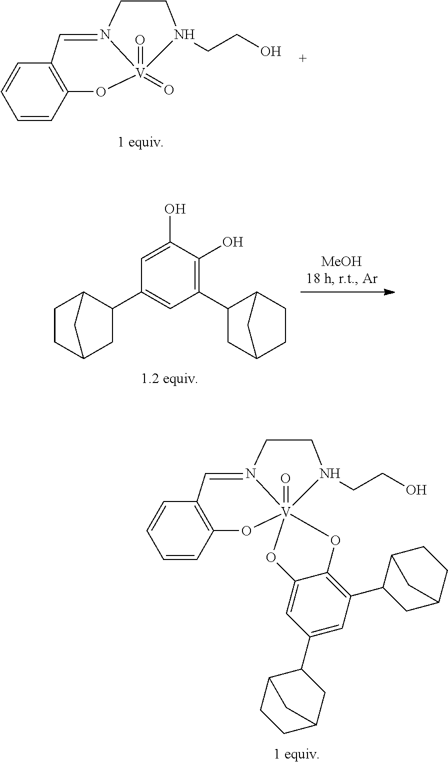

[VO(HSHED)(DNC)]. To 12.5 mL of degassed methanol, the [VO₂(HSHED)] precursor (X g, 0.50 mmol, 1 equiv.) and the 3,5-di-norbornane-catechol ligand (0.179 g, 0.60 mmol, 1 equiv.) were added. The reaction mixture was allowed to stir for 20 h at r.t. under argon. The resulting product was vacuum filtered, washed with cold methanol and dried under high vacuum. Yield: 0.029 g (10%). Characterization: δ¹H NMR (d₆-DMSO, 400.3 MHz): 8.1 (s, 1H), 7.65 (d, 2H), 7.41 (t, 1H), 7.16 (t, 1H), 6.92 (d, 1H), 6.44 (s, 1H), 6.03 (s, 1H), 3.48 (t, 2H), 2.88 (t, 2H), 2.78 (t, 2H), 1.65 (m, 4H), 1.54 (m, 8H), 1.50 (m, 4H), 1.43 (d, 2H), 1.42 (m, 2H), 1.34 (m, 4H), 1.12 (d, 2H). δ⁵¹V NMR (d₆-DMSO, 105.2 MHz): −500 ppm (major), −520 ppm (minor). λₘₐₓ (0.05 mM in DMSO)=561 nm.

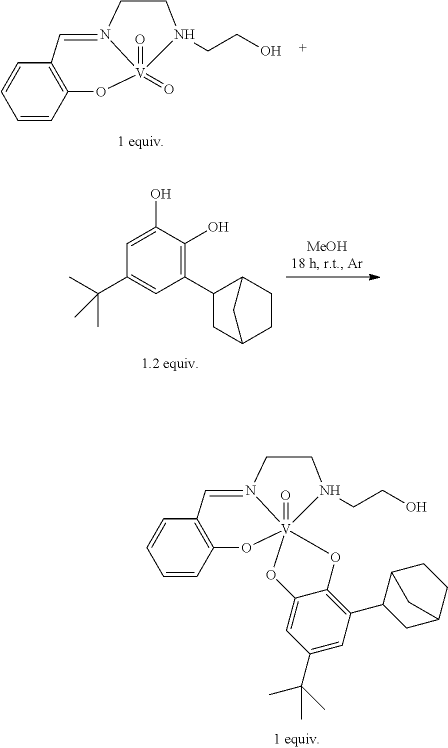

[VO(HSHED)(TBNC)]. To 12 mL of degassed methanol, the [VO₂(HSHED)] precursor (0.145 g, 0.50 mmol, 1 equiv.) and the 3-norbornane-5-tert-butylcatechol ligand (0.156 g, 0.60 mmol, 1 equiv.) were added. The reaction mixture was allowed to stir for 20 h at r.t. under argon. The resulting product was vacuum filtered, washed with cold methanol and dried under high vacuum. Yield: 25% (0.067 g). Characterization: δ¹H NMR (d₆-DMSO, 400 MHz) δ 8.74 (s, 1H), 7.51 (d, 1H), 7.41 (m, 1H), 6.76 (dd, J=30.5, 7.4 Hz, 2H), 6.16 (s, 1H), 4.84 (s, 1H), 4.78 (d, J=45.3 Hz, 1H), 3.75 (d, J=21.3 Hz, 2H), 3.12 (t, 2H), 2.90 (m, 2H), 1.58 (m, 2H), 1.49-1.38 (m, 9H), 1.31 (d, J=7.6 Hz, 2H), 1.22 (q, J=7.4 Hz, 9H). δ⁵¹V NMR (d₆-DMSO, 105.2 MHz): −527 ppm. λₘₐₓ (0.05 mM in DMSO)=561 nm.

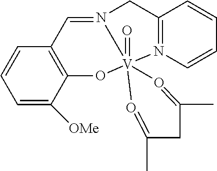

[V^{V}O(3-OMeSALIMP)(acac)]. To a 100 mL round bottom Schlenk flask, ethanol (32.5 mL) was degassed with argon for 20 minutes. This was followed by the addition of 2-(2-aminoethyl)pyridine (0.865 mL, 8.85 mmol) and 2-Hydroxy-3-methoxybenzaldehyde (1.35 g, 8.85 mmol). After the reaction mixture was stirred under reflux for 1 hour under argon. During the stirring of the first reaction mixture, to a 50 mL round bottom Schlenk flask, vanadyl acetylacetonate (2.24 g, 8.85 mmol) was dissolved in ethanol (15 mL) that was degassed with argon for 20 minutes. After the first reaction mixture was completed, it was cooled to room temperature. The two reaction mixtures were combined and stirred under reflux for 45 minutes under argon. Within 1 minute, there was a color change from a yellow mixture and blue solution to a reddish-brown color. The solution was cooled to room temperature and vacuum filtered. The brown product was washed with ethanol until the filtrate was colorless. The residue was washed with pentanes and left to dry under high vacuum for 3 days. Yield: 3.08 g (85.1%). ADD UV-Vis data if have

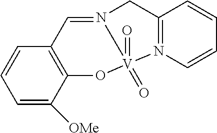

To a 100 mL round bottom Schlenk flask, the dry, brown product (0.408 g, 1.00 mmol) was dissolved in methanol (50 mL) and cooled to 0° C. 30% H₂O₂ (0.141 mL, 2.00 mmol) was added dropwise over a 5-minute time period to the reaction mixture and stirred for 30 minutes at room temperature. The product was vacuum filtered and then concentrated to dryness under reduced pressure to yield a red-orange solid. Yield: 0.315 g (97.2%).

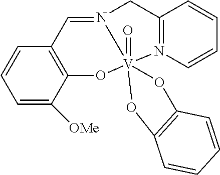

[VO(3-OMeSALIMP)(cat)]. To a 250 mL round bottom Schlenk flask, dichloromethane (100 mL) was and degassed with argon for 20 minutes. [VO(3-OMeSALIMP)(acac)] (0.408 g, 1.00 mmol) and catechol (0.110 g, 1.00 mmol) were added and then stirred under reflux for 1 hour under argon. The reaction mixture changed from a brown to purple solid in 10 minutes. The reaction mixture was cooled to room temperature and left to stir overnight. The product was vacuum filtered and then concentrated to dryness under reduced pressure at room temperature. The purple residue was dissolved in a minimal amount of acetone and then n-hexane (100 mL) was added. The solution was stored at −20° C. freezer overnight. The purple precipitate was vacuum filtered, washed with cold hexanes (<0° C., 2×25 mL), and dried under vacuum for 3 days to yield a purple solid. Yield: 0.342 g (82.1%). δ⁵¹V NMR (101 MHz, CD₃CN): 368 ppm (major); 423 ppm (minor), 456 ppm (minor), 478 ppm (minor). δ¹H NMR (400 MHz, CDCl₃): 8.46 ppm (s, 1H), 6.96 ppm (m, 3H), 6.64 ppm (t, 1H), 6.17 ppm (m, 1H), 4.02-3.89 ppm (m, 4H), 3.72 ppm (m, 1H), 3.63 ppm (s, 3H), 3.55 ppm (m, 1H), 3.44 ppm (m, 2H), 3.36 ppm (m, 1H), 3.24 ppm (m, 1H), 2.83-2.75 ppm (m, 2H), 1.32 ppm (s, 6H), 1.20 ppm (s, 3H), 1.17 ppm (s, 6H), 1.16 ppm (s, 3H). UV-vis λₘₐₓ: 553 nm, 869 nm.

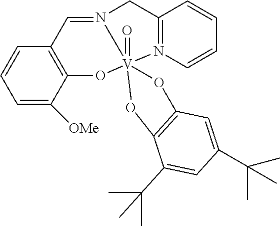

[VO(3-OMeSALIMP)(dtb)]. To a 250 mL round bottom Schlenk flask, dichloromethane (100 mL) was and degassed with argon for 20 minutes. [VO(3-OMeSALIMP)(acac)] (0.652 g, 1.60 mmol) and 3,5-di-tert-butylcatechol (0.356 g, 1.60 mmol) were added and then stirred under reflux for 1 hour under argon. The reaction mixture changed from a brown to purple color within 5 minutes. The reaction mixture was cooled to room temperature and left to stir overnight. The product was vacuum filtered and then concentrated to dryness under reduced pressure at room temperature. The purple residue was dissolved in a minimal amount of acetone and then n-hexane (100 mL) was added. The solution was stored at −20° C. freezer overnight. The purple precipitate was vacuum filtered, washed with cold hexanes (<0° C., 2×25 mL), and dried under vacuum for 3 days to yield a purple solid. Yield: 0.697 g (82.4%).

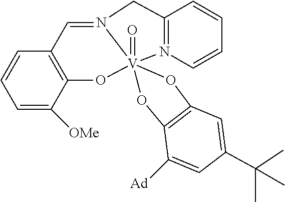

[VO(3-OMeSALIMP)(tbad)]. To a 250 mL round bottom Schlenk flask, dichloromethane (100 mL) was and degassed with argon for 20 minutes. [VO(3-OMeSALIMP)(acac)] (0.408 g, 1.00 mmol) and 3-adamantyl-5-tert-butylcatechol (0.300 g, 1.00 mmol) were added and then stirred under reflux for 1 hour under argon. The reaction mixture changed from a brown to purple color in 5 minutes. The reaction mixture was cooled to room temperature and left to stir overnight. The product was vacuum filtered and then concentrated to dryness under reduced pressure at room temperature. The purple residue was dissolved in a minimal amount of acetone and then n-hexane (100 mL) was added. The solution was stored at −20° C. freezer overnight. The precipitate was vacuum filtered, washed with cold hexanes (<0° C., 2×25 mL), and dried under vacuum for 3 days to yield a purple solid. Yield: 0.533 g (87.9%).

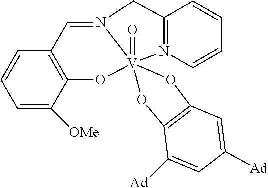

[VO(3-OMeSALIMP)(dad)]. To a 250 mL round bottom Schlenk flask, dichloromethane (100 mL) was and degassed with argon for 20 minutes. [VO(3-OMeSALIMP)(acac)] (0.408 g, 1.00 mmol) and 3,5-diadamantylcatechol (0.379 g, 1.00 mmol) were added and then stirred under reflux for 1 hour under argon. The reaction mixture changed from a brown to purple color in 5 minutes. The reaction mixture was cooled to room temperature and left to stir overnight. The product was vacuum filtered and then concentrated to dryness under reduced pressure at room temperature. The color residue was dissolved in a minimal amount of acetone and then n-hexane (100 mL) was added. The solution was stored at −20° C. freezer overnight. The purple precipitate was vacuum filtered, washed with cold hexanes (<0° C., 2×25 mL), and dried under vacuum for 3 days to yield a purple solid. Yield: 0.574 g (83.9%).

[VO(SALIMP)]-Catechol Complexes

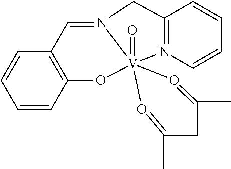

[VO(SALIMP)(acac)]. To a 100 mL round bottom Schlenk flask, ethanol (32.5 mL) was degassed with argon for 20 minutes. This was followed by the addition of 2-(2-aminoethyl)pyridine (0.865 mL, 8.85 mmol) and salicylaldehyde (0.922 mL, 8.85 mmol). After the reaction mixture was stirred under reflux for 1 hour under argon. During the stirring of the first reaction mixture, to a 50 mL round bottom Schlenk flask, vanadyl acetylacetonate (2.24 g, 8.85 mmol) was dissolved in ethanol (15 mL) that was degassed with argon for 20 minutes. After the first reaction mixture was completed, it was cooled to room temperature. The two reaction mixtures were combined and stirred under reflux for 45 minutes under argon. Within 1 minute, there was a color change from a yellow mixture and blue solution to a reddish-brown color. The solution was cooled to room temperature and vacuum filtered. The brown product was washed with ethanol until the filtrate was colorless. The residue was washed with pentanes and left to dry under high vacuum for 3 days. Yield: 2.82 g (84.3%).

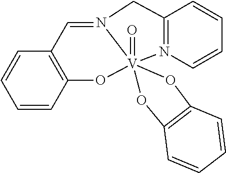

[VO(SALIMP)(cat)]. To a 250 mL round bottom Schlenk flask, dichloromethane (100 mL) was and degassed with argon for 20 minutes. [VO(SALIMP)(acac)] (0.378 g, 1.00 mmol) and catechol (0.110 g, 1.00 mmol) were added and then stirred under reflux for 1 hour under argon. The reaction mixture changed from a brown to purple solid in 10 minutes. The reaction mixture was cooled to room temperature and left to stir overnight. The product was vacuum filtered and then concentrated to dryness under reduced pressure at room temperature. The purple residue was dissolved in a minimal amount of acetone and then n-hexane (100 mL) was added. The solution was stored at −20° C. freezer overnight. The purple precipitate was vacuum filtered, washed with cold hexanes (<0° C., 2×25 mL), and dried under vacuum for 3 days to yield purple solid. Yield: 0.0757 g (20.0%).

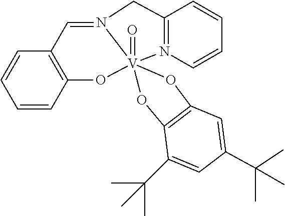

[VO(SALIMP)(dtb)]. To a 250 mL round bottom Schlenk flask, dichloromethane (100 mL) was and degassed with argon for 20 minutes. [VO(SALIMP)(acac)] (0.530 g, 1.40 mmol) and 3,5-di-tert-butylcatechol (0.311 g, 1.40 mmol) were added and then stirred under reflux for 1 hour under argon. The reaction mixture changed from a brown to dark blue/purple color. The reaction mixture was cooled to room temperature and left to stir overnight. The product was vacuum filtered and then concentrated to dryness under reduced pressure at room temperature. The purple residue was dissolved in a minimal amount of acetone and then n-hexane (100 mL) was added. The solution was stored at −20° C. freezer overnight. The purple precipitate was vacuum filtered, washed with cold hexanes (<0° C., 2×25 mL), and dried under vacuum for 3 days to yield a purple solid. Yield: 0.425 g (60.9%).

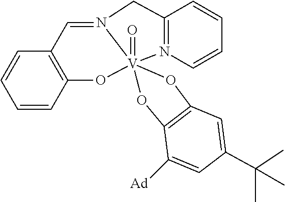

[VO(SALIMP)(tbad)]. To a 250 mL round bottom Schlenk flask, dichloromethane (100 mL) was and degassed with argon for 20 minutes. [VO(SALIMP)(acac)] (0.378 g, 1.00 mmol) and 3-adamantyl-5-tert-butylcatechol (0.300 g, 1.00 mmol) were added and then stirred under reflux for 1 hour under argon. The reaction mixture changed from a brown to purple color in 3 minutes. The reaction mixture was cooled to room temperature and left to stir overnight. The product was vacuum filtered and then concentrated to dryness under reduced pressure at room temperature. The purple residue was dissolved in a minimal amount of acetone and then n-hexane (100 mL) was added. The solution was stored at −20° C. freezer overnight. The purple precipitate was vacuum filtered, washed with cold hexanes (<0° C., 2×25 mL), and dried under vacuum for 3 days to yield a purple solid. Yield: 0.508 g (83.7%).

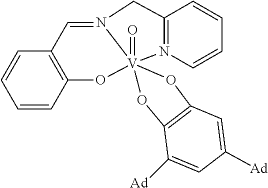

[VO(SALIMP)(dad)]. To a 250 mL round bottom Schlenk flask, dichloromethane (100 mL) was and degassed with argon for 20 minutes. [VO(SALIMP)(acac)] (0.160 g, 0.500 mmol) and 3,5-diadamantylcatechol (0.189 g, 0.500 mmol) were added and then stirred under reflux for 1 hour under argon. The reaction mixture changed from a brown to purple within 5 minutes. The reaction mixture was cooled to room temperature and left to stir overnight. The product was vacuum filtered and then concentrated to dryness under reduced pressure at room temperature. The color residue was dissolved in a minimal amount of acetone and then n-hexane (100 mL) was added. The solution was stored at −20° C. freezer overnight. The precipitate was vacuum filtered, washed with cold hexanes (<0° C., 2×25 mL), and dried under vacuum for 3 days to yield a purple solid. Yield: 0.176 g (53.6%).

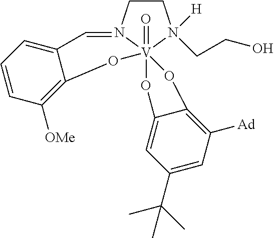

[VO(3-OmeHshed)(dad)]. To a 250 mL round bottom Schlenk flask, ethyl acetate (100 mL), which was then degassed with argon for 15 minutes. [VO(3-Ome-HSHED)] (0.325 g, 1.00 mmol) was added, followed by the addition of 3,5-diadamantylcatechol (0.379 g, 1.00 mmol). The reaction mixture changed from a brown to deep purple within 5 minutes and the solution was stirred for 24 h at an ambient temperature under Ar. The reaction mixture was vacuum filtered and then concentrated to dryness under reduced pressure at room temperature. The purple residue was dissolved in a minimal amount of acetone and then n-hexane (100 mL) was added. The solution was stored at −20° C. freezer overnight. The purple precipitate was vacuum filtered, washed with cold hexanes (<0° C., 2×25 mL), and dried under vacuum for 3 days to yield a purple solid. Yield: 0.361 g (53%). δ⁵¹V NMR (101 MHz, CD₃CN): 380 ppm (major), 458 ppm (minor). δ¹H NMR (400 MHz, CDCl₃): 8.46 ppm (s, 1H), 6.96 ppm (t, 2H), 6.64 ppm (t, 1H), 6.57 ppm (s, 1H), 6.11 ppm (s, 1H), 4.01-3.92 ppm (m, 4H), 3.72 ppm (m, 1H), 3.64 ppm (s, 3H), 3.54 ppm (m, 1H), 3.44 ppm (m, 1H), 3.35 ppm (m, 2H), 2.82-2.74 ppm (m, 2H), 1.75-1.60 ppm (m, 18H). UV-vis λₘₐₓ: 552 nm, 882 nm

Evaluation of the Biological Activity of Selected Vanadium(V) Complexes

Cell Culture and Growth Conditions. The well-established human glioblastoma multiforme cell line (T98 g) and human foreskin fibroblast (HFF-1) cell lines were purchased from American Type Culture Collection (ATCC, cat. no. CRL-1690 and SCRC-1041). Due to their limited life span in culture, HFF-1 cells were used at passages four to six. The cells were cultured in Advanced DMEM (Thermo Fisher Scientific cat. no. 12491-015), supplemented with L-glutamine (2.0 mM), antibiotic-antimycotic mixture (100 U mL⁻¹ penicillin, 100 mg mL⁻¹ streptomycin, and 0.25 mg mL⁻¹ amphotericin B), and fetal calf serum (FCS; heat-inactivated; 2% vol). For proliferation and cytotoxicity experiments, cells were seeded in 96-well plates at an initial density of 1.5×10³ viable cells per well in 100 μL medium and left to attach overnight.

Freshly prepared stock solutions of V(V) complexes (10 mM in DMSO) were used for cell assays. These solutions were further diluted so that all the cell treatments, including controls, contained 1.0% (vol) of DMSO, which did not significantly affect the cell growth during the assays. Stock solutions of the treatment complexes were diluted with fully supplemented cell culture media to the required final concentrations, and the resultant media were either added to the cells within 1 min (fresh solutions) or left in cell culture incubator (310 K, 5% CO₂) for 24 h prior to the cell treatments (aged solutions).

Each treatment included six replicate wells with cells and two background wells without cells that contained the same components. After the addition of treatment complexes, the plates were incubated for 72 h at 310 K and 5% CO₂, then MTT reagent [1-(4,5-dimethylthiazol-2-yl)-3,5-diphenylformazan, Sigma M5655] was added (50 μL per well of freshly prepared 2.0 mg/mL solution in complete medium), and incubation was continued for 4-6 h. After that, the medium was removed, the blue formazan crystals were dissolved in 0.10 mL per well of DMSO, and the absorbance at 600 nm was measured using a Victor V3 plate reader. Typically, the treatment complexes were applied in a series of nine two-fold dilutions, starting from (100±20) M V, plus the vehicle control. Exact V concentrations in the assays were verified by ICP-MS measurements using samples of cell culture media and used in the calculations of the IC50 values. Fitting of the experimental data and calculations of the IC50 values were performed using Origin 6.1 software (Microcal Origin, 1999). For all the cell assays, consistent results were obtained in at least two independent experiments using different passages of cells and varying stock solutions of the treatment complexes.

IC₅₀ values for 72 h treatments with fresh example vanadium(V) compounds in T98 g cells are included below. In cases where multiple measurements were performed, the value determined for each measurement are reported.

| Vanadium Complex | IC₅₀ Value(s) (μM) |
|---|---|
| 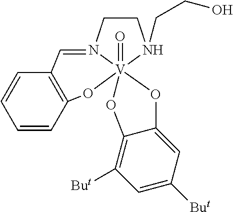 | 1.9 ± 0.2 2.7 ± 0.7 |
| 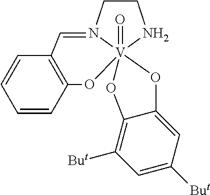 | 15 ± 1 |
| 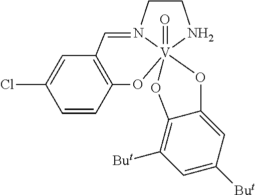 | 4.3 ± 0.5 |
| 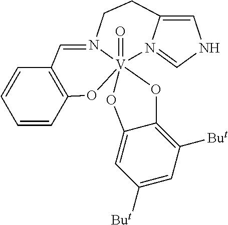 | 6.9 ± 0.9 |
| 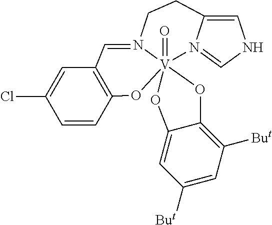 | 14 ± 2 |
| 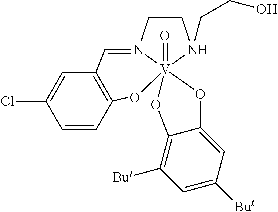 | 4.1 ± 0.5 |
| 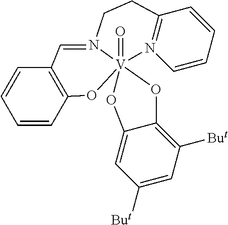 | 6.7 ± 0.9 |
| 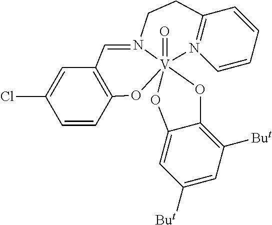 | 12 ± 1.5 18 ± 3 |
| 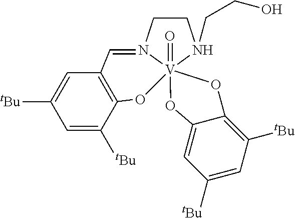 | 1.0 ± 0.1 1.0 ± 0.4 1.9 ± 0.3 |
| 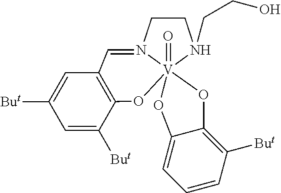 | 5.0 ± 0.7 5.2 ± 0.6 |
| 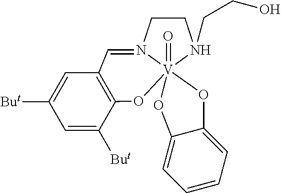 | 7.7 ± 0.9 4.8 ± 1.2 |
| 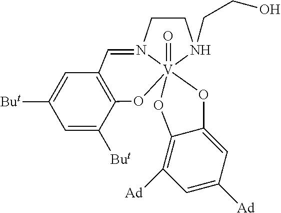 | 32 ± 5 (aged 5.8 ± 0.3) 14 ± 4 (aged 10 ± 1) |
| 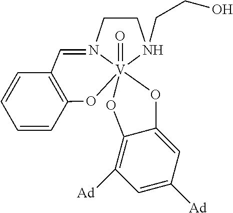 | 14 ± 1 11 ± 2 |
| 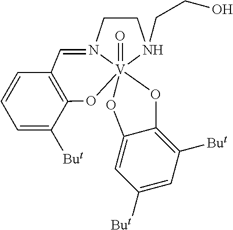 | 1.4 ± 0.2 |
| 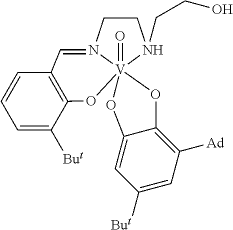 | 0.95 ± 0.09 |
| 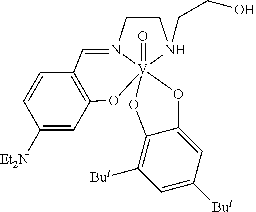 | — |
| 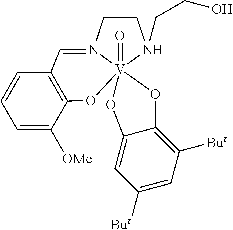 | 14 ± 2 |
| 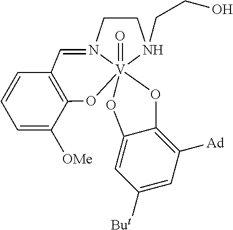 | 2.2 ± 0.1 |
| 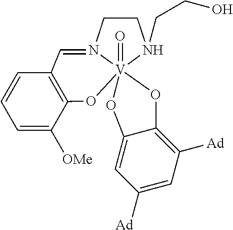 | 4.2 ± 0.3 |
| 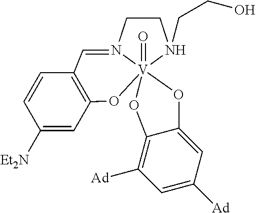 | ~20 |
| 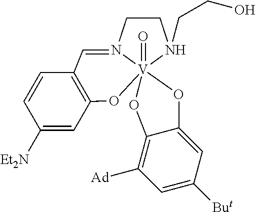 | 1.6 ± 0.1 ~6.5 |
| 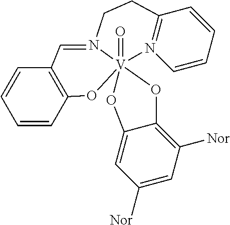 | 74 ± 6 |
| 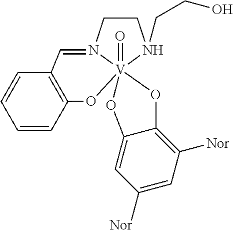 | 5.5 ± 1.5 15 ± 1 |
| 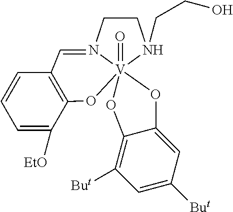 | 2.4 ± 0.3 |
| 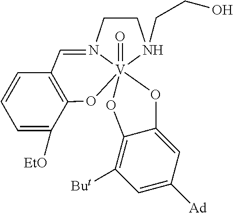 | 2.1 ± 0.3 |
| 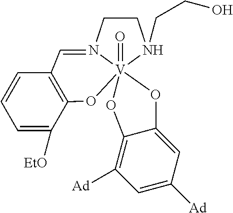 | 3.3 ± 0.4 |
| 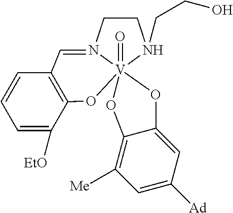 | 14 ± 2 |
| 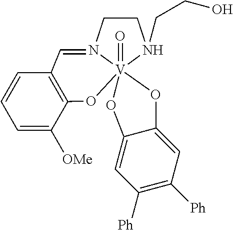 | 60 ± 5 |
| 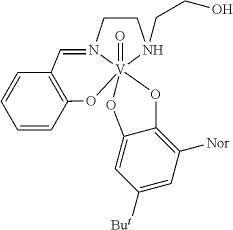 | 8 ± 1 |
| 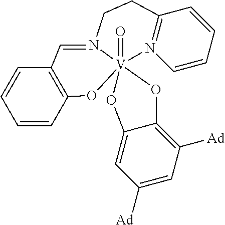 | 30 ± 4 |
| 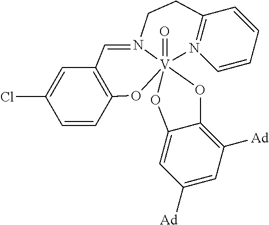 | 7.5 ± 1.2 |
| 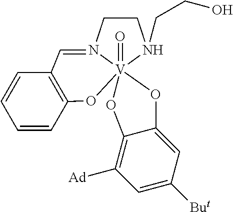 | 9.3 ± 2.1 |
| 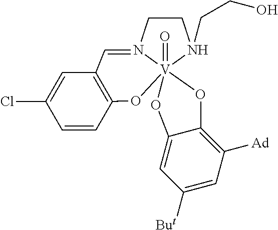 | 3.4 ± 0.2 |
| 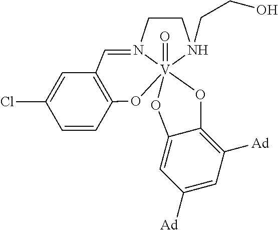 | 11 ± 1 |
| 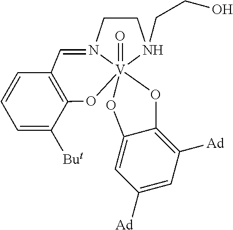 | 5.3 ± 1.6 |
| 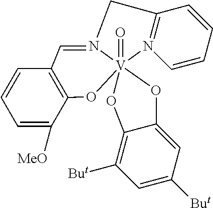 | 0.8 ± 0.2 |
| 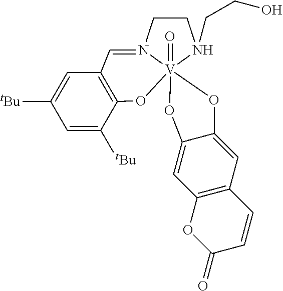 | 4.2 ± 0.9 6.9 ± 1.2 |

The compounds, compositions, and methods of the appended claims are not limited in scope by the specific compounds, compositions, and methods described herein, which are intended as illustrations of a few aspects of the claims. Any compounds, compositions, and methods that are functionally equivalent are intended to fall within the scope of the claims. Various modifications of the compounds, compositions, and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A vanadium complex defined by Formula I, Formula II, or Formula III 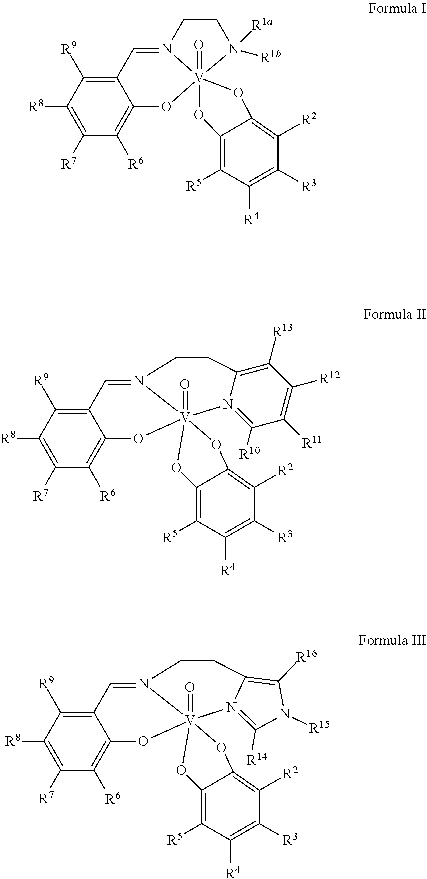
wherein
R^{1a} is selected from C₁₋₆ alkyl, C₁₋₄haloalkyl, C₂₋₆ alkenyl, C₂₋₆ alkynyl, C₃₋₁₀ cycloalkyl, C₃₋₁₀ cycloalkyl-C₁₋₄ alkylene, 6-10 membered aryl, and 6-10 membered aryl-C₁₋₄ alkylene, each of which is optionally substituted by 1, 2, 3, or 4 independently selected R^{A} groups;
R^{1b} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups;
R², R³, R⁴, and R⁵ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R² and R³, R³ and R⁴, and/or R⁴ and R⁵, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R⁶, R⁷, R⁸, and R⁹ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R⁶ and R⁷, R⁷ and R⁸, and/or R⁸ and R⁹, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁰, R¹¹, R¹², and R¹³ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R¹⁰ and R¹¹, R¹¹ and R¹², and/or R¹² and R¹³, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁴ and R¹⁶ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups;
R¹⁵ is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups; and
each R^{A} is independently selected from OH, NO₂, CN, halo, C₁₋₆ alkyl, C₂₋₆ alkenyl, C₂-6 alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

2. The complex of claim 1, wherein one or more of R⁶, R⁷, R⁸, and R⁹ is not hydrogen.

3. The complex of claim 1, wherein one or more of R⁶, R⁷, R⁸, and R⁹ is halo, such as chloro.

4. The complex of claim 1, wherein one or more of R⁶, R⁷, R⁸, and R⁹ is C₁₋₆ alkyl, such as t-butyl.

5. The complex of claim 1, wherein one or more of R⁶, R⁷, R⁸, and R⁹ is di(C₁₋₆ alkyl)amino, such as diethylamino.

6. The complex of claim 1, wherein one or more of R⁶, R⁷, R⁸, and R⁹ is C₁₋₆ alkoxy, such as methoxy.

7. The complex of claim 1, wherein R⁸ is halo, such as chloro.

8. The complex of claim 1, wherein R⁶ and R⁸ are each C₁₋₆ alkyl, such as t-butyl.

9. The complex of claim 1, wherein R⁷ is di(C₁₋₆ alkyl)amino, such as diethylamino.

10. The complex of claim 1, wherein R⁶ is C₁₋₆ alkoxy, such as methoxy.

11. The complex of claim 1, wherein one or more of R², R³, R⁴, and R⁵ is not hydrogen.

12. The complex of claim 1, wherein one or more of R², R³, R⁴, and R⁵ is C₁₋₆ alkyl, such as t-butyl.

13. The complex of claim 1, wherein one or more of R², R³, R⁴, and R⁵ is a 3-10 membered cycloalkyl group, such as adamantly or norbornyl.

14. The complex of claim 1, wherein R³ and R⁵ are C₁₋₆ alkyl, such as t-butyl.

15. The complex of claim 1, wherein R³ and R⁵ are a 3-10 membered cycloalkyl group, such as adamantly or norbornyl.

16. The complex of claim 1, wherein Rib is hydrogen.

17. The complex of claim 1, wherein R^{1a} is a C₁₋₆ alkyl group substituted by a hydroxy group.

18. The complex of claim 1, wherein one or more of R¹⁰, R¹¹, R¹², and R¹³ are all hydrogen.

19. The complex of claim 1, wherein one or more of R¹⁴, R¹⁵, and R¹⁶ are all hydrogen.

20. The complex of claim 1, wherein the complex is not one of the complexes below

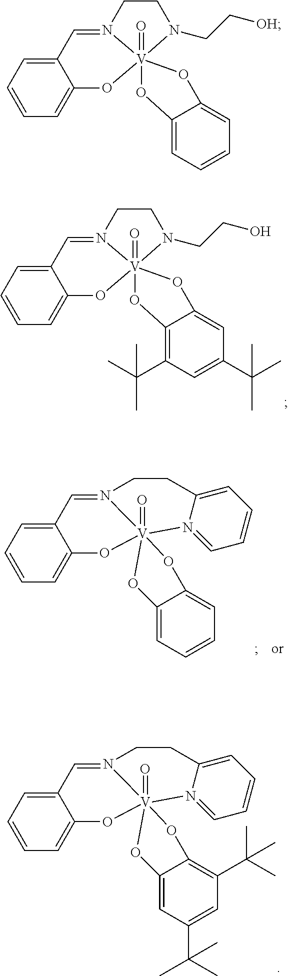

21. The complex of claim 1, wherein the complex is one of the complexes shown below

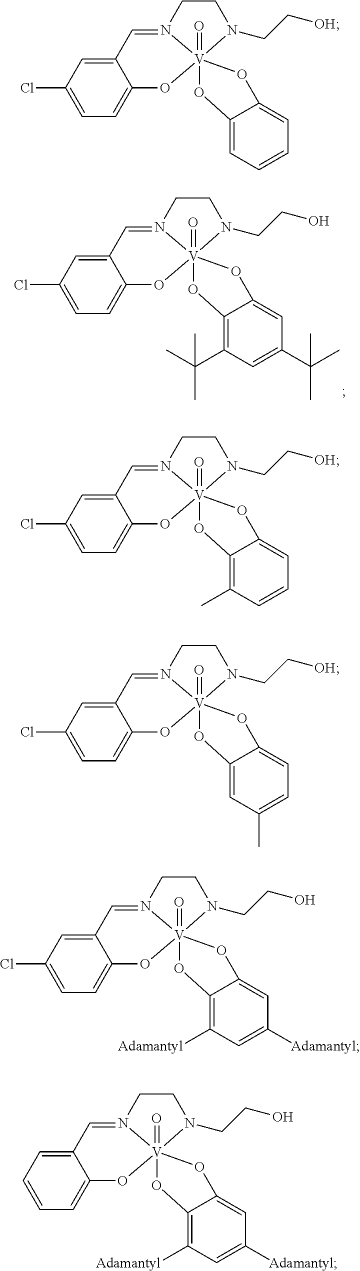

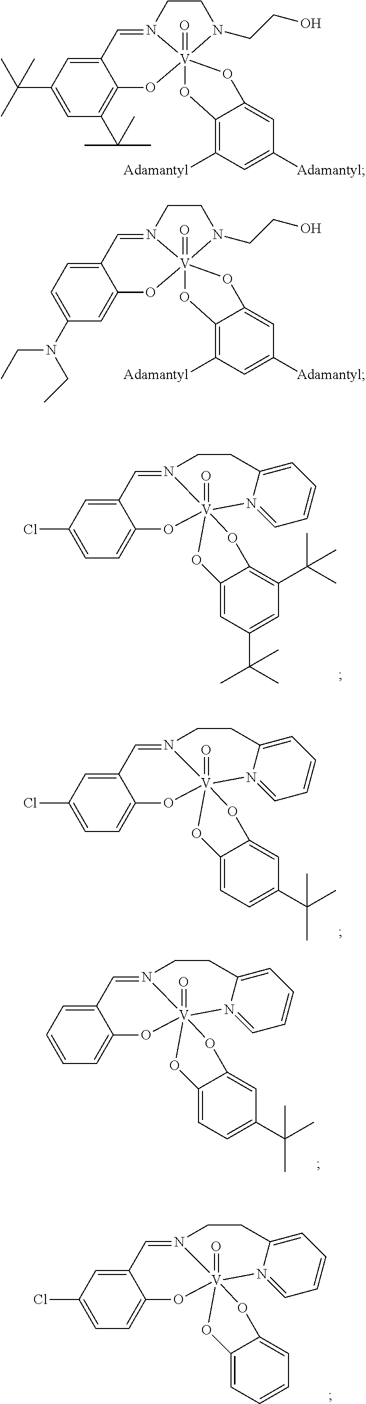

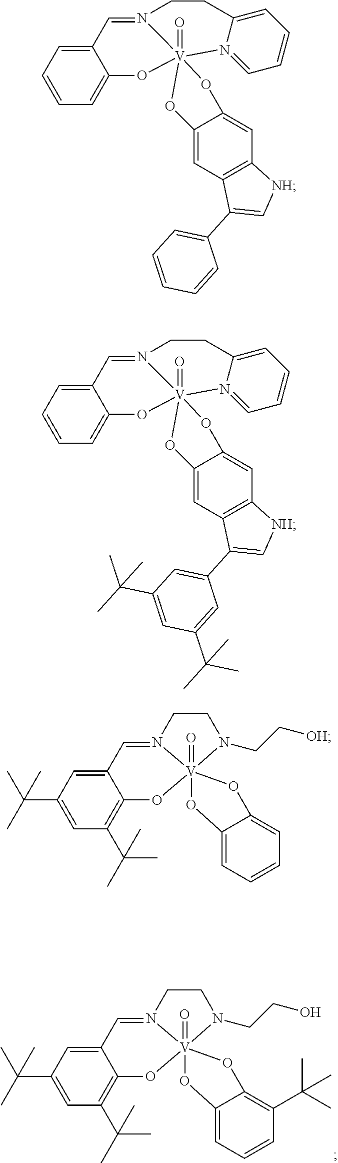

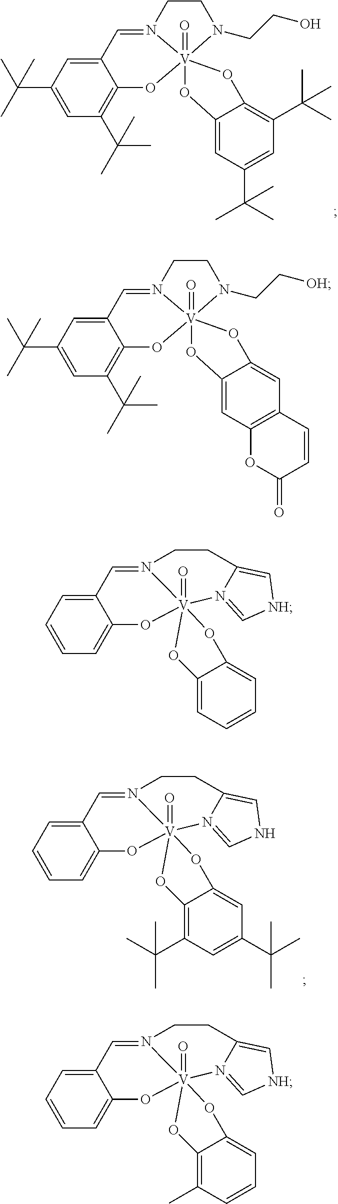

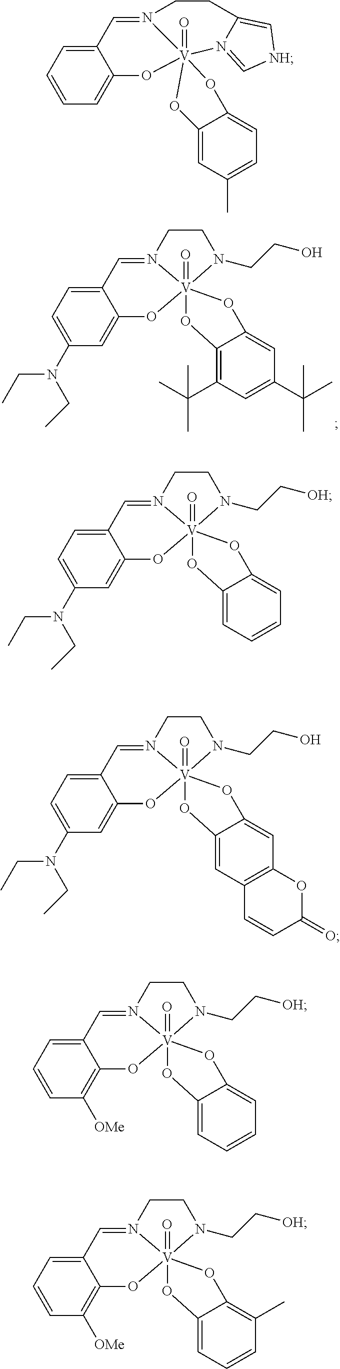

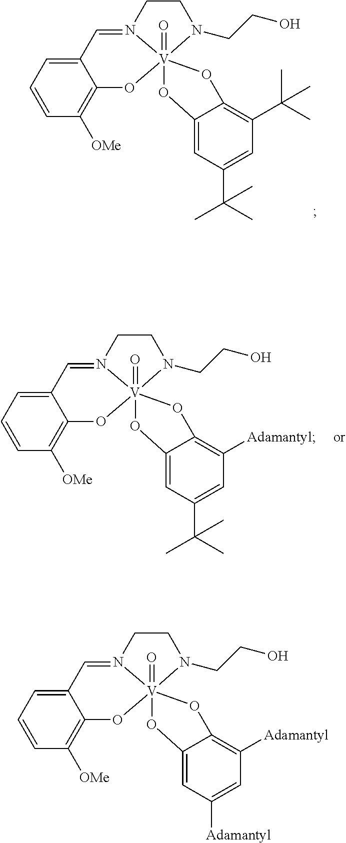

22. A vanadium complex defined by Formula A 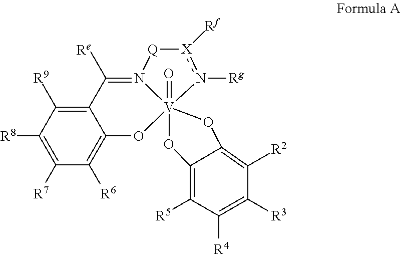
wherein
Q is selected from 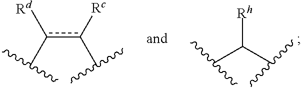
X is absent, or is selected from N, C, or CH when Q is 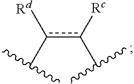
or X is selected
from N, C, or CH when Q is 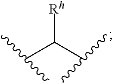
R^{h} is selected from H, ═O, halo, C₁₋₆ alkyl, or C₁₋₄haloalkyl, or R^{h} and R^{f}, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R^{c} and R^{d} are independently selected from H, ═O, halo, C₁₋₆ alkyl, or C₁₋₄haloalkyl; or R^{c} and R^{d} and/or R^{e} and R^{g}, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R^{e} is selected from H, C₁₋₆ alkyl, C₁₋₄haloalkyl; [symbol]
represents individually for each occurence, as valence permits, a single bond or a double bond;
R^{f} and R^{g}, together with the atoms to which they are attached, form a 3-10 membered heterocycloalkyl group or a 5-10 membered heteroaryl group, each optionally substituted with 1, 2, or 3 independently selected R^{A} groups;
R², R³, R⁴, and R⁵ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R² and R³, R³ and R⁴, and/or R⁴ and R⁵, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R⁶, R⁷, R⁸, and R⁹ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R⁶ and R⁷, R⁷ and R⁸, and/or R⁸ and R⁹, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups; and
each R^{A} is independently selected from OH, NO₂, CN, halo, C₁₋₆ alkyl, C₂₋₆ alkenyl, C₂-6 alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

23-49. (canceled)

50. A vanadium complex defined by Formula B, Formula C, or Formula D 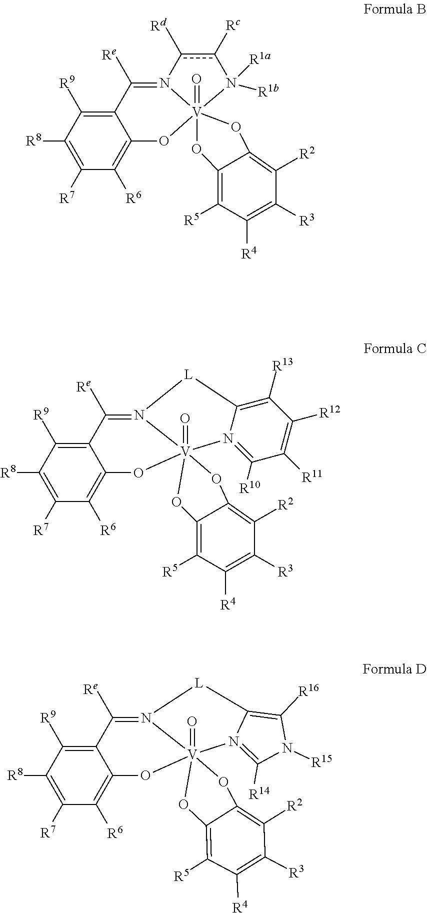
wherein
L is selected from 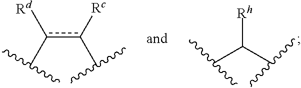
R^{h} is selected from H, ═O, halo, C₁₋₆ alkyl, or C₁₋₄haloalkyl; R^{c} and R^{d} are independently selected from H, ═O, halo, C₁₋₆ alkyl, or C₁₋₄haloalkyl; or R^{e} and R^{d} and/or R^{e} and R^{g}, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R^{e} is selected from H, C₁₋₆ alkyl, C₁₋₄haloalkyl; [symbol]
represents individually for each occurrence, as valence permits, a single bond or a double bond;
R^{1a} is selected from C₁₋₆ alkyl, C₁₋₄haloalkyl, C₂₋₆ alkenyl, C₂₋₆ alkynyl, C₃₋₁₀ cycloalkyl, C₃₋₁₀ cycloalkyl-C₁₋₄ alkylene, 6-10 membered aryl, and 6-10 membered aryl-C₁₋₄ alkylene, each of which is optionally substituted by 1, 2, 3, or 4 independently selected R^{A} groups;
R^{1b} is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups;
R², R³, R⁴, and R⁵ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R² and R³, R³ and R⁴, and/or R⁴ and R⁵, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R⁶, R⁷, R⁸, and R⁹ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R⁶ and R⁷, R⁷ and R⁸, and/or R⁸ and R⁹, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁰, R¹¹, R¹², and R¹³ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄ alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups; or
alternatively, R¹⁰ and R¹¹, R¹¹ and R¹², and/or R¹² and R¹³, together with the atoms to which they are attached, form a 3-10 membered cycloalkyl group, a 3-10 membered heterocycloalkyl group, 5-10 membered heteroaryl group, or a 6-10 membered aryl group, each optionally substituted with 1, 2, or 3 independently selected R^{A} groups;
R¹⁴ and R¹⁶ are independently selected from H, OH, NO₂, CN, halo, C₁₋₆ alkyl, C₂₋₆ alkenyl, C₂₋₆ alkynyl, C₁₋₄haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, 5-10 membered heteroaryl-C₁₋₄alkylene, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino; wherein the C₁₋₆ alkyl, C₂₋₆ alkenyl, C₂₋₆ alkynyl, C₁₋₄ haloalkyl, C₃₋₁₀ cycloalkyl, 6-10 membered aryl, 5-10 membered heteroaryl, 4-10 membered heterocycloalkyl, C₃₋₁₀ cycloalkyl-C₁₋₄ alkylene, 4-10 membered heterocycloalkyl-C₁₋₄ alkylene, 6-10 membered aryl-C₁₋₄ alkylene, and 5-10 membered heteroaryl-C₁₋₄ alkylene are each optionally substituted with 1, 2, 3, or 4 independently selected R^{A} groups;
R¹⁵ is selected from hydrogen, C₁₋₆ alkyl, C₁₋₄haloalkyl, C₂₋₆ alkenyl, C₂₋₆ alkynyl, C₃₋₁₀ cycloalkyl, and 6-10 membered aryl, wherein the C₁₋₆ alkyl, C₁₋₄haloalkyl, C₂₋₆ alkenyl, C₂₋₆ alkynyl, C₃₋₁₀ cycloalkyl, and 6-10 membered aryl are each optionally substituted by 1, 2, 3, or 4 independently selected R^{A} groups; and
each R^{A} is independently selected from OH, NO₂, CN, halo, C₁₋₆ alkyl, C₂₋₆ alkenyl, C₂-6 alkynyl, C₁₋₄ haloalkyl, C₁₋₆ alkoxy, C₁₋₆ haloalkoxy, cyano-C₁₋₃ alkyl, HO—C₁₋₃ alkyl, amino, C₁₋₆ alkylamino, di(C₁₋₆ alkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino

51-78. (canceled)

79. A pharmaceutical composition comprising a therapeutically effective amount of a complex defined by claim 1.

80. (canceled)

81. A method of treating cancer comprising administering to a subjected a therapeutically effective amount of a complex defined by any of the preceding claims or the pharmaceutical composition of claim 79.

82. (canceled)

83. (canceled)
